SEQUENCE LISTING (156 sequences)

SEQ ID NO: 1 - protein, 5 aa, Homo sapiens
  VARIANT 1: note = X = R or N
XYGIH

SEQ ID NO: 2 - protein, 17 aa, Homo sapiens
  VARIANT 6: note = X = G or A
  VARIANT 9: note = X =K or T or D
AIWYDXSKXYYADSVKG

SEQ ID NO: 3 - protein, 16 aa, Homo sapiens
  VARIANT 8: note = X = R or G
  VARIANT 9: note = X = Y or H
  VARIANT 11: note = X = Q or S
  VARIANT 12: note = X = Y or H
  VARIANT 14: note = X = I or L
  VARIANT 15: note = X = N or D
SNYDILTXXYXXGXXV

SEQ ID NO: 4 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLYLQMNSLRAEDTAVYYCARSNYDILTRYYQYGINVWGQGTTVTVSS

SEQ ID NO: 5 - protein, 7 aa, Homo sapiens
  VARIANT 7: note = X =N or S
DASSLEX

SEQ ID NO: 6 - protein, 9 aa, Homo sapiens
  VARIANT 4: note = X = K or N
QQFXSYPRT

SEQ ID NO: 7 - protein, 125 aa, Homo sapiens
  VARIANT 16: note = X = R or G or E
  VARIANT 31: note = X = R or N
  VARIANT 44: note = X = G or C
  VARIANT 55: note = X = G or A
  VARIANT 58: note = X = K or T or D
  VARIANT 76: note = X = K or E or S
  VARIANT 80: note = X = Y or D
  VARIANT 106: note = X = R or G
  VARIANT 107: note = X = Y or H
  VARIANT 109: note = X = Q or S
  VARIANT 110: note = X = Y or H
  VARIANT 112: note = X = I or L
  VARIANT 113: note = X = N or D
QVQLVESGGGVVQPGXSLRLSCAASGFTFSXYGIHWVRQAPGKXLEWVAAIWYDXSKXYYADSVKGRFTISRDNSXNTLXLQMNSLRAEDTAVYYCARSNYDILTXXYXXGXXVWGQGTTVTVSS

SEQ ID NO: 8 - protein, 107 aa, Homo sapiens
  VARIANT 56: note = X = N or S
  VARIANT 92: note = X = K or N
  VARIANT 100: note = X = Q or C
AIQLTQSPSSLSASVGDRVTITCRASQGISSALAWYQQKPGKAPKLLIYDASSLEXGVPSRFSGSGSGTDFTLTISSLQPEDFATYYCQQFXSYPRTFGXGTKVEIK

SEQ ID NO: 9 - protein, 5 aa, Homo sapiens
RYGIH

SEQ ID NO: 10 - protein, 17 aa, Homo sapiens
AIWYDGSKKYYADSVKG

SEQ ID NO: 11 - protein, 16 aa, Homo sapiens
SNYDILTRYYQYGINV

SEQ ID NO: 12 - protein, 5 aa, Homo sapiens
NYGIH

SEQ ID NO: 13 - protein, 17 aa, Homo sapiens
AIWYDASKKYYADSVKG

SEQ ID NO: 14 - protein, 16 aa, Homo sapiens
SNYDILTRYYQYGLDV

SEQ ID NO: 15 - protein, 17 aa, Homo sapiens
AIWYDASKTYYADSVKG

SEQ ID NO: 16 - protein, 16 aa, Homo sapiens
SNYDILTRYYSHGINV

SEQ ID NO: 17 - protein, 17 aa, Homo sapiens
AIWYDASKDYYADSVKG

SEQ ID NO: 18 - protein, 16 aa, Homo sapiens
SNYDILTGHYQYGINV

SEQ ID NO: 19 - protein, 11 aa, Homo sapiens
RASQGISSALA

SEQ ID NO: 20 - protein, 7 aa, Homo sapiens
DASSLEN

SEQ ID NO: 21 - protein, 9 aa, Homo sapiens
QQFKSYPRT

SEQ ID NO: 22 - protein, 7 aa, Homo sapiens
DASSLES

SEQ ID NO: 23 - protein, 9 aa, Homo sapiens
QQFNSYPRT

SEQ ID NO: 24 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDILTRYYQYGINVWGQGTTVTVSS

SEQ ID NO: 25 - protein, 125 aa, Homo sapiens
QVQLVESGGGVVQPGRSLRLSCAASGFTFSNYGIHWVRQAPGKGLEWVAAIWYDASKKYYADSVKGRFTISRDNSKNTLYLQMNSLRAEDTAVYYCARSNYDILTRYYQYGINVWGQGTTVTVSS

SEQ ID NO: 26 - protein, 125 aa, Homo sapiens
QVQLVESGGGVVQPGRSLRLSCAASGFTFSRYGIHWVRQAPGKGLEWVAAIWYDASKKYYADSVKGRFTISRDNSKNTLYLQMNSLRAEDTAVYYCARSNYDILTRYYQYGLDVWGQGTTVTVSS

SEQ ID NO: 27 - protein, 125 aa, Homo sapiens
QVQLVESGGGVVQPGRSLRLSCAASGFTFSRYGIHWVRQAPGKGLEWVAAIWYDASKKYYADSVKGRFTISRDNSKNTLYLQMNSLRAEDTAVYYCARSNYDILTRYYSHGINVWGQGTTVTVSS

SEQ ID NO: 28 - protein, 125 aa, Homo sapiens
QVQLVESGGGVVQPGGSLRLSCAASGFTFSRYGIHWVRQAPGKCLEWVAAIWYDASKTYYADSVKGRFTISRDNSKNTLYLQMNSLRAEDTAVYYCARSNYDILTRYYQYGLDVWGQGTTVTVSS

SEQ ID NO: 29 - protein, 125 aa, Homo sapiens
QVQLVESGGGVVQPGGSLRLSCAASGFTFSRYGIHWVRQAPGKGLEWVAAIWYDASKTYYADSVKGRFTISRDNSKNTLYLQMNSLRAEDTAVYYCARSNYDILTRYYQYGLDVWGQGTTVTVSS

SEQ ID NO: 30 - protein, 125 aa, Homo sapiens
QVQLVESGGGVVQPGGSLRLSCAASGFTFSRYGIHWVRQAPGKGLEWVAAIWYDASKTYYADSVKGRFTISRDNSKNTLYLQMNSLRAEDTAVYYCARSNYDILTRYYSHGINVWGQGTTVTVSS

SEQ ID NO: 31 - protein, 125 aa, Homo sapiens
QVQLVESGGGVVQPGGSLRLSCAASGFTFSRYGIHWVRQAPGKCLEWVAAIWYDASKTYYADSVKGRFTISRDNSKNTLYLQMNSLRAEDTAVYYCARSNYDILTRYYSHGINVWGQGTTVTVSS

SEQ ID NO: 32 - protein, 125 aa, Homo sapiens
QVQLVESGGGVVQPGGSLRLSCAASGFTFSRYGIHWVRQAPGKGLEWVAAIWYDASKKYYADSVKGRFTISRDNSKNTLYLQMNSLRAEDTAVYYCARSNYDILTRYYQYGLDVWGQGTTVTVSS

SEQ ID NO: 33 - protein, 125 aa, Homo sapiens
QVQLVESGGGVVQPGESLRLSCAASGFTFSRYGIHWVRQAPGKGLEWVAAIWYDASKDYYADSVKGRFTISRDNSKNTLYLQMNSLRAEDTAVYYCARSNYDILTRYYQYGLDVWGQGTTVTVSS

SEQ ID NO: 34 - protein, 125 aa, Homo sapiens
QVQLVESGGGVVQPGGSLRLSCAASGFTFSRYGIHWVRQAPGKGLEWVAAIWYDASKTYYADSVKGRFTISRDNSSNTLYLQMNSLRAEDTAVYYCARSNYDILTRYYQYGLDVWGQGTTVTVSS

SEQ ID NO: 35 - protein, 125 aa, Homo sapiens
QVQLVESGGGVVQPGESLRLSCAASGFTFSRYGIHWVRQAPGKGLEWVAAIWYDASKDYYADSVKGRFTISRDNSENTLYLQMNSLRAEDTAVYYCARSNYDILTRYYQYGLDVWGQGTTVTVSS

SEQ ID NO: 36 - protein, 125 aa, Homo sapiens
QVQLVESGGGVVQPGGSLRLSCAASGFTFSRYGIHWVRQAPGKGLEWVAAIWYDASKKYYADSVKGRFTISRDNSSNTLYLQMNSLRAEDTAVYYCARSNYDILTRYYQYGLDVWGQGTTVTVSS

SEQ ID NO: 37 - protein, 125 aa, Homo sapiens
QVQLVESGGGVVQPGESLRLSCAASGFTFSRYGIHWVRQAPGKGLEWVAAIWYDASKKYYADSVKGRFTISRDNSENTLYLQMNSLRAEDTAVYYCARSNYDILTRYYQYGLDVWGQGTTVTVSS

SEQ ID NO: 38 - protein, 125 aa, Homo sapiens
QVQLVESGGGVVQPGGSLRLSCAASGFTFSRYGIHWVRQAPGKGLEWVAAIWYDASKTYYADSVKGRFTISRDNSKNTLYLQMNSLRAEDTAVYYCARSNYDILTGHYQYGINVWGQGTTVTVSS

SEQ ID NO: 39 - protein, 107 aa, Homo sapiens
AIQLTQSPSSLSASVGDRVTITCRASQGISSALAWYQQKPGKAPKLLIYDASSLENGVPSRFSGSGSGTDFTLTISSLQPEDFATYYCQQFKSYPRTFGQGTKVEIK

SEQ ID NO: 40 - protein, 107 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFKSYPRTFGQGTKVEIK

SEQ ID NO: 41 - protein, 107 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYPRTFGQGTKVEIK

SEQ ID NO: 42 - protein, 107 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFKSYPRTFGCGTKVEIK

SEQ ID NO: 43 - protein, 107 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYPRTFGCGTKVEIK

SEQ ID NO: 44 - protein, 226 aa, Homo sapiens
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 226 aa, Homo sapiens
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 46 - protein, 226 aa, Homo sapiens
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 226 aa, Homo sapiens
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 48 - protein, 227 aa, Homo sapiens
ERKSCVECPPCPAPPVAGKSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPCREEMTKNQVSLWCLVKGFYPSDIAVEWESNGQPENNYKTTPPMLDSDGSFFLYSKLTVDKSRWQQGNVFSCSVMHEALHNHYTQKSLSLSPG

SEQ ID NO: 49 - protein, 227 aa, Homo sapiens
ERKSCVECPPCPAPPVAGKSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVCTLPPSREEMTKNQVSLSCAVKGFYPSDIAVEWESNGQPENNYKTTPPMLDSDGSFFLVSKLTVDKSRWQQGNVFSCSVMHEALHNHYTQKSLSLSPG

SEQ ID NO: 50 - protein, 103 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 51 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 52 - protein, 107 aa, Homo sapiens [sequence omitted]

SEQ ID NO: 53 - protein, 125 aa, Homo sapiens
QVQLVESGGGVVQPGRSLRLSCAASGFTFSRYGIHWVRQAPGKGLEWVAAIWYDSSKKYYADSVKGRFTISRDNSKNTLDLQMNSLRAEDTAVYYCARSNYDILTRYYQYGINVWGQGTTVTVSS

SEQ ID NO: 54 - protein, 125 aa, Homo sapiens
QVQLVESGGGVVQPGRSLRLSCAASGFTFSRYGIHWVRQAPGKGLEWVAAIWYDASKKYYADSVKGRFTISRDNSKNTLDLQMNSLRAEDTAVYYCARSNYDILTRYYQYGINVWGQGTTVTVSS

SEQ ID NO: 55 - protein, 125 aa, Homo sapiens
QVQLVESGGGVVQPGRSLRLSCAASGFTFSRYGIHWVRQAPGKGLEWVAAIWYDGSHKYYADSVKGRFTISRDNSKNTLDLQMNSLRAEDTAVYYCARSNYDILTRYYQYGINVWGQGTTVTVSS

SEQ ID NO: 56 - protein, 125 aa, Homo sapiens
QVQLVESGGGVVQPGRSLRLSCAASGFTFSRYGIHWVRQAPGKGLEWVAAIWYDGSNKYYADSVKGRFTISRDNSKNTLDLQMNSLRAEDTAVYYCARSNYDILTRYYQYGINVWGQGTTVTVSS

SEQ ID NO: 57 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDILTRHYQYGINVWGQGTTVTVSS

SEQ ID NO: 58 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDILTGHYQYGINVWGQGTTVTVSS

SEQ ID NO: 59 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDILTGYYQYGINVWGQGTTVTVSS

SEQ ID NO: 60 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDILTRYYQHGINVWGQGTTVTVSS

SEQ ID NO: 61 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDILTRYYSHGINVWGQGTTVTVSS

SEQ ID NO: 62 - protein, 125 aa, Homo sapiens
QVQLVESGGGVVQPGRSLRLSCAASGFTFSNYGIHWVRQAPGKGLEWVAAIWYDGSKKYYADSVKGRFTISRDNSKNTLDLQMNSLRAEDTAVYYCARSNYDILTRYYQYGINVWGQGTTVTVSS

SEQ ID NO: 63 - protein, 125 aa, Homo sapiens
QVQLVESGGGVVQPGRSLRLSCAASGFTFSNHGIHWVRQAPGKGLEWVAAIWYDGSKKYYADSVKGRFTISRDNSKNTLDLQMNSLRAEDTAVYYCARSNYDILTRYYQYGINVWGQGTTVTVSS

SEQ ID NO: 64 - protein, 125 aa, Homo sapiens
QVQLVESGGGVVQPGRSLRLSCAASGFTFSRHGIHWVRQAPGKGLEWVAAIWYDGSKKYYADSVKGRFTISRDNSKNTLDLQMNSLRAEDTAVYYCARSNYDILTRYYQYGINVWGQGTTVTVSS

SEQ ID NO: 65 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNFDILTRYYQYGINVWGQGTTVTVSS

SEQ ID NO: 66 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNFDVLTRYYQYGINVWGQGTTVTVSS

SEQ ID NO: 67 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDVLTRYYQYGINVWGQGTTVTVSS

SEQ ID NO: 68 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDILTRYYQYGLDVWGQGTTVTVSS

SEQ ID NO: 69 - protein, 125 aa, Homo sapiens
QVQLVESGGGVVQPGRSLRLSCAASGFTFSRYGIHWVRQAPGKGLEWVAAIWYDASKKYYADSVKGRFTISRDNSKNTLYLQMNSLRAEDTAVYYCARSNYDILTRYYQYGINVWGQGTTVTVSS

SEQ ID NO: 70 - protein, 125 aa, Homo sapiens
QVQLVESGGGVVQPGRSLRLSCAASGFTFSRYGIHWVRQAPGKGLEWVAAIWYDASHKYYADSVKGRFTISRDNSKNTLYLQMNSLRAEDTAVYYCARSNYDILTRYYQYGINVWGQGTTVTVSS

SEQ ID NO: 71 - protein, 125 aa, Homo sapiens
QVQLVESGGGVVQPGRSLRLSCAASGFTFSRYGIHWVRQAPGKGLEWVAAIWYDASNKYYADSVKGRFTISRDNSKNTLYLQMNSLRAEDTAVYYCARSNYDILTRYYQYGINVWGQGTTVTVSS

SEQ ID NO: 72 - protein, 125 aa, Homo sapiens
QVQLVESGGGVVQPGRSLRLSCAASGFTFSRYGIHWVRQAPGKGLEWVAAIWYDASKKYYADSVKGRFTISRDNSKNTLYLQMNSLRAEDTAVYYCARSNYDILTGHYQYGINVWGQGTTVTVSS

SEQ ID NO: 73 - protein, 125 aa, Homo sapiens
QVQLVESGGGVVQPGRSLRLSCAASGFTFSRYGIHWVRQAPGKGLEWVAAIWYDASKKYYADSVKGRFTISRDNSKNTLYLQMNSLRAEDTAVYYCARSNYDILTGYYQYGINVWGQGTTVTVSS

SEQ ID NO: 74 - protein, 125 aa, Homo sapiens
QVQLVESGGGVVQPGRSLRLSCAASGFTFSNHGIHWVRQAPGKGLEWVAAIWYDASKKYYADSVKGRFTISRDNSKNTLYLQMNSLRAEDTAVYYCARSNYDILTRYYQYGINVWGQGTTVTVSS

SEQ ID NO: 75 - protein, 125 aa, Homo sapiens
QVQLVESGGGVVQPGRSLRLSCAASGFTFSRYGIHWVRQAPGKGLEWVAAIWYDASKKYYADSVKGRFTISRDNSKNTLYLQMNSLRAEDTAVYYCARSNYDVLTRYYQYGINVWGQGTTVTVSS

SEQ ID NO: 76 - protein, 107 aa, Homo sapiens; identical to SEQ ID NO: 40

SEQ ID NO: 77 - protein, 107 aa, Homo sapiens
AIQLTQSPSSLSASVGDRVTITCRASQGISSALAWYQQKPGKAPKLLIYDASSLENGVPSRFSGSGSGTDFTLTISSLQPEDFATYYCQQFNSYPRTFGQGTKVEIK

SEQ ID NO: 78 - protein, 107 aa, Homo sapiens; identical to SEQ ID NO: 41

SEQ ID NO: 79 - protein, 107 aa, Homo sapiens
AIQLTQSPSSLSASVGDRVTITCRASQGISSALAWYQQKPGKAPKLLIYDASGLENGVPSRFSGSGSGTDFTLTISSLQPEDFATYYCQQFKSYPRTFGQGTKVEIK

SEQ ID NO: 80 - protein, 107 aa, Homo sapiens
AIQLTQSPSSLSASVGDRVTITCRASQGISSALAWYQQKPGKAPKLLIYDASGLESGVPSRFSGSGSGTDFTLTISSLQPEDFATYYCQQFNSYPRTFGQGTKVEIK

SEQ ID NO: 81 - protein, 107 aa, Homo sapiens; identical to SEQ ID NO: 41

SEQ ID NO: 82 - protein, 186 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 83 - protein, 125 aa, Homo sapiens
  VARIANT 16: note = X = R or G or E
  VARIANT 31: note = X = R or N
  VARIANT 44: note = X = G or C
  VARIANT 58: note = X = K or T or D
  VARIANT 76: note = X = K or E or S
  VARIANT 106: note = X = R or G
  VARIANT 107: note = X = Y or H
  VARIANT 109: note = X = Q or S
  VARIANT 110: note = X = Y or H
  VARIANT 112: note = X = I or L
  VARIANT 113: note = X = N or D
QVQLVESGGGVVQPGXSLRLSCAASGFTFSXYGIHWVRQAPGKXLEWVAAIWYDASKXYYADSVKGRFTISRDNSXNTLYLQMNSLRAEDTAVYYCARSNYDILTXXYXXGXXVWGQGTTVTVSS

SEQ ID NO: 84 - protein, 107 aa, Homo sapiens
  VARIANT 92: note = X = K or N
  VARIANT 100: note = X = Q or C
AIQLTQSPSSLSASVGDRVTITCRASQGISSALAWYQQKPGKAPKLLIYDASSLESGVPSRFSGSGSGTDFTLTISSLQPEDFATYYCQQFXSYPRTFGXGTKVEIK

SEQ ID NO: 85 - protein, 17 aa, Homo sapiens
  VARIANT 9: note = X = K, T or D
AIWYDASKXYYADSVKG

SEQ ID NO: 86 - protein, 231 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 87 - protein, 231 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 231 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 89 - protein, 231 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 90 - protein, 231 aa, Homo sapiens
EPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 91 - protein, 231 aa, Homo sapiens
EPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 92 - protein, 231 aa, Homo sapiens
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 93 - protein, 231 aa, Homo sapiens
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 94 - protein, 5 aa, Homo sapiens
GGGGS

SEQ ID NO: 95 - protein, 10 aa, Homo sapiens
GGGGSGGGGS

SEQ ID NO: 96 - protein, 15 aa, Homo sapiens
GGGGSGGGGSGGGGS

SEQ ID NO: 97 - skipped

SEQ ID NO: 98 - protein, 18 aa, Homo sapiens
GSTSGSGKPGSGEGSTKG

SEQ ID NO: 99 - protein, 21 aa, Homo sapiens
SRGGGGSGGGGSGGGGSLEMA

SEQ ID NO: 100 - protein, 22 aa, Homo sapiens
GSRGGGGSGGGGSGGGGSLEMA

SEQ ID NO: 101 - protein, 98 aa, Homo sapiens
ASTKGPSVFPLAPSSKSTSGGTAWLGCEVTDYFPEPVTVSWNSGALTSGVHTFPAVLESSGLYSLSSVVTVPSSSLGTQTYICNVNHKPSNTKVDKKV

SEQ ID NO: 102 - protein, 98 aa, Homo sapiens
ASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKKV

SEQ ID NO: 103 - protein, 105 aa, Homo sapiens
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 104 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 125 aa, Homo sapiens
  VARIANT 16: note = X = R or G or E
  VARIANT 31: note = X = R or N
  VARIANT 32: note = X = Y or H
  VARIANT 44: note = X = G or C
  VARIANT 55: note = X = G or A
  VARIANT 57: note = X = K or H or N
  VARIANT 58: note = X = K or T or D
  VARIANT 76: note = X = K or E or S
  VARIANT 80: note = X = Y or D
  VARIANT 101: note = X = Y or F
  VARIANT 103: note = X = I or V
  VARIANT 106: note = X = R or G
  VARIANT 107: note = X = Y or H
  VARIANT 109: note = X =Q or S
  VARIANT 110: note = X = Y or H
  VARIANT 112: note = X = I or L
  VARIANT 113: note = X = N or D
QVQLVESGGGVVQPGXSLRLSCAASGFTFSXXGIHWVRQAPGKXLEWVAAIWYDXSXXYYADSVKGRFTISRDNSXNTLXLQMNSLRAEDTAVYYCARSNXDXLTXXYXXGXXVWGQGTTVTVSS

SEQ ID NO: 106 - protein, 107 aa, Homo sapiens
  VARIANT 53: note = X = S or G
  VARIANT 56: note = X = N or S
  VARIANT 92: note = X = K or N
  VARIANT 100: note = X = Q or C
AIQLTQSPSSLSASVGDRVTITCRASQGISSALAWYQQKPGKAPKLLIYDASXLEXGVPSRFSGSGSGTDFTLTISSLQPEDFATYYCQQFXSYPRTFGXGTKVEIK

SEQ ID NO: 107 - skipped

SEQ ID NO: 108 - protein, 17 aa, Homo sapiens
  VARIANT 6: note = X = G or A
  VARIANT 8: note = X = K or H or N
  VARIANT 9: note = X = K or T or D
AIWYDXSXXYYADSVKG

SEQ ID NO: 109 - protein, 16 aa, Homo sapiens
  VARIANT 3: note = X = Y or F
  VARIANT 5: note = X = I or V
  VARIANT 8: note = X = R or G
  VARIANT 9: note = X = Y or H
  VARIANT 11: note = X = Q or S
  VARIANT 12: note = X = Y or H
  VARIANT 14: note = X = I or L
  VARIANT 15: note = X = N or D
SNXDXLTXXYXXGXXV

SEQ ID NO: 110 - protein, 7 aa, Homo sapiens
  VARIANT 4: note = X = S or G
  VARIANT 7: note = X = N or S
DASXLEX

SEQ ID NO: 111 - protein, 5 aa, Homo sapiens
NHGIH

SEQ ID NO: 112 - protein, 5 aa, Homo sapiens
RHGIH

SEQ ID NO: 113 - protein, 17 aa, Homo sapiens
AIWYDSSKKYYADSVKG

SEQ ID NO: 114 - protein, 17 aa, Homo sapiens
AIWYDGSHKYYADSVKG

SEQ ID NO: 115 - protein, 17 aa, Homo sapiens
AIWYDGSNKYYADSVKG

SEQ ID NO: 116 - protein, 17 aa, Homo sapiens
AIWYDASHKYYADSVKG

SEQ ID NO: 117 - protein, 17 aa, Homo sapiens
AIWYDASNKYYADSVKG

SEQ ID NO: 118 - protein, 16 aa, Homo sapiens
SNYDILTRHYQYGINV

SEQ ID NO: 119 - protein, 16 aa, Homo sapiens
SNYDILTGYYQYGINV

SEQ ID NO: 120 - protein, 16 aa, Homo sapiens
SNYDILTRYYQHGINV

SEQ ID NO: 121 - protein, 16 aa, Homo sapiens
SNFDILTRYYQYGINV

SEQ ID NO: 122 - protein, 16 aa, Homo sapiens
SNFDVLTRYYQYGINV

SEQ ID NO: 123 - protein, 16 aa, Homo sapiens
SNYDVLTRYYQYGINV

SEQ ID NO: 124 - protein, 7 aa, Homo sapiens
DASGLEN

SEQ ID NO: 125 - protein, 7 aa, Homo sapiens
DASGLES

SEQ ID NO: 126 - protein, 125 aa, Homo sapiens
QVQLVESGGGVVQPGESLRLSCAASGFTFSRYGIHWVRQAPGKGLEWVAAIWYDASKKYYADSVKGRFTISRDNSKNTLYLQMNSLRAEDTAVYYCARSNYDILTRYYQYGLDVWGQGTTVTVSS

SEQ ID NO: 127 - protein, 14 aa, Homo sapiens
ZDGNEEMGGITQTP

SEQ ID NO: 128 - protein, 247 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDILTRYYQYGINVWGQGTTVTVSSGGGGSGGGGSGGGGSAIQLTQSPSSLSASVGDRVTITCRASQGISSALAWYQQKPGKAPKLLIYDASSLENGVPSRFSGSGSGTDFTLTISSLQPEDFATYYCQQFKSYPRTFGQGTKVEIK

SEQ ID NO: 129 - protein, 247 aa, Homo sapiens
QVQLVESGGGVVQPGRSLRLSCAASGFTFSRYGIHWVRQAPGKGLEWVAAIWYDASKKYYADSVKGRFTISRDNSKNTLYLQMNSLRAEDTAVYYCARSNYDILTRYYQYGLDVWGQGTTVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFKSYPRTFGQGTKVEIK

SEQ ID NO: 130 - protein, 247 aa, Homo sapiens
QVQLVESGGGVVQPGGSLRLSCAASGFTFSRYGIHWVRQAPGKCLEWVAAIWYDASKTYYADSVKGRFTISRDNSKNTLYLQMNSLRAEDTAVYYCARSNYDILTRYYQYGLDVWGQGTTVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFKSYPRTFGCGTKVEIK

SEQ ID NO: 131 - protein, 247 aa, Homo sapiens
QVQLVESGGGVVQPGGSLRLSCAASGFTFSRYGIHWVRQAPGKCLEWVAAIWYDASKTYYADSVKGRFTISRDNSKNTLYLQMNSLRAEDTAVYYCARSNYDILTRYYSHGINVWGQGTTVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPRTFGCGTKVEIK

SEQ ID NO: 132 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 133 - protein, 454 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDILTRYYQYGINVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 134 - protein, 214 aa, Homo sapiens
AIQLTQSPSSLSASVGDRVTITCRASQGISSALAWYQQKPGKAPKLLIYDASSLENGVPSRFSGSGSGTDFTLTISSLQPEDFATYYCQQFKSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 454 aa, Homo sapiens
QVQLVESGGGVVQPGRSLRLSCAASGFTFSNYGIHWVRQAPGKGLEWVAAIWYDASKKYYADSVKGRFTISRDNSKNTLYLQMNSLRAEDTAVYYCARSNYDILTRYYQYGINVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 136 - protein, 214 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFKSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 454 aa, Homo sapiens
QVQLVESGGGVVQPGRSLRLSCAASGFTFSRYGIHWVRQAPGKGLEWVAAIWYDASKKYYADSVKGRFTISRDNSKNTLYLQMNSLRAEDTAVYYCARSNYDILTRYYQYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 138 - protein, 454 aa, Homo sapiens
QVQLVESGGGVVQPGRSLRLSCAASGFTFSRYGIHWVRQAPGKGLEWVAAIWYDASKKYYADSVKGRFTISRDNSKNTLYLQMNSLRAEDTAVYYCARSNYDILTRYYSHGINVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 139 - protein, 214 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 454 aa, Homo sapiens
QVQLVESGGGVVQPGGSLRLSCAASGFTFSRYGIHWVRQAPGKCLEWVAAIWYDASKTYYADSVKGRFTISRDNSKNTLYLQMNSLRAEDTAVYYCARSNYDILTRYYQYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 141 - protein, 214 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFKSYPRTFGCGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 454 aa, Homo sapiens
QVQLVESGGGVVQPGGSLRLSCAASGFTFSRYGIHWVRQAPGKGLEWVAAIWYDASKTYYADSVKGRFTISRDNSKNTLYLQMNSLRAEDTAVYYCARSNYDILTRYYQYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 143 - protein, 454 aa, Homo sapiens
QVQLVESGGGVVQPGGSLRLSCAASGFTFSRYGIHWVRQAPGKGLEWVAAIWYDASKTYYADSVKGRFTISRDNSKNTLYLQMNSLRAEDTAVYYCARSNYDILTRYYSHGINVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 144 - protein, 454 aa, Homo sapiens
QVQLVESGGGVVQPGGSLRLSCAASGFTFSRYGIHWVRQAPGKCLEWVAAIWYDASKTYYADSVKGRFTISRDNSKNTLYLQMNSLRAEDTAVYYCARSNYDILTRYYSHGINVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 145 - protein, 214 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYPRTFGCGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 454 aa, Homo sapiens
QVQLVESGGGVVQPGGSLRLSCAASGFTFSRYGIHWVRQAPGKGLEWVAAIWYDASKKYYADSVKGRFTISRDNSKNTLYLQMNSLRAEDTAVYYCARSNYDILTRYYQYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 147 - protein, 454 aa, Homo sapiens
QVQLVESGGGVVQPGESLRLSCAASGFTFSRYGIHWVRQAPGKGLEWVAAIWYDASKKYYADSVKGRFTISRDNSKNTLYLQMNSLRAEDTAVYYCARSNYDILTRYYQYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 148 - protein, 454 aa, Homo sapiens
QVQLVESGGGVVQPGESLRLSCAASGFTFSRYGIHWVRQAPGKGLEWVAAIWYDASKDYYADSVKGRFTISRDNSKNTLYLQMNSLRAEDTAVYYCARSNYDILTRYYQYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 149 - protein, 454 aa, Homo sapiens
QVQLVESGGGVVQPGGSLRLSCAASGFTFSRYGIHWVRQAPGKGLEWVAAIWYDASKTYYADSVKGRFTISRDNSSNTLYLQMNSLRAEDTAVYYCARSNYDILTRYYQYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 150 - protein, 454 aa, Homo sapiens
QVQLVESGGGVVQPGESLRLSCAASGFTFSRYGIHWVRQAPGKGLEWVAAIWYDASKDYYADSVKGRFTISRDNSENTLYLQMNSLRAEDTAVYYCARSNYDILTRYYQYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 151 - protein, 454 aa, Homo sapiens
QVQLVESGGGVVQPGGSLRLSCAASGFTFSRYGIHWVRQAPGKGLEWVAAIWYDASKKYYADSVKGRFTISRDNSSNTLYLQMNSLRAEDTAVYYCARSNYDILTRYYQYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 152 - protein, 454 aa, Homo sapiens
QVQLVESGGGVVQPGESLRLSCAASGFTFSRYGIHWVRQAPGKGLEWVAAIWYDASKKYYADSVKGRFTISRDNSENTLYLQMNSLRAEDTAVYYCARSNYDILTRYYQYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 153 - protein, 454 aa, Homo sapiens
QVQLVESGGGVVQPGGSLRLSCAASGFTFSRYGIHWVRQAPGKGLEWVAAIWYDASKTYYADSVKGRFTISRDNSKNTLYLQMNSLRAEDTAVYYCARSNYDILTGHYQYGINVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 154 - protein, 17 aa, Homo sapiens
  VARIANT 6: note = X = G or A or S
  VARIANT 8: note = X = K or H or N
  VARIANT 9: note = X = K or T or D
AIWYDXSXXYYADSVKG

SEQ ID NO: 155 - protein, 9 aa, Homo sapiens
  VARIANT 4: note = X = R or N or K
QQFXSYPRT

SEQ ID NO: 156 - protein, 125 aa, Homo sapiens; identical to SEQ ID NO: 105
  VARIANT 16: note = X = R, G, or E
  VARIANT 31: note = X = R or N
  VARIANT 32: note = X = Y or H
  VARIANT 44: note = X = G or C
  VARIANT 55: note = X = G or A or S
  VARIANT 57: note = X = K, H, or N
  VARIANT 58: note = X = K, T, or D
  VARIANT 76: note = X = K, E, or S
  VARIANT 80: note = X = Y or D
  VARIANT 101: note = X = Y or F
  VARIANT 103: note = X = I or V
  VARIANT 106: note = X = R or G
  VARIANT 107: note = X = Y or H
  VARIANT 109: note = X = Q or S
  VARIANT 110: note = X = Y or H
  VARIANT 112: note = X = I or L
  VARIANT 113: note = X = N or D

(19) United States
(12) Patent Application Publication
(10) Pub. No.: US 2026/0356948 A1
(43) Pub. Date: Nov. 12, 2026

(54) CD3 BINDING MOLECULES AND RELATED METHODS AND USES

(71) Applicant: Bristol-Myers Squibb Company, Princeton, NJ (US)

(72) Inventors: Lore K. FLORIN, Redwood City, CA (US); Haichun HUANG, Fremont, CA (US); Winse MORISHIGE, Newark, CA (US); Arvind RAJPAL, San Francisco, CA (US); Pavel STROP, San Mateo, CA (US); Feng WANG, Belmont, CA (US); Sean Matthew WEST, Redwood City, CA (US); Matthew WHEELER, San Francisco, CA (US)

(73) Assignee: c/o Bristol-Myers Squibb Company, Princeton, NJ (US)

(21) Appl. No.: 19/454,173

(22) Filed: Jan. 20, 2026

Related U.S. Application Data
(60) Provisional application No. 63/747,480, filed on Jan. 21, 2025.

(51) Int. Cl. C07K 16/28

(52) U.S. Cl. CPC C07K 16/2809; C07K 2317/21; C07K 2317/31; C07K 2317/56; C07K 2317/565; C07K 2317/622; C07K 2317/624; C07K 2317/92

(57) ABSTRACT

Provided are binding molecules that bind to CD3, in particular, binding molecules that incorporate human antibodies, including antibody fragments, specific for CD3. In some aspects, the provided binding molecules are bispecific antibodies directed to CD3 and a second antigen, such as a B cell antigen or a tumor antigen. Also provided are polynucleotides that encode the binding molecules, including the antibodies or bispecific antibodies. Also provided are pharmaceutical compositions and methods and uses of such binding molecules for treatment of diseases in which specific targeting, and in some aspects T-cell-mediated killing, of cells that express the second antigen is desired.

### CROSS-REFERENCE TO RELATED APPLICATIONS

This application claims priority from U.S. provisional application No. 63/747,480, filed Jan. 21, 2025, entitled “CD3 BINDING MOLECULES AND RELATED METHODS AND USES”, the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 21737-20005.00.xml created Jan. 17, 2026, which is 189,388 bytes in size. The information in the electronic format of the Sequence Listing is incorporated by reference in its entirety.

### FIELD OF DISCLOSURE

The present disclosure relates in some aspects to binding molecules that bind to CD3 and in particular, binding molecules that incorporate human antibodies, including antibody fragments, specific for CD3. In some aspects, among the binding molecules of the present disclosure are bispecific antibodies directed to CD3 and a second antigen, such as a B cell antigen or a tumor antigen. The disclosure further relates to polynucleotides that encode the binding molecules, including the antibodies or bispecific antibodies. The present disclosure further relates to pharmaceutical compositions, kits and methods and uses of such binding molecules for treatment of diseases in which specific targeting, and in some aspects T-cell-mediated killing, of cells that express the second antigen is desired.

### BACKGROUND

Binding molecules, including bispecific antibodies, that target CD3 on T cells have been used as a T cell engager (TCE) to recruit cytolytic T cells to kill tumor cells. In many cases, CD3-targeting antibodies are high-affinity antibodies which can favor their binding to circulating T cells or in secondary lymphoid organs. This may limit their use due to issues such as unfavorable pharmacokinetics and non-specific binding leading to activation of peripheral T cells in a tumor-target independent fashion. Improved CD3 binding molecules are needed including for use in a TCE.

### SUMMARY

An aspect of the invention provides a binding molecule comprising a CD3 binding domain comprising a heavy chain variable (VH) region and a light chain variable (VL) region, wherein: the VH region comprises a heavy chain complementarity determining region 1 (CDR-H1) set forth in SEQ ID NO: 1, a heavy chain complementarity determining region 2 (CDR-H2) set forth in SEQ ID NO: 2, and a heavy chain complementarity determining region 3 (CDR-H3) set forth in SEQ ID NO:3; and the VL region comprises a light chain complementarity determining region 1 (CDR-L1) set forth in SEQ ID NO: 19, a light chain complementarity determining region 2 (CDR-L2) set forth in SEQ ID NO: 5, and a light chain complementarity determining region 3 (CDR-L3) set forth within SEQ ID NO: 6.

In certain embodiments, the VH region comprises an amino acid sequence of the consensus sequence QVQLVESGGGVVQPGX₁SLRLSCAASGFTFSX₂YGIHWVRQAPGKX₃LEWVAAIWYD X₄SKX₅YYADSVKGRFTISRDNSX₆NTLX₇LQMNSLRAEDTAVYYCARSNYDILTX₈X₉YX₁₀X₁₁GX₁₂X₁₃VWGQGTTVTVSS, wherein X₁ is R, G, or E; X₂ is R or N; X₃ is G or C; X₄ is G or A; X₅ is K, T, or D; X₆ is K, E, or S; X₇ is Y or D; X₈ is R or G; X₉ is Y or H; X₁₀ is Q or S; X₁₁ is Y or H; X₁₂ is I or L; and X₁₃ is N or D; (SEQ ID NO:7); and the VL region comprises an amino acid sequence of the consensus sequence set forth in AIQLTQSPSSLSASVGDRVTITCRASQGISSALAWYQQKPGKAPKLLIYDASSLEX₁GV PSRFSGSGSGTDFTLTISSLQPEDFATYYCQQFX₂SYPRTFGX₃GTKVEIK, wherein X₁ is N or S; X₂ is K or N; X₃ is Q or C; (SEQ ID NO:8).

In certain embodiments, the VH region comprises an amino acid sequence of the consensus sequence QVQLVESGGGVVQPGX₁SLRLSCAASGFTFSX₂YGIHWVRQAPGKX₃LEWVAAIWYD ASKX₄YYADSVKGRFTISRDNSX₅NTLYLQMNSLRAEDTAVYYCARSNYDILTX₆X₇Y X₈X₉GX₁₀X₁₁VWGQGTTVTVSS, wherein X₁=R or G or E; X₂=R or N; X₃=G or C; X₄=K or T or D; X₅=K or E or S; X₆=R or G; X₇=Y or H; X₈=Q or S; X₉=Y or H; X₁₀=I or L; and X₁₁=N or D (SEQ ID NO: 83); and the VL region comprises an amino acid sequence of the consensus sequence set forth in AIQLTQSPSSLSASVGDRVTITCRASQGISSALAWYQQKPGKAPKLLIYDASSLESGVP SRFSGSGSGTDFTLTISSLQPEDFATYYCQQFX₁SYPRTFGX₂GTKVEIK (SEQ ID NO: 84), wherein X₁=K or N; and X₂=Q or C.

In certain embodiments, the binding molecule is a bispecific antibody comprising the CD3 binding domain and a second antigen binding domain. In certain embodiments, the second antigen binding domain comprises a means for binding a second antigen wherein the second antigen is on a tumor or a B cell.

An aspect of the invention provides a binding molecule comprising a CD3 binding domain and a second antigen binding domain, wherein the binding molecule is bispecific for binding CD3 and the second antigen, wherein: (a) the CD3 binding domain comprises a heavy chain variable (VH) region and a light chain variable (VL) region, wherein: the VH region comprises a heavy chain complementarity determining region 1 (CDR-H1) set forth in SEQ ID NO: 1, a heavy chain complementarity determining region 2 (CDR-H2) set forth in SEQ ID NO: 2, and a heavy chain complementarity determining region 3 (CDR-H3) set forth in SEQ ID NO:3, and the VL region comprises a light chain complementarity determining region 1 (CDR-L1) set forth in SEQ ID NO: 19, a light chain complementarity determining region 2 (CDR-L2) set forth in SEQ ID NO: 5, and a light chain complementarity determining region 3 (CDR-L3) set forth within SEQ ID NO: 6; and

In certain embodiments, the VH region comprises a CDR-H1 set forth in SEQ ID NO: 1, a CDR-H2 set forth in SEQ ID NO: 85, and a CDR-H3 set forth in SEQ ID NO:3; and the VL region comprises a CDR-L1 set forth in SEQ ID NO: 19, a CDR-L2 set forth in SEQ ID NO: 22, and a CDR-L3 set forth within SEQ ID NO: 6.

In certain embodiments, the VH region comprises an amino acid sequence of the consensus sequence QVQLVESGGGVVQPGX₁SLRLSCAASGFTFSX₂YGIHWVRQAPGKX₃LEWVAAIWYD X₄SKX₅YYADSVKGRFTISRDNSX₆NTLX₇LQMNSLRAEDTAVYYCARSNYDILTRYY X₈X₉GX₁₀X₁₁VWGQGTTVTVSS, wherein X₁ is R, G, or E; X₂ is R or N; X₃ is G or C; X₄ is G or A; X₅ is K, T, or D; X₆ is K, E, or S; X₇ is Y or D; X₈ is Q or S; X₉ is Y or H; X₁₀ is I or L; X₁₁ is N or D; (SEQ ID NO:7); and the VL region comprising an amino acid sequence of the consensus sequence set forth in AIQLTQSPSSLSASVGDRVTITCRASQGISSALAWYQQKPGKAPKLLIYDASSLEX₁GV PSRFSGSGSGTDFTLTISSLQPEDFATYYCQQFX₂SYPRTFGX₃GTKVEIK, wherein X₁ is N or S; X₂ is K or N; X₃ is Q or C; (SEQ ID NO:8).

In certain embodiments, the VH region comprises an amino acid sequence of the consensus sequence QVQLVESGGGVVQPGX₁SLRLSCAASGFTFSX₂YGIHWVRQAPGKX₃LEWVAAIWYD ASKX₄YYADSVKGRFTISRDNSX₅NTLYLQMNSLRAEDTAVYYCARSNYDILTX₆X₇Y X₈X₉GX₁₀X₁₁VWGQGTTVTVSS, wherein X₁=R or G or E; X₂=R or N; X₃=G or C; X₄=K or T or D; X₅=K or E or S; X₆=R or G; X₇=Y or H; X₈=Q or S; X₉=Y or H; X₁₀=I or L; and X₁₁=N or D (SEQ ID NO: 83); and the VL region comprises an amino acid sequence of the consensus sequence set forth in AIQLTQSPSSLSASVGDRVTITCRASQGISSALAWYQQKPGKAPKLLIYDASSLESGVP SRFSGSGSGTDFTLTISSLQPEDFATYYCQQFX₁SYPRTFGX₂GTKVEIK (SEQ ID NO: 84), wherein X₁=K or N; and X₂=Q or C.

In certain embodiments, the CD3 binding domain binds to CD3 epsilon with a dissociation constant (KD) of 5 nM or more.

In certain embodiments, the CD3 binding domain binds to CD3 epsilon with a dissociation constant (KD) of 10 nM to 70 nM.

In certain embodiments, the VH comprises one or more amino acid residues selected from the group consisting of: an asparagine (N) at position 31, an alanine (A) at position 55, a tyrosine (Y) at position 80, a glycine (G) at position 106, a histidine (H) at position 107, a serine(S) at position 109, a histidine (H) at position 110, a leucine (L) at position 112, and an aspartic acid (D) at position 113 or any combination thereof, wherein the numbering is relative to SEQ ID NO: 24.

In certain embodiments, the VH comprises: (a) amino acid residues asparagine (N) at position 31, alanine (A) at position 55, and tyrosine (Y) at position 80, wherein the numbering is relative to SEQ ID NO: 24; (b) alanine (A) at position 55, tyrosine (Y) at position 80, leucine (L) at position 112, and aspartic acid (D) at position 113, wherein the numbering is relative to SEQ ID NO: 24; or (c) alanine (A) at position 55, tyrosine (Y) at position 80, serine(S) at position 109, and histidine (H) at position 110, wherein the numbering is relative to SEQ ID NO: 24.

In certain embodiments, the VL region comprises one or more amino acid residues selected from the group consisting of: a serine(S) at position 56, an asparagine (N) at position 92, and a cysteine (C) at position 100, an asparagine (N) at position 108, or any combination thereof, wherein the numbering is relative to SEQ ID NO: 39.

In certain embodiments, the VL region comprise: (a) serine(S) at position 56, wherein the numbering is relative to SEQ ID NO:39; or (b) serine(S) at position 56 and asparagine (N) at position 92, wherein the numbering is relative to SEQ ID NO: 39.

In certain embodiments, the CD3 binding domain exhibits low polyreactivity to extracellular matrix proteins.

In certain embodiments, the CD3 binding domain exhibits an extracellular matrix (ECM) score of less than 14, less than 13, less than 12, less than 11, or less than 10.

In certain embodiments, the VH comprises glycine (G) at position 16, a glutamic acid (E) at position 16, a threonine (T) at position 58, an aspartic acid (D) at position 58, a glutamic acid (E) at position 76, a serine(S) at position 76 in relation to SEQ ID NO: 24.

In certain embodiments, the VH comprises glycine (G) at position 16 and/or threonine (T) at position 58, optionally glycine (G) at position 16 and threonine (T) at position 58 in relation to SEQ ID NO: 24.

In certain embodiments, the CD3 binding domain is an antigen binding antibody fragment. In some embodiments, the CD3 binding domain is selected from the group consisting of Fab, Fab′, F(ab′)2, Fv, and scFv. In some embodiments, the CD3 binding domain is a Fab. In some embodiments, the CD3 binding domain is a scFv or is an Fv. In certain embodiments, the CD3 binding domain is an scFv that is a disulfide-stabilized scFv. In some of any embodiments, the antigen-binding fragment is an scFv. In some embodiments, the scFv comprises a VH-linker-VL arrangement. In some embodiments, the scFv comprises a VL-linker-VH arrangement. In some of any embodiments, the linker is a peptide linker comprising (Gly4Ser)3 (SEQ ID NO: 96) In some of any embodiments, the linker is a peptide linker comprising Gly4Ser (SEQ ID NO: 94). In some of any embodiments, the linker is a peptide linker comprising (Gly4Ser)2 (SEQ ID NO: 95).

In certain embodiments, the VH region CD3 binding domain comprises cysteine (C) at position 44 and the VL region of the CD3 binding domain comprises a cysteine (C) at position 100 in relation to SEQ ID NO: 39.

In certain embodiments, the VH region comprises the CDR-H1, the CDR-H2, and the CDR-H3 contained within any one of SEQ ID NOs: 24-38, and the VL region comprises the CDR-L1, the CDR-L2, and the CDR-L3 contained within any one of SEQ ID NOs: 39-43.

In certain embodiments, the CDR-H1 comprises the amino acid sequence set forth in any one of SEQ ID NOs: 9 or 12, the CDR-H2 comprises the sequence set forth in any one of SEQ ID NOs: 10, 13, 15, or 17, and the CDR-H3 comprises the amino acid sequence set forth in any one of SEQ ID NOs: 11, 14, 16, or 18; and the CDR-L1 comprises the amino acid sequence set forth in SEQ ID NOs: 19, the CDR-L2 comprises the amino acid sequence set forth in any one of SEQ ID NOs: 20 or 22, and the CDR-L3 comprises the amino acid sequence set forth in any one of SEQ ID NOs: 21 or 23.

In certain embodiments, the VH region comprises the CDR-H1, the CDR-H2, and the CDR-H3 contained within SEQ ID NO: 24, and the VL region comprises the CDR-L1, the CDR-L2, and the CDR-L3 contained within SEQ ID NO: 39; the VH region comprises the CDR-H1, the CDR-H2, and the CDR-H3 contained within SEQ ID NO: 25, and the VL region comprises the CDR-L1, the CDR-L2, and the CDR-L3 contained within SEQ ID NO: 40; the VH region comprises the CDR-H1, the CDR-H2, and the CDR-H3 contained within SEQ ID NO: 26, and the VL region comprises the CDR-L1, the CDR-L2, and the CDR-L3 contained within SEQ ID NO: 40; the VH region comprises the CDR-H1, the CDR-H2, and the CDR-H3 contained within SEQ ID NO: 27, and the VL region comprises the CDR-L1, the CDR-L2, and the CDR-L3 contained within SEQ ID NO: 41; the VH region comprises the CDR-H1, the CDR-H2, and the CDR-H3 contained within SEQ ID NO: 28, and the VL region comprises the CDR-L1, the CDR-L2, and the CDR-L3 contained within SEQ ID NO: 42; the VH region comprises the CDR-H1, the CDR-H2, and the CDR-H3 contained within SEQ ID NO: 29, and the VL region comprises the CDR-L1, the CDR-L2, and the CDR-L3 contained within SEQ ID NO: 40; the VH region comprises the CDR-H1, the CDR-H2, and the CDR-H3 contained within SEQ ID NO: 30, and the VL region comprises the CDR-L1, the CDR-L2, and the CDR-L3 contained within SEQ ID NO: 41; the VH region comprises the CDR-H1, the CDR-H2, and the CDR-H3 contained within SEQ ID NO: 31, and the VL region comprises the CDR-L1, the CDR-L2, and the CDR-L3 contained within SEQ ID NO: 43; the VH region comprises the CDR-H1, the CDR-H2, and the CDR-H3 contained within SEQ ID NO: 32, and the VL region comprises the CDR-L1, the CDR-L2, and the CDR-L3 contained within SEQ ID NO: 40; the VH region comprises the CDR-H1, the CDR-H2, and the CDR-H3 contained within SEQ ID NO: 33, and the VL region comprises the CDR-L1, the CDR-L2, and the CDR-L3 contained within SEQ ID NO: 40; the VH region comprises the CDR-H1, the CDR-H2, and the CDR-H3 contained within SEQ ID NO: 34, and the VL region comprises the CDR-L1, the CDR-L2, and the CDR-L3 contained within SEQ ID NO: 40; the VH region comprises the CDR-H1, the CDR-H2, and the CDR-H3 contained within SEQ ID NO: 35, and the VL region comprises the CDR-L1, the CDR-L2, and the CDR-L3 contained within SEQ ID NO: 40; the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NO: 40; the VH region comprises the CDR-H1, the CDR-H2, and the CDR-H3 contained within SEQ ID NO: 37, and the VL region comprises the CDR-L1, the CDR-L2, and the CDR-L3 contained within SEQ ID NO: 40; the VH region comprises the CDR-H1, the CDR-H2, and the CDR-H3 contained within SEQ ID NO: 38, and the VL region comprises the CDR-L1, the CDR-L2, and the CDR-L3 contained within SEQ ID NO: 41; or the VH region comprises the CDR-H1, the CDR-H2, and the CDR-H3 contained within SEQ ID NO: 126, and the VL region comprises the CDR-L1, the CDR-L2, and the CDR-L3 contained within SEQ ID NO: 40.

In certain embodiments, the VH region comprises the CDR-H1, the CDR-H2, and the CDR-H3 comprising the sequence set forth in SEQ ID NOs: 9, 10, and 11, respectively, and the VL region comprises the CDR-L1, the CDR-L2, and the CDR-L3 comprising the sequence set forth in SEQ ID NOs: 19, 20, and 21, respectively; the VH region comprises the CDR-H1, the CDR-H2, and the CDR-H3 comprising the sequence set forth in SEQ ID NOs: 12, 13, and 11, respectively, and the VL region comprises the CDR-L1, the CDR-L2, and the CDR-L3 comprising the sequence set forth in SEQ ID NOs: 19, 22, and 21, respectively; the VH region comprises the CDR-H1, the CDR-H2, and the CDR-H3 comprising the sequence set forth in SEQ ID NOs: 9, 13, and 14, respectively, and the VL region comprises the CDR-L1, the CDR-L2, and the CDR-L3 comprising the sequence set forth in SEQ ID NOs: 19, 22, and 21, respectively; the VH region comprises the CDR-H1, the CDR-H2, and the CDR-H3 comprising the sequence set forth in SEQ ID NOs: 9, 13, and 11, respectively, and the VL region comprises the CDR-L1, the CDR-L2, and the CDR-L3 comprising the sequence set forth in SEQ ID NOs: 19, 22, and 23, respectively; the VH region comprises the CDR-H1, the CDR-H2, and the CDR-H3 comprising the sequence set forth in SEQ ID NOs: 9, 15, and 14, respectively, and the VL region comprises the CDR-L1, the CDR-L2, and the CDR-L3 comprising the sequence set forth in SEQ ID NOs: 19, 22, and 21, respectively; the VH region comprises the CDR-H1, the CDR-H2, and the CDR-H3 comprising the sequence set forth in SEQ ID NOs: 9, 15, and 16, respectively, and the VL region comprises the CDR-L1, the CDR-L2, and the CDR-L3 comprising the sequence set forth in SEQ ID NOs: 19, 22, and 23, respectively; the VH region comprises the CDR-H1, the CDR-H2, and the CDR-H3 comprising the sequence set forth in SEQ ID NOs: 9, 17, and 14, respectively, and the VL region comprises the CDR-L1, the CDR-L2, and the CDR-L3 comprising the sequence set forth in SEQ ID NOs: 19, 22, and 23, respectively; the VH region comprises the CDR-H1, the CDR-H2, and the CDR-H3 comprising the sequence set forth in SEQ ID NOs: 9, 15, and 18, respectively, and the VL region comprises the CDR-L1, the CDR-L2, and the CDR-L3 comprising the sequence set forth in SEQ ID NOs: 19, 22, and 23, respectively.

In certain embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 25, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 26, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 27, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 28, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 42; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 29, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 30, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 31, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 32, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 33, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 34, and the VL region comprises an amino acid sequence that has at least at or about 85%, 86%, 87%, 88%, 89%, 90%, 91%, 92%, 93%, 94%, 95%, 96%, 97%, 98% or 99% sequence identity to ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 35, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 38, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41; or the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 126, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40.

In certain embodiments, the VH region and the VL region comprise the sequences set forth in SEQ ID NO: 24 and 39, respectively; the VH region and the VL region comprise the sequences set forth in SEQ ID NO: 25 and 40, respectively; the VH region and the VL region comprise the sequences set forth in SEQ ID NO: 26 and 40, respectively; the VH region and the VL region comprise the sequences set forth in SEQ ID NO: 27 and 41, respectively; the VH region and the VL region comprise the sequences set forth in SEQ ID NO: 28 and 42, respectively; the VH region and the VL region comprise the sequences set forth in SEQ ID NO: 29 and 40, respectively; the VH region and the VL region comprise the sequences set forth in SEQ ID NO: 30 and 41, respectively; the VH region and the VL region comprise the sequences set forth in SEQ ID NO: 31 and 43, respectively; the VH region and the VL region comprise the sequences set forth in SEQ ID NO: 32 and 40, respectively; the VH region and the VL region comprise the sequences set forth in SEQ ID NO: 33 and 40, respectively; the VH region and the VL region comprise the sequences set forth in SEQ ID NO: 34 and 40, respectively; the VH region and the VL region comprise the sequences set forth in SEQ ID NO: 35 and 40, respectively; the VH region and the VL region comprise the sequences set forth in SEQ ID NO: 36 and 40, respectively; the VH region and the VL region comprise the sequences set forth in SEQ ID NO: 37 and 40, respectively; the VH region and the VL region comprise the sequences set forth in SEQ ID NO: 38 and 41, respectively; or the VH region and the VL region comprise the sequences set forth in SEQ ID NO: 126 and 40, respectively.

In certain embodiments, the CD3 binding domain comprises the VH region that comprises the CDR-H1, a CDR-H2, and a CDR-H3 comprising the sequence set forth in SEQ ID NOs: 9, 15, and 14, respectively, and the VL region comprises a CDR-L1, a CDR-L2, and a CDR-L3 comprising the sequence set forth in SEQ ID NOs: 19, 22, and 21, respectively.

In certain embodiments, the CD3 binding domain comprises the VH region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 28, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 42.

In certain embodiments, the CD3 binding domain comprises the VH region and the VL region that comprises the sequence or are the sequence set forth in SEQ ID NO: 28 and 42, respectively.

In certain embodiments, the CD3 binding domain comprises the VH region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 29, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 40.

In certain embodiments, the CD3 binding domain comprises the VH region and the VL region that comprises the sequence or are the sequence set forth in SEQ ID NO: 29 and 40, respectively.

In certain embodiments, the CD3 binding domain comprises the VH region that comprises the CDR-H1, a CDR-H2, and a CDR-H3 comprising the sequence set forth in SEQ ID NOs: 9, 15, and 16, respectively, and the VL region comprises a CDR-L1, a CDR-L2, and a CDR-L3 comprising the sequence set forth in SEQ ID NOs: 19, 22, and 23, respectively.

In certain embodiments, the CD3 binding domain comprises the VH region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 30, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 41.

In certain embodiments, the CD3 binding domain comprises the VH region and the VL region that comprises the sequence or are the sequence set forth in SEQ ID NO: 30 and 41, respectively.

In certain embodiments, the CD3 binding domain comprises the VH region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 31, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 43.

In certain embodiments, the CD3 binding domain comprises the VH region and the VL region that comprises the sequence or are the sequence set forth in SEQ ID NO: 31 and 43, respectively.

An aspect of the invention provides a binding molecule comprising a CD3 binding domain comprising a VH region and a VL region, wherein: the VH region comprises a CDR-H1 set forth in SEQ ID NO: 107, a CDR-H2 set forth in SEQ ID NO: 154, and a CDR-H3 set forth in SEQ ID NO: 109; and the VL region comprises a CDR-L1 set forth in SEQ ID NO: 19, a CDR-L2 set forth in SEQ ID NO: 110, and a CDR-L3 set forth within SEQ ID NO: 155.

An aspect of the invention provides a binding molecule comprising a CD3 binding domain comprising a VH region and a VL region, wherein: the VH region comprises a CDR-H1 set forth in SEQ ID NO: 107, a CDR-H2 set forth in SEQ ID NO: 108, and a CDR-H3 set forth in SEQ ID NO:109; and the VL region comprises a CDR-L1 set forth in SEQ ID NO: 19, a CDR-L2 set forth in SEQ ID NO: 110, and a CDR-L3 set forth within SEQ ID NO: 6.

In certain embodiments, the VH region of the CD3 binding domain and the VL region of the CD3 binding domain comprises, respectively: an amino acid sequence of the consensus sequence QVQLVESGGGVVQPGX₁SLRLSCAASGFTFSX₂X₃GIHWVRQAPGKX₄LEWVAAIWY DX₅SX₆X₇YYADSVKGRFTISRDNSX₈NTLX₉LQMNSLRAEDTAVYYCARSNX₁₀DX₁₁L TX₁₂X₁₃YX₁₄X₁₅GX₁₆X₁₇VWGQGTTVTVSS, wherein X₁ is R, G, or E; X₂ is R or N; X₃ is Y or H, X₄ is G or C; X₅ is G, A, or S; X₆ is K, H, or N, X₇ is K, T, or D; X₈ is K, E, or S; X₉ is Y or D; X₁₀ is Y or F, X₁₁ is I or V, X₁₂ is R or G; X₁₃ is Y or H; X₁₄ is Q or S; X₁₅ is Y or H; X₁₆ is I or L; and X₁₇ is N or D; (SEQ ID NO: 156); and an amino acid sequence of the consensus sequence set forth in AIQLTQSPSSLSASVGDRVTITCRASQGISSALAWYQQKPGKAPKLLIYDASX₁LEX₂G VPSRFSGSGSGTDFTLTISSLQPEDFATYYCQQFX₃SYPRTFGX₄GTKVEIK, wherein X₁ is S or G, X₂ is N or S; X₃ is K or N; and X₄ is Q or C; (SEQ ID NO: 106).

The binding molecule of any one of claims 1-3, wherein the VH region of the CD3 binding domain and the VL region of the CD3 binding domain comprises, respectively: an amino acid sequence of the consensus sequence QVQLVESGGGVVQPGX₁SLRLSCAASGFTFSX₂X₃GIHWVRQAPGKX₄LEWVAAIWY DX₅SX₆X₇YYADSVKGRFTISRDNSX₈NTLX₉LQMNSLRAEDTAVYYCARSNX₁₀DX₁₁L TX₁₂X₁₃YX₁₄X₁₅GX₁₆X₁₇VWGQGTTVTVSS, wherein X₁ is R, G, or E; X₂ is R or N; X₃ is Y or H, X₄ is G or C; X₅ is G or A or S; X₆ is K, H, or N, X₇ is K, T, or D; X₈ is K, E, or S; X₉ is Y or D; X₁₀ is Y or F, X₁₁ is I or V, X₁₂ is R or G; X₁₃ is Y or H; X₁₄ is Q or S; X₁₅ is Y or H; X₁₆ is I or L; and X₁₇ is N or D; (SEQ ID NO: 105); and sequence of the consensus sequence set forth in AIQLTQSPSSLSASVGDRVTITCRASQGISSALAWYQQKPGKAPKLLIYDASX₁LEX₂G VPSRFSGSGSGTDFTLTISSLQPEDFATYYCQQFX₃SYPRTFGX₄GTKVEIK, wherein X₁ is S or G, X₂ is N or S; X₃ is K or N; and X₄ is Q or C; (SEQ ID NO: 106).

In certain embodiments, the CDR-H1 comprises the amino acid sequence set forth in any one of SEQ ID NOs: 9, 12, 111, or 112, the CDR-H2 comprises the amino acid sequence set forth in any one of SEQ ID NOs: 10, 13, 15, 17, or 113-117, and the CDR-H3 comprises the amino acid sequence set forth in any one of SEQ ID NOs: 11, 14, 16, 18, 118-123; and the CDR-L1 comprises the amino acid sequence set forth in SEQ ID NOs: 19, the CDR-L2 comprises the amino acid sequence set forth in any one of SEQ ID NOs: 20, 22, 124, or 125, and the CDR-L3 comprises the amino acid sequence set forth in any one of SEQ ID NOs: 21 or 23.

In certain embodiments, in the CD3 binding region: the VH region comprises the CDR-H1, the CDR-H2, and the CDR-H3 contained within SEQ ID NO: 24, and the VL region comprises the CDR-L1, the CDR-L2, and the CDR-L3 contained within SEQ ID NO: 39; the VH region comprises the CDR-H1, the CDR-H2, and the CDR-H3 contained within SEQ ID NO: 25, and the VL region comprises the CDR-L1, the CDR-L2, and the CDR-L3 contained within SEQ ID NO: 40; the VH region comprises the CDR-H1, the CDR-H2, and the CDR-H3 contained within SEQ ID NO: 26, and the VL region comprises the CDR-L1, the CDR-L2, and the CDR-L3 contained within SEQ ID NO: 40; the VH region comprises the CDR-H1, the CDR-H2, and the CDR-H3 contained within SEQ ID NO: 27, and the VL region comprises the CDR-L1, the CDR-L2, and the CDR-L3 contained within SEQ ID NO: 41; the VH region comprises the CDR-H1, the CDR-H2, and the CDR-H3 contained within SEQ ID NO: 28, and the VL region comprises the CDR-L1, the CDR-L2, and the CDR-L3 contained within SEQ ID NO: 42; the VH region comprises the CDR-H1, the CDR-H2, and the CDR-H3 contained within SEQ ID NO: 29, and the VL region comprises the CDR-L1, the CDR-L2, and the CDR-L3 contained within SEQ ID NO: 40; the VH region comprises the CDR-H1, the CDR-H2, and the CDR-H3 contained within SEQ ID NO: 30, and the VL region comprises the CDR-L1, the CDR-L2, and the CDR-L3 contained within SEQ ID NO: 41; the VH region comprises the CDR-H1, the CDR-H2, and the CDR-H3 contained within SEQ ID NO: 31, and the VL region comprises the CDR-L1, the CDR-L2, and the CDR-L3 contained within SEQ ID NO: 43; the VH region comprises the CDR-H1, the CDR-H2, and the CDR-H3 contained within SEQ ID NO: 32, and the VL region comprises the CDR-L1, the CDR-L2, and the CDR-L3 contained within SEQ ID NO: 40; the VH region comprises the CDR-H1, the CDR-H2, and the CDR-H3 contained within SEQ ID NO: 33, and the VL region comprises the CDR-L1, the CDR-L2, and the CDR-L3 contained within SEQ ID NO: 40; the VH region comprises the CDR-H1, the CDR-H2, and the CDR-H3 contained within SEQ ID NO: 34, and the VL region comprises the CDR-L1, the CDR-L2, and the CDR-L3 contained within SEQ ID NO: 40; the VH region comprises the CDR-H1, the CDR-H2, and the CDR-H3 contained within SEQ ID NO: 35, and the VL region comprises the CDR-L1, the CDR-L2, and the CDR-L3 contained within SEQ ID NO: 40; the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NO: 40; the VH region comprises the CDR-H1, the CDR-H2, and the CDR-H3 contained within SEQ ID NO: 37, and the VL region comprises the CDR-L1, the CDR-L2, and the CDR-L3 contained within SEQ ID NO: 40; the VH region comprises the CDR-H1, the CDR-H2, and the CDR-H3 contained within SEQ ID NO: 38, and the VL region comprises the CDR-L1, the CDR-L2, and the CDR-L3 contained within SEQ ID NO: 41; the VH region comprises the CDR-H1, CDR-H2 and the CDR-H3 contained within SEQ ID NO: 126, and the VL region comprises the CDR-L1, the CDR-L2, and the CDR-L3 contained within SEQ ID NO: 40; the VH region comprises the CDR-H1, CDR-H2 and the CDR-H3 contained within SEQ ID NO: 4, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 53, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 54, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 55, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 56, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 57, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 58, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 59, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 60, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 61, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 62, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 63, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 64, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 65, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 66, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 67, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 68, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 69, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 70, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 71, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 72, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 73, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 74, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 75, and the VL region comprises the CDR-L1, the CDR-L2, and the CDR-L3 contained within SEQ ID NOs: 39, 40, or 41; the VH region comprises the CDR-H1, CDR-H2 and the CDR-H3 contained within SEQ ID NO: 24, and the VL region comprises the CDR-L1, the CDR-L2, and the CDR-L3 contained within SEQ ID NOs: 40; the VH region comprises the CDR-H1, CDR-H2 and the CDR-H3 contained within SEQ ID NO: 24, and the VL region comprises the CDR-L1, the CDR-L2, and the CDR-L3 contained within SEQ ID NOs: 77; the VH region comprises the CDR-H1, CDR-H2 and the CDR-H3 contained within SEQ ID NO: 24, and the VL region comprises the CDR-L1, the CDR-L2, and the CDR-L3 contained within SEQ ID NOs: 78; the VH region comprises the CDR-H1, CDR-H2 and the CDR-H3 contained within SEQ ID NO: 24, and the VL region comprises the CDR-L1, the CDR-L2, and the CDR-L3 contained within SEQ ID NOs: 79; the VH region comprises the CDR-H1, CDR-H2 and the CDR-H3 contained within SEQ ID NO: 24, and the VL region comprises the CDR-L1, the CDR-L2, and the CDR-L3 contained within SEQ ID NOs: 80; the VH region comprises the CDR-H1, the CDR-H2, and the CDR-H3 contained within SEQ ID NO: 27, and the VL region comprises the CDR-L1, the CDR-L2, and the CDR-L3 contained within SEQ ID NOs: 39; the VH region comprises the CDR-H1, the CDR-H2, and the CDR-H3 contained within SEQ ID NO: 25, and the VL region comprises the CDR-L1, the CDR-L2, and the CDR-L3 contained within SEQ ID NOs: 39; or the VH region comprises the CDR-H1, the CDR-H2, and the CDR-H3 contained within SEQ ID NO: 26, and the VL region comprises the CDR-L1, the CDR-L2, and the CDR-L3 contained within SEQ ID NOs: 39.

In certain embodiments, the VH region of the CD3 binding domain and the VL region of the CD3 binding domain comprises: the CDR-H1, the CDR-H2, and the CDR-H3 comprising the amino acid sequence set forth in SEQ ID NOs: 9, 10, and 11, respectively, and the CDR-L1, the CDR-L2, and the CDR-L3 comprising the amino acid sequence set forth in SEQ ID NOs: 19, 20, and 21, respectively; the CDR-H1, the CDR-H2, and the CDR-H3 comprising the amino acid sequence set forth in SEQ ID NOs: 12, 13, and 11, respectively, and t the CDR-L1, the CDR-L2, and the CDR-L3 comprising the amino acid sequence set forth in SEQ ID NOs: 19, 22, and 21, respectively; the CDR-H1, the CDR-H2, and the CDR-H3 comprising the amino acid sequence set forth in SEQ ID NOs: 9, 13, and 14, respectively, and the CDR-L1, the CDR-L2, and the CDR-L3 comprising the amino acid sequence set forth in SEQ ID NOs: 19, 22, and 21, respectively; the CDR-H1, the CDR-H2, and the CDR-H3 comprising the amino acid sequence set forth in SEQ ID NOs: 9, 13, and 11, respectively, and the CDR-L1, the CDR-L2, and the CDR-L3 comprising the amino acid sequence set forth in SEQ ID NOs: 19, 22, and 23, respectively; the CDR-H1, the CDR-H2, and the CDR-H3 comprising the amino acid sequence set forth in SEQ ID NOs: 9, 15, and 14, respectively, and the CDR-L1, the CDR-L2, and the CDR-L3 comprising the amino acid sequence set forth in SEQ ID NOs: 19, 22, and 21, respectively; the CDR-H1, the CDR-H2, and the CDR-H3 comprising the amino acid sequence set forth in SEQ ID NOs: 9, 15, and 16, respectively, and the CDR-L1, the CDR-L2, and the CDR-L3 comprising the amino acid sequence set forth in SEQ ID NOs: 19, 22, and 23, respectively; the CDR-H1, the CDR-H2, and the CDR-H3 comprising the amino acid sequence set forth in SEQ ID NOs: 9, 17, and 14, respectively, and the CDR-L1, the CDR-L2, and the CDR-L3 comprising the amino acid sequence set forth in SEQ ID NOs: 19, 22, and 21, respectively; the CDR-H1, the CDR-H2, and the CDR-H3 comprising the amino acid sequence set forth in SEQ ID NOs: 9, 15, and 18, respectively, and the CDR-L1, the CDR-L2, and the CDR-L3 comprising the amino acid sequence set forth in SEQ ID NOs: 19, 22, and 23, respectively; the CDR-H1, CDR-H2 and the CDR-H3 comprising the amino acid sequence set forth in SEQ ID NOs: 9, 10, and 11, respectively, and the VL region comprises the CDR-L1, the CDR-L2, and the CDR-L3 comprising the amino acid sequence set forth in SEQ ID NOs: 19, 20, and 21, respectively; the VH region comprises the CDR-H1, CDR-H2 and the CDR-H3 comprising the amino acid sequence set forth in SEQ ID NOs: 9, 113, and 11, respectively, and the VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 9, 13, and 11, respectively, and VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 9, 114, and 11, respectively, and VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 9, 115, and 11, respectively, and VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 9, 10, and 118, respectively, and VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 9, 10, and 18, respectively, and VL region the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 9, 10, and 119, respectively, and VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 9, 10, and 120, respectively, and VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 9, 10, and 16, respectively, and VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 12, 10, and 11, respectively, and VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 111, 10, and 11, respectively, and VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 112, 10, and 11, respectively, and VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 9, 10, and 121, respectively, and VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 9, 10, and 122, respectively, and VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 9, 10, and 123, respectively, and VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 9, 10, and 14, respectively, and VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 9, 116, and 11, respectively, and VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 9, 117, and 11, respectively, and VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 9, 13, and 11, respectively, and VL region comprises the CDR-L1, the CDR-L2, and the CDR-L3 comprising the amino acid sequence set forth in SEQ ID NOs: 19, 20, and 21, respectively; the CDR-H1, the CDR-H2, and the CDR-H3 comprising the sequence set forth in SEQ ID NOs: 9, 13, and 18, respectively, and the VL region comprises the CDR-L1, the CDR-L2, and the CDR-L3 comprising the sequence set forth in SEQ ID NOS: 19, 20, and 21, respectively; the CDR-H1, CDR-H2 and the CDR-H3 comprising the amino acid sequence set forth in SEQ ID NOs: 9, 13, and 119, respectively, and VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 111, 13, and 11, respectively, and VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 9, 113, and 123, respectively, and the VL region comprises the CDR-L1, the CDR-L2, and the CDR-L3 comprising the amino acid sequence set forth in SEQ ID NOs: 19, 20, and 21, respectively; the CDR-H1, CDR-H2 and the CDR-H3 comprising the amino acid sequence set forth in SEQ ID NOs: 9, 10, and 11, respectively, and VL region comprises the CDR-L1, the CDR-L2, and the CDR-L3 comprising the amino acid sequence set forth in SEQ ID NOs: 19, 22, and 21, respectively; the CDR-H1, CDR-H2 and the CDR-H3 comprising the amino acid sequence set forth in SEQ ID NOs: 9, 10, and 11, respectively, and VL region comprises the CDR-L1, the CDR-L2, and the CDR-L3 comprising the amino acid sequence set forth in SEQ ID NOs: 19, 20, and 23, respectively; the CDR-H1, CDR-H2 and the CDR-H3 comprising the amino acid sequence set forth in SEQ ID NOs: 9, 10, and 11, respectively, and VL region comprises the CDR-L1, the CDR-L2, and the CDR-L3 comprising the amino acid sequence set forth in SEQ ID NOs: 19, 22, and 23, respectively; the CDR-H1, CDR-H2 and the CDR-H3 comprising the amino acid sequence set forth in SEQ ID NOs: 9, 10, and 11, respectively, and VL region comprises the CDR-L1, the CDR-L2, and the CDR-L3 comprising the amino acid sequence set forth in SEQ ID NOs: 19, 124, and 21, respectively; the CDR-H1, CDR-H2 and the CDR-H3 comprising the amino acid sequence set forth in SEQ ID NOs: 9, 10, and 11, respectively, and VL region comprises the CDR-L1, the CDR-L2, and the CDR-L3 comprising the amino acid sequence set forth in SEQ ID NOs: 19, 125, and 23, respectively; the CDR-H1, CDR-H2 and the CDR-H3 comprising the sequence set forth in SEQ ID NOs: 9, 13, and 16, respectively, and VL region comprises the CDR-L1, the CDR-L2, and the CDR-L3 comprising the sequence set forth in SEQ ID NOs: 19, 20, and 21, respectively; the CDR-H1, CDR-H2 and the CDR-H3 comprising the sequence set forth in SEQ ID NOs: 12, 13, and 11, respectively, and VL region comprises the CDR-L1, the CDR-L2, and the CDR-L3 comprising the sequence set forth in SEQ ID NOs: 19, 20, and 21, respectively; or the CDR-H1, CDR-H2 and the CDR-H3 comprising the sequence set forth in SEQ ID NOs: 9, 13, and 14, respectively, and VL region comprises the CDR-L1, the CDR-L2, and the CDR-L3 comprising the sequence set forth in SEQ ID NOs: 19, 20, and 21, respectively.

In certain embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 25, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 26, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 27, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 28, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 42; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 29, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 30, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 31, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 32, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 33, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 34, and the VL region comprises an amino acid sequence that has at least at or about 85%, 86%, 87%, 88%, 89%, 90%, 91%, 92%, 93%, 94%, 95%, 96%, 97%, 98% or 99% sequence identity to ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 35, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 38, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 126, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 4, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 53, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 54, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 55, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 56, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 57, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 58, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 59, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 60, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 61, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 62, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 63, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 64, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 65, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 66, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 67, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 68, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 69, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 70, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 71, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 72, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 73, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 74, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 75, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 99; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 100; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 101; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 27, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39; or the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 26, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39.

In certain embodiments, in the CD3 binding domain: the VH region and the VL region comprise the amino acid sequences set forth in SEQ ID NO: 24 and 39, respectively; the VH region and the VL region comprise the amino acid sequences set forth in SEQ ID NO: 24 and 40, respectively; the VH region and the VL region comprise the amino acid sequences set forth in SEQ ID NO: 26 and 40, respectively; the VH region and the VL region comprise the amino acid sequences set forth in SEQ ID NO: 27 and 41, respectively; the VH region and the VL region comprise the amino acid sequences set forth in SEQ ID NO: 28 and 42, respectively; the VH region and the VL region comprise the amino acid sequences set forth in SEQ ID NO: 29 and 40, respectively; the VH region and the VL region comprise the amino acid sequences set forth in SEQ ID NO: 30 and 41, respectively; the VH region and the VL region comprise the amino acid sequences set forth in SEQ ID NO: 31 and 43, respectively; the VH region and the VL region comprise the amino acid sequences set forth in SEQ ID NO: 32 and 40, respectively; the VH region and the VL region comprise the amino acid sequences set forth in SEQ ID NO: 33 and 40, respectively; the VH region and the VL region comprise the amino acid sequences set forth in SEQ ID NO: 34 and 40, respectively; the VH region and the VL region comprise the amino acid sequences set forth in SEQ ID NO: 35 and 40, respectively; the VH region and the VL region comprise the amino acid sequences set forth in SEQ ID NO: 36 and 40, respectively; the VH region and the VL region comprise the amino acid sequences set forth in SEQ ID NO: 37 and 40, respectively; the VH region and the VL region comprise the amino acid sequences set forth in SEQ ID NO: 38 and 41, respectively; the VH region and the VL region comprise the sequences set forth in SEQ ID NO: 126 and 40, respectively; VH region and the VL region comprise the sequences set forth in SEQ ID NO: 4 and 39, respectively; the VH region and the VL region comprise the sequences set forth in SEQ ID NO: 83 and 39, respectively; the VH region and the VL region comprise the sequences set forth in SEQ ID NO: 54 and 39, respectively; the VH region and the VL region comprise the sequences set forth in SEQ ID NO: 55 and 39, respectively; the VH region and the VL region comprise the sequences set forth in SEQ ID NO: 56 and 39, respectively; the VH region and the VL region comprise the sequences set forth in SEQ ID NO: 57 and 39, respectively; the VH region and the VL region comprise the sequences set forth in SEQ ID NO: 58 and 39, respectively; the VH region and the VL region comprise the sequences set forth in SEQ ID NO: 59 and 39, respectively; the VH region and the VL region comprise the amino acid sequences set forth in SEQ ID NO: 76 and 39, respectively; the VH region and the VL region comprise the amino acid sequences set forth in SEQ ID NO: 61 and 39, respectively; the VH region and the VL region comprise the amino acid sequences set forth in SEQ ID NO: 62 and 39, respectively; the VH region and the VL region comprise the amino acid sequences set forth in SEQ ID NO: 63 and 39, respectively; the VH region and the VL region comprise the amino acid sequences set forth in SEQ ID NO: 64, and 39, respectively; the VH region and the VL region comprise the amino acid sequences set forth in SEQ ID NO: 65 and 39, respectively; the VH region and the VL region comprise the amino acid sequences set forth in SEQ ID NO: 66 and 39, respectively; the VH region and the VL region comprise the amino acid sequences set forth in SEQ ID NO: 67 and 39, respectively; the VH region and the VL region comprise the amino acid sequences set forth in SEQ ID NO: 68 and 39, respectively; the VH region and the VL region comprise the amino acid sequences set forth in SEQ ID NO: 69 and 39, respectively; the VH region and the VL region comprise the amino acid sequences set forth in SEQ ID NO: 70 and 39, respectively; the VH region and the VL region comprise the amino acid sequences set forth in SEQ ID NO: 71 and 39, respectively; the VH region and the VL region comprise the amino acid sequences set forth in SEQ ID NO: 72 and 39, respectively; the VH region and the VL region comprise the amino acid sequences set forth in SEQ ID NO: 73 and 39, respectively; the VH region and the VL region comprise the amino acid sequences set forth in SEQ ID NO: 74 and 39, respectively; the VH region and the VL region comprise the amino acid sequences set forth in SEQ ID NO: 75 and 39, respectively; the VH region and the VL region comprise the amino acid sequences set forth in SEQ ID NO: 24 and 40, respectively; the VH region and the VL region comprise the amino acid sequences set forth in SEQ ID NO: 24 and 77, respectively; the VH region and the VL region comprise the amino acid sequences set forth in SEQ ID NO: 24 and 81, respectively; the VH region and the VL region comprise the amino acid sequences set forth in SEQ ID NO: 24 and 79, respectively; the VH region and the VL region comprise the amino acid sequences set forth in SEQ ID NO: 24 and 80, respectively; the VH region and the VL region comprise the sequences set forth in SEQ ID NO: 27 and 39, respectively; the VH region and the VL region comprise the sequences set forth in SEQ ID NO: 24 and 39, respectively; or the VH region and the VL region comprise the sequences set forth in SEQ ID NO: 26 and 39, respectively.

In certain embodiments, the binding molecule is a full length antibody. For example, the full length antibody comprises a constant region (Fc) from an IgA1, IgA2, IgD, IgE, IgG1, IgG2, IgG3, IgG4, or IgM. In certain embodiments, the constant region is a human IgG1 heavy chain constant region. In certain embodiments, the constant region comprises the amino acid sequence set forth in any of SEQ ID NOs: 44-47. In certain embodiments, the constant region comprises the amino acid sequence set forth in any of SEQ ID NO: 48-51.

In certain embodiments, the binding molecule comprises an amino acid sequence described herein including those containing sequences listed in Table E2, Table E3, Table E4, and/or Table E12. In certain embodiments, the binding molecule comprises an amino acid sequence listed in the Examples 1-7.

Provided herein is a polynucleotide comprising a nucleic acid sequence(s) encoding the binding molecule or antibody or antigen-binding fragment disclosed herein. An aspect of the invention provides a binding molecule comprising an amino acid sequence described herein. An aspect of the invention provides a polynucleotide comprising a nucleic acid encoding a binding molecule described herein. In certain embodiments, the nucleic acid encodes a binding molecule comprising an amino acid sequence described herein including those containing sequences listed in Table E2, Table E3, Table E4, and/or Table E12. In certain embodiments, the nucleic acid encodes a binding molecule listed in the Examples 1-7.

An aspect of the invention provides a vector comprising any of the polynucleotides described herein. In some embodiments, the vector is an expression vector. In certain embodiments, the vector is a viral vector. For example, the viral vector is a retroviral vector or a lentiviral vector. In some embodiments, the vector useful for expressing a binding molecule described herein comprising the polynucleotide.

An aspect of the inventions provides a cell comprising any of the polynucleotides described herein, or any of the vectors of described herein. In certain embodiments, the cell is a mammalian cell. In certain embodiments, the cell is a CHO cell. In certain embodiments, the cell is a NS0 cell. In certain embodiments, the cell is a SP2/0 cell. In certain embodiments, the cell is a HEK293 cell. In certain embodiments, the cell is a PER.C6 cell. In certain embodiments, the cell is a BHK-21 cell.

An aspect of the invention provides a method of producing any of the antibodies described herein comprising culturing the cell under a condition that produces the antibody. In certain embodiments, the method, further comprises recovering the antibody produced by the cell.

An aspect of the inventions provides an antibody or antigen-binding fragment thereof produced by any of the methods described herein.

An aspect of the invention provides a composition comprising any of the binding molecules described herein. In certain embodiments, the binding molecule comprises an amino acid sequence described herein including any containing sequences listed in Table E2, Table E3, Table E4, and/or Table E12. In certain embodiments, the binding molecule comprises an amino acid sequence listed in the Examples 1-7. In certain embodiments, the composition further comprises a pharmaceutically acceptable excipient.

An aspect of the invention provides a method for treating a subject with a disease or condition, the method comprising administering a therapeutically effective amount of any of the binding molecules described herein. In certain embodiments, the binding molecule comprises an amino acid sequence described herein including any containing sequences listed in Table E2, Table E3, Table E4, and/or Table E12. In certain embodiments, the binding molecule comprises an amino acid sequence listed in the Examples 1-7.

In certain embodiments, wherein the disease or condition is an infectious disease or disorder, an autoimmune disease, an inflammatory disease, or a tumor or a cancer. In certain embodiments, the disease or condition is a tumor or a cancer.

An aspect of the invention provides a kit comprising any of the binding molecules described herein and, optionally, instructions for use.

### DETAILED DESCRIPTION

Provided are CD3-binding molecules, such as anti-CD3 antibodies including antigen-binding antibody fragments that specifically bind to CD3 proteins, such as a human CD3 protein. The binding molecules also include proteins that include any of the provided anti-CD3 antibodies, including bispecific antibodies. In particular embodiments, provided are bispecific T cell engaging antibodies that bind and target CD3 and a second antigen to mediate specific targeting and T cell-mediated killing of cells that express the second antigen. Also provided are polynucleotides containing nucleic acids sequences encoding all or a portion of such binding molecules, including polynucleotides that encode polypeptide chains of the bispecific antibodies. Among the provided embodiments are approaches for targeting cells expressing the second antigen useful in the treatment of diseases and conditions, including cancers or autoimmune diseases or conditions.

The human CD3 T cell antigen receptor protein complex is composed of six distinct chains: a CD3γ chain (SwissProt P09693), a CD36 chain (SwissProt P04234), two CD3ε chains (SwissProt P07766), and one CD3ζ chain homodimer (SwissProt P20963) (εγ:εδ:ζζ), which is associated with the T cell receptor α and β chain. This complex plays an important role in coupling antigen recognition to several intracellular signal-transduction pathways. The CD3 complex mediates signal transduction, resulting in T cell activation and proliferation. CD3 is required for immune response. Among provided embodiments, the binding molecules target or bind CD3 epsilon (CD3e or CD3ε). In some embodiments, the antibodies provided herein bind to human CD3ε set forth in SEQ ID NO:82.

Therapies targeting CD3 that specifically engage T cells for cytolytic killing of target cells are needed. Such therapies include TCE bispecific antibody therapies for targeted killing of antigen-expression cells. Among TCE therapies are therapies that direct T cells to tumors that selectively recruit T cells to tumor cells such that the targeted antibody therapy delivers cytotoxic cells specifically to the antigen-expressing cancer cells. Similar approaches are useful for targeting autoreactive T and/or B cells in connection with autoimmune diseases or indications.

Yet, TCE approaches have not been entirely satisfactory. In many instances, current therapies may exhibit undesired tonic signaling either in the absence of a second target antigen or due to high affinity binding to CD3 on T cells. This risk of tonic signaling may exacerbate the chance of toxicities, such as cytokine release syndrome (CRS), to the antigen-directed therapy. CRS is a concern for T cell modality therapies including T cell engagers. While on target T cell killing of tumor cells results in cytokine release, tonic signaling and cytokine release of T cells through CD3 binding independent of tumor associated antigen binding is not desired. Improved strategies are needed for optimal responses to antibody or T cell-mediated therapies. Provided are embodiments that meet such needs.

Among the provided embodiments are CD3 binding molecules that include an anti-CD3 antibody or antigen-binding fragment. In some aspects, provided binding molecules contain a detuned anti-CD3 targeting domain that has reduced affinity to CD3 compared to other T cell engagers, thereby minimizing risks of tonic signaling and CRS. Also among the provided embodiments are T cell engager bispecific antibodies directed against CD3 and a second antigen that is expressed on a target cell desired to be targeted for cytolytic killing. In some embodiments, the target cell is a tumor or cancer cell. In some embodiments, the target cell is an autoreactive B cell. Due to the lower affinity, and in some cases low tonic signaling, the provided binding molecules, including bispecific antibodies, are promising candidates for engaging T cell activity including in connection with mediating cytolytic killing of antigen-expressing cells.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising,” “consisting essentially of,” and “consisting of” may be optionally replaced with either of the other two terms, thus describing alternative aspects of the scope of the subject matter. The invention illustratively described herein suitably may be practiced in the absence of any element or elements, limitation or limitations which is not specifically disclosed herein.

As used herein, the singular forms “a,” “an,” and “the” include plural referents unless the context clearly dictates otherwise. The use of “or” or “and” means “and/or” unless stated otherwise. Furthermore, use of the term “including” as well as other forms, such as “include,” “includes,” and “included,” is not limiting.

The term “about” as used herein when referring to a measurable value such as an amount, a temporal duration and the like, encompasses variations of up to ±10% from the specified value. Unless otherwise indicated, all numbers expressing e.g., quantities of ingredients or properties (e.g., molecular weight, reaction conditions) described herein are to be understood as being modified by the term “about”.

As used herein, “and/or” is to be taken as specific disclosure of each of the two specified features or components with or without the other. Thus, the term “and/or” as used in a phrase such as “A and/or B” includes “A and B,” “A or B,” “A” alone, and “B” alone. Likewise, the term “and/or” as used in a phrase such as “A, B, and/or C” encompasses each of the following: A, B, and C; A, B, or C; A or C; A or B; B or C; A and C; A and B; B and C; A alone; B alone; and C alone.

As used herein, recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3% are expressly enumerated in this specification. These are only examples of what is specifically intended, and all possible combinations of numerical values between and including the lowest value and the highest value enumerated are to be considered to be expressly stated in this disclosure.

The term “binding domain” characterizes in connection with the present disclosure a domain which (specifically) binds to/interacts with/recognizes a given target epitope or a given target side on the target molecules (antigens), e.g., CD3. The structure and function of the first binding domain (recognizing CD3), and preferably also the structure and/or function of the second binding domain (such as those described in section II.B.1), is/are based on the structure and/or function of an antibody, e.g. of a full-length or whole immunoglobulin molecule and/or is/are drawn from the variable heavy chain (VH) and/or variable light chain (VL) domains of an antibody or fragment thereof. Typically, the first binding domain is characterized by the presence of three light chain CDRs (i.e. CDR1, CDR2 and CDR3 of the VL region) and/or three heavy chain CDRs (i.e. CDR1, CDR2 and CDR3 of the VH region). The second binding domain typically also comprises the minimum structural requirements of an antibody which allow for the target binding. More typically, the second binding domain comprises at least three light chain CDRs (i.e. CDR1, CDR2 and CDR3 of the VL region) and three heavy chain CDRs (i.e. CDR1, CDR2 and CDR3 of the VH region).

The term “antibody” as used herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, heavy chain variable (VH) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

In some embodiments, the antibody constructs of the invention are “isolated” or “substantially pure” antibody constructs. “Isolated” or “substantially pure”, when used to describe the antibody constructs disclosed herein, means an antibody construct that has been identified, separated and/or recovered from a component of its production environment. Preferably, the antibody construct is free or substantially free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. The antibody constructs may e.g. constitute at least about 5%, or at least about 50% by weight of the total protein in a given sample. It is understood that the isolated protein may constitute from 5% to 99.9% by weight of the total protein content, depending on the circumstances. The polypeptide may be made at a significantly higher concentration through the use of an inducible promoter or high expression promoter, such that it is made at increased concentration levels. The definition includes the production of an antibody construct in a wide variety of organisms and/or host cells that are known in the art. In preferred embodiments, the antibody construct will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated antibody construct will be prepared by at least one purification step.

An antigen-binding site of an antibody provided herein contains six CDRs which contribute in varying degrees to the affinity of the binding site for antigen. There are three heavy chain variable domain CDRs (referred to as CDR-H1, CDR-H2 and CDR-H3) and three light chain variable domain CDRs (referred to as CDR-L1, CDR-L2 and CDR-L3). A skilled artisan is familiar with CDR and framework regions (FRs) of antibodies, and can determine the boundaries of such regions using various known schemes.

The terms “complementarity determining region,” and “CDR,” synonymous with “hypervariable region” or “HVR,” are known to refer to non-contiguous sequences of amino acids within antibody variable regions, which confer antigen specificity and/or binding affinity. In general, there are three CDRs in each heavy chain variable region (i.e., CDR-H1, CDR-H2, CDR-H3) and three CDRs in each light chain variable region (i.e., CDR-L1, CDR-L2, CDR-L3). “Framework regions” and “FR” are known to refer to the non-CDR portions of the variable regions of the heavy and light chains. In general, there are four FRs in each full-length heavy chain variable region (i.e., FR-H1, FR-H2, FR-H3, and FR-H4), and four FRs in each full-length light chain variable region (i.e., FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., J Mol Biol, 1997; 273 (4): 927-48 (“Chothia” numbering scheme); MacCallum et al., J. Mol. Biol, 1996; 262:732-745.” (“Contact” numbering scheme); Lefranc M P et al., Dev Comp Immunol, 2003; 27 (1): 55-77 (“IMGT” numbering scheme); Honegger A and Plückthun A, J Mol Biol, 2001; 309 (3): 657-70, (“Aho” numbering scheme); Martin et al., PNAS, 1989; 86 (23): 9268-9272, (“AbM” numbering scheme); and Ye et al., Nucleic Acids Res. 2013; 41 (Web Server issue): W34-40, (“IgBLAST numbering scheme). Details regarding various numbering schemes are also described in, for example, Jarasch et al., Proteins, 2017; 85 (1): 65-71; Martin et al., Bioinformatics tools for antibody engineering. In: Dübel, S. (editor) Handbook of Therapeutic Antibodies, Vol. 1. Wiley-VCH, Weinheim, Germany; Martin, A.C.R. (2010). Protein Sequence and Structure Analysis of Antibody Variable Domains. In: Kontermann, R., Dübel, S. (eds) Antibody Engineering. Springer Protocols Handbooks. Springer, Berlin, Heidelberg, and Martin, ACR, Antibody Information: How to identify the CDRs by looking at a sequence [online] <http://www.bioinf.org.uk/abs/info.html>, all of which are incorporated by reference in their entireties. Various prediction algorithm tools are available and known for numbering antibody residues and CDRs (e.g., AbYsis, Abnum, AbYmod, AbRSA, IgBLAST, IMGT, or ANARCI).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, in some cases with insertions. Insertions in the sequence relative to the standard numbering scheme are indicated using insertion letter codes. For example, residues that are inserted between residues L30 and L31 are indicated as L31A, L31B, etc. Deletions in the sequence relative to the standard scheme are accommodated by skipping numbers. The two schemes place certain insertions and deletions (“indels”) at different positions, resulting in differential numbering. For instance, the Chothia numbering scheme is nearly identical to the Kabat numbering scheme, except that insertions are placed at structural positions and topologically equivalents residues do get assigned the same numbers. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software. The IgBLAST scheme is based on matching to germline V, D and J genes, and can be determined using National Center for Biotechnology Information (NCBI)'s IgBLAST tool.

In some embodiments, reference to CDRs herein is by Kabat numbering. In some embodiments, Kabat numbering can be determined by known sequence rules as described in, for example, Kabat et al. (1991), “Sequences of Proteins of Immunological Interest,” 5th Ed. Public Health Svice, National Institutes of Health, Bethesda, MD. In some embodiments, the Kabat numbering scheme in some aspects can include any of the following rules to designate CDRs: CDR-L1 starts at approximately residue 24 of the light chain, always has a preceding C residue, and always has a following W residue; the end of CDR-L1 is defined by a stretch of 3 residues, where the W residue can be followed by Y, L, or F, followed by Q or L; CDR-1 has a length of 10 to 17 residues; CDR-L2 always starts 16 residues after the end of CDR-L1; the two residues before CDR-L2 are I and Y but can also be V and Y, I and K, or I and F; CDR-L2 is always 7 residues long; CDR-L3 always starts 33 residues after the end of CDR-L2, always has a preceding C residue, and is strictly followed by a F-G-X-G sequence motif, where X is any amino acid; CDR-L3 has a length of 7 to 11 residues; CDR-H1 starts at approximately position 26 of the heavy chain; the first amino acid in CDR-H1 is always 9 residues after a conserved C residue; CDR-H1 is followed by an invariant W residue followed by V, I, or A; CDR-H1 has a length of 5 to 7 residues; CDR-H2 always starts at 15 residues after the end of CDR-H1; the first residue in CDR-H2 is usually preceded by the sequence motif L-E-W-I-G but a number of variations exist; the end of CDR-H2 is defined by a motif of 3 residues—the first residue of the motif of 3 residues can be either K or R, the second residue of the motif of 3 residues can be L, I, V, F, T, or A, the third residue of the motif of 3 residues can be T, S, I, or A; CDR-H2 has a length of 16 to 19 residues; CDR-H3 always starts 33 residues after the end of CDR-H2 and is always 3 residues after a C residue—the first residue of CDR-H3 is preceded by the conserved C residue followed by two residues, which are usually A-R; the residues following CDR-H3 is strictly followed by a W-G-X-G sequence motif, where the X is any amino acid; CDR-H3 typically has a length of 3 to 25 residues; CDR-H3 can be much longer than 25 residues.

In some embodiments, reference to CDRs herein is by Chothia numbering. In some cases, according to the Chothia numbering scheme, exact boundary positions of certain CDRs can differ based on different definitions for the CDRs (See e.g., Martin, ACR, Antibody Information: How to identify the CDRs by looking at a sequence [online] <http://www.bioinf.org.uk/abs/info.html>). For example, in some instances, the boundary positions for CDR-L1 according to Chothia numbering can be L26-L32 (Chothia et al., Science, 1986; 233 (4765): 755-8 and Chothia C. and Lesk A. M. J Mol Biol, 1987; 196 (4): 901-17). In some instances, the boundary positions for CDR-L1 can be L25-L32 (Al-Lazikani et al., J Mol Biol, 1997; 273 (4): 927-48). In some instances, the boundary positions for CDR-L2 can be L50-L52 and for CDR-L3 can be L91-L96 (Chothia et al., Science, 1986; 233 (4765): 755-8; Chothia C. and Lesk A. M. J Mol Biol, 1987; 196 (4): 901-17; Al-Lazikani et al., J Mol Biol, 1997; 273 (4): 927-48). In some instances, the boundary positions for CDR-H1 according to Chothia numbering can be H26-H32 (Chothia et al., Science, 1986; 233 (4765): 755-8; Chothia C. and Lesk A. M. J Mol Biol, 1987; 196 (4): 901-17; Al-Lazikani et al., J Mol Biol, 1997; 273 (4): 927-48). In some instances, the boundary positions for CDR-H2 can be H53-H55 (Chothia et al., Science, 1986; 233 (4765): 755-8 and Chothia C. and Lesk A. M. J Mol Biol, 1987, 196 (4): 901-17); H52a-H55 (Tramontano et al., J Mol Biol, 1990, 215 (1): 175-82). In some instances, the boundary positions for CDR-H2 can be H52-H56 (Al-Lazikani et al., J Mol Biol., 1997; 273 (4): 927-48). In some instances, the boundary positions for CDR-H3 can be H96-H101 (Chothia et al., Science, 1986; 233 (4765): 755-8 and Chothia C. and Lesk A. M. J Mol Biol., 1987; 196 (4): 901-17). In some instances, the boundary positions for CDR-H3 can be H92-H104 (Morea et al., Biophys Chem, 1997; 68 (1-3): 9-16 and Morea et al., J Mol Biol., 1998; 275 (2): 269-94).

Table 1, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact | IMGT |
|---|---|---|---|---|---|
| CDR-L1 | L24--L34 | L24--L34 | L24--L34 | L30--L36 | L27-L38 |
| CDR-L2 | L50--L56 | L50--L56 | L50--L56 | L46--L55 | L56-L65 |
| CDR-L3 | L89--L97 | L89--L97 | L89--L97 | L89--L96 | L105-L117 |
| CDR-H1 | H31--H35B | H26--H32.34 | H26--H35B | H30--H35B | H27-H38 |
| (Kabat Numbering¹) | | | | | |
| CDR-H1 | H31--H35 | H26--H32 | H26--H35 | H30--H35 | H27-H38 |
| (Chothia Numbering²) | | | | | |
| CDR-H2 | H50--H65 | H52--H56 | H50--H58 | H47--H58 | H56-H65 |
| CDR-H3 | H95--H102 | H95--H102 | H95--H102 | H93--H101 | H105-H117 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, the provided binding molecule, e.g., an antibody or antigen-binding fragment thereof comprises a CDR-H1, a CDR-H2, and a CDR-H3 as contained within a given V_{H} region amino acid sequence and defined by any of the aforementioned schemes, such as Kabat, Chothia, AbM, IgBLAST, IMGT, or Contact method, or other known schemes, and a CDR-L1, a CDR-L2, and a CDR-L3 as contained within a given V_{L} region amino acid sequence and defined by any of the aforementioned schemes, such as Kabat, Chothia, AbM, IgBLAST, IMGT, or Contact method, or other known schemes. In some embodiments, specific CDR sequences are specified. In some embodiments, specific CDR sequences are specified. Exemplary CDR sequences of provided antibodies are described using various numbering schemes (see e.g. Table 1), although it is understood that a provided antibody can include CDRs as described according to any of the other aforementioned numbering schemes or other known numbering schemes.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM, IgBLAST, IMGT, or Contact method, or other known schemes. In other cases, the particular amino acid sequence of a CDR or FR is given.

As used herein, the term “framework” or “FR” residues refers to those variable domain residues other than the hypervariable region residues defined herein as CDR residues. Unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM, IgBLAST, IMGT, or Contact method, or other known schemes. In other cases, the particular amino acid sequence of a CDR or FR is given.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Among the provided antibodies are antibody fragments. An “antibody fragment” or “antigen-binding fragment” refers to a molecule other than an intact or full-length antibody that comprises a portion of an intact or full-length antibody that binds the antigen to which the intact antibody binds. The term thus refers to one or more fragments of an antibody that retain the ability to specifically bind to an antigen (e.g., human CD3). It has been shown that the antigen-binding function of an antibody can be performed by fragments of a full-length antibody. Examples of antibody fragments include but are not limited to Fv, Fab, Fab′, Fab′-SH, F(ab′)₂; diabodies; linear antibodies; V_{H} regions, single-chain antibody molecules such as scFvs and single-domain antibodies comprising only the V_{H} region; and multispecific antibodies formed from antibody fragments. In some embodiments, the antibody is or comprises an antibody fragment comprising a V_{H} and a V_{L} region. In some embodiments, the antibodies are single-chain antibody fragments comprising a heavy chain variable V_{H} region and/or a V_{L} region, such as Fvs or scFvs. In some embodiments, the antibodies are Fab composed of an entire light chain (i.e. V_{L} region and a constant light chain (CL)) and the V_{H} region and the first constant domain of one heavy chain (CH1).

Antibody fragments can be made by various techniques, including but not limited to proteolytic digestion of an intact antibody as well as production by recombinant host cells. For instance, papain digestion of full-length antibodies produces two identical antigen-binding fragments, called “Fab” fragments, and a residual “Fc” fragment, a designation reflecting the ability to crystallize readily. Pepsin treatment of an antibody yields a single large F(ab′)2 fragment which roughly corresponds to two disulfide linked Fab fragments having different antigen-binding activity and is still capable of cross-linking antigen. Fab′ fragments differ from Fab fragments by having a few additional residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known. In some embodiments, the antibodies are recombinantly-produced fragments. For instance, the chains of an antibody or antigen-binding fragment can be incorporated into an expression vector and expressed from a host cell to produce the antibody. In some instances, a recombinantly-produced antibody fragments also can include fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs. For example, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Antigen-binding portions can be produced by recombinant DNA techniques, or by enzymatic or chemical cleavage of intact immunoglobulins.

The term “human antibody”, “human antibody construct” and “human binding domain” includes antibodies, antibody constructs and binding domains having antibody regions such as variable and constant regions or domains which correspond substantially to human germline immunoglobulin sequences known in the art, including, for example, those described by Kabat et al. (1991) (loc. cit.). The human antibodies, antibody constructs or binding domains of the invention may include amino acid residues not encoded by human germline immunoglobulin sequences (e.g., mutations introduced by random or side-specific mutagenesis in vitro or by somatic mutation in vivo), for example in the CDRs, and in particular, in CDR3. The human antibodies, antibody constructs or binding domains can have at least one, two, three, four, five, or more positions replaced with an amino acid residue that is not encoded by the human germline immunoglobulin sequence. The definition of human antibodies, antibody constructs and binding domains as used herein, however, also contemplates “fully human antibodies”, which include only non-artificially and/or genetically altered human sequences of antibodies as those can be derived by using technologies or systems such as the Xenomouse. Preferably, a “fully human antibody” does not include amino acid residues not encoded by human germline immunoglobulin sequences.

Furthermore, the definition of the term “antibody construct” includes monovalent, bivalent and polyvalent/multivalent constructs and, thus, bispecific constructs, the latter which specifically bind to only two antigenic structures, as well as polyspecific/multispecific constructs, which specifically bind more than two antigenic structures, e.g. three, four or more, through distinct binding domains. Moreover, the definition of the term “antibody construct” includes molecules consisting of only one polypeptide chain as well as molecules consisting of more than one polypeptide chain, which chains can be either identical (homodimers, homotrimers or homo oligomers) or different (heterodimer, heterotrimer or heterooligomer). Examples for the above identified antibodies and variants or derivatives thereof are described inter alia in Harlow and Lane, Antibodies a laboratory manual, CSHL Press (1988) and Using Antibodies: a laboratory manual, CSHL Press (1999), Kontermann and Dübel, Antibody Engineering, Springer, 2nd ed. 2010 and Little, Recombinant Antibodies for Immunotherapy, Cambridge University Press 2009.

The term “bispecific” as used herein refers to an antibody construct that comprises at least a first binding domain and a second binding domain, wherein the first binding domain binds to one antigen or target (e.g., CD3 as disclosed herein), and the second binding domain binds to another antigen or target (e.g., a second antigen, such as those described in Section II.B.1). Accordingly, antibody constructs according to the invention comprise specificities for at least two different antigens or targets. A bispecific antibody can be a monoclonal antibody that has binding specificity for two different epitopes. Bispecific antibodies can be prepared as full length antibodies or antibody fragments. Given that the antibody constructs are (at least) bispecific, they do not occur naturally and they are markedly different from naturally occurring products. A “bispecific” antibody construct or immunoglobulin is hence an artificial hybrid antibody or immunoglobulin having at least two distinct binding sides with different specificities. Bispecific antibody constructs can be produced by a variety of methods including fusion of hybridomas or linking of Fab′ fragments. See, e.g., Songsivilai & Lachmann, Clin. Exp. Immunol. 79:315-321 (1990).

The term “target cell surface antigen” refers to an antigenic structure expressed by a cell and which is present at the cell surface such that it is accessible for an antibody construct as described herein. It may be a protein, preferably the extracellular portion of a protein, and/or a carbohydrate structure, preferably a carbohydrate structure of a protein, such as a glycoprotein. It is preferably a tumor antigen.

As used herein, “administering” refers to the physical introduction of a CD3 binding molecule, such as an anti-CD3 antibody, antigen binding portion thereof, bispecific molecule, multispecific molecule or a nucleic acid or expression vector as described herein encoding the same, alone or in combination with another therapeutic agent to a subject, using any of the various methods and delivery systems known to those skilled in the art. Preferred routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intra-lymphatic, intralesional, intracapsular, intra-orbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The terms “treat,” “treating,” and “treatment,” as used herein, refer to any type of intervention or process performed on, or administering an active agent (e.g., an anti-CD3 binding molecule, such as an anti-CD3 antibody and anti-CD3 bispecific antibody, including antibody fragments thereof) to the subject with the objective of preventing, reversing, alleviating, ameliorating, inhibiting, or slowing down or preventing the progression, development, severity or recurrence of a symptom, complication, condition or biochemical indicia associated with a disease. Treatment can be of a subject having a disease or a subject who does not have a disease (e.g., for prophylaxis).

As used herein, the term “subject” includes any human or non-human animal. For example, the methods and compositions described herein can be used to treat a subject having a B cell disorder. The term “non-human animal” includes all vertebrates, e.g., a mammals and non-mammals, such as non-human primates, sheep, cats, dogs, cows, chickens, amphibians, and reptiles.

The terms “detection” or “detected”, as used herein refer to qualitative and/or quantitative detection (measuring levels) with or without reference to a control.

The term “diagnosing”, as used herein, means the determination of the nature of a medical condition intended to identify a pathology which affects the subject from a number of collected data.

The terms “(specifically) binds to”, (specifically) recognizes”, “is (specifically) directed to”, and “(specifically) reacts with” mean in accordance with this invention that a binding domain interacts or specifically interacts with a given epitope or a given target side on the target molecules (antigens), e.g., CD3 as disclosed herein.

The term “epitope” refers to a side on an antigen to which a binding domain, such as an antibody or immunoglobulin, or a derivative, fragment or variant of an antibody or an immunoglobulin, specifically binds. An “epitope” is antigenic and thus the term epitope is sometimes also referred to herein as “antigenic structure” or “antigenic determinant”. Thus, the binding domain is an “antigen interaction side”. Said binding/interaction is also understood to define a “specific recognition”.

“Epitopes” can be formed both by contiguous amino acids or non-contiguous amino acids juxtaposed by tertiary folding of a protein. A “linear epitope” is an epitope where an amino acid primary sequence comprises the recognized epitope. A linear epitope typically includes at least 3 or at least 4, and more usually, at least 5 or at least 6 or at least 7, for example, about 8 to about 10 amino acids in a unique sequence.

A “conformational epitope”, in contrast to a linear epitope, is an epitope wherein the primary sequence of the amino acids comprising the epitope is not the sole defining component of the epitope recognized (e.g., an epitope wherein the primary sequence of amino acids is not necessarily recognized by the binding domain). With regard to recognition of conformational epitopes, the binding domain recognizes a three-dimensional structure of the antigen, preferably a peptide or protein or fragment thereof (in the context of the present invention, the antigenic structure for one of the binding domains is comprised within the target cell surface antigen protein). For example, when a protein molecule folds to form a three-dimensional structure, certain amino acids and/or the polypeptide backbone forming the conformational epitope become juxtaposed enabling the antibody to recognize the epitope. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy.

“Appreciable affinity” includes binding with an affinity of about 10⁻⁶ M (KD) or stronger. Preferably, binding is considered specific when the binding affinity is about 10⁻¹² to 10⁻⁸ M, 10⁻¹² to 10⁻⁹ M, 10⁻¹² to 10⁻¹⁰ M, 10⁻¹¹ to 10⁻⁸ M, preferably of about 10⁻¹¹ to 10⁻⁹ M. Whether a binding domain specifically reacts with or binds to a target can be tested readily by, inter alia, comparing the reaction of said binding domain with a target protein or antigen with the reaction of said binding domain with proteins or antigens other than the CD3. Preferably, a binding domain of the invention does not essentially or substantially bind to proteins or antigens other than CD3 (i.e., the first binding domain is not capable of binding to proteins other than CD3). It is an envisaged characteristic of the antibody constructs according to the present invention to have superior affinity characteristics in comparison to other HLE formats. Such a superior affinity, in consequence, suggests a prolonged half-life in vivo. The longer half-life of the antibody constructs according to the present invention may reduce the duration and frequency of administration which typically contributes to improved patient compliance. This is of particular importance as the antibody constructs of the present invention are particularly beneficial for highly weakened or even treatment resistant B cell disorder patients.

The term “does not essentially/substantially bind” or “is not capable of binding” means that a binding domain of the present invention does not bind a protein or antigen other than the CD3 and the second antigen as described in Section II B 1, i.e., does not show reactivity of more than 30%, preferably not more than 20%, more preferably not more than 10%, particularly preferably not more than 9%, 8%, 7%, 6% or 5% with proteins or antigens other than CD3 and the second antigen, whereby binding to CD3 or the second antigen, respectively, is set to be 100%.

As used herein, “isotype” refers to the antibody class (e.g., IgG (including IgG1, IgG2, IgG3, and IgG4), IgM, IgA (including IgAQ1 and IgA2), IgD, and IgE antibody) that is encoded by the heavy chain constant region genes of the antibody.

An antibody may be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., IgG1, exist in several allotypes, which differ from each other in at most a few amino acids.

As used herein, the term “allotype” refers to naturally occurring variants within a specific isotype group, where the variants differ in a few amino acids. Anti-antibodies described herein can be of any allotype. The mutation of certain residues would modulate (i.e., eliminate or decrease) the binding of the antibodies to Fcγ receptors and/or C1q, and thus reduce activator efficacy of the Fc domain of the IgG component of an antibody.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 80% to 85%, 85% to 90% or 90% to 95%, and more preferably at least about 98% to 99.5% of the nucleotides. Alternatively, substantial homology exists when the segments will hybridize under selective hybridization conditions, to the complement of the strand. For polypeptides, the term “substantial homology” indicates that two polypeptides, or designated sequences thereof, when optimally aligned and compared, are identical, with appropriate amino acid insertions or deletions, in at least about 80% of the amino acids, usually at least about 80% to 85%, 85% to 90%, 90% to 95%, and more preferably at least about 98% to 99.5% of the amino acids.

The percent identity between two sequences is a function of the number of identical positions shared by the sequences (i.e., % identity=# of identical positions/total # of positions×100), considering the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or two amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48): 444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

The term “monoclonal antibody” (mAb) or monoclonal antibody construct as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic side or determinant on the antigen, in contrast to conventional (polyclonal) antibody preparations which typically include different antibodies directed against different determinants (or epitopes). The modifier “monoclonal” indicates the character of the antibody as being obtained from a substantially homogeneous population of antibodies, and is not to be construed as requiring production of the antibody by any particular method. A monoclonal antibody may be made by a variety of techniques, including but not limited to generation from a hybridoma, recombinant DNA methods, phage-display and other antibody display methods.

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

Amino acid modifications include, for example, deletions from, and/or insertions into, and/or substitutions of, residues within the amino acid sequences of the antibody constructs. Any combination of deletion, insertion, and substitution is made to arrive at the final construct, provided that the final construct possesses the desired characteristics. The amino acid changes also may alter post-translational processes of the antibody constructs, such as changing the number or position of glycosylation sites.

For example, 1, 2, 3, 4, 5, or 6 amino acids may be inserted, substituted or deleted in each of the CDRs (of course, dependent on their length), while 1, 2, 3, 4, 5, 6, 7, 8, 9, 10, 11, 12, 13, 14, 15, 16, 17, 18, 19, 20, or 25 amino acids may be inserted, substituted or deleted in each of the FRs. Preferably, amino acid sequence insertions into the binding molecule or antibody construct include amino- and/or carboxyl-terminal fusions ranging in length from 1, 2, 3, 4, 5, 6, 7, 8, 9 or 10 residues to polypeptides containing a hundred or more residues, as well as intra-sequence insertions of single or multiple amino acid residues. Corresponding modifications may also performed within the third domain of the antibody construct of the invention. An insertional variant of the antibody construct of the invention includes the fusion to the N-terminus or to the C-terminus of the antibody construct of an enzyme or the fusion to a polypeptide.

The term “polypeptide” as used herein describes a group of molecules, which usually consist of more than 30 amino acids. Polypeptides may further form multimers such as dimers, trimers and higher oligomers, i.e., consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a hereteromultimer is an antibody molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is affected e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below.

### II. CD3 BINDING MOLECULES

Provided are CD3-binding molecules containing an antibody binding domain that binds CD3 (also called anti-CD3 antibodies), including chimeric or bispecific molecules containing the same. Such antibody binding domains specifically bind to CD3 epsilon (CD3ε) proteins, such as a human CD3ε protein. Among the CD3-binding molecules are or include antibodies (including antigen-binding fragments). In some embodiments, the antibodies or antigen-binding fragments include a heavy chain variable region (V_{H}) and a light chain variable region (V_{L}), including full-length antibodies as well as functional antigen-binding fragments such as Fabs or single chain Fv fragments (scFvs). The antibodies include antibodies that specifically bind to CD3ε, e.g., human CD3ε. Among the provided anti-CD3 antibodies are human antibodies, or antibodies that are modified from or variant of human antibodies. The antibodies include isolated antibodies.

Also among the binding molecules are polypeptides containing such antibodies, including bispecific antibodies. The CD3-binding molecules that incorporate the provided antibodies (e.g., antigen-binding antibody fragments) specifically bind to CD3ε, such as human CD3ε protein. Among the provided bispecific molecules are TCEs that incorporate an anti-CD3 antibody or antigen-binding fragment and antibody for specific targeting of T cells and a binding domain with means for binding an antigen on the surface of a tumor cell (also called a tumor-associated antigen or TAA).

In some aspects, the CD3-binding molecules include isolated molecules.

Also provided are polynucleotides containing nucleic acids sequences encoding all or a portion of any of the provided binding molecules, including any of the provided antibodies or antigen-binding fragments or other binding molecules incorporating the same. The provided polynucleotides can be incorporated into constructs, such as deoxyribonucleic acid (DNA) or ribonucleic acid (RNA) constructs, such as those that can be introduced into cells for expression thereof.

### A. CD3 Antibody Binding Domain

Provided are binding molecules that contain a CD3 antibody binding domain, including full-length antibodies and functional antigen-binding fragments. In some embodiments, the antibodies include antibodies that specifically bind to CD3ε, e.g., human CD3ε. Exemplary antibodies are described below. In some embodiments, the antibody, e.g., the anti-CD3 antibody, such as a full-length antibody or antigen-binding antibody fragment, contains a heavy chain variable region (V_{H}) sequence and a light chain variable region (V_{L}) sequence as described. In some embodiments, the anti-CD3 antibody is an antigen-binding antibody fragment that is a single chain fragment, such as a single chain Fv (scFv) fragment. In some aspects, the scFv comprises a V_{H} region and a V_{L} region. In some embodiments, the anti-CD3 antibody is an antigen-binding antibody fragment that is an scFv fragment containing a V_{H} region and a V_{L} region. In some embodiments, the scFv fragment is a disulfide-stabilized Fv (dsFv). In some embodiments, the anti-CD3 antibody is an antigen-binding antibody fragment that is a Fab. In some embodiments, the antibodies provided herein bind to human CD3ε set forth in SEQ ID NO:82 (UniProt P07766).

The anti-CD3 binding region of the disclosure agonize, stimulate, activate, and/or otherwise augment CD3-mediated T cell activation. Biological activities of CD3 include, for example, T cell activation and other signaling through interaction between CD3 and the antigen-binding subunits of the T-Cell Receptor (TCR). For example, the anti-CD3 binding domains of the disclosure completely or partially activate T cells via engagement of CD3 (e.g. CD3ε) on T cells by partially or completely modulating, e.g., agonizing, stimulating, activating or otherwise augmenting CD3-mediated T cell activation.

In some embodiments, the anti-CD3 binding region includes one or more copies of an antibody or an antigen-binding fragment thereof selected from the group consisting of a Fab fragment, a F(ab′)2 fragment, an Fv fragment, or an a scFv. In some embodiments, the anti-CD3 binding region is monovalent for binding CD3. In some embodiments, the anti-CD3 binding region is a Fab. In some embodiments, the anti-CD3 binding region is a disulfide stabilized Fv fragment.

Among the provided anti-CD3 antibodies are human antibodies.

Among the provided antibodies, e.g., antigen-binding fragments, are human antibodies. In some embodiments of a provided human anti-CD3 antibody, e.g., antigen-binding fragments, the human antibody contains a V_{H} region that comprises a portion having at least 95%, 96%, 97%, 98%, 99%, or 100% sequence identity to an amino acid sequence encoded by a germline nucleotide human heavy chain V segment, a portion having at least 95%, 96%, 97%, 98%, 99%, or 100% sequence identity to an amino acid sequence encoded by a germline nucleotide human heavy chain D segment, and/or a portion having at least 95%, 96%, 97%, 98%, 99%, or 100% sequence identity to an amino acid sequence encoded by a germline nucleotide human heavy chain J segment; and/or contains a V_{L} region that comprises a portion having at least 95%, 96%, 97%, 98%, 99%, or 100% sequence identity to an amino acid sequence encoded by a germline nucleotide human kappa or lambda chain V segment, and/or a portion having at least 95%, 96%, 97%, 98%, 99%, or 100% sequence identity to an amino acid sequence encoded by a germline nucleotide human kappa or lambda chain J segment.

Among the provided antibodies, e.g., antigen-binding fragments, are human antibodies. In some embodiments of a provided human anti-CD3 antibody, e.g., antigen-binding fragments, the human antibody contains a V_{H} region that comprises a portion having at least 95%, 96%, 97%, 98%, 99%, or 100% sequence identity to an amino acid sequence encoded by a germline nucleotide human heavy chain V segment, a portion having at least 95%, 96%, 97%, 98%, 99%, or 100% sequence identity to an amino acid sequence encoded by a germline nucleotide human heavy chain D segment, and/or a portion having at least 95%, 96%, 97%, 98%, 99%, or 100% sequence identity to an amino acid sequence encoded by a germline nucleotide human heavy chain J segment; and contains a V_{L} region that comprises a portion having at least 95%, 96%, 97%, 98%, 99%, or 100% sequence identity to an amino acid sequence encoded by a germline nucleotide human kappa or lambda chain V segment, and/or a portion having at least 95%, 96%, 97%, 98%, 99%, or 100% sequence identity to an amino acid sequence encoded by a germline nucleotide human kappa or lambda chain J segment. In some embodiments, the portion of the V_{H} region corresponds to the CDR-H1, the CDR-H2 and/or the CDR-H3. In some embodiments, the portion of the V_{H} region corresponds to the CDR-H1, the CDR-H2 and the CDR-H3. In some embodiments, the portion of the V_{L} region corresponds to the CDR-L1, the CDR-L2 and/or the CDR-L3. In some embodiments, the portion of the V_{L} region corresponds to the CDR-L1, the CDR-L2 and the CDR-L3.

Among the provided antibodies are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

In some embodiments, the anti-CD3 antibody, e.g., antigen-binding antibody fragment, contains a V_{H} region sequence that contains a heavy chain complementarity determining region 1 (CDR-H1), a CDR-H2 and a CDR-H3 as described and contains a V_{L} region sequence that contains a CDR-L1, a CDR-L2 and/or a CDR-L3 as described. Also among the provided antibodies and fragment thereof are those having sequences at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98%, or at or about 99% identical to such a sequence, e.g., any of the CDR-H1, CDR-H2, CDR-H3, CDR-L1, CDR-L2, CDR-L3 sequences of the antibodies described herein. Also among the provided antibodies and fragment thereof are those having sequences at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98%, or at or about 99% identical to such a sequence, e.g., any of the V_{H} and V_{L} sequences, respectively, of the antibodies described herein.

In some embodiments, provided are anti-CD3 antibodies, including antibody fragments, that have a V_{H} region that comprises a CDR-H1 set forth in SEQ ID NO: 107, a CDR-H2 set forth in SEQ ID NO: 108, and a CDR-H3 set forth in SEQ ID NO: 109; and a VL region that comprises a CDR-L1 set forth in SEQ ID NO: 19, a CDR-L2 set forth in SEQ ID NO: 110, and a CDR-L3 set forth within SEQ ID NO: 6.

In some embodiments, among provided anti-CD3 antibodies, including antibody fragments, include a V_{H} region that comprises an amino acid sequence of the consensus sequence QVQLVESGGGVVQPGX₁SLRLSCAASGFTFSX₂X₃GIHWVRQAPGKX₄LEWVAAIWY DX₅SX₆X₇YYADSVKGRFTISRDNSX₈NTLX₉LQMNSLRAEDTAVYYCARSNX₁₀DX₁₁L TX₁₂X₁₃YX₁₄X₁₅GX₁₆X₁₇VWGQGTTVTVSS, wherein X₁ is R, G, or E; X₂ is R or N; X₃ is Y or H, X₄ is G or C; X₅ is G, A, or S; X₆ is K, H, or N, X₇ is K, T, or D; X₈ is K, E, or S; X₉ is Y or D; X₁₀ is Y or F, X₁₁ is I or V, X₁₂ is R or G; X₁₃ is Y or H; X₁₄ is Q or S; X₁₅ is Y or H; X₁₆ is I or L; and X₁₇ is N or D; (SEQ ID NO: 156).

In some embodiments, among provided anti-CD3 antibodies, including antibody fragments, include a V_{H} region that comprises an amino acid sequence of the consensus sequence QVQLVESGGGVVQPGX₁SLRLSCAASGFTFSX₂X₃GIHWVRQAPGKX₄LEWVAAIWY DX₅SX₆X₇YYADSVKGRFTISRDNSX₈NTLX₉LQMNSLRAEDTAVYYCARSNX₁₀DX₁₁L TX₁₂X₁₃YX₁₄X₁₅GX₁₆X₁₇VWGQGTTVTVSS, wherein X₁ is R, G, or E; X₂ is R or N; X₃ is Y or H, X₄ is G or C; X₅ is G or A or S; X₆ is K, H, or N, X₇ is K, T, or D; X₈ is K, E, or S; X₉ is Y or D; X₁₀ is Y or F, X₁₁ is I or V, X₁₂ is R or G; X₁₃ is Y or H; X₁₄ is Q or S; X₁₅ is Y or H; X₁₆ is I or L; and X₁₇ is N or D; (SEQ ID NO: 105).

In some embodiments, among provided anti-CD3 antibodies, including antibody fragments, include a V_{L} region that comprises an amino acid sequence of the consensus sequence AIQLTQSPSSLSASVGDRVTITCRASQGISSALAWYQQKPGKAPKLLIYDASX₁LEX₂G VPSRFSGSGSGTDFTLTISSLQPEDFATYYCQQFX₃SYPRTFGX₄GTKVEIK, wherein X₁ is S or G, X₂ is N or S; X₃ is K or N; and X₄ is Q or C; (SEQ ID NO: 106).

In some embodiments, the CDRH1 comprises the sequence set forth in set forth in any one of SEQ ID NOs: 9, 12, 111, or 112, the CDR-H2 comprises the sequence set forth in any one of SEQ ID NOs: 10, 13, 15, 17, or 113-117, and the CDR-H3 comprises the sequence set forth in any one of SEQ ID NOs: 11, 14, 16, 18, 118-123. In some embodiments, the CDR-L1 comprises the sequence set forth in SEQ ID NOs: 19, the CDR-L2 comprises the sequence set forth in any one of SEQ ID NOs: 20, 22, 124, or 125, and the CDR-L3 comprises the sequence set forth in any one of SEQ ID NOs: 21 or 23.

In some embodiments, in the CD3 binding domain, the V_{H} region comprises the CDR-H1, the CDR-H2, and the CDR-H3 contained within SEQ ID NO: 24, and the VL region comprises the CDR-L1, the CDR-L2, and the CDR-L3 contained within SEQ ID NO: 39. In some embodiments, in the CD3 binding domain, the VH region comprises the CDR-H1, the CDR-H2, and the CDR-H3 contained within SEQ ID NO: 25, and the VL region comprises the CDR-L1, the CDR-L2, and the CDR-L3 contained within SEQ ID NO: 40. In some embodiments, in the CD3 binding domain, the VH region comprises the CDR-H1, the CDR-H2, and the CDR-H3 contained within SEQ ID NO: 26, and the VL region comprises the CDR-L1, the CDR-L2, and the CDR-L3 contained within SEQ ID NO: 40. In some embodiments, in the CD3 binding domain, the VH region comprises the CDR-H1, the CDR-H2, and the CDR-H3 contained within SEQ ID NO: 27, and the VL region comprises the CDR-L1, the CDR-L2, and the CDR-L3 contained within SEQ ID NO: 41. In some embodiments, in the CD3 binding domain, the VH region comprises the CDR-H1, the CDR-H2, and the CDR-H3 contained within SEQ ID NO: 28, and the VL region comprises the CDR-L1, the CDR-L2, and the CDR-L3 contained within SEQ ID NO: 42. In some embodiments, in the CD3 binding domain, the VH region comprises the CDR-H1, the CDR-H2, and the CDR-H3 contained within SEQ ID NO: 29, and the VL region comprises the CDR-L1, the CDR-L2, and the CDR-L3 contained within SEQ ID NO: 40. In some embodiments, in the CD3 binding domain, the VH region comprises the CDR-H1, the CDR-H2, and the CDR-H3 contained within SEQ ID NO: 30, and the VL region comprises the CDR-L1, the CDR-L2, and the CDR-L3 contained within SEQ ID NO: 41. In some embodiments, in the CD3 binding domain, the VH region comprises the CDR-H1, the CDR-H2, and the CDR-H3 contained within SEQ ID NO: 31, and the VL region comprises the CDR-L1, the CDR-L2, and the CDR-L3 contained within SEQ ID NO: 43. In some embodiments, in the CD3 binding domain, the VH region comprises the CDR-H1, the CDR-H2, and the CDR-H3 contained within SEQ ID NO: 32, and the VL region comprises the CDR-L1, the CDR-L2, and the CDR-L3 contained within SEQ ID NO: 40. In some embodiments, in the CD3 binding domain, the VH region comprises the CDR-H1, the CDR-H2, and the CDR-H3 contained within SEQ ID NO: 33, and the VL region comprises the CDR-L1, the CDR-L2, and the CDR-L3 contained within SEQ ID NO: 40. In some embodiments, in the CD3 binding domain, the VH region comprises the CDR-H1, the CDR-H2, and the CDR-H3 contained within SEQ ID NO: 34, and the VL region comprises the CDR-L1, the CDR-L2, and the CDR-L3 contained within SEQ ID NO: 40. In some embodiments, in the CD3 binding domain, the VH region comprises the CDR-H1, the CDR-H2, and the CDR-H3 contained within SEQ ID NO: 35, and the VL region comprises the CDR-L1, the CDR-L2, and the CDR-L3 contained within SEQ ID NO: 40. In some embodiments, in the CD3 binding domain, the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NO: 40. In some embodiments, in the CD3 binding domain, the VH region comprises the CDR-H1, the CDR-H2, and the CDR-H3 contained within SEQ ID NO: 37, and the VL region comprises the CDR-L1, the CDR-L2, and the CDR-L3 contained within SEQ ID NO: 40. In some embodiments, in the CD3 binding domain, the VH region comprises the CDR-H1, the CDR-H2, and the CDR-H3 contained within SEQ ID NO: 38, and the VL region comprises the CDR-L1, the CDR-L2, and the CDR-L3 contained within SEQ ID NO: 41. In some embodiments, in the CD3 binding domain, the VH region comprises the CDR-H1, CDR-H2 and the CDR-H3 contained within SEQ ID NO: 126, and the VL region comprises the CDR-L1, the CDR-L2, and the CDR-L3 contained within SEQ ID NO: 40. In some embodiments, in the CD3 binding domain, the VH region comprises the CDR-H1, CDR-H2 and the CDR-H3 contained within SEQ ID NO: 4, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 53, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 54, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 55, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 56, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 57, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 58, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 59, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 60, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 61, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 62, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 63, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 64, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 65, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 66, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 67, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 68, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 69, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 70, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 71, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 72, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 73, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 74, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 75, and the VL region comprises the CDR-L1, the CDR-L2, and the CDR-L3 contained within SEQ ID NOs: 39, 40, or 41. In some embodiments, in the CD3 binding domain, the VH region comprises the CDR-H1, CDR-H2 and the CDR-H3 contained within SEQ ID NO: 24, and the VL region comprises the CDR-L1, the CDR-L2, and the CDR-L3 contained within SEQ ID NOs: 40. In some embodiments, in the CD3 binding domain, the VH region comprises the CDR-H1, CDR-H2 and the CDR-H3 contained within SEQ ID NO: 24, and the VL region comprises the CDR-L1, the CDR-L2, and the CDR-L3 contained within SEQ ID NOs: 77. In some embodiments, in the CD3 binding domain, the VH region comprises the CDR-H1, CDR-H2 and the CDR-H3 contained within SEQ ID NO: 24, and the VL region comprises the CDR-L1, the CDR-L2, and the CDR-L3 contained within SEQ ID NOs: 78. In some embodiments, in the CD3 binding domain, the VH region comprises the CDR-H1, CDR-H2 and the CDR-H3 contained within SEQ ID NO: 24, and the VL region comprises the CDR-L1, the CDR-L2, and the CDR-L3 contained within SEQ ID NOs: 79. In some embodiments, in the CD3 binding domain, the VH region comprises the CDR-H1, CDR-H2 and the CDR-H3 contained within SEQ ID NO: 24, and the VL region comprises the CDR-L1, the CDR-L2, and the CDR-L3 contained within SEQ ID NOs: 80. In some embodiments, in the CD3 binding domain, the VH region comprises the CDR-H1, the CDR-H2, and the CDR-H3 contained within SEQ ID NO: 27, and the VL region comprises the CDR-L1, the CDR-L2, and the CDR-L3 contained within SEQ ID NOs: 39. In some embodiments, in the CD3 binding domain, the VH region comprises the CDR-H1, the CDR-H2, and the CDR-H3 contained within SEQ ID NO: 25, and the VL region comprises the CDR-L1, the CDR-L2, and the CDR-L3 contained within SEQ ID NOs: 39. In some embodiments, in the CD3 binding domain, the VH region comprises the CDR-H1, the CDR-H2, and the CDR-H3 contained within SEQ ID NO: 26, and the VL region comprises the CDR-L1, the CDR-L2, and the CDR-L3 contained within SEQ ID NOs: 39.

In some embodiments, the VH region comprises the CDR-H1, the CDR-H2, and the CDR-H3 comprising the sequence set forth in SEQ ID NOs: 9, 10, and 11, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, the CDR-H2, and the CDR-H3 comprising the sequence set forth in SEQ ID NOs: 12, 13, and 11, respectively, and the VL region comprises the CDR-L1, the CDR-L2, and the CDR-L3 comprising the sequence set forth in SEQ ID NOs: 19, 22, and 21, respectively. In some embodiments, the VH region comprises the CDR-H1, the CDR-H2, and the CDR-H3 comprising the sequence set forth in SEQ ID NOs: 9, 13, and 14, respectively, and the VL region comprises the CDR-L1, the CDR-L2, and the CDR-L3 comprising the sequence set forth in SEQ ID NOs: 19, 22, and 21, respectively. In some embodiments, the VH region comprises the CDR-H1, the CDR-H2, and the CDR-H3 comprising the sequence set forth in SEQ ID NOs: 9, 13, and 11, respectively, and the VL region comprises the CDR-L1, the CDR-L2, and the CDR-L3 comprising the sequence set forth in SEQ ID NOs: 19, 22, and 23, respectively. In some embodiments, the VH region comprises the CDR-H1, the CDR-H2, and the CDR-H3 comprising the sequence set forth in SEQ ID NOs: 9, 15, and 14, respectively, and the VL region comprises the CDR-L1, the CDR-L2, and the CDR-L3 comprising the sequence set forth in SEQ ID NOs: 19, 22, and 21, respectively. In some embodiments, the VH region comprises the CDR-H1, the CDR-H2, and the CDR-H3 comprising the sequence set forth in SEQ ID NOs: 9, 15, and 16, respectively, and the VL region comprises the CDR-L1, the CDR-L2, and the CDR-L3 comprising the sequence set forth in SEQ ID NOs: 19, 22, and 23, respectively. In some embodiments, the VH region comprises the CDR-H1, the CDR-H2, and the CDR-H3 comprising the sequence set forth in SEQ ID NOs: 9, 13, and 14, respectively, and the VL region comprises the CDR-L1, the CDR-L2, and the CDR-L3 comprising the sequence set forth in SEQ ID NOs: 19, 22, and 23, respectively. In some embodiments, the VH region comprises the CDR-H1, the CDR-H2, and the CDR-H3 comprising the sequence set forth in SEQ ID NOs: 9, 17, and 14, respectively, and the VL region comprises the CDR-L1, the CDR-L2, and the CDR-L3 comprising the sequence set forth in SEQ ID NOs: 19, 22, and 21, respectively. In some embodiments, the VH region comprises the CDR-H1, the CDR-H2, and the CDR-H3 comprising the sequence set forth in SEQ ID NOs: 9, 15, and 14, respectively, and the VL region comprises the CDR-L1, the CDR-L2, and the CDR-L3 comprising the sequence set forth in SEQ ID NOs: 19, 22, and 23, respectively. In some embodiments, the VH region comprises the CDR-H1, the CDR-H2, and the CDR-H3 comprising the sequence set forth in SEQ ID NOs: 9, 15, and 18, respectively, and the VL region comprises the CDR-L1, the CDR-L2, and the CDR-L3 comprising the sequence set forth in SEQ ID NOs: 19, 22, and 23, respectively. In some embodiments, the VH region comprises the CDR-H1, CDR-H2 and the CDR-H3 comprising the sequence set forth in SEQ ID NOs: 9, 10, and 11, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 9, 113, and 11, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 9, 13, and 11, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 9, 114, and 11, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 9, 115, and 11, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 9, 10, and 118, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 9, 10, and 18, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 9, 10, and 119, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 9, 10, and 120, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 9, 10, and 16, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 12, 10, and 11, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 111, 10, and 11, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 112, 10, and 11, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 9, 10, and 121, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 9, 10, and 122, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 9, 10, and 123, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 9, 10, and 14, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 9, 116, and 11, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 9, 117, and 11, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 9, 13, and 11, respectively, and the VL region comprises the CDR-L1, the CDR-L2, and the CDR-L3 comprising the sequence set forth in SEQ ID NOs: 19, 20, and 21, respectively. In some embodiments, the CDR-H1, the CDR-H2, and the CDR-H3 comprising the sequence set forth in SEQ ID NOs: 9, 13, and 18, respectively, and the VL region comprises the CDR-L1, the CDR-L2, and the CDR-L3 comprising the sequence set forth in SEQ ID NOS: 19, 20, and 21, respectively. In some embodiments, the VH region comprises the CDR-H1, CDR-H2 and the CDR-H3 comprising the sequence set forth in SEQ ID NOs: 9, 13, and 119, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 111, 13, and 11, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 9, 113, and 123, respectively, and the VL region comprises the CDR-L1, the CDR-L2, and the CDR-L3 comprising the sequence set forth in SEQ ID NOs: 19, 20, and 21, respectively. In some embodiments, the VH region comprises the CDR-H1, CDR-H2 and the CDR-H3 comprising the sequence set forth in SEQ ID NOs: 9, 10, and 11, respectively, and the VL region comprises the CDR-L1, the CDR-L2, and the CDR-L3 comprising the sequence set forth in SEQ ID NOs: 19, 22, and 21, respectively. In some embodiments, the VH region comprises the CDR-H1, CDR-H2 and the CDR-H3 comprising the sequence set forth in SEQ ID NOs: 9, 10, and 11, respectively, and the VL region comprises the CDR-L1, the CDR-L2, and the CDR-L3 comprising the sequence set forth in SEQ ID NOs: 19, 20, and 23, respectively. In some embodiments, the VH region comprises the CDR-H1, CDR-H2 and the CDR-H3 comprising the sequence set forth in SEQ ID NOs: 9, 10, and 11, respectively, and the VL region comprises the CDR-L1, the CDR-L2, and the CDR-L3 comprising the sequence set forth in SEQ ID NOs: 19, 22, and 23, respectively. In some embodiments, the VH region comprises the CDR-H1, CDR-H2 and the CDR-H3 comprising the sequence set forth in SEQ ID NOs: 9, 10, and 11, respectively, and the VL region comprises the CDR-L1, the CDR-L2, and the CDR-L3 comprising the sequence set forth in SEQ ID NOs: 19, 124, and 21, respectively. In some embodiments, or the VH region comprises the CDR-H1, CDR-H2 and the CDR-H3 comprising the sequence set forth in SEQ ID NOs: 9, 10, and 11, respectively, and the VL region comprises the CDR-L1, the CDR-L2, and the CDR-L3 comprising the sequence set forth in SEQ ID NOs: 19, 125, and 23, respectively. In some embodiments, the CDR-H1, CDR-H2 and the CDR-H3 comprising the sequence set forth in SEQ ID NOs: 9, 13, and 16, respectively, and VL region comprises the CDR-L1, the CDR-L2, and the CDR-L3 comprising the sequence set forth in SEQ ID NOs: 19, 20, and 21, respectively. In some embodiments, the CDR-H1, CDR-H2 and the CDR-H3 comprising the sequence set forth in SEQ ID NOs: 12, 13, and 11, respectively, and VL region comprises the CDR-L1, the CDR-L2, and the CDR-L3 comprising the sequence set forth in SEQ ID NOs: 19, 20, and 21, respectively. In some embodiments, the CDR-H1, CDR-H2 and the CDR-H3 comprising the sequence set forth in SEQ ID NOs: 9, 13, and 14, respectively, and VL region comprises the CDR-L1, the CDR-L2, and the CDR-L3 comprising the sequence set forth in SEQ ID NOs: 19, 20, and 21, respectively.

In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 25, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 26, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 27, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 28, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 42. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 29, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 30, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41. In some embodiments, in the CD3 binding domain: de VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 31, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 32, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 33, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 34, and the VL region comprises an amino acid sequence that has at least at or about 85%, 86%, 87%, 88%, 89%, 90%, 91%, 92%, 93%, 94%, 95%, 96%, 97%, 98% or 99% sequence identity to ID NO: 40. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 35, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 38, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 126, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 4, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 53, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 54, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 55, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 56, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 57, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 58, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 59, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 60, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 61, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 62, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 63, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 64, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 65, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 66, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 67, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 68, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 69, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 70, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 71, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 72, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 73, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 74, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 75, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 99. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 100. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 101. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 27, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, in the CD3 binding domain: the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 26, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39.

In some embodiments, in the CD3 binding domain, the VH region and the VL region comprise the sequences set forth in SEQ ID NO: 24 and 39, respectively. In some embodiments, in the CD3 binding domain, the VH region and the VL region comprise the sequences set forth in SEQ ID NO: 24 and 40, respectively. In some embodiments, in the CD3 binding domain, the VH region and the VL region comprise the sequences set forth in SEQ ID NO: 26 and 40, respectively. In some embodiments, in the CD3 binding domain, the VH region and the VL region comprise the sequences set forth in SEQ ID NO: 27 and 41, respectively. In some embodiments, in the CD3 binding domain, the VH region and the VL region comprise the sequences set forth in SEQ ID NO: 28 and 42, respectively. In some embodiments, in the CD3 binding domain, the VH region and the VL region comprise the sequences set forth in SEQ ID NO: 29 and 40, respectively. In some embodiments, in the CD3 binding domain, the VH region and the VL region comprise the sequences set forth in SEQ ID NO: 30 and 41, respectively. In some embodiments, in the CD3 binding domain, the VH region and the VL region comprise the sequences set forth in SEQ ID NO: 31 and 43, respectively. In some embodiments, in the CD3 binding domain, the VH region and the VL region comprise the sequences set forth in SEQ ID NO: 32 and 40, respectively. In some embodiments, in the CD3 binding domain, the VH region and the VL region comprise the sequences set forth in SEQ ID NO: 33 and 40, respectively. In some embodiments, in the CD3 binding domain, the VH region and the VL region comprise the sequences set forth in SEQ ID NO: 34 and 40, respectively. In some embodiments, in the CD3 binding domain, the VH region and the VL region comprise the sequences set forth in SEQ ID NO: 35 and 40, respectively. In some embodiments, in the CD3 binding domain, the VH region and the VL region comprise the sequences set forth in SEQ ID NO: 36 and 40, respectively. In some embodiments, in the CD3 binding domain, the VH region and the VL region comprise the sequences set forth in SEQ ID NO: 37 and 40, respectively. In some embodiments, in the CD3 binding domain, or the VH region and the VL region comprise the sequences set forth in SEQ ID NO: 38 and 41, respectively. In some embodiments, in the CD3 binding domain, the VH region and the VL region comprise the sequences set forth in SEQ ID NO: 126 and 40, respectively. In some embodiments, in the CD3 binding domain, the VH region and the VL region comprise the sequences set forth in SEQ ID NO: 60 and 39, respectively. In some embodiments, in the CD3 binding domain, the VH region and the VL region comprise the sequences set forth in SEQ ID NO: 61 and 39, respectively. In some embodiments, in the CD3 binding domain, the VH region and the VL region comprise the sequences set forth in SEQ ID NO: 62 and 39, respectively. In some embodiments, in the CD3 binding domain, the VH region and the VL region comprise the sequences set forth in SEQ ID NO: 63 and 39, respectively. In some embodiments, in the CD3 binding domain, the VH region and the VL region comprise the sequences set forth in SEQ ID NO: 64, and 39, respectively. In some embodiments, in the CD3 binding domain, the VH region and the VL region comprise the sequences set forth in SEQ ID NO: 65 and 39, respectively. In some embodiments, in the CD3 binding domain, the VH region and the VL region comprise the sequences set forth in SEQ ID NO: 66 and 39, respectively. In some embodiments, in the CD3 binding domain, the VH region and the VL region comprise the sequences set forth in SEQ ID NO: 67 and 39, respectively. In some embodiments, in the CD3 binding domain, the VH region and the VL region comprise the sequences set forth in SEQ ID NO: 68 and 39, respectively. In some embodiments, in the CD3 binding domain, the VH region and the VL region comprise the sequences set forth in SEQ ID NO: 69 and 39, respectively. In some embodiments, in the CD3 binding domain, the VH region and the VL region comprise the sequences set forth in SEQ ID NO: 70 and 39, respectively. In some embodiments, in the CD3 binding domain, the VH region and the VL region comprise the sequences set forth in SEQ ID NO: 71 and 39, respectively. In some embodiments, in the CD3 binding domain, the VH region and the VL region comprise the sequences set forth in SEQ ID NO: 72 and 39, respectively. In some embodiments, in the CD3 binding domain, the VH region and the VL region comprise the sequences set forth in SEQ ID NO: 73 and 39, respectively. In some embodiments, in the CD3 binding domain, the VH region and the VL region comprise the sequences set forth in SEQ ID NO: 74 and 39, respectively. In some embodiments, in the CD3 binding domain, the VH region and the VL region comprise the sequences set forth in SEQ ID NO: 75 and 39, respectively. In some embodiments, in the CD3 binding domain, the VH region and the VL region comprise the sequences set forth in SEQ ID NO: 24 and 40, respectively. In some embodiments, in the CD3 binding domain, the VH region and the VL region comprise the sequences set forth in SEQ ID NO: 24 and 77, respectively. In some embodiments, in the CD3 binding domain, the VH region and the VL region comprise the sequences set forth in SEQ ID NO: 24 and 81, respectively. In some embodiments, in the CD3 binding domain, the VH region and the VL region comprise the sequences set forth in SEQ ID NO: 24 and 79, respectively. In some embodiments, in the CD3 binding domain. In some embodiments, in the CD3 binding domain, the VH region and the VL region comprise the sequences set forth in SEQ ID NO: 24 and 80, respectively. In some embodiments, in the CD3 binding domain, the VH region and the VL region comprise the sequences set forth in SEQ ID NO: 4 and 39, respectively. In some embodiments, in the CD3 binding domain, the VH region and the VL region comprise the sequences set forth in SEQ ID NO: 83 and 39, respectively. In some embodiments, in the CD3 binding domain, the VH region and the VL region comprise the sequences set forth in SEQ ID NO: 54 and 39, respectively. In some embodiments, in the CD3 binding domain, the VH region and the VL region comprise the sequences set forth in SEQ ID NO: 55 and 39, respectively. In some embodiments, in the CD3 binding domain, the VH region and the VL region comprise the sequences set forth in SEQ ID NO: 56 and 39, respectively. In some embodiments, in the CD3 binding domain, the VH region and the VL region comprise the sequences set forth in SEQ ID NO: 57 and 39, respectively. In some embodiments, in the CD3 binding domain, the VH region and the VL region comprise the sequences set forth in SEQ ID NO: 58 and 39, respectively. In some embodiments, in the CD3 binding domain, the VH region and the VL region comprise the sequences set forth in SEQ ID NO: 59 and 39, respectively. In some embodiments, in the CD3 binding domain, the VH region and the VL region comprise the sequences set forth in SEQ ID NO: 27 and 39, respectively. In some embodiments, in the CD3 binding domain, the VH region and the VL region comprise the sequences set forth in SEQ ID NO: 24 and 39, respectively. In some embodiments, in the CD3 binding domain, the VH region and the VL region comprise the sequences set forth in SEQ ID NO: 26 and 39, respectively.

The disclosed binding molecules include anti-CD3 antibodies, including antigen binding fragments, comprising specified paratope resides that interact with the human CD3 epsilon. For example, in certain embodiments, the antigen binding domain that specifically binds human CD3 epsilon comprises a heavy chain variable region having at least one amino acid, in the specified position selected from the group consisting of G33, H35, 151, W52, Y53, S99, Y101, I103, R106, Q109, and Y110 of SEQ ID NO:24 and a light chain variable region having at least one amino acid in the specified position selected from the group consisting of S30, F91, K92, Y94, and R96 of SEQ ID NO:39.

In another embodiment, the antigen binding domain that specifically binds human CD3 epsilon comprises a heavy chain variable region having amino acids in the specified positions of G33, H35, 151, W52, Y53, S99, Y101, I103, R106, Q109, and Y110 of SEQ ID NO: 24 and a light chain variable region having amino acids in the specified positions of S30, F91, K92, Y94, and R96 of SEQ ID NO:39.

In some embodiments, provided are anti-CD3 antibodies, including antibody fragments, that have a V_{H} region that comprises a -H1 set forth in SEQ ID NO: 1, a CDR-H2 set forth in SEQ ID NO: 2, and a CDR-H3 set forth in SEQ ID NO:3; and a VL region that comprises a CDR-L1 set forth in SEQ ID NO: 19, a CDR-L2 set forth in SEQ ID NO: 5, and a CDR-L3 set forth within SEQ ID NO: 6.

In some embodiments, among provided anti-CD3 antibodies, including antibody fragments, include a V_{H} region that comprises a sequence of the consensus sequence QVQLVESGGGVVQPGX₁SLRLSCAASGFTFSX₂YGIHWVRQAPGKX₃LEWVAAIWYD X₄SKX₅YYADSVKGRFTISRDNSX₆NTLX₇LQMNSLRAEDTAVYYCARSNYDILTX₈X₉YX₁₀X₁₁GX₁₂X₁₃VWGQGTTVTVSS, wherein X₁ is R, G, or E; X₂ is R or N; X₃ is G or C; X₄ is G or A; X₅ is K, T, or D; X₆ is K, E, or S; X₇ is Y or D; X₈ is R or G; X₉ is Y or H; X₁₀ is Q or S; X₁₁ is Y or H; X₁₂ is I or L; and X₁₃ is N or D; (SEQ ID NO:7).

In some embodiments, among provided anti-CD3 antibodies, including antibody fragments, include a V_{L} region that comprises a sequence of the consensus sequence AIQLTQSPSSLSASVGDRVTITCRASQGISSALAWYQQKPGKAPKLLIYDASSLEX₁GV PSRFSGSGSGTDFTLTISSLQPEDFATYYCQQFX₂SYPRTFGX₃GTKVEIK, wherein X₁ is N or S; X₂ is K or N; X₃ is Q or C; (SEQ ID NO:8).

In some embodiments, provided anti-CD3 antibodies, including antibody fragments, comprises a V_{H} region comprising a CDR-H1, a CDR-H2 and a CDR-H3 present in (or contained in) a V_{H} sequence set forth in any one of SEQ ID NOs: 24-38, and a V_{L} region comprising CDR-L1, a CDR-L2, and a CDR-L3 present in (or contained in) a V_{L} sequence set forth in any one of SEQ ID NO:39-43.

In some embodiments, the V_{H} region of an antibody or antigen-binding fragment thereof comprises a CDR-H1, a CDR-H2 and a CDR-H3 according to Kabat numbering. In some embodiments, the V_{L} region of an antibody or antigen-binding fragment thereof comprises a CDR-L1, a CDR-L2 and a CDR-L3 according to Kabat numbering.

In some embodiments, the CDRH1 comprises the amino acid sequence set forth in set forth in any one of SEQ ID NOs: 9 or 12, the CDR-H2 comprises the amino acid sequence set forth in any one of SEQ ID NOs: 10, 13, 15, or 17, and the CDR-H3 comprises the amino acid sequence set forth in any one of SEQ ID NOs: 11, 14, 16, or 18. In some embodiments, the CDR-L1 comprises the amino acid sequence set forth in SEQ ID NOs: 19, the CDR-L2 comprises the amino acid sequence set forth in any one of SEQ ID NOs: 20 or 22, and the CDR-L3 comprises the amino acid sequence set forth in any one of SEQ ID NOs: 21 or 23.

In some embodiments, the V_{H} region comprises the CDR-H1, the CDR-H2, and the CDR-H3 contained within SEQ ID NO: 24, and the V_{L} region comprises the CDR-L1, the CDR-L2, and the CDR-L3 contained within SEQ ID NO: 39. In some embodiments, the V_{H} region comprises the CDR-H1, the CDR-H2, and the CDR-H3 comprising the sequence set forth in SEQ ID NOs: 9, 10, and 11, respectively, and the V_{L} region comprises the CDR-L1, the CDR-L2, and the CDR-L3 comprising the sequence set forth in SEQ ID NOs: 19, 20, and 21, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, the V_{H} region and the V_{L} region comprise the sequences set forth in SEQ ID NO: 24 and 39, respectively. In some embodiments, the CD3 binding domain comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 128. In some embodiments, the binding domain comprises an the amino acid sequence of SEQ ID NO: 128.

In some embodiments, among provided binding molecules provided herein are binding molecules in which an antibody, including antigen binding fragment, disclosed herein may comprise one or more amino acid substitutions, insertions and/or deletions in the framework and/or CDR regions of the heavy and light chain variable domains as compared to a reference human antibody or compared to the corresponding germline sequences from which the antibodies were derived. Such mutations can be readily ascertained by comparing the amino acid sequences disclosed herein to the reference sequence or to germline sequences available from, for example, public antibody sequence databases.

In some embodiments, the binding molecules provided herein include antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FRI or within the last 8 amino acids of FR4, or only the mutated residues found within CDR1, CDR2or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

In some embodiments, the binding molecules provided herein include antibodies, and antigen-binding fragments thereof, comprising variants in which the framework and/or CDR regions are mutated with one or more conservative substitutions. For example, the present invention includes antibodies and antibody fragments having framework and/or CDR regions amino acid sequences with, e.g., 10 or fewer, 8 or fewer, 6 or fewer, 4 or fewer, etc. conservative amino acid substitutions relative to any of the framework and/or CDR regions amino acid sequences set forth in the tables herein.

In some embodiments, the anti-CD3 antibody can be chosen as having a particular or desired binding affinity. In some embodiments, the anti-CD3 antibody has a CD3 dissociation constant (Kd) of <1 μM, <100 nM, <10 nM, <1 nM, <0.1 nM, <0.01 nM, or <0.001 nM (e.g., 10⁻⁸ M or less, e.g., from 10⁻⁸ M to 10⁻¹³ M, e.g., from 10⁻⁹ M to 10⁻¹³ M). In some embodiments, the provided binding molecules, including bispecific antibodies, incorporate anti-CD3 antibodies that exhibit a medium binding affinity for binding to CD3. In some embodiments, an anti-CD3 antibody exhibits a binding affinity (Kd) that is greater than 5 nM. In some embodiments, an anti-CD3 antibody incorporated in bispecific antibodies herein exhibits a binding affinity (Kd) that is greater than 10 nM. In some embodiments, the Kd of an anti-CD3 antibody is at or about 5 nM, 7.5 nM, 10 nM, 15 nM, 20 nM, 25 nM, 30 nM, 40 nM, 50 nM, 60 nM, 70 nM, 80 nM, 90 nM, 100 nM, or any value between any of the foregoing. In some embodiments, the Kd of an anti-CD3 antibody is between 10 nM and 70 nM.

In a particular embodiments, among provided antibodies are antibodies, including antigen binding fragments, that comprise one or more amino acid substitution in the framework and/or CDR region relative to the antibody designated CD3-1, for example, an anti-CD3 antibody comprising in which the V_{H} region and the V_{L} region comprise the sequences set forth in SEQ ID NO: 24 and 39, respectively.

In some embodiments, the V_{H} region comprises a CDR-H1 set forth in SEQ ID NO: 1, a CDR-H2 set forth in SEQ ID NO: 85, and a CDR-H3 set forth in SEQ ID NO:3; and the V_{L} region comprises a CDR-L1 set forth in SEQ ID NO: 19, a CDR-L2 set forth in SEQ ID NO: 22, and a CDR-L3 set forth within SEQ ID NO: 6.

In some embodiments, among provided anti-CD3 antibodies, including antibody fragments, include a V_{H} region that comprises a sequence of the consensus sequence QVQLVESGGGVVQPGX₁SLRLSCAASGFTFSX₂YGIHWVRQAPGKX₃LEWVAAIWYD ASKX₄YYADSVKGRFTISRDNSX₅NTLYLQMNSLRAEDTAVYYCARSNYDILTX₆X₇Y X₈X₉GX₁₀X₁₁VWGQGTTVTVSS, wherein X₁=R or G or E; X₂=R or N; X₃-G or C; X₄=K or T or D; X₅=K or E or S; X₆=R or G; X₇=Y or H; X₈=Q or S; X₉=Y or H; X₁₀=I or L; and X₁₁=N or D (SEQ ID NO: 83).

In some embodiments, among provided anti-CD3 antibodies, including antibody fragments, include a light chain variable (V_{L}) region that comprises a sequence of the consensus sequence AIQLTQSPSSLSASVGDRVTITCRASQGISSALAWYQQKPGKAPKLLIYDASSLESGVP SRFSGSGSGTDFTLTISSLQPEDFATYYCQQFX₁SYPRTFGX₂GTKVEIK (SEQ ID NO: 84), wherein X₁=K or N and X₂=Q or C.

In some embodiments, the V_{H} region comprises one or more amino acid residues selected from the group consisting of: an asparagine (N) at position 31, an alanine (A) at position 55, a tyrosine (Y) at position 80, a glycine (G) at position 106, a histidine (H) at position 107, a serine(S) at position 109, a histidine (H) at position 110, a leucine (L) at position 112, and an aspartic acid (D) at position 113 or any combination thereof, wherein the numbering is relative to SEQ ID NO: 24. In some embodiments, the VL region comprises one or more amino acid residues selected from the group consisting of: a serine(S) at position 56 and an asparagine (N) at position 92, a cysteine (C) at position 100, and an asparagine (N) at position 108, or any combination thereof, wherein the numbering is relative to SEQ ID NO: 39. In some embodiments, the V_{H} region comprises one or more amino acid residues selected from the group consisting of: an asparagine (N) at position 31, an alanine (A) at position 55, a tyrosine (Y) at position 80, a glycine (G) at position 106, a histidine (H) at position 107, a serine(S) at position 109, a histidine (H) at position 110, a leucine (L) at position 112, and an aspartic acid (D) at position 113 or any combination thereof, wherein the numbering is relative to SEQ ID NO: 24, and the VL region comprises one or more amino acid residues selected from the group consisting of: a serine(S) at position 56 and an asparagine (N) at position 92, or any combination thereof, wherein the numbering is relative to SEQ ID NO: 39. In some of any such embodiments, the Kd of an anti-CD3 antibody is between 10 nM and 70 nM, such as 10 nM, 15 nM, 20 nM, 25 nM, 30 nM, 40 nM, 50 nM, 60 nM, or 70 nM, or any value between any of the foregoing.

In some embodiments, the V_{H} region comprises amino acid residues alanine (A) at position 55 and tyrosine (Y) at position 80, wherein the numbering is relative to SEQ ID NO: 24.

In some embodiments, the V_{H} region comprises amino acid residues asparagine (N) at position 31, alanine (A) at position 55, and tyrosine (Y) at position 80, wherein the numbering is relative to SEQ ID NO: 24. In some embodiments, the V_{H} region comprises amino acid residues alanine (A) at position 55, tyrosine (Y) at position 80, leucine (L) at position 112, and aspartic acid (D) at position 113, wherein the numbering is relative to SEQ ID NO: 24. In some embodiments, the V_{H} region comprises amino acid residues alanine (A) at position 55, tyrosine (Y) at position 80, serine(S) at position 109, and histidine (H) at position 110, wherein the numbering is relative to SEQ ID NO: 24. In some embodiments, the VL region comprises serine(S) at position 56, wherein the numbering is relative to SEQ ID NO: 39. In some embodiments, the VL region comprises serine(S) at position 56 and asparagine (N) at position 92, wherein the numbering is relative to SEQ ID NO: 39. In some embodiments, the V_{H} region comprises amino acid residues asparagine (N) at position 31, alanine (A) at position 55, and tyrosine (Y) at position 80, wherein the numbering is relative to SEQ ID NO: 24, and the VL region comprises serine(S) at position 56, wherein the numbering is relative to SEQ ID NO:39. In some embodiments, the V_{H} region comprises amino acid residues alanine (A) at position 55, tyrosine (Y) at position 80, leucine (L) at position 112, and aspartic acid (D) at position 113, wherein the numbering is relative to SEQ ID NO: 24, and the VL region comprises serine(S) at position 56, wherein the numbering is relative to SEQ ID NO:39. In some embodiments, the V_{H} region comprises amino acid residues alanine (A) at position 55, tyrosine (Y) at position 80, serine(S) at position 109, and histidine (H) at position 110, wherein the numbering is relative to SEQ ID NO: 24, and the VL region comprises serine(S) at position 56 and asparagine (N) at position 92, wherein the numbering is relative to SEQ ID NO: 39. In some of any such embodiments, the Kd of an anti-CD3 antibody is between 10 nM and 70 nM, such as 10 nM, 15 nM, 20 nM, 25 nM, 30 nM, 40 nM, 50 nM, 60 nM, or 70 nM, or any value between any of the foregoing. In some of any such embodiments, the anti-CD3 antibody has a Kd of 5 to 75 nM to CD3. In some of any such embodiments, the anti-CD3 antibody has a Kd of 10 to 70 nM to CD3.

In some embodiments, the V_{H} region comprises the CDR-H1, the CDR-H2, and the CDR-H3 contained within SEQ ID NO: 25, and the V_{L} region comprises the CDR-L1, the CDR-L2, and the CDR-L3 contained within SEQ ID NO: 40. In some embodiments, the V_{H} region comprises the CDR-H1, the CDR-H2, and the CDR-H3 comprising the sequence set forth in SEQ ID NOs: 12, 13 and 11, respectively, and the V_{L} region comprises the CDR-L1, the CDR-L2, and the CDR-L3 comprising the sequence set forth in SEQ ID NOs: 19, 22 and 21, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 25, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, the V_{H} region comprises amino acid residues asparagine (N) at position 31, alanine (A) at position 55, and tyrosine (Y) at position 80, wherein the numbering is relative to SEQ ID NO: 24, and the VL region comprises serine(S) at position 56, wherein the numbering is relative to SEQ ID NO:39. In some embodiments, the V_{H} region and the V_{L} region comprise the sequences set forth in SEQ ID NO: 25 and 40, respectively. In some embodiments, the binding domain comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 129. In some embodiments, the binding domain comprises an the amino acid sequence of SEQ ID NO: 129.

In some embodiments, the V_{H} region comprises the CDR-H1, the CDR-H2, and the CDR-H3 contained within SEQ ID NO: 26, and the V_{L} region comprises the CDR-L1, the CDR-L2, and the CDR-L3 contained within SEQ ID NO: 40. In some embodiments, the V_{H} region comprises the CDR-H1, the CDR-H2, and the CDR-H3 comprising the sequence set forth in SEQ ID NOs: 9, 13 and 14, respectively, and the V_{L} region comprises the CDR-L1, the CDR-L2, and the CDR-L3 comprising the sequence set forth in SEQ ID NOs: 19, 22 and 21, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 26, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, the V_{H} region comprises amino acid residues alanine (A) at position 55, tyrosine (Y) at position 80, leucine (L) at position 112, and aspartic acid (D) at position 113, wherein the numbering is relative to SEQ ID NO: 24, and the VL region comprises serine(S) at position 56, wherein the numbering is relative to SEQ ID NO:39. In some embodiments, the V_{H} region and the V_{L} region comprise the sequences set forth in SEQ ID NO: 26 and 40, respectively.

In some embodiments, the V_{H} region comprises the CDR-H1, the CDR-H2, and the CDR-H3 contained within SEQ ID NO: 27, and the V_{L} region comprises the CDR-L1, the CDR-L2, and the CDR-L3 contained within SEQ ID NO: 41. In some embodiments, the V_{H} region comprises the CDR-H1, the CDR-H2, and the CDR-H3 comprising the sequence set forth in SEQ ID NOs: 9, 13 and 11, respectively, and the V_{L} region comprises the CDR-L1, the CDR-L2, and the CDR-L3 comprising the sequence set forth in SEQ ID NOs: 19, 22 and 23, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 27, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41. In some embodiments, the V_{H} region comprises amino acid residues alanine (A) at position 55, tyrosine (Y) at position 80, serine(S) at position 109, and histidine (H) at position 110, wherein the numbering is relative to SEQ ID NO: 24, and the V_{L} region comprises serine(S) at position 56 and asparagine (N) at position 108, wherein the numbering is relative to SEQ ID NO: 39. In some embodiments, the V_{H} region and the V_{L} region comprise the sequences set forth in SEQ ID NO: 27 and 41, respectively.

In some embodiments, the provided anti-CD3 antibodies or antigen-binding fragments thereof low polyreactivity. As used herein, an antibody or antigen-binding fragment thereof that exhibits “low polyreactivity” exhibits low non-specific binding to extracellular matrix proteins. Thus, in some embodiments, the provided anti-CD3 antibodies and antigen-binding fragments thereof exhibit low polyreactivity to extracellular matrix (ECM) proteins. This is advantageous because low polyreactivity with ECM proteins can reduce off-target effects caused by the antibody binding to ECM proteins, and can reduce or avoid the negative effects that polyreactivity can have on pharmacokinetics and bioavailability of the antibody.

Accordingly, in some embodiments, the anti-CD3 antibody or antigen-binding fragment thereof exhibits low non-specific binding to extracellular matrix proteins, such as by having an extracellular matrix (ECM) score of less than 14, less than 13, less than 12, less than 11, or less than 10. Low polyreactivity is advantageous because it can, e.g., reduce off-target effects caused by the antibody binding to extracellular matrix proteins, for instance; and it can also reduce or avoid the negative effects that polyreactivity can have on pharmacokinetics and bioavailability of the therapeutic antibody. Polyreactivity can be assessed by, for instance, reference to an ECM score that is calculated based on an ECM ELISA assay, where the ECM score is calculated by dividing the absorbance value of an ECM-coated sample well containing the antibody or antigen-binding fragment thereof by the absorbance of an ECM-coated sample well that does not contain the antibody or antigen-binding fragment thereof. In some embodiments, an ECM score of 12 at a concentration of 1 μM antibody or antigen-binding fragment thereof is used to determine whether an antibody or antigen-binding fragment thereof has a high or low ECM score, with a high ECM score being at or higher than 12, and a low ECM score being less than 12. In some embodiments, the anti-CD3 antibody or antigen-binding fragment thereof has an ECM score of less than 14, less than 13, less than 12, less than 11, or less than 10. In some embodiments, the anti-CD3 antibody or antigen-binding fragment thereof has an ECM score of less than 12.

In some embodiments, the V_{H} region comprises one or more amino acid residues selected from glycine (G) at position 16, a glutamic acid (E) at position 16, a threonine (T) at position 58, an aspartic acid (D) at position 58, a glutamic acid (E) at position 76, a serine(S) at position 76, wherein the numbering is relative to SEQ ID NO: 24. In some embodiments, the V_{H} region comprises glycine (G) at position 16, wherein the numbering is relative to SEQ ID NO: 24. In some embodiments, the V_{H} region comprises a threonine (T) at position 58, wherein the numbering is relative to SEQ ID NO: 24. In some embodiments, the V_{H} region comprises glycine (G) at position 16 and a threonine (T) at position 58, wherein the numbering is relative to SEQ ID NO: 24. In some of any such embodiments, the anti-CD3 antibody or antigen-binding fragment thereof has a low polyreactivity with an ECM score of less than 14, less than 13, less than 12, less than 11, or less than 10. In particular embodiments, the ECM score is less than 12.

In some embodiments, any of the provided antibodies or antigen-binding fragments are single chain variable fragments (scFv). In some embodiments, the scFv is a disulfide-stablized scFv (dsFv) wherein the VH includes a first engineered cysteine residue and the VL includes a second engineered cysteine residue and wherein the first engineered cysteine residue and the second engineered cysteine residue form a disulfide bond. In some embodiments, the first engineered cysteine residue is at position 44 (e.g., G44C amino acid substitution) in the VH, wherein the numbering is relative to SEQ ID NO: 24. In some embodiments, the second engineered cysteine residue is at position 100 (e.g., Q100C amino acid substitution) in the VL, wherein the numbering is relative to SEQ ID NO: 39. In some embodiments, the first engineered cysteine residue is at position 44 (e.g, G44C amino acid substitution) in the VH (wherein the numbering is relative to SEQ ID NO: 24) and the second engineered cysteine residue is at position 100 (e.g., Q100C amino acid substitution) in the VL (wherein the numbering is relative to SEQ ID NO: 39).

In some embodiments, the V_{H} region comprises the CDR-H1, the CDR-H2, and the CDR-H3 contained within SEQ ID NO: 28, and the V_{L} region comprises the CDR-L1, the CDR-L2, and the CDR-L3 contained within SEQ ID NO: 42. In some embodiments, the V_{H} region comprises the CDR-H1, the CDR-H2, and the CDR-H3 comprising the sequence set forth in SEQ ID NOs: 9, 15 and 14, respectively, and the V_{L} region comprises the CDR-L1, the CDR-L2, and the CDR-L3 comprising the sequence set forth in SEQ ID NOs: 19, 22 and 21, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 28, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 42. In some embodiments, the VH comprises glycine (G) at position 16, cysteine (C) at position 44, a threonine (T) at position 58, alanine (A) at position 55, tyrosine (Y) at position 80, leucine (L) at position 112, and aspartic acid (D) at position 113, wherein the numbering is relative to SEQ ID NO: 24, and the VL region comprises serine(S) at position 56 and cysteine (C) at position 100, wherein the numbering is relative to SEQ ID NO: 39. In some embodiments, the V_{H} region and the V_{L} region comprise the sequences set forth in SEQ ID NO: 28 and 42, respectively. In some embodiments, the CD3 binding domain comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 130. In some embodiments, the CD3 binding domain comprises an amino acid sequence of SEQ ID NO: 130.

In some embodiments, the V_{H} region comprises the CDR-H1, the CDR-H2, and the CDR-H3 contained within SEQ ID NO: 29, and the V_{L} region comprises the CDR-L1, the CDR-L2, and the CDR-L3 contained within SEQ ID NO: 40. In some embodiments, the V_{H} region comprises the CDR-H1, the CDR-H2, and the CDR-H3 comprising the sequence set forth in SEQ ID NOs: 9, 15 and 14, respectively, and the V_{L} region comprises the CDR-L1, the CDR-L2, and the CDR-L3 comprising the sequence set forth in SEQ ID NOs: 19, 22 and 21, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 29, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, the VH comprises amino acid residues glycine (G) at position 16, alanine (A) at position 55, threonine (T) at position 58, tyrosine (Y) at position 80, leucine (L) at position 112, and aspartic acid (D) at position 113, wherein the numbering is relative to SEQ ID NO: 24, and the VL region comprises serine(S) at position 56, wherein the numbering is relative to SEQ ID NO: 39. In some embodiments, the V_{H} region and the V_{L} region comprise the sequences set forth in SEQ ID NO: 29 and 40, respectively.

In some embodiments, the V_{H} region comprises the CDR-H1, the CDR-H2, and the CDR-H3 contained within SEQ ID NO: 30, and the V_{L} region comprises the CDR-L1, the CDR-L2, and the CDR-L3 contained within SEQ ID NO: 41. In some embodiments, the V_{H} region comprises the CDR-H1, the CDR-H2, and the CDR-H3 comprising the sequence set forth in SEQ ID NOs: 9, 15 and 16, respectively, and the V_{L} region comprises the CDR-L1, the CDR-L2, and the CDR-L3 comprising the sequence set forth in SEQ ID NOs: 19, 22 and 23, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 30, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41. In some embodiments, the VH comprises amino acid residues glycine (G) at position 16, alanine (A) at position 55, threonine (T) at position 58, tyrosine (Y) at position 80, serine(S) at position 109, and histidine (H) at position 110, wherein the numbering is relative to SEQ ID NO: 24, and the VL region comprises serine(S) at position 56 and asparagine (N) at position 92, wherein the numbering is relative to SEQ ID NO: 39. In some embodiments, the V_{H} region and the V_{L} region comprise the sequences set forth in SEQ ID NO: 29 and 40, respectively.

In some embodiments, the V_{H} region comprises the CDR-H1, the CDR-H2, and the CDR-H3 contained within SEQ ID NO: 31, and the V_{L} region comprises the CDR-L1, the CDR-L2, and the CDR-L3 contained within SEQ ID NO: 43. In some embodiments, the V_{H} region comprises the CDR-H1, the CDR-H2, and the CDR-H3 comprising the sequence set forth in SEQ ID NOs: 9, 15 and 16, respectively, and the V_{L} region comprises the CDR-L1, the CDR-L2, and the CDR-L3 comprising the sequence set forth in SEQ ID NOs: 19, 22 and 23, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 31, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43. In some embodiments, the VH comprises amino acid residues glycine (G) at position 16, cysteine (C) at position 44, alanine (A) at position 55, threonine (T) at position 58, tyrosine (Y) at position 80, serine(S) at position 109, and histidine (H) at position 110, wherein the numbering is relative to SEQ ID NO: 24, and the VL region comprises serine(S) at position 56, asparagine (N) at position 92 and cysteine (C) at position 100, wherein the numbering is relative to SEQ ID NO: 39. In some embodiments, the V_{H} region and the V_{L} region comprise the sequences set forth in SEQ ID NO: 31 and 43, respectively. In some embodiments, the CD3 binding domain comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 131. In some embodiments, the CD3 binding domain comprises the amino acid sequence of SEQ ID NO: 131.

In some embodiments, the V_{H} region comprises the CDR-H1, the CDR-H2, and the CDR-H3 contained within SEQ ID NO: 32, and the V_{L} region comprises the CDR-L1, the CDR-L2, and the CDR-L3 contained within SEQ ID NO: 40. In some embodiments, the V_{H} region comprises the CDR-H1, the CDR-H2, and the CDR-H3 comprising the sequence set forth in SEQ ID NOs: 9, 13 and 14, respectively, and the V_{L} region comprises the CDR-L1, the CDR-L2, and the CDR-L3 comprising the sequence set forth in SEQ ID NOs: 19, 22 and 21, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 32, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, the VH comprises amino acid residues glycine (G) at position 16, alanine (A) at position 55, tyrosine (Y) at position 80, leucine (L) at position 112, and aspartic acid (D) at position 113, wherein the numbering is relative to SEQ ID NO: 24, and the VL region comprises serine(S) at position 56, wherein the numbering is relative to SEQ ID NO: 39. In some embodiments, the V_{H} region and the V_{L} region comprise the sequences set forth in SEQ ID NO: 32 and 40, respectively.

In some embodiments, the V_{H} region comprises the CDR-H1, the CDR-H2, and the CDR-H3 contained within SEQ ID NO: 26, and the V_{L} region comprises the CDR-L1, the CDR-L2, and the CDR-L3 contained within SEQ ID NO: 40. In some embodiments, the V_{H} region comprises the CDR-H1, the CDR-H2, and the CDR-H3 comprising the sequence set forth in SEQ ID NOs: 9, 13 and 14, respectively, and the V_{L} region comprises the CDR-L1, the CDR-L2, and the CDR-L3 comprising the sequence set forth in SEQ ID NOs: 19, 22 and 21, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 26, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, the VH comprises amino acid residues a glutamic acid (E) at position 16, alanine (A) at position 55, tyrosine (Y) at position 80, leucine (L) at position 112, and aspartic acid (D) at position 113, wherein the numbering is relative to SEQ ID NO: 24, and the VL region comprises serine(S) at position 56, wherein the numbering is relative to SEQ ID NO: 39. In some embodiments, the V_{H} region and the V_{L} region comprise the sequences set forth in SEQ ID NO: 26 and 40, respectively.

In some embodiments, the V_{H} region comprises the CDR-H1, the CDR-H2, and the CDR-H3 contained within SEQ ID NO: 33, and the V_{L} region comprises the CDR-L1, the CDR-L2, and the CDR-L3 contained within SEQ ID NO: 40. In some embodiments, the V_{H} region comprises the CDR-H1, the CDR-H2, and the CDR-H3 comprising the sequence set forth in SEQ ID NOs: 9, 17 and 14, respectively, and the V_{L} region comprises the CDR-L1, the CDR-L2, and the CDR-L3 comprising the sequence set forth in SEQ ID NOs: 19, 22 and 21, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 33, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, the VH comprises amino acid residues a glutamic acid (E) at position 16, alanine (A) at position 55, aspartic acid (D) at position 58, tyrosine (Y) at position 80, leucine (L) at position 112, and aspartic acid (D) at position 113, wherein the numbering is relative to SEQ ID NO: 24, and the VL region comprises serine(S) at position 56, wherein the numbering is relative to SEQ ID NO: 39. In some embodiments, the V_{H} region and the V_{L} region comprise the sequences set forth in SEQ ID NO: 33 and 40, respectively.

In some embodiments, the V_{H} region comprises the CDR-H1, the CDR-H2, and the CDR-H3 contained within SEQ ID NO: 34, and the V_{L} region comprises the CDR-L1, the CDR-L2, and the CDR-L3 contained within SEQ ID NO: 40. In some embodiments, the V_{H} region comprises the CDR-H1, the CDR-H2, and the CDR-H3 comprising the sequence set forth in SEQ ID NOs: 9, 15 and 14, respectively, and the V_{L} region comprises the CDR-L1, the CDR-L2, and the CDR-L3 comprising the sequence set forth in SEQ ID NOs: 19, 22 and 21, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 34, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, the VH comprises amino acid residues a glycine (G) at position 16, alanine (A) at position 55, threonine (T) at position 58, serine(S) at position 76, tyrosine (Y) at position 80, leucine (L) at position 112, and aspartic acid (D) at position 113, wherein the numbering is relative to SEQ ID NO: 24, and the VL region comprises serine(S) at position 56, wherein the numbering is relative to SEQ ID NO: 39. In some embodiments, the V_{H} region and the V_{L} region comprise the sequences set forth in SEQ ID NO: 34 and 40, respectively.

In some embodiments, the V_{H} region comprises the CDR-H1, the CDR-H2, and the CDR-H3 contained within SEQ ID NO: 35, and the V_{L} region comprises the CDR-L1, the CDR-L2, and the CDR-L3 contained within SEQ ID NO: 40. In some embodiments, the V_{H} region comprises the CDR-H1, the CDR-H2, and the CDR-H3 comprising the sequence set forth in SEQ ID NOs: 9, 17 and 14, respectively, and the V_{L} region comprises the CDR-L1, the CDR-L2, and the CDR-L3 comprising the sequence set forth in SEQ ID NOs: 19, 22 and 21, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 35, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, the VH comprises amino acid residues a glutamic acid (E) at position 16, alanine (A) at position 55, aspartic acid (D) at position 58, serine(S) at position 76, tyrosine (Y) at position 80, leucine (L) at position 112, and aspartic acid (D) at position 113, wherein the numbering is relative to SEQ ID NO: 24, and the VL region comprises serine(S) at position 56, wherein the numbering is relative to SEQ ID NO: 39. In some embodiments, the V_{H} region and the V_{L} region comprise the sequences set forth in SEQ ID NO: 35 and 40, respectively.

In some embodiments, the V_{H} region comprises the CDR-H1, the CDR-H2, and the CDR-H3 contained within SEQ ID NO: 36, and the V_{L} region comprises the CDR-L1, the CDR-L2, and the CDR-L3 contained within SEQ ID NO: 40. In some embodiments, the V_{H} region comprises the CDR-H1, the CDR-H2, and the CDR-H3 comprising the sequence set forth in SEQ ID NOs: 9, 13 and 14, respectively, and the V_{L} region comprises the CDR-L1, the CDR-L2, and the CDR-L3 comprising the sequence set forth in SEQ ID NOs: 19, 22 and 21, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, the VH comprises amino acid residues a glycine (G) at position 16, alanine (A) at position 55, serine(S) at position 76, tyrosine (Y) at position 80, leucine (L) at position 112, and aspartic acid (D) at position 113, wherein the numbering is relative to SEQ ID NO: 24, and the VL region comprises serine(S) at position 56, wherein the numbering is relative to SEQ ID NO: 39. In some embodiments, the V_{H} region and the V_{L} region comprise the sequences set forth in SEQ ID NO: 36 and 40, respectively.

In some embodiments, the V_{H} region comprises the CDR-H1, the CDR-H2, and the CDR-H3 contained within SEQ ID NO: 37, and the V_{L} region comprises the CDR-L1, the CDR-L2, and the CDR-L3 contained within SEQ ID NO: 40. In some embodiments, the V_{H} region comprises the CDR-H1, the CDR-H2, and the CDR-H3 comprising the sequence set forth in SEQ ID NOs: 9, 13 and 14, respectively, and the V_{L} region comprises the CDR-L1, the CDR-L2, and the CDR-L3 comprising the sequence set forth in SEQ ID NOs: 19, 22 and 21, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40. In some embodiments, the VH comprises amino acid residues a glutamic acid (E) at position 16, alanine (A) at position 55, glutamic acid (E) at position 76, tyrosine (Y) at position 80, leucine (L) at position 112, and aspartic acid (D) at position 113, wherein the numbering is relative to SEQ ID NO: 24, and the VL region comprises serine(S) at position 56, wherein the numbering is relative to SEQ ID NO: 39. In some embodiments, the V_{H} region and the V_{L} region comprise the sequences set forth in SEQ ID NO: 37 and 40, respectively.

In some embodiments, the V_{H} region comprises the CDR-H1, the CDR-H2, and the CDR-H3 contained within SEQ ID NO: 38, and the V_{L} region comprises the CDR-L1, the CDR-L2, and the CDR-L3 contained within SEQ ID NO: 41. In some embodiments, the V_{H} region comprises the CDR-H1, the CDR-H2, and the CDR-H3 comprising the sequence set forth in SEQ ID NOs: 9, 15 and 18, respectively, and the V_{L} region comprises the CDR-L1, the CDR-L2, and the CDR-L3 comprising the sequence set forth in SEQ ID NOs: 19, 22 and 23, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 38, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41. In some embodiments, the VH comprises amino acid residues a glycine (G) at position 16, alanine (A) at position 55, threonine (T) at position 58, tyrosine (Y) at position 80, glycine (G) at position 106, and histidine (H) at position 107, wherein the numbering is relative to SEQ ID NO: 24, and the VL region comprises serine(S) at position 56 and asparagine (N) at position 92, wherein the numbering is relative to SEQ ID NO: 39. In some embodiments, the V_{H} region and the V_{L} region comprise the sequences set forth in SEQ ID NO: 38 and 41, respectively.

In some embodiments, a provided binding molecule is an antibody or an antigen-binding fragment containing any of the above V_{H} and V_{L} regions, and their combinations, described above. In some embodiments, the antibody or antigen-binding fragment further includes human immunoglobulin constant regions. In some embodiments, the antibody or antigen-binding fragment is an antigen-binding fragment. In some embodiments, the antigen-binding fragment format retains CD3 binding conferred by the VH and VL regions. In some embodiments, the antigen-binding fragment may be an Fv, scFv, Fab, Fab′, F(ab′)2, or other antigen-binding fragment of antibodies. In some embodiments, the antibody or antigen-binding fragment is a full-length antibody that comprises human immunoglobulin constant regions, including an Fc region.

Antigen-binding fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that may not be produced by enzyme digestion of a naturally-occurring intact antibody.

In some embodiments, the antibody or antigen-binding fragment is an scFv containing any of the above V_{H} and V_{L} regions. In some embodiments, the scFv includes a linker joining the two antibody V_{H} and V_{L} region. The linker typically is a peptide linker, e.g., a flexible and/or soluble peptide linker, such as one rich in glycine and serine. In some aspects, the linker rich in glycine and serine (and/or threonine) includes at least 80%, 85%, 90%, 91%, 92%, 93%, 94%, 95%, 96%, 97%, 98%, or 99% glycine, serine, and/or threonine amino acid(s). In some embodiments, the linker includes at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO: 94) or GGGS (3GS; SEQ ID NO: 97), such as between 2, 3, 4 and 5 repeats of such a sequence. Exemplary linkers include those having the sequence set forth in SEQ ID NO: 96 (GGGGSGGGGSGGGGS). Exemplary linkers further include those having the sequence set forth in SEQ ID NO: 98 (GSTSGSGKPGSGEGSTKG). Exemplary linkers further include those having or consisting of the sequence set forth in SEQ ID NO: 99 (SRGGGGSGGGGSGGGGSLEMA). An exemplary linker includes those having or consisting of the sequence set forth in SEQ ID NO: 100 (GSRGGGGSGGGGSGGGGSLEMA). In some embodiments, the V_{H} region may be amino terminal to the V_{L} region. In some embodiments, the V_{H} region may be carboxy terminal to the V_{L} region. In particular embodiments, the fragment, e.g., scFv, may include a V_{H} region or portion thereof, followed by the linker, followed by a V_{L} region or portion thereof. In other embodiments, the fragment, e.g., the scFv, may include the V_{L} region or portion thereof, followed by the linker, followed by the V_{H} region or portion thereof.

In some embodiments, the scFv is a disulfide-stabilized Fv (ds-scFv) in which the VH and VL contain a first and second engineered cysteine residue, respectively, that form a disulfide bond. In some embodiments, the first engineered cysteine residue is at position 44 (e.g, G44C amino acid substitution) in the VH, wherein the numbering is relative to SEQ ID NO: 24, and the second engineered cysteine residue is at position 100 (e.g., G100C amino acid substitution) in the VL, wherein the numbering is relative to SEQ ID NO: 39.

In some embodiments, the antibody or antigen-binding fragment is an scFv comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 128. In some embodiments, the scFv comprises the amino acid sequence of SEQ ID NO: 128. In some embodiments, the antibody or antigen-binding fragment is an scFv comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 129. In some embodiments, the scFv comprises the amino acid sequence of SEQ ID NO: 129. In some embodiments, the antibody or antigen-binding fragment is an scFv comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 130. In some embodiments, the scFv comprises the amino acid sequence of SEQ ID NO: 130. In some embodiments, the antibody or antigen-binding fragment is an scFv comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 131. In some embodiments, the scFv comprises the amino acid sequence of SEQ ID NO: 131.

In some such embodiments, the antibody is a full-length antibody that also contains a heavy chain and light chain constant region. In some embodiments, the antibody comprises a constant region from an IgG1 or IgG2. In some embodiments, the constant region is a human IgG1 heavy chain constant region. In some embodiments, the antibody includes at least a portion of a hinge region or a variant thereof. In some embodiments, the antibody includes a CH2 and/or CH3 domain, such as an Fc region. In some embodiments, the Fc region is an Fc region of a human IgG, such as an IgG1 or IgG4. In some embodiments, the heavy chain constant portion is derived from an IgG1 immunoglobulin constant chain. In some embodiments, the light chain CL is from a kappa or lambda light chain sequence.

In some embodiments, the constant light chain is a 2 (lambda) light chain. In some embodiments, the constant light (CL) is set forth by the sequence in SEQ ID NO: 103 or a sequence having at least at or about 85%, 86%, 87%, 88%, 89%, 90%, 91%, 92%, 93%, 94%, 95%, 96%, 97%, 98% or 99% identity to SEQ ID NO:103.

In some embodiments, the constant light chain is a κ (kappa) light chain. In some embodiments, the CL is set forth by the sequence in SEQ ID NO: 52 or a sequence having at least at or about 85%, 86%, 87%, 88%, 89%, 90%, 91%, 92%, 93%, 94%, 95%, 96%, 97%, 98% or 99% identity to SEQ ID NO:52.

In some embodiments, the constant light chain is a κ light chain. In some embodiments, the CL is set forth by the sequence in SEQ ID NO: 104 or a sequence having at least at or about 85%, 86%, 87%, 88%, 89%, 90%, 91%, 92%, 93%, 94%, 95%, 96%, 97%, 98% or 99% identity to SEQ ID NO:104.

In some embodiments, the antibody or antigen-binding fragment is an antigen-binding fragment that further comprises at least a portion of an immunoglobulin constant region of the heavy chain and at least a portion of an immunoglobulin constant region of the light chain. In some embodiments, the CD3 binding domain is an anti-CD3 antibody that is a Fab containing any of the above V_{H} and V_{L} regions. In some embodiments, the Fab contains a heavy chain comprising any one of the above V_{H} regions and an immunoglobulin constant heavy chain domain 1 (CH1 domain) and a light chain comprising any one of the above V_{L} regions and an immunoglobulin CL.

In some embodiments, the CH1 is from an IgG1 antibody. In some embodiments, the CH1 is set forth by the sequence in SEQ ID NO: 101 or a sequence having at least at or about 85%, 86%, 87%, 88%, 89%, 90%, 91%, 92%, 93%, 94%, 95%, 96%, 97%, 98%, 99% identity to SEQ ID NO:101. In some embodiments, the CH1 is set forth by the sequence in SEQ ID NO: 102 or a sequence having at least at or about 85%, 86%, 87%, 88%, 89%, 90%, 91%, 92%, 93%, 94%, 95%, 96%, 97%, 98% or 99% identity to SEQ ID NO: 102. In some embodiments, the CH1 is set forth by the sequence in SEQ ID NO: 50 or a sequence having at least at or about 85%, 86%, 87%, 88%, 89%, 90%, 91%, 92%, 93%, 94%, 95%, 96%, 97%, 98% or 99% identity to SEQ ID NO:50.

In some embodiments, the CH1 is from an IgG2 antibody. n some embodiments, the CH1 is set forth by the sequence in SEQ ID NO: 51 or a sequence having at least at or about 85%, 86%, 87%, 88%, 89%, 90%, 91%, 92%, 93%, 94%, 95%, 96%, 97%, 98%, 99% identity to SEQ ID NO:51.

In some embodiments, the CL is a kappa constant domain. In some embodiments, the CL is set forth by the sequence in SEQ ID NO: 52 or a sequence having at least at or about 85%, 86%, 87%, 88%, 89%, 90%, 91%, 92%, 93%, 94%, 95%, 96%, 97%, 98% or 99% identity to SEQ ID NO:52. In some embodiments, the CL is set forth by the sequence in SEQ ID NO: 104 or a sequence having at least at or about 85%, 86%, 87%, 88%, 89%, 90%, 91%, 92%, 93%, 94%, 95%, 96%, 97%, 98% or 99% identity to SEQ ID NO: 104.

In some embodiments, the CL is a lambda constant domain. In some embodiments, the CL is set forth by the sequence in SEQ ID NO: 103 or a sequence having at least at or about 85%, 86%, 87%, 88%, 89%, 90%, 91%, 92%, 93%, 94%, 95%, 96%, 97%, 98% or 99% identity to SEQ ID NO: 103.

In some embodiments, the CD3 binding domain is an anti-CD3 antibody that is a Fab composed of a heavy chain comprising any one of the above V_{H} regions and an CH1 domain set forth in SEQ ID NO: 50, and a light chain comprising any one of the above V_{L} regions and an immunoglobulin CL set forth in SEQ ID NO: 52. In some embodiments, the CD3 binding domain is a Fab comprising a heavy chain comprising a VH set forth by the sequence of SEQ ID NO:29 and a CH1 comprising the sequence set forth in SEQ ID NO:50, and a light chain comprising a VL set forth by the sequence of SEQ ID NO: 76 and a CL comprising the sequence set forth in SEQ ID NO: 52. In some embodiments, the CD3 binding domain is a Fab comprising a heavy chain comprising a VH set forth by the sequence of SEQ ID NO:30 and a CH1 comprising the sequence set forth in SEQ ID NO:50, and a light chain comprising a VL set forth by the sequence of SEQ ID NO: 81 and a CL comprising the sequence set forth in SEQ ID NO: 52.

In some such embodiments, the antibody is a full-length antibody that also contains a heavy chain and light chain constant region. In some embodiments, the anti-CD3 antibody, or an antigen binding fragment thereof, is a human IgG. In some embodiments, the antibody comprises a constant region from an IgG1 or IgG2. In some embodiments, the constant region is a human IgG1 heavy chain constant region. In some embodiments, the antibody includes at least a portion of a hinge region or a variant thereof. In some embodiments, the antibody includes a CH2 and/or CH3 domain, such as an Fc region. In some embodiments, the Fc region is an Fc region of a human IgG, such as an IgG1 or IgG4. In some embodiments, the heavy chain constant portion is derived from an IgG1 immunoglobulin constant chain. In some embodiments, the light chain CL is from a kappa or lambda light chain sequence.

In full-length antibody embodiments, the antibody comprises two heavy chains and two light chains (H2L2), wherein each heavy chain comprises a VH region and a heavy-chain constant region and each light chain comprises a VL region and a light-chain constant region, and the heavy chain may include an Fc region comprising at least part of the hinge, CH2, and CH3 domains. Among the provided antibodies are full-length antibodies containing a heavy chain with any one of the VH regions provided herein combined with a human heavy chain constant region; and a light chain with any one of the VL regions provided herein combined with a human light chain constant region.

In some embodiments, the constant region of the heavy chain is a human IgG1 heavy chain constant region. In some embodiments, the heavy chain constant region is set forth in SEQ ID NO: 86 or a sequence that has at least 85%, 86%, 87%, 88%, 89%, 90%, 91%, 92%, 93%, 94%, 95%, 96%, 97%, 98%, or 99% sequence identity to SEQ ID NO: 86. In some embodiments, the heavy chain constant region is set forth in SEQ ID NO: 86. In some embodiments, the heavy chain constant region is set forth in SEQ ID NO: 87 or a sequence that has at least 85%, 86%, 87%, 88%, 89%, 90%, 91%, 92%, 93%, 94%, 95%, 96%, 97%, 98%, or 99% sequence identity to SEQ ID NO: 87. In some embodiments, the heavy chain constant region is set forth in SEQ ID NO: 87.

In some embodiments, the constant light chain is a λ (lambda) light chain. In some embodiments, the constant light (CL) is set forth by the sequence in SEQ ID NO: 103 or a sequence having at least at or about 85%, 86%, 87%, 88%, 89%, 90%, 91%, 92%, 93%, 94%, 95%, 96%, 97%, 98% or 99% identity to SEQ ID NO:103.

In some embodiments, the constant light chain is a κ (kappa) light chain. In some embodiments, the constant light chain (CL) is set forth by the sequence in SEQ ID NO: 52 or a sequence having at least at or about 85%, 86%, 87%, 88%, 89%, 90%, 91%, 92%, 93%, 94%, 95%, 96%, 97%, 98% or 99% identity to SEQ ID NO:52.

In some embodiments, the constant light chain is a κ (kappa) light chain. In some embodiments, the constant light chain (CL) is set forth by the sequence in SEQ ID NO: 104 or a sequence having at least at or about 85%, 86%, 87%, 88%, 89%, 90%, 91%, 92%, 93%, 94%, 95%, 96%, 97%, 98% or 99% identity to SEQ ID NO: 104.

In some embodiments, the heavy chain constant region is mutated or modified relative to a wild-type immunoglobulin constant region (e.g., a human IgG constant region) to provide a modified (or variant) Fc region, when an Fc region or portion thereof is present. In some embodiments, the modified constant region comprises a modified Fc region (e.g., a human IgG1, IgG2, IgG3, or IgG4 Fc region) comprising one or more amino acid modifications (e.g., substitutions and/or deletions) at one or more amino acid positions. In some embodiments, the Fc region comprises a hinge region, CH2, and CH3.

In some cases, the mutations include one or more amino acid substitutions in the heavy chain constant region, such as the heavy chain constant region of IgG1, to reduce effector activity of the heavy chain constant region. In some embodiments, the Fc region is mutated or modified to reduce or eliminate one or more Fc effector functions (e.g., Fcγ receptor binding and/or C1q binding). In some embodiments, such mutation or modification substantially retains FcRn binding. Exemplary mutations in an Fc that can be included to reduce or eliminate one or more Fc effector functions, optionally while retaining FcRn binding are described in Section II.B.2. Any other mutation (e.g., substitution) described in Section II.B.2 may also be included an a Fc constant region sequence of a provided antibody.

In some embodiments, a provided antibody or antigen-binding fragment is a full length antibody that is capable of forming a dimer via Fc-mediated association. In some embodiments, the Fc region comprises a first Fc-containing polypeptide chain and a second Fc-containing polypeptide chain that associate to form a homodimer comprising two identical Fc-containing polypeptide chains. In some embodiments, dimerization is mediated by interactions within one or more constant-region domains of the Fc region (e.g., hinge, CH2, and/or CH3) and, where present, inter-chain disulfide bonds in the hinge. In some embodiments, the Fc region is formed by Fc domains that are mutated or modified to promote heterodimerization in which different polypeptides can be dimerized to yield a heterodimer. Thus, in some embodiments, the dimer is a heterodimer in which two polypeptide chains of the antibody are different. Exemplary modifications to promote heterodimerization are known, including any as described below in Section II.B.2.

In some embodiments, the anti-CD3 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 133; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 134. In some embodiments, the anti-CD3 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 135; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 136. In some embodiments, the anti-CD3 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 137; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 136. In some embodiments, the anti-CD3 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 138; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 139. In some embodiments, the anti-CD3 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 140; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 141. In some embodiments, the anti-CD3 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 142; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 136. In some embodiments, the anti-CD3 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 143; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 139. In some embodiments, the anti-CD3 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 144; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 145. In some embodiments, the anti-CD3 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 146; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 136. In some embodiments, the anti-CD3 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 147; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 136. In some embodiments, the anti-CD3 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 148; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 136. In some embodiments, the anti-CD3 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 149; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 136. In some embodiments, the anti-CD3 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 150; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 136. In some embodiments, the anti-CD3 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 151; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 136. In some embodiments, the anti-CD3 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 152; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 136. In some embodiments, the anti-CD3 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 153; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 136.

In some embodiments, the anti-CD3 antibody, or an antigen binding fragment thereof comprises: (i) a heavy chain comprising the amino acid sequence of SEQ ID NO: 133; and (ii) a light chain comprising the amino acid sequence of SEQ ID NO: 134. In some embodiments, the anti-CD3 antibody, or an antigen binding fragment thereof comprises: heavy chain comprising the amino acid sequence of SEQ ID NO: 135; and (ii) a light chain comprising the amino acid sequence of SEQ ID NO: 136. In some embodiments, the anti-CD3 antibody, or an antigen binding fragment thereof comprises: a heavy chain comprising the amino acid sequence of SEQ ID NO: 137; and (ii) a light chain comprising the amino acid sequence of SEQ ID NO: 136. In some embodiments, the anti-CD3 antibody, or an antigen binding fragment thereof comprises: a heavy chain comprising the amino acid sequence of SEQ ID NO: 138; and (ii) a light chain comprising the amino acid sequence of SEQ ID NO: 139. In some embodiments, the anti-CD3 antibody, or an antigen binding fragment thereof comprises: a heavy chain comprising the amino acid sequence of SEQ ID NO: 140; and (ii) a light chain comprising the amino acid sequence of SEQ ID NO: 141. In some embodiments, the anti-CD3 antibody, or an antigen binding fragment thereof comprises: a heavy chain comprising the amino acid sequence of SEQ ID NO: 142; and (ii) a light chain comprising the amino acid sequence of SEQ ID NO: 136. In some embodiments, the anti-CD3 antibody, or an antigen binding fragment thereof comprises: a heavy chain comprising the amino acid sequence of SEQ ID NO: 143; and (ii) a light chain comprising the amino acid sequence of SEQ ID NO: 139. In some embodiments, the anti-CD3 antibody, or an antigen binding fragment thereof comprises: a heavy chain comprising the amino acid sequence of SEQ ID NO: 144; and (ii) a light chain comprising the amino acid sequence of SEQ ID NO: 145. In some embodiments, the anti-CD3 antibody, or an antigen binding fragment thereof comprises: a heavy chain comprising the amino acid sequence of SEQ ID NO: 146; and (ii) a light chain comprising the amino acid sequence of SEQ ID NO: 136. In some embodiments, the anti-CD3 antibody, or an antigen binding fragment thereof comprises: a heavy chain comprising the amino acid sequence of SEQ ID NO: 147; and (ii) a light chain comprising the amino acid sequence of SEQ ID NO: 136. In some embodiments, the anti-CD3 antibody, or an antigen binding fragment thereof comprises: a heavy chain comprising the amino acid sequence of SEQ ID NO: 148; and (ii) a light chain comprising the amino acid sequence of SEQ ID NO: 136. In some embodiments, the anti-CD3 antibody, or an antigen binding fragment thereof comprises: (i) a heavy chain comprising the amino acid sequence of SEQ ID NO: 149; and (ii) a light chain comprising the amino acid sequence of SEQ ID NO: 136. In some embodiments, the anti-CD3 antibody, or an antigen binding fragment thereof comprises: (i) a heavy chain comprising the amino acid sequence of SEQ ID NO: 150; and (ii) a light chain comprising the amino acid sequence of SEQ ID NO: 136. In some embodiments, the anti-CD3 antibody, or an antigen binding fragment thereof comprises: (i) a heavy chain comprising the amino acid sequence of SEQ ID NO: 151; and (ii) a light chain comprising the amino acid sequence of SEQ ID NO: 136. In some embodiments, the anti-CD3 antibody, or an antigen binding fragment thereof comprises: (i) a heavy chain comprising the amino acid sequence of SEQ ID NO: 152; and (ii) a light chain comprising the amino acid sequence of SEQ ID NO: 136. In some embodiments, the anti-CD3 antibody, or an antigen binding fragment thereof comprises: (i) a heavy chain comprising the amino acid sequence of SEQ ID NO: 153; and (ii) a light chain comprising the amino acid sequence of SEQ ID NO: 136.

Also provided are methods of making the CD3 binding molecules, including anti-CD3 antibodies (including antigen-binding fragments) or a binding molecule incorporating such antibodies or fragments. For recombinant production of the binding molecule, such as an anti-CD3 antibody, a polynucleotide sequence or a polynucleotide encoding an antibody, e.g., as described above, may be isolated and inserted into one or more vectors for further cloning and/or expression in a host cell. Such polynucleotide sequences may be readily isolated and sequenced using conventional procedures (e.g., by using oligonucleotide probes that are capable of binding specifically to genes encoding the heavy and light chains of the antibody). In some embodiments, a method of making the CD3 binding molecules, such as anti-CD3 antibody, is provided, wherein the method comprises culturing a host cell comprising a polynucleotide sequence encoding the binding molecule, as provided above, under conditions suitable for expression of the binding molecule, and optionally recovering the binding molecule from the host cell (or host cell culture medium).

Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells; and NSO cells. In some embodiments, the antibody heavy chains and/or light chains (e.g., VH region and/or VL region) may be expressed in yeast. See, e.g., U.S. Publication No. US 2006/0270045 A1. In some embodiments, a particular eukaryotic host cell is selected based on its ability to make desired post-translational modifications to the heavy chains and/or light chains (e.g., VH region and/or VL region). For example, in some embodiments, CHO cells produce polypeptides that have a higher level of sialylation than the same polypeptide produced in 293 cells.

In some embodiments, the antibody or antigen-binding fragment provided herein is produced in a cell-free system. Exemplary cell-free systems are described, e.g., in Sitaraman et al., Methods Mol. Biol. 498:229-44 (2009); Spirin, Trends Biotechnol. 22:538-45 (2004); Endo et al., Biotechnol. Adv. 21:695-713 (2003).

In some of any of such embodiments, the anti-CD3 antibody or antigen-binding fragment thereof comprises at least one post-translational modification of the amino acid sequence. Post-translational modifications can include, e.g., glycosylation, phosphorylation, citrullination, isomerization, ubiquitination, acetylation, hydroxylation, methylation, AMPylation, prenylation, deamidation, eliminylation, carbamylation and carbamoylation. In some embodiments, the post-translational modification is the modification of an amino acid side chain, such as conversion of an N-terminal amino acid (e.g., glutamate or glutamine) to pyroglutamate. In some such embodiments, this modification occurs at the N-terminus of the heavy chain. In some embodiments, the post-translational modification includes a post-translational modification of a heavy chain N-terminal glutamine (Q) to a pyroglutamate. In some embodiments, in a composition of an antibody or antigen-binding fragment thereof as disclosed herein, such a post-translational modification to pyroglutamate (e.g., at the N-terminus of the heavy chain) is a dominant post-translational modification; in some such embodiments, the antibody or antigen binding fragment thereof in which an N-terminal pyroglutamate is present represents greater than 50%, greater than 60%, greater than 70%, greater than 80%, greater than 90%, or greater than 95% (w/w) of the antibody or antigen binding fragment thereof in the composition.

In some embodiments, the post-translational modification is the cleavage of one or more, particularly one or two, amino acids from the C-terminus of the heavy chain. For instance, an antibody produced by expression of a specific nucleic acid molecule encoding a full-length heavy chain (e.g., by expression in a host cell) may include the full-length heavy chain, or it may include a cleaved variant of the full-length heavy chain. This may be the case where the final two C-terminal amino acids of the heavy chain are glycine and lysine, respectively. Therefore, the C-terminal lysine, or the C-terminal glycine and lysine, of the Fc region may or may not be present. Thus, an anti-CD3 antibody may include a heavy chain constant domain with both a C-terminal glycine and lysine, without the C-terminal lysine, or without both the C-terminal glycine and lysine. In some embodiments, in a composition of an antibody or antigen-binding fragment thereof as disclosed herein, glycine cleavage is not a dominant post-translational modification; in some such embodiments, the antibody or antigen binding fragment thereof in which the C-terminal lysine is cleaved from the heavy chain constant domain represents less than 10%, less than 5%, or less than 4% (w/w) of the antibody or antigen binding fragment thereof in the composition.

### B. Bispecific Binding Molecules

In some embodiments, provided herein are multispecific antigen-binding molecules that include any of the CD3 binding domains described herein. In some embodiments, the multispecific antigen-binding molecules are bispecific antigen-binding molecules.

In some embodiments, any of the exemplary binding molecules can include an anti-CD3 antibody or antigen binding antibody fragment comprised in a multispecific antibody composed of at least one anti-CD3 antibody that is formatted with at least one additional antibody directed against a second target antigen. In some embodiments, the multispecific antibody is a bispecific T cell engager specific for CD3 and the second target antigen. In some embodiments, the multspecific antibody is composed of at least one anti-CD3 antibody or antigen-binding fragment (e.g., anti-CD3 scFv or Fab) and an antibody or antigen-binding fragment (e.g., Fab or scFv) directed against a second antigen. In any of such embodiments, the binding molecule is a bispecific antigen binding molecule, such as a bispecific antibody, that has binding specificity for both CD3 and the second antigen.

The term “bispecific antibody” as used herein means a monoclonal antibody that has binding specificity for two different epitopes. Bispecific antibodies can be prepared as full-length antibodies or antibody fragments.

In some embodiments, the anti-CD3 antibody activates T cells via engagement of CD3, such as CD3ε, on the T cells. The anti-CD3 antibody exhibits activity to agonize, stimulate, activate, and/or otherwise augment CD3-mediated T cell activation. Biological activities of CD3 include, for example, T cell activation and other signaling through interaction between CD3 and the antigen-binding subunits of the T-Cell Receptor (TCR). Any of a variety of methods are known for measuring T cell activation. In some embodiments, T cell activation can be assessed by determining the ability of a provided bispecific antibody to stimulate cytokine production, proliferation or cytotoxic activity in T cell cultures. In some embodiments, the activity is present in co-cultures of T cells with antigen-expressing target cells but not present in the absence of antigen-expressing cells. In some embodiments, the bispecific antibodies elicit potent T cell activation or T cell killing when also engaged by or bound to the target (second) antigen, such as target antigen-expressing target cells (e.g., target antigen-expresssing tumor cells).

In some embodiments, the binding molecule containing a CD3 binding domain provides for a T cell engaging therapy. The T cell-engaging therapy is a bispecific antibody containing at least one antigen-binding domain binding to an activating component of the T cell such as CD3 and at least one antigen-binding domain binding to a second antigen that is present on a target cell desired to be targeted. In some embodiments, the simultaneous or near simultaneous binding of such an antibody to both of its targets can result in a temporary interaction between the target cell and T cell, thereby resulting in activation, e.g. cytotoxic activity, of the T cell and subsequent lysis of the target cell.

The provided bispecific antibodies provide a targeted antibody therapy for delivering cytotoxic T cells specifically to the target cells expressing the second antigen, such as target-expressing cancer cells. Hence, in some aspects the provided bispecific antibodies may also be called TCEs. In particular embodiments, the provided bispecific antibodies can be used in therapeutic settings in which specific targeting and T cell-mediated killing of cells that express the second antigen is desired.

In some embodiments, biological activity or functional activity of a provided bispecific antibody, such as cytotoxic activity, can be measured using any of a number of known methods. The activity can be assessed or determined either in vitro or in vivo. In some embodiments, activity can be assessed once the bispecific antibody is administered to the subject (e.g., human). Parameters to assess include specific binding of the bispecific antibody to a T cell (e.g., natural T cell) or target antigen-expressing cell, e.g., in vivo, e.g., by imaging, or ex vivo, e.g., by ELISA or flow cytometry. In certain embodiments, the ability of the bispecific antibody to destroy target cells can be measured using any suitable known methods, such as cytotoxicity assays described in, for example, Kochenderfer et al., J. Immunotherapy, 32 (7): 689-702 (2009), and Herman et al. J. Immunological Methods, 285 (1): 25-40 (2004). In certain embodiments, the biological activity of the bispecific antibody also can be measured by assaying expression and/or secretion of certain cytokines, such as interlekukin-2 (IL-2), interferon-gamma (IFNγ), interleukin-1 beta (IL-1β), interleukin-4 (IL-4), TNF-alpha (TNFα), interleukin-6 (IL-6), interleukin-10 (IL-10), interleukin-12 (IL-12), interleukin-13 (IL-13), granulocyte-macrophage colony-stimulating factor (GM-CSF), CD107a, and/or TGF-beta (TGFβ). Assays to measure cytokines are well known, and include but are not limited to, ELISA, intracellular cytokine staining, cytometric bead array, RT-PCR, ELISPOT, flow cytometry and bio-assays in which cells responsive to the relevant cytokine are tested for responsiveness (e.g., proliferation) in the presence of a test sample. In some aspects the biological activity can be measured using an animal model of the disease or condition, such as a tumor xenograft model, and assessing the reduction in tumor burden or load and/or survival. In some aspects the biological activity is measured by assessing clinical outcome, such as reduction in tumor burden or load.

A bispecific antibody provided herein is not limited to any particular bispecific format or method of producing it. Bispecific antibodies can be prepared as full length antibodies or antibody fragments. Among the multispecific antibodies are multispecific single-chain antibodies, e.g., diabodies, triabodies, and tetrabodies, tandem di-scFvs, and tandem tri-scFvs. In some embodiments, the bispecific antibody is formatted as a bivalent antibody. In some embodiments, the bispecific antibody is formatted as a trivalent antibody.

In some embodiments, each antigen-binding domains, including the anti-CD3 binding domain and the second antigen binding domain, comprise an antibody or an antigen-binding fragment. In some embodiments, the multispecific antibody contains one CD3 binding domain and one second antigen binding domain.

In some embodiments, the CD3 binding domain includes an antibody or an antigen-binding fragment thereof selected from the group consisting of a Fab fragment, a F(ab′)2 fragment, an Fv fragment, an scFv such as a dsFv, a scAb, a dAb, a single domain heavy chain antibody, and a single domain light chain antibody. In some embodiments, the CD3 binding domain is a Fab. In some embodiments, the CD3 binding domain is an scFv, such as a dsFv. In some embodiments, the CD3 binding domain can include any of the antibodies or antigen-binding fragments as described in Section I.A.

In some embodiments, the second antigen binding domain includes an antibody or an antigen-binding fragment thereof selected from the group consisting of a Fab fragment, a F(ab′)2 fragment, an Fv fragment, an scFv such as a dsFv, a scAb, a dAb, a single domain heavy chain antibody, and a single domain light chain antibody. In some embodiments, the second antigen binding domain is a Fab. In some embodiments, the second antigen binding domain is an scFv, such as a dsFv.

Methods of producing bi-specific T cell engagers are known, including fusion of two different hybridomas (Milstein and Cuello, Nature 1983; 305:537-540), chemical tethering though heterobifunctional cross linkers (Staerz et al. Nature 1985; 314:628-631), manufacturing of scFv-based agents of multivalency and multispecificity by varying the linker length (e.g. U.S. Pat. Nos. 5,844,094, 5,837,242 and WO 98/44001), diabodies including Dual-Affinity Re-Targeting (DART®) (see, e.g., Moore et al., 2011, Blood 117:4542-51; Veri et al, 2010, Arthritis Rheum 62:1933-43), and tetravalent and bispecific antibody-like proteins known as DVD-Igs which are engineered from two monoclonal antibodies (Wu, C. et al., Nature Biotechnology, 25, p 1290-1297, 2007). Exemplary bispecific formats are known, see e.g. Brinkmann et al. MAbs 2017, 9:182-212.

For instance, among such exemplary bispecific antibodies are bispecific T cell engager (BiTE) molecules, which contain tandem scFv molecules fused by a flexible linker (see e.g. Nagorsen and Bauerle, Exp Cell Res 317, 1255-1260 (2011); tandem scFv molecules fused to each other via, e.g. a flexible linker, and that further contain an Fc domain composed of a first and a second subunit capable of stable association (WO2013026837); diabodies and derivatives thereof, including tandem diabodies (Holliger et al, Prot Eng 9, 299-305 (1996); Kipriyanov et al, J Mol Biol 293, 41-66 (1999)); dual affinity retargeting (DART) molecules that can include the diabody format with a C-terminal disulfide bridge; or triomabs that include whole hybrid mouse/rat IgG molecules (Seimetz et al, Cancer Treat Rev 36, 458-467 (2010); and trivalent bispecific antibodies (e.g. Mayer et al. 2015 Int. J Mol Sci, 16:27497-27507 and U.S. patent publication No. US20200062826).

Examples of bispecific antibody molecules formats which may be used include (i) a single antibody that has two arms comprising different antigen-binding regions; (ii) a single chain antibody that has specificity to two different epitopes, e.g., via two scFvs linked in tandem by an extra peptide linker; (iii) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (iv) a chemically-linked bispecific (Fab′)2 fragment; (v) a Tandab, which is a fusion of two single chain diabodies resulting in a tetravalent bispecific antibody that has two binding sites for each of the target antigens; (vi) a flexibody, which is a combination of scFvs with a diabody resulting in a multivalent molecule; (vii) a so-called “dock and lock” molecule, based on the “dimerization and docking domain” in Protein Kinase A, which, when applied to Fabs, can yield a trivalent bispecific binding protein consisting of two identical Fab fragments linked to a different Fab fragment; (viii) a so-called Scorpion molecule, comprising, e.g., two scFvs fused to both termini of a human Fab-arm; and (ix) a diabody.

In some embodiments, bispecific antibodies are bivalent bispecific antibodies. In some embodiments, bivalent bispecific antibody include two antigen binding sites, namely one antigen binding sites for the second antigen and one antigen binding site for CD3. In some embodiments, a bivalent bispecific antibody is monovalent for binding to the second antigen and monovalent for binding to CD3.

In some embodiments, the bispecific antibody provided herein is composed of a CD3 binding domain that is a fragment of an antibody that specifically binds to CD3, a second antigen binding domain with a fragment of an antibody that specifically binding to a second antigen, and a Fc region. In some embodiments, the CD3 binding domain and the second antigen binding domain are linked via their C-termini to the hinge region of the Fc region. In some embodiments, the Fc region acts to multimerize the two heavy chain polypeptides of the binding molecule. Hence, the Fc region is a multimerization domain. In some embodiments, the Fc region is a heterodimeric Fc region that facilitates formation of a heterodimer containing two different heavy chain polypeptides in the bispecific binding molecule.

In some embodiments, the bispecific antibody provided herein is composed of a CD3 binding domain that is a Fab fragment of an antibody specifically binding to CD3 (further named also as “CD3-Fab”), a second antigen binding domain with an antibody means for binding to the second antigen, and a Fc region. In some embodiments, the CD3 binding domain and the second antigen binding domain are linked via their C-termini to the hinge region of the Fc part.

In some embodiments, the bispecific antibody provided herein is composed of a CD3 binding domain that is a scFv fragment, such as a dsFv, specifically binding to CD3 (further named also as “CD3-scFv”, such as “CD3-dsFv”), a second antigen binding domain with an antibody means for binding to the second antigen, and a Fc part. In some embodiments, the CD3 binding domain and the second antigen binding domain are linked via their C-termini to the hinge region of the Fc part.

In some of any of such embodiments, the bispecific binding molecule, including the anti-CD3 antibody or antigen-binding fragment thereof and/or the second antigen antibody or antigen-binding fragment thereof, comprises at least one post-translational modification of the amino acid sequence. Post-translational modifications can include, e.g., glycosylation, phosphorylation, citrullination, isomerization, ubiquitination, acetylation, hydroxylation, methylation, AMPylation, prenylation, deamidation, eliminylation, carbamylation and carbamoylation. In some embodiments, the post-translational modification is the modification of an amino acid side chain, such as conversion of an N-terminal amino acid (e.g., glutamate or glutamine) to pyroglutamate. In some such embodiments, this modification occurs at the N-terminus of the heavy chain. In some embodiments, the post-translational modification includes a post-translational modification of a heavy chain N-terminal glutamine (Q) to a pyroglutamate. In some embodiments, in a composition of an antibody or antigen-binding fragment thereof as disclosed herein, such a post-translational modification to pyroglutamate (e.g., at the N-terminus of the heavy chain) is a dominant post-translational modification; in some such embodiments, the antibody or antigen binding fragment thereof in which an N-terminal pyroglutamate is present represents greater than 50%, greater than 60%, greater than 70%, greater than 80%, greater than 90%, or greater than 95% (w/w) of the antibody or antigen binding fragment thereof in the composition.

In some embodiments, the post-translational modification is the cleavage of one or more, particularly one or two, amino acids from the C-terminus of the heavy chain. For instance, an antibody produced by expression of a specific nucleic acid molecule encoding a full-length heavy chain (e.g., by expression in a host cell) may include the full-length heavy chain, or it may include a cleaved variant of the full-length heavy chain. This may be the case where the final two C-terminal amino acids of the heavy chain are glycine and lysine, respectively. Therefore, the C-terminal lysine, or the C-terminal glycine and lysine, of the Fc region may or may not be present. Thus, an antibody may include a heavy chain constant domain with both a C-terminal glycine and lysine, without the C-terminal lysine, or without both the C-terminal glycine and lysine. In some embodiments, in a composition of an antibody or antigen-binding fragment thereof as disclosed herein, glycine cleavage is not a dominant post-translational modification; in some such embodiments, the antibody or antigen binding fragment thereof in which the C-terminal lysine is cleaved from the heavy chain constant domain represents less than 10%, less than 5%, or less than 4% (w/w) of the antibody or antigen binding fragment thereof in the composition.

### 1. Second Antigen Binding Domain

In certain embodiments, the multispecific antibodies, such as bispecific antibodies, incorporate any of the provided CD3 binding domains, such as anti-CD3 antibodies including antigen-binding fragments, and a second antigen binding domain that has specificity for a second antigen. In some embodiments, the second antigen is an antigen expressed on a target cell for which targeted killing is desired. For example, the second antigen is a target cell surface antigen.

In some embodiments, the target cell is an antigen on a B cell, such as an autoreactive B cell or a B cell malignancy. In some embodiments, the B cell antigen is an antigen selected from the group consisting of CD20, CD38, CD22, BAFF-R, and TACI. In some embodiments, the second antigen binding domain binds CD20, CD38, CD22, BAFF-R, or TACI. In some embodiments, the second antigen binding domain is an antibody or antigen binding fragment with means for binding the second antigen (e.g., CD20, CD38, CD22, BAFF-R, and TACI). In some embodiments, such provided binding molecules can be used in methods and uses for treating autoimmune disease. In some embodiments, such provided binding molecules can be used in methods and uses for treating a cancer, such as hematological malignancies or blood cancers, for example leukemia, multiple myeloma (MM), non-Hodgkin lymphoma (NHL) or Hodgkin lymphoma.

In some embodiments, the target cell is an antigen on a cancer or tumor cell. In some embodiments, the second antigen is HER2 or EGFR. In some embodiments, such provided binding molecules can be used in methods and uses for treating a cancer.

### 2. Multimerizing Domain (e.g., Fc Domain)

Bispecific antigen-binding molecules of the present invention will typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

Any bispecific antibody format or technology may be used to make the bispecific antigen-binding molecules of the present invention. For example, an antibody or antigen-binding fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mabe bispecific formats (See, for example, Spiess et al. (2015) Mol Immunol 67 (2 Pt A): 95-106 and Brinkmann et al. (2017) MAbs 9 (2): 182-212, and the references cited therein, for a review of the foregoing formats).

In some embodiments, the bispecific antibody is a full length antibody, such as a full length IgG1 antibody. In some embodiments, the full-length IgG1 antibody is a full length IgG1, λ (lambda), κ (kappa) antibody or IgG1, κ (kappa), κ (kappa) antibody. In some embodiments, the full-length antibody includes an Fc region, e.g. composed of at least a hinge region, a CH2 domain, and a CH3 domain.

In the context of bispecific antigen-binding molecules of the present invention, the multimerizing domains, e.g., Fc domains, may comprise one or more amino acid changes (e.g., insertions, deletions or substitutions) as compared to the wild-type, naturally occurring version of the Fc domain.

In some embodiments, the bispecific antibody is a dimer formed by polypeptides, each containing an Fc region. In some embodiments, the Fc region is formed by Fc domains that are mutated or modified to promote heterodimerization in which different polypeptides can be dimerized to yield a heterodimer. Thus, in some embodiments, the dimer is a heterodimer in which two polypeptide chains of the multispecific polypeptide construct are different. Exemplary modifications to promote heterodimerization are known, including any as described below.

In one aspect, a bispecific antibody provided herein is composed of a heterodimeric Fc region that facilitates interactions of two different heavy chain polypeptides in which each heavy chain polypeptide contains at least one different binding arm of the bispecific antibody. In some embodiments, the bispecific antibody includes a first heavy chain containing at least one anti-CD3 antibody or antigen-binding fragment and a first polypeptide chain of the heterodimeric Fc and a second heavy chain containing a antigen-binding domain directed against the second antigen and the second polypeptide chain of the heterodimeric Fc. In some embodiments the first polypeptide chain of the heterodimeric Fc-region comprises a first CH3 region, and the second polypeptide chain of the heterodimeric Fc region comprises a second CH3 region, wherein the sequences of the first and second CH3 regions are different and are such that the heterodimeric interaction between said first and second CH3 regions is stronger than each of the homodimeric interactions of said first and second CH3 regions.

In some embodiments, each polypeptide of an immunoglobulin Fc region comprises a human IgG1 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 86. In some embodiments, each polypeptide of an immunoglobulin Fc region comprises a human IgG1 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 87. In some embodiments, the wild-type Fc region is a wild-type IgG1 Fc region comprising the amino acid sequence set forth in SEQ ID NO: 132.

In the context of the provided disclosure the following notations are, unless otherwise indicated, used to describe a mutation; i) substitution of an amino acid in a given position is written as e.g. T350V which means a substitution of a Threonine in position 350 with a Valine; and ii) for specific variants the specific three or one letter codes are used, including the codes Xaa and X to indicate any amino acid residue. Thus, the substitution of Threonine with Valine in position 350 is designated as: T350V.

In some embodiments, an IgG1 Fc polypeptide or a variant thereof such as any described below can be made in a G1 ml or G1 m3 allotype. In some embodiments, the Fc region can contain amino acids of the human G1 ml allotype, such as residues containing Asp (D) and Leu (L) at positions 356 and 358, e.g. as set forth in SEQ ID NO:87. In some cases, an Fc polypeptide can contain amino acid substitutions E356D and M358L to reconstitute residues of allotype G1 m1. In other embodiments, the Fc region can contain amino acids of the human G1 m3 allotype, such as residues Glu (E) and Met (M) at positions 356 and 358 by EU numbering, e.g. as set forth in SEQ ID NO: 86. In some cases, an Fc polypeptide can contain amino acid substitutions D356E and L358M to reconstitute residues of allotype G1 m3.

In some embodiments, a bispecific antibody is formatted with an Fc region that lacks a C-terminal Lys (K) residue. In some embodiments, the human IgG1 Fc region lacks Lys447 (EU index of Kabat et al 1991 Sequences of Proteins of Immunological Interest).

In particular embodiments of bispecific antibodies provided herein, the human IgG Fc region is modified to induce heterodimerization. Various methods are known for promoting heterodimerization of complementary Fc polypeptides, see e.g. Ridgway et al, Protein Eng. 9:617-621 (1996); Merchant et al, Nat. Biotechnol. 16 (7): 677-81 (1998); Moore et al. (2011) MAbs, 3:546-57; Von Kreudenstein et al. MAbs, (2013) 5:646-54; Gunasekaran et al. (2010) J. Biol. Chem., 285:19637-46; Leaver-Fay et al. (2016) Structure, 24:641-51; Ha et al. (2016) Frontiers in Immunology, 7:1; Davis et al. (2010) Protein Eng Des Sel, 23:195-202; published international PCT Appl. No. WO 1998/050431, WO 2009/089004, WO2011143545 WO 2014/067011, WO 2012/058768, WO2018027025; published U.S. patent Appl. No. US20140363426, US20150307628, US20180016354, US20150239991; and U.S. patent Nos. U.S. Pat. Nos. 5,731,168, 7,183,076, 9,701,759, 9,605,084, and 9,650,446. Methods to promote heterodimerization of Fc chains include mutagenesis of the Fc region, such as by including a set of “knob-into-hole” mutations or including mutations to effect electrostatic steering of the Fc to favor attractive interactions among different polypeptide chains. For example, in some embodiments, the Fc polypeptides of a heterodimer includes a mutation to alter charge polarity across the Fc dimer interface such that coexpression of electrostatically matched Fc chains support favorable attractive interactions thereby promoting desired Fc heterodimer formation, whereas unfavorable repulsive charge interactions suppress unwanted Fc homodimer formation (Guneskaran et al. (2010) JBC, 285:19637-19646). When co-expressed in a cell, association between the chains is possible but the chains do not substantially self-associate due to charge repulsion. Other strategies for generating a heterodimeric Fc include mixing human IgG and IgA CH3 domain segments to create a complementary CH3 heterodimer, which is referred to as a SEED Fc.

Methods and variants for heterodimerization also include those described in published international PCT App. WO2014/145806, including “knobs and holes” mutations (also called “skew” variants), mutations that relate to “electrostatic steering” or “charge pairs,” and pI variants. Heterodimeric variants also include any as described in U.S. published Appl. No. US2012/0149876 or US2018/011883.

In some embodiments, modifications include introduction of a protuberance (knob) into a first Fc polypeptide and a cavity (hole) into a second Fc polypeptide such that the protuberance is positionable in the cavity to promote complexing of the first and second Fc-containing polypeptides. Amino acids targeted for replacement and/or modification to create protuberances or cavities in a polypeptide are typically interface amino acids that interact or contact with one or more amino acids in the interface of a second polypeptide. In some embodiments, modifications include a knob-into-hole modification and disulfide bond pairing mutation.

In some embodiments, a first Fc polypeptide that is modified to contain protuberance (hole) amino acids include replacement of a native or original amino acid with an amino acid that has at least one side chain which projects from the interface of the first Fc polypeptide and is therefore positionable in a compensatory cavity (hole) in an adjacent interface of a second polypeptide. Most often, the replacement amino acid is one which has a larger side chain volume than the original amino acid residue. One of skill in the art knows how to determine and/or assess the properties of amino acid residues to identify those that are ideal replacement amino acids to create a protuberance. In some embodiments, the replacement residues for the formation of a protuberance are naturally occurring amino acid residues and include, for example, arginine (R), phenylalanine (F), tyrosine (Y), or tryptophan (W). In some examples, the original residue identified for replacement is an amino acid residue that has a small side chain such as, for example, alanine, asparagine, aspartic acid, glycine, serine, threonine, or valine.

In some embodiments, a second Fc polypeptide that is modified to contain a cavity (hole) is one that includes replacement of a native or original amino acid with an amino acid that has at least one side chain that is recessed from the interface of the second polypeptide and thus is able to accommodate a corresponding protuberance from the interface of a first polypeptide. Most often, the replacement amino acid is one which has a smaller side chain volume than the original amino acid residue. One of skill in the art knows how to determine and/or assess the properties of amino acid residues to identify those that are ideal replacement residues for the formation of a cavity. Generally, the replacement residues for the formation of a cavity are naturally occurring amino acids and include, for example, alanine (A), serine(S), threonine (T) and valine (V). In some examples, the original amino acid identified for replacement is an amino acid that has a large side chain such as, for example, tyrosine, arginine, phenylalanine, or tryptophan.

The CH3 interface of human IgG1, for example, involves sixteen residues on each domain located on four anti-parallel β-strands which buries 1090 Å2 from each surface (see e.g., Deisenhofer et al. (1981) Biochemistry, 20:2361-2370; Miller et al., (1990) J Mol. Biol., 216, 965-973; Ridgway et al., (1996) Prot. Engin., 9:617-621; U.S. Pat. No. 5,731,168). Modifications of a CH3 domain to create protuberances or cavities are described, for example, in U.S. Pat. No. 5,731,168; International Patent Applications WO98/50431 and WO 2005/063816; and Ridgway et al., (1996) Prot. Engin., 9:617-621. In some examples, modifications of a CH3 domain to create protuberances or cavities are typically targeted to residues located on the two central anti-parallel β-strands. The aim is to minimize the risk that the protuberances which are created can be accommodated by protruding into the surrounding solvent rather than being accommodated by a compensatory cavity in the partner CH3 domain.

In some embodiments, to promote heterodimerization both polypeptides of the Fc heterodimer contain paired or complementary amino acid modifications. Exemplary paired amino acid modification of polypeptides of an Fc fusion are set forth in Table 2.

TABLE 2
Paired amino acids of Heterodimeric Fc

| First Fc polypeptide | Second Fc Polypeptide |
|---|---|
| T366W | T366S/L368W/Y407V |
| T366W/S354C | T366S/L368A/Y407V/Y349C |
| S364H/F405A | Y349T/Y349F |
| T350V/L351Y/F405A/Y407V | T350V/T366L/K392L/T394W |
| K360D/D399M/Y407A | E345R/Q347R/T366V/K409V |
| K409D/K392D | D399K/E356K |
| K360E/K409W | Q347R/D399V/F405T |
| L360E/K409W/Y349C | Q347R/399V/F405T/S354C |
| K370E/K409W | E357N/D399V/F405T |

In general, the Fc region is responsible for effector functions, such as complement-dependent cytotoxicity (CDC) and antibody-dependent cell cytotoxicity (ADCC), in addition to the antigen-binding capacity, which is the main function of immunoglobulins. Additionally, the FcRn sequence present in the Fc region plays the role of regulating the IgG level in serum by increasing the in vivo half-life by conjugation to an in vivo FcRn receptor. In some embodiments, such functions can be altered, such as reduced or enhanced, in an Fc for use with the provided multispecific polypeptide constructs.

In some embodiments, the Fc region includes an Fc polypeptide that is mutated or modified to alter one or more effector functions. Various examples of mutations to Fc polypeptides to alter, such as reduce, effector function are known, including any as described below. In some embodiments, reference to amino acid substitutions in an Fc region is by EU numbering by Kabat (also called Kabat numbering) unless described with reference to a specific SEQ ID NO. EU numbering is known and is according to the most recently updated IMGT Scientific Chart (IMGT®, the international ImMunoGeneTics Information System®, http://www.imgt.org/IMGTScientificChart/Numbering/Hu_IGHGnber.html (created: 17 May 2001, last updated: 10 Jan. 2013) and the EU index as reported in Kabat, E. A. et al. Sequences of Proteins of Immunological interest. 5th ed. US Department of Health and Human Services, NIH publication No. 91-3242 (1991).

In some embodiments, the human IgG1 Fc region is modified to alter antibody-dependent cellular cytotoxicity (ADCC) and/or complement-dependent cytotoxicity (CDC), e.g., the amino acid modifications described in Natsume et al., 2008 Cancer Res, 68 (10): 3863-72; Idusogie et al., 2001 J Immunol, 166 (4): 2571-5; Moore et al., 2010 mAbs, 2 (2): 181-189; Lazar et al., 2006 PNAS, 103 (11): 4005-4010, Shields et al., 2001 JBC, 276 (9): 6591-6604; Stavenhagen et al., 2007 Cancer Res, 67 (18): 8882-8890; Stavenhagen et al., 2008 Advan. Enzyme Regul., 48:152-164; Alegre et al, 1992 J Immunol, 148:3461-3468; Reviewed in Kaneko and Niwa, 2011 Biodrugs, 25 (1): 1-11; U.S. Pat. Nos. 8,101,720; 9,475,881; and 10,851,164, the contents of each of which are hereby incorporated by reference in their entireties.

In some embodiments, the Fc region, such as the human IgG1 Fc region is modified to enhance ADCC activity or CDC activity. Examples of mutations that enhance ADCC include modification at Ser239 and Ile332, for example Ser239Asp and Ile332Glu (S239D, 1332E). Examples of mutations that enhance CDC include modifications at Lys326 and Glu333. In some embodiments, the Fc region is modified at one or both of these positions, for example Lys326Ala and/or Glu333Ala (K326A and E333A) using the Kabat numbering system.

In some embodiments, the Fc region is altered to provide reduced Fc-mediated effector functions, such as via reduced Fc receptor binding, e.g. binding to FcγR binding but generally not FcRn binding. In some embodiments, the human IgG1 Fc region fusion proteins of the present disclosure lack or have reduced Fucose attached to the N-linked glycan-chain at N297. There are numerous ways to prevent fucosylation, including but not limited to production in a FUT8 deficient cell line; addition inhibitors to the mammalian cell culture media, for example Castanospermine; and metabolic engineering of the production cell line. In some embodiments, the human IgG1 Fc region is modified at amino acid Asn297 to prevent glycosylation of the fusion protein, e.g., Asn297Ala (N297A) or Asn297Asp (N297D).

In some embodiments, the Fc region of the fusion protein is altered at one or more of the following positions to reduce Fc receptor binding: Leu 234 (L234), Leu235 (L235), Asp265 (D265), Asp270 (D270), Ser298 (S298), Asn297 (N297), Asn325 (N325) or Ala327 (A327). For example, Leu 234Ala (L234A), Leu235Ala (L235A), Leu235Glu (L235E), Gly237Ala (G237A) Asp265Asn (D265N), Ser267Lys (S267K), Asp270Asn (D270N), Arg292Cys (R292C), Asn297Gly (N297G), Ser298Asn (S298N), Asn297Ala (N297A), Val302Cys (V302C), Asn325Glu (N325E), Ala327Ser (A327S) or Pro329Gly (P329G). In some embodiments, the Fc region of the fusion protein is modified at amino acid Leu235 to alter Fc receptor interactions, e.g., Leu235Glu (L235E) or Leu235Ala (L235A). In some embodiments, the Fc region of the fusion protein is modified at amino acid Leu234 to alter Fc receptor interactions, e.g., Leu234Ala (L234A). In some embodiments, the Fc region of the fusion protein is altered at both amino acid 234 and 235, e.g., Leu234Ala and Leu235Ala (L234A/L235A) or Leu234Val and Leu235Ala (L234V/L235A). In particular embodiments, the Fc region contains LALA mutations Leu234Ala and Leu235Ala (L234A/L235A). In some embodiments, modifications within the Fc region reduce binding to Fc-receptor-gamma receptors while have minimal impact on binding to the neonatal Fc receptor (FcRn).

In some embodiments, the Fc comprises amino acid modification of at least one of L234, L235, or D265. In another embodiment, the Fc comprises amino acid modification of at least L234, L235 and D265. In another embodiment, the Fc comprises the amino acid modification L234A, L235A and D265S. In particular aspects, the IgG1 has been modified to comprise or consist of an L234A, an L235A, and a D265S mutation as identified using Kabat numbering to render the Fc effector-less.

In some embodiments, the Fc comprises a P329G mutation in the Fc together with L234A-L235A (LALA) mutations. In particular aspects, the IgG1 has been modified to comprise or consist of an L234A, an L235A, and a P329G mutation as identified using Kabat numbering to render the Fc effector-less. In some embodiments, P329G is able to disrupt the interaction between hIgG and hFcγR, which combined with the effectorless mutations L234A/L235A mutations create a triple mutant L234A/L235A/P329G with no detectable binding to C1q or FcγRs, resulting in abrogated ADCC. In some embodiments, such an Fc disrupts the interaction with Fcγ-receptors (FcγRs) while maintaining the binding to neonatal Fc-receptor (nFcR) and therefore wildtype-antibody-like pharmacokinetics in subjects.

In some embodiments, the bispecific antigen-binding molecules comprising one or more modifications in the Fc domain that results in a modified Fc domain having a modified binding interaction (e.g., enhanced or diminished) between Fc and FcRn. In some embodiments, the human IgG Fc region is modified to enhance FcRn binding. In one embodiment, the bispecific antigen-binding molecule comprises a modification in a CH2 or a CH3 region, wherein the modification increases the affinity of the Fc domain to FcRn in an acidic environment (e.g., in an endosome where pH ranges from about 5.5 to about 6.0). Examples of Fc mutations that enhance binding to FcRn are Met252Tyr, Ser254Thr, Thr256Glu (M252Y, S254T, T256E, respectively) (Kabat numbering, Dall'Acqua et al 2006, J. Biol Chem Vol. 281 (33) 23514-23524), Met428Leu and Asn434Ser (M428L, N434S) (Zalevsky et al 2010 Nature Biotech, Vol. 28 (2) 157-159) (EU index of Kabat et al 1991 Sequences of Proteins of Immunological Interest). Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., L/R/S/P/Q or K) and/or 434 (e.g., H/F or Y); or a modification at position 250 and/or 428; or a modification at position 307 or 308 (e.g., 308F, V308F), and 434. In some embodiments, the mutated or modified Fc polypeptide includes the following mutations: Met252Tyr and Met428Leu or Met252Tyr and Met428Val (M252Y, M428L, or M252Y, M428V) using the Kabat numbering system. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a307 and/or 308 modification (e.g., 308F or 308P).

In some embodiments, the Fc region is mutated in one or more of the following positions to reduce Fc receptor binding: Glu233 (E233), Leu234 (L234), or Leu235 (L235). The one or more mutations can include E233P, L234V and/or L235A.

In some embodiments, the Fc region of the fusion protein is altered at Gly236 to reduce Fc receptor binding. For example, wherein Gly236 is deleted from the fusion protein. In some embodiments, the human IgG1 Fc region is modified at amino acid Gly236 to enhance the interaction with CD32A, e.g., Gly236Ala (G236A).

In particular embodiments, the mutations of the Fc region to reduce Fc effector function, e.g., via reducing Fc receptor binding to FcγR, include mutations from among any of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G or E233P/L234V/L235A/G236del.

In some embodiments, any of the provided Fc regions may include a mutation to eliminate or reduce the effector function of any antibody by mutating P238, e.g., to P238K, such as in an IgG1 or IgG2 Fc. In some embodiments, in addition to the modifications such as mutations described herein to antibodies to enhance their biological activity or reduce effector function, further mutations can be made, e.g., to the CH1, hinge, CH2 or CH3 domain, e.g., to further reduce the effector function, binding to FcγRs, and/or the stability of the antibodies. For example, any of the mutations described herein may be combined with a P238, e.g., P238K, mutation, such as in an IgG1 or IgG2 Fc. In some embodiments, Fc regions with P238K mutation demonstrate essentially no detectable binding signal towards the low affinity FcγRs hCD32a-H131, hCD32a-R131, hCD32b, hCD16a-V158 or hCD16b-NA2 and/or reduced binding affinity and a faster off-rate to the high affinity FcγR CD64 (see e.g., U.S. published patent application No. US20240141059).

In some embodiments, the bispecific antibody contains a heterodimeric Fc region in which the first Fc polypeptide of the heterodimeric Fc has at least 85%, 90%, 95% or 98% sequence identity to the Fc polypeptide set forth in SEQ ID NO:44 and contains the mutations S354C, T366W and L234A, L235E, G237A; and the second Fc polypeptide of the heterodimeric Fc has at least 85%, 90%, 95% or 98% sequence identity to the Fc polypeptide set forth in SEQ ID NO:45 and contains the mutations Y349C, T366S, L368A, Y407V and L234A, L235E, G237A. In some embodiments, the bispecific antibody contains a heterodimeric Fc region in which the first Fc polypeptide of the heterodimeric Fc is set forth in SEQ ID NO:44 and the second Fc polypeptide of the heterodimeric Fc is set forth in SEQ ID NO: 45.

In some embodiments, the bispecific antibody contains a heterodimeric Fc region in which the first Fc polypeptide of the heterodimeric Fc has at least 85%, 90%, 95% or 98% sequence identity to the Fc polypeptide set forth in SEQ ID NO:46 and contains the mutations S354C, T366W and L234A, L235A, D265S; and the second Fc polypeptide of the heterodimeric Fc has at least 85%, 90%, 95% or 98% sequence identity to the Fc polypeptide set forth in SEQ ID NO:47 and contains the mutations Y349C, T366S, L368A, Y407V and L234A, L235A, D265S. In some embodiments, the bispecific antibody contains a heterodimeric Fc region in which the first Fc polypeptide of the heterodimeric Fc is set forth in SEQ ID NO:46 and the second Fc polypeptide of the heterodimeric Fc is set forth in SEQ ID NO: 47.

In some embodiments, the bispecific antibody contains a heterodimeric Fc region in which the first Fc polypeptide of the heterodimeric Fc has at least 85%, 90%, 95% or 98% sequence identity to the Fc polypeptide set forth in SEQ ID NO:88 and contains the mutations T350V, L351Y, F405A and Y407V; and the second Fc polypeptide of the heterodimeric Fc has at least 85%, 90%, 95% or 98% sequence identity to the Fc polypeptide set forth in SEQ ID NO:89. In some embodiments, the bispecific antibody contains a heterodimeric Fc region in which the first Fc polypeptide of the heterodimeric Fc is set forth in SEQ ID NO:88 and the second Fc polypeptide of the heterodimeric Fc is set forth in SEQ ID NO: 89.

In some embodiments, the bispecific antibody contains a heterodimeric Fc region in which the first Fc polypeptide has at least 85%, 90%, 95% or 98% sequence identity to the Fc polypeptide set forth in SEQ ID NO:90 and contains the mutations T350V, L351Y, F405A and Y407V and the mutations L234A, L235A and D265S; and the second Fc polypeptide of the heterodimeric Fc region has at least 85%, 90%, 95% or 98% sequence identity to the Fc polypeptide set forth in SEQ ID NO:91 and contains the mutations T350V, T366L, K392L and T394W and the mutations L234A, L235A and D265S. In some embodiments, the bispecific antibody contains a heterodimeric Fc region in which the first Fc polypeptide is set forth in SEQ ID NO:90 and the second Fc polypeptide is set forth in SEQ ID NO:91.

In some embodiments, the bispecific antibody contains a heterodimeric Fc region in which the first Fc polypeptide has at least 85%, 90%, 95% or 98% sequence identity to the Fc polypeptide set forth in SEQ ID NO:92 and contains the mutations T350V, L351Y, F405A and Y407V and the mutations L234A, L235A and P329G; and the second Fc polypeptide of the heterodimeric Fc region has at least 85%, 90%, 95% or 98% sequence identity to the Fc polypeptide set forth in SEQ ID NO:93 and contains the mutations T350V, T366L, K392L and T394W and the mutations L234A, L235A and P329G. In some embodiments, the bispecific antibody contains a heterodimeric Fc region in which the first Fc polypeptide is set forth in SEQ ID NO:92 and the second Fc polypeptide is set forth in SEQ ID NO:93.

In some embodiments, the bispecific antibody contains a heterodimeric Fc region in which the first Fc polypeptide of the heterodimeric Fc has at least 85%, 90%, 95% or 98% sequence identity to the Fc polypeptide set forth in SEQ ID NO:48 and contains the mutations S354C, T366W, P238K, D356E, and L358M; and the second Fc polypeptide of the heterodimeric Fc has at least 85%, 90%, 95% or 98% sequence identity to the Fc polypeptide set forth in SEQ ID NO:49 and contains the mutations Y349C, T366S, L368A, Y407V, P238K, D356E, and L358M. In some embodiments, the bispecific antibody contains a heterodimeric Fc region in which the first Fc polypeptide of the heterodimeric Fc is set forth in SEQ ID NO:48 and the second Fc polypeptide of the heterodimeric Fc is set forth in SEQ ID NO: 49.

In some embodiments, the antibody contains an: (i) amino acid substitution T366W in the first polypeptide of the heterodimeric Fc and amino acid substitutions T366S, L368A and Y407V in the second polypeptide of the heterodimeric Fc; and/or (ii) amino acid substitution S354C on the first polypeptide of the heterodimeric Fc and amino acid substitution Y349C on the second polypeptide of the heterodimeric Fc.

In some embodiments, the first Fc polypeptide is an IgG1 Fc comprising amino acid substitutions L234A, L235A, D265S, S354C and T366W, and the second Fc polypeptide is an IgG1 Fc comprising amino acid substitutions L234A, L235A, D265S, Y349C, T366S, L368A and Y407V.

### C. Polynucleotides, Vectors, Cells and Methods of Producing Antibodies

The present disclosure provides a nucleic acid or nucleic acids encoding any one of the CD3 antibody binding molecules, such as CD3 monospecific antibody binding domains and also bispecific binding molecules having a CD3 antibody binding domain specific to CD3 and a second antigen, including full-length antibodies and antigen-binding fragments of any such molecule as described herein. The polypeptide sequences may be used to determine appropriate nucleic acid sequences encoding the binding domains or binding molecules disclosed thereby. The nucleic acid sequence may be optimized to reflect particular codon “preferences” for various expression systems according to standard methods well known to those of skill in the art.

Among the provided nucleic acid molecules are those that comprise polynucleotides that encode one or more polypeptides (e.g., an immunoglobulin heavy chain or light chain) of a provided antibody binding molecule, such as monospecific or bispecific antibody molecules, including full-length antibodies and functional antigen-binding fragments. In some embodiments, a nucleic acid molecule comprises a polynucleotide that encodes a heavy chain or a light chain of a CD3 binding molecule, such as monospecific or bispecific antibody molecules, including full-length antibodies and functional antigen-binding fragments. In some embodiments, a nucleic acid molecule comprises both a polynucleotide that encodes a heavy chain and a polynucleotide that encodes a light chain of any of the described CD3 binding domain or a binding molecule containing the same. In some embodiments, a first nucleic acid molecule comprises a first polynucleotide that encodes a heavy chain and a second nucleic acid molecule comprises a second polynucleotide that encodes a light chain. Also provided herein are nucleic acid molecules comprising polynucleotides that encode one or more polypeptides of a binding molecule containing any of the provided CD3 binding domains or a bispecific immune cell engager comprising the CD3 binding domain as described herein.

In some embodiments, the nucleic acid(s) is an isolated nucleic acid(s). The polynucleotides may include those encompassing natural and/or non-naturally occurring nucleotides and bases, e.g., including those with backbone modifications. The terms “nucleic acid molecule”, “nucleic acid”, “sequence of nucleotides”, and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain variable domain fragment and a light chain variable domain fragment linked together.

This disclosure is not intended to be limited with regard to the source of the antibody or the manner in which it is made. Various procedures known within the art may be used for the production of monoclonal antibodies directed against CD3 (See, for example, Antibodies: A Laboratory Manual, Harlow E, and Lane D, 1988, Cold Spring Harbor Laboratory Press, Cold Spring Harbor, NY, incorporated herein by reference). In some embodiments, the CD3 binding domain, and antigen binding fragments thereof, are recombinantly expressed and produced.

In some embodiments, a nucleic acid of the present disclosure may be operably linked to a transcriptional control element, e.g., a promoter, and enhancer, etc. Suitable promoter and enhancer elements are known to those of skill in the art.

In some embodiments, a polynucleotide encoding a heavy chain or light chain of a provided binding molecule, including full-length antibody binding domains or antigen binding fragments or bispecific antibodies as described, comprises a nucleotide sequence that encodes a leader sequence, which, when translated, is located at the N terminus of the heavy chain or light chain. The leader sequence may be the native heavy or light chain leader sequence, or may be another heterologous leader sequence.

Also provided are polynucleotides that have been optimized for codon usage and/or to eliminate splice sites, such as cryptic splice sites. In some embodiments, the polynucleotides are modified to optimize codon usage. In some embodiments, the polynucleotides are codon optimized for expression in a mammalian cell, such as a CHO cell, or a human cell.

Nucleic acid molecules may be constructed using conventional recombinant DNA techniques well-known in the art. In some embodiments, a nucleic acid molecule is an expression vector that is suitable for expression in a selected host cell.

In some embodiments, the polynucleotides are comprised in a vector. Vectors comprising polynucleotides that encode CD3 binding molecules, including heavy chains and/or light chains of the binding molecules, are provided herein. Vectors comprising polynucleotides that encode CD3 binding molecules, including heavy chains and/or light chains of the binding molecules, are also provided herein. In certain embodiments, the vector is selected from the group consisting of a DNA vector, an RNA vector, a plasmid, a lentiviral vector, an adenoviral vector, an adeno-associated viral vector, and a retroviral vector. In certain embodiments, the vector is an expression vector. A nucleic acid of the present disclosure may be present within an expression vector and/or a cloning vector.

An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. An expression vector may be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors. In one embodiment, nucleic acid is comprised in a naked DNA or RNA vector, including, for example, a linear expression element (as described in for instance Sykes and Johnston, Nat Biotech 17, 355-59 (1997)), a compacted nucleic acid vector (as described in for instance U.S. Pat. No. 6,077,835 and/or WO 00/70087), a plasmid vector such as pBR322, pUC 19/18, or pUC 118/119, a “midge” minimally-sized nucleic acid vector (as described in for instance Schakowski et al., Mol Ther 3, 793-800 (2001)), or as a precipitated nucleic acid vector construct, such as a CaP04-precipitated construct (as described in for instance WO200046147, Benvenisty and Reshef, PNAS USA 83, 9551-55 (1986), Wigler et al., Cell 14, 725 (1978), and Coraro and Pearson, Somatic Cell Genetics 7, 603 (1981)). Such nucleic acid vectors and the usage thereof are well known in the art (see for instance U.S. Pat. Nos. 5,589,466 and 5,973,972).

In one embodiment, the vector is suitable for expression of the polypeptide chains in a bacterial cell. Examples of such vectors include expression vectors such as BlueScript (Stratagene), pIN vectors (Van Heeke & Schuster, J Biol Chem 264, 5503-5509 (1989), pET vectors (Novagen, Madison Wis.) and the like).

An expression vector may also or alternatively be a vector suitable for expression in a yeast system. Any vector suitable for expression in a yeast system may be employed. Suitable vectors include, for example, vectors comprising constitutive or inducible promoters such as alpha factor, alcohol oxidase and PGH (reviewed in: F. Ausubel et al., ed. Current Protocols in Molecular Biology, Greene Publishing and Wiley InterScience New York (1987), and Grant et al., Methods in Enzymol 153, 516-544 (1987)).

An expression vector may also or alternatively be a vector suitable for expression in mammalian cells, e.g., a vector comprising glutamine synthetase as a selectable marker, such as the vectors described in Bebbington (1992) Biotechnology (NY) 10:169-175.

The following vectors are provided by way of example and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35:2543-2549; Borras et al., Gene Ther. (1999) 6:515-524; Li and Davidson, Proc. Natl. Acad. Sci. USA (1995) 92:7700-7704; Sakamoto et al., H. Gene Ther. (1999) 5:1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9:81-86, Flannery et al., Proc. Natl. Acad. Sci. USA (1997) 94:6916-6921; Bennett et al., Invest. Opthalmol. Vis. Sci. (1997) 38:2857-2863; Jomary et al., Gene Ther. (1997) 4:683 690, Rolling et al., Hum. Gene Ther. (1999) 10:641-648; Ali et al., Hum. Mol. Genet. (1996) 5:591-594; Srivastava in WO 93/09239, Samulski et al., J. Vir. (1989) 63:3822-3828; Mendelson et al., Virol. (1988) 166:154-165; and Flotte et al., Proc. Natl. Acad. Sci. USA (1993) 90:10613-10617); SV40; herpes simplex virus; human immunodeficiency virus (see, e.g., Miyoshi et al., Proc. Natl. Acad. Sci. USA (1997) 94:10319-23; Takahashi et al., J. Virol. (1999) 73:7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

The vectors can include viral vectors or eukaryotic expression vectors. In some embodiments, the viral vector is a vector derived from simian virus 40 (SV40), adenoviruses, adeno-associated virus (AAV), or retroviral or lentiviral vectors.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A nucleic acid and/or vector may also comprises a nucleic acid sequence encoding a secretion/localization sequence, which can target a polypeptide, such as a nascent polypeptide chain, to the periplasmic space or into cell culture media. Such sequences are known in the art, and include secretion leader or signal peptides.

The expression vector may comprise or be associated with any suitable promoter, enhancer, and other expression-facilitating elements. Examples of such elements include strong expression promoters (e.g., human CMV IE promoter/enhancer as well as RSV, SV40, SL3-3, MMTV, and HIV LTR promoters), effective poly (A) termination sequences, an origin of replication for plasmid product in E. coli, an antibiotic resistance gene as selectable marker, and/or a convenient cloning site (e.g., a polylinker). Nucleic acids may also comprise an inducible promoter as opposed to a constitutive promoter such as CMV IE.

In a further embodiment, a host cell comprising such polynucleotides is provided. In one such embodiment, a host cell comprises (e.g., has been transformed with) a vector or vectors that comprise one or more polynucleotides that encodes an amino acid sequence comprising the binding molecule or a chain thereof. In some embodiments, a host cell comprises (e.g., has been transformed with) one or more vectors comprising one or more polynucleotide that encodes one or more an amino acid sequence comprising one or more antibodies and/or portions thereof, e.g., antigen-binding fragments thereof. In some embodiments, a host cell comprises (e.g., has been transformed with) (1) a vector comprising a polynucleotide that encodes an amino acid sequence comprising the VL region of the antibody and an amino acid sequence comprising the VH region of the antibody, or (2) a first vector comprising a polynucleotide that encodes an amino acid sequence comprising the VL region of the antibody and a second vector comprising a polynucleotide that encodes an amino acid sequence comprising the VH region of the antibody. In some embodiments, one or more such host cells are provided. In some embodiments, a composition containing one or more such host cells are provided.

In some embodiments, the host cell may comprise the nucleic acid molecules encoding the polypeptides stably integrated into the cellular genome. In another embodiment, the cell is transformed to contain non-integrated nucleic acid, such as a plasmid, cosmid, phagemid, or linear expression element.

In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain variable domain fragment and light chain variable domain fragment are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

Also provided is a host cell comprising any of the vectors or nucleic acids disclosed herein. The host cell may be of eukaryotic, prokaryotic, mammalian, or bacterial origin. In various embodiments, heavy chains and/or light chains may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; HEK293 cells, including HEK293-6E cells; CHO cells, including CHO-S, DG44. Lec13 CHO cells, and FUT8 CHO cells; PER.C6® cells (Crucell); and NSO cells. In some embodiments, heavy chains and/or light chains may be expressed in yeast. See, e.g., U.S. Publication No. US 2006/0270045 A1. In some embodiments, a particular eukaryotic host cell is selected based on its ability to make desired post-translational modifications to the encoded polypeptide including to heavy chains and/or light chain domains. For example, in some embodiments, CHO cells produce polypeptides that have a higher level of sialylation than the same polypeptide produced in HEK293 cells.

In some embodiments, the antibody or antigen-binding fragment provided herein is produced in a cell-free system. Exemplary cell-free systems are described, e.g., in Sitaraman et al., Methods Mol. Biol. 498:229-44 (2009); Spirin, Trends Biotechnol. 22:538-45 (2004); Endo et al., Biotechnol. Adv. 21:695-713 (2003).

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, e.g., in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

Also provided herein are host cells comprising any of the polynucleotides or vectors described herein. In some embodiments host cells are capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtilis) and yeast (such as S. cerevisiae, S. pombe; or K. lactis).

A method of producing a binding molecule, such as a CD3 monospecific antibody binding domain or a bispecific binding molecule having a CD3 antibody binding domain specific to CD3 and an antibody binding domain specific to a second antigen, including an antibody or antigen-binding fragment of any such molecules, is also provided herein, wherein the method comprises culturing any of the provided host cells. In some embodiments, the nucleic acids encoding a subject binding molecule, including an antibody or antigen-binding fragment, are introduced directly into a host cell, and the cell incubated under conditions sufficient to induce expression of the encoded antibody. Expression may conveniently be achieved by culturing under appropriate conditions recombinant host cells containing the nucleic acid. Following production by expression, the binding molecule, including an antibody or antigen-binding fragment thereof, may be isolated and/or purified using any suitable technique, and then used as desired.

The provided binding molecules, including bispecific antibodies, may be purified by any suitable method. Such methods include, but are not limited to, the use of affinity matrices or hydrophobic interaction chromatography. Suitable affinity ligands such as ligands that bind antibody constant regions. For example, a Protein A, Protein G, Protein A/G, or an antibody affinity column may be used to bind the constant region and to purify a CD3 binding domain. Hydrophobic interactive chromatography, for example, a butyl or phenyl column, may also be suitable for purifying some polypeptides such as antibodies. Ion exchange chromatography (e.g., anion exchange chromatography and/or cation exchange chromatography) may also be suitable for purifying some polypeptides such as antibodies. Mixed-mode chromatography (e.g., reversed phase/anion exchange, reversed phase/cation exchange, hydrophilic interaction/anion exchange, hydrophilic interaction/cation exchange, etc.) may also be suitable for purifying some polypeptides such as antibodies. Many methods of purifying polypeptides are known in the art.

Antibodies or antigen-binding fragments thereof as provided herein, and encoding nucleic acid molecules and vectors, may be isolated and/or purified, e.g. from their natural environment, in substantially pure or homogeneous form, or, in the case of nucleic acid, free or substantially free of nucleic acid or genes of origin other than the sequence encoding a polypeptide with the desired function.

In some embodiments, the bispecific antibodies provided herein can be obtained by co-expression of constructs encoding the polypeptides of the bispecific antibody in a single host cell. In some embodiments, the methods include co-expressing the first polypeptide comprising the first heavy chain, the second polypeptide comprising the second heavy chain, and the polypeptide(s) comprising the light chain. In some embodiments, nucleic acids encoding the polypeptide chains are incorporated into expression vectors for introducing into the host cell as described. By virtue of the Fc region of the first heavy chain and second heavy chain being a different polypeptide chain of a heterodimeric Fc each containing different mutations in their CH3 region to promote heterodimeric interactions, the polypeptide chains of the first and second heavy chain will associate to form the bispecific antibody. Moreover, the light chain also will associate with the respective heavy chains. The resulting bispecific antibodies can be purified by any suitable method such as, for example, by affinity chromatography over Protein A or Protein G columns. Where two nucleic acid molecules encoding different polypeptides are transformed into cells, formation of homo- and heterodimers will occur. Conditions for expression can be adjusted so that heterodimer formation is favored over homodimer formation.

Techniques for recovery of heterodimers from homodimers based on a differential affinity of the heterodimers for an affinity reagent are known. In some aspects, such techniques include designing a heterodimer so that one of the Fc polypeptide chains does not bind to the affinity reagent protein A. In some cases, one of the polypeptide chain can contain one or more amino acid substitution to abrogate or reduce affinity for the protein A reagent in one of the polypeptides of the Fc heterodimer, see e.g. WO2017134440, WO2010151792, Jendeberg et al. (Jendeberg et al., (1997) J. Immunol. Methods, 201 (1): 25-34. In some of these embodiments, the Fc region may be modified at the protein-A binding site on one member of the heterodimer so as to prevent protein-A binding and thereby enable more efficient purification of the heterodimeric fusion protein.

In some embodiments, separate plasmids encoding each chain of the bispecific antibody are transfected at an equimolar ratio into mammalian cells (such as HEK293 or CHO). Recombinant protein is secreted into the supernatant and can be collected, such as after 3-7 days. In some embodiments, the antibody is purified by protein A chromatography, followed by either preparative size exclusion chromatography (SEC) or flow-through hydrophobic interaction chromatography (HIC). In some embodiments, a second chromatography step may be carried out, such as on SEC (AKTA with Superdex-200 resin) or FT-HIC (AKTA with butyl/phenyl sepharose), to remove undesired cross-paired species. In some embodiments, the bispecific antibody is a purified bispecific antibody.

### III. PHARMACEUTICAL COMPOSITIONS

Provided herein are compositions containing any of the provided antibodies or antigen-binding fragments or binding molecules containing same as described herein. The pharmaceutical composition can further comprise a pharmaceutically acceptable excipient. For example, the pharmaceutical composition can contain one or more excipients for modifying, maintaining or preserving, for example, the pH, osmolarity, viscosity, clarity, color, isotonicity, odor, sterility, stability, rate of dissolution or release, adsorption, or penetration of the composition. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

In some embodiments, the pharmaceutical composition is a solid, such as a powder, capsule, or tablet. For example, the components of the pharmaceutical composition can be lyophilized. In some embodiments, the solid pharmaceutical composition is reconstituted or dissolved in a liquid prior to administration.

In some embodiments, the pharmaceutical composition is a liquid, for example a provided protein dissolved in an aqueous solution (such as physiological saline or Ringer's solution). In some embodiments, the pH of the pharmaceutical composition is between about 4.0 and about 8.5 (such as between about 4.0 and about 5.0, between about 4.5 and about 5.5, between about 5.0 and about 6.0, between about 5.5 and about 6.5, between about 6.0 and about 7.0, between about 6.5 and about 7.5, between about 7.0 and about 8.0, or between about 7.5 and about 8.5).

In some embodiments, the pharmaceutical composition comprises a pharmaceutically-acceptable excipient, for example a filler, binder, coating, preservative, lubricant, flavoring agent, sweetening agent, coloring agent, a solvent, a buffering agent, a chelating agent, or stabilizer. Examples of pharmaceutically-acceptable fillers include cellulose, dibasic calcium phosphate, calcium carbonate, microcrystalline cellulose, sucrose, lactose, glucose, mannitol, sorbitol, maltol, pregelatinized starch, corn starch, or potato starch. Examples of pharmaceutically-acceptable binders include polyvinylpyrrolidone, starch, lactose, xylitol, sorbitol, maltitol, gelatin, sucrose, polyethylene glycol, methyl cellulose, or cellulose. Examples of pharmaceutically-acceptable coatings include hydroxypropyl methylcellulose (HPMC), shellac, corn protein zein, or gelatin. Examples of pharmaceutically-acceptable disintegrants include polyvinylpyrrolidone, carboxymethyl cellulose, or sodium starch glycolate. Examples of pharmaceutically-acceptable lubricants include polyethylene glycol, magnesium stearate, or stearic acid. Examples of pharmaceutically-acceptable preservatives include methyl parabens, ethyl parabens, propyl paraben, benzoic acid, or sorbic acid. Examples of pharmaceutically-acceptable sweetening agents include sucrose, saccharine, aspartame, or sorbitol. Examples of pharmaceutically-acceptable buffering agents include carbonates, citrates, gluconates, acetates, phosphates, or tartrates.

In some embodiments, the pharmaceutical composition further comprises an agent for the controlled or sustained release of the product, such as injectable microspheres, bio-erodible particles, polymeric compounds (polylactic acid, polyglycolic acid), beads, or liposomes.

In some embodiments, the pharmaceutical composition is sterile. Sterilization may be accomplished by filtration through sterile filtration membranes or radiation. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

A pharmaceutically acceptable carrier may be a pharmaceutically acceptable material, composition, or vehicle. For example, the carrier may be a liquid or solid filler, diluent, excipient, solvent, or encapsulating material, or some combination thereof. Each component of the carrier must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits.

In some embodiments, the pharmaceutical composition is for administration to a subject. Generally, dosages and routes of administration of the pharmaceutical composition are determined according to the size and condition of the subject, according to standard pharmaceutical practice. For example, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans. The exact dosage will be determined in light of factors related to the subject requiring treatment. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy.

Long-acting pharmaceutical compositions may be administered every 3 to 4 days, every week, or biweekly depending on the half-life and clearance rate of the particular formulation. The frequency of dosing will depend upon the pharmacokinetic parameters of the molecule in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

In some embodiments, the pharmaceutical composition is for administration to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, or intraspinally.

A provided pharmaceutical formulation may, for example, be in a form suitable for intravenous infusion.

In some embodiments, the dosage of the pharmaceutical composition is a single dose or a repeated dose.

### IV. METHODS OF USE

The CD3 binding domains and antigen-binding fragments or binding molecules containing the same described herein, including pharmaceutical compositions thereof, can be used in a variety of therapeutic applications, such as the treatment of a disease. Such methods and uses include therapeutic methods and uses, for example, involving administration of the molecules or compositions containing the same, to a subject having a disease or disorder. In some embodiments, the binding molecule is administered in an effective amount to effect treatment of the disease or disorder. Uses include uses of the CD3 binding domains and antigen-binding fragments or binding molecules containing the same described herein, including pharmaceutical compositions thereof, in such methods and treatments, and in the preparation of a medicament in order to carry out such therapeutic methods. In some embodiments, the methods are carried out by administering the CD3 binding domains and antigen-binding fragments or binding molecules containing the same, including pharmaceutical compositions thereof, or compositions comprising the same, to the subject having or suspected of having the disease or disorder. In some embodiments, the methods thereby treat the disease or disorder in the subject. Also provided are any methods or uses involving administering a provided binding molecule as described, or pharmaceutical compositions containing the same, for treating a disease or disorder.

Illustrative subjects include mammalian subjects, such as farm animals, domestic animals, and human patients. In particular embodiments, the subject is a human subject. The terms subject and patient are used interchangeably herein.

In some cases, a subject is selected that is known, suspected or that has been identified as having a tumor. A provided CD3 binding domain or antigen-binding fragment or binding molecule as described herein, or pharmaceutical composition comprising the same, is administered to the subject. The administration to the subject will generally have an effect due to its binding with the CD3 target.

Generally, alleviation or treatment of a disease or disorder involves the lessening of one or more symptoms or medical problems associated with the disease or disorder. For example, in the case of cancer, the therapeutically effective amount of the drug can accomplish one or a combination of the following: reduce the number of cancer cells; reduce the tumor size; inhibit (i.e., to decrease to some extent and/or stop) cancer cell infiltration into peripheral organs; inhibit tumor metastasis; inhibit, to some extent, tumor growth; and/or relieve to some extent one or more of the symptoms associated with the cancer. In some embodiments, a composition of this disclosure can be used to prevent the onset or reoccurrence of the disease or disorder in the subject.

In some embodiments, the CD3 binding domains or antigen-binding fragment or binding molecule as described herein, or pharmaceutical composition comprising the same, can be used to inhibit growth of mammalian cancer cells (such as human cancer cells). A method of treating cancer can include administering an effective amount of any of the pharmaceutical compositions described herein to a subject with cancer. The effective amount of the pharmaceutical composition can be administered to inhibit, halt, or reverse progression of cancers. Human cancer cells can be treated in vivo, or ex vivo. In ex vivo treatment of a human patient, tissue or fluids containing cancer cells are treated outside the body and then the tissue or fluids are reintroduced back into the patient. In some embodiments, the cancer is treated in a human patient in vivo by administration of the therapeutic composition into the patient.

In some embodiments, the CD3 binding domains or antigen-binding fragment or binding molecule as described herein, or pharmaceutical composition comprising the same, are useful in treating, alleviating a symptom of, ameliorating and/or delaying the progression of a disease or disorder. In some embodiments, the disease or disorder is a cancer, tumor or other neoplastic condition. In some embodiments, the cancer is bladder cancer, breast cancer, gastric cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, and virus-related cancer. In some embodiments, the cancer is ovarian cancer. In certain embodiments, the cancer is a metastatic cancer, refractory cancer, or recurrent cancer.

Compositions of the invention can be administered in dosages and routes and at times to be determined in appropriate pre-clinical and clinical experimentation and trials. Compositions may be administered multiple times at dosages within these ranges. Administration of the compositions may be combined with other methods useful to treat the desired disease or condition as determined by those of skill in the art. Typically, precise amount of the compositions of the present disclosure to be administered can be determined by a physician with consideration of individual differences in age, weight, tumor size, extent of infection or metastasis, and condition of the patient (subject).

In some embodiments, a therapeutically effective dose may be, by way of nonlimiting example, using body weight.

In some embodiments, a therapeutic amount of a binding molecule composition of the present disclosure is administered. Binding molecule compositions may also be administered multiple times at these dosages. Any suitable method or route can be used to administer a presently disclosed binding molecules. Routes of administration include, for example, oral, intravenous, intraperitoneal, subcutaneous, or intramuscular administration. It should be emphasized, however, that the presently disclosed subject matter is not limited to any particular method or route of administration. The optimal dosage and treatment regime for a particular patient can readily be determined by one skilled in the art of medicine by monitoring the patient for signs of disease and adjusting the treatment accordingly.

The CD3 binding domains or antigen-binding fragment or binding molecule, or pharmaceutical composition comprising the same, of the present disclosure can be administered alone or in combination with other modes of treatment, such as other anti-cancer agents. They can be provided before, substantially contemporaneous with, or after other modes of treatment (i.e., concurrently or sequentially). In some embodiments, the method of treatment described herein can further include administering: radiation therapy, chemotherapy, vaccination, targeted tumor therapy, CAR-T therapy, oncolytic virus therapy, cancer immunotherapy, cytokine therapy, surgical resection, chromatin modification, ablation, cryotherapy, an antisense agent against a tumor target, a siRNA agent against a tumor target, a microRNA agent against a tumor target or an anti-cancer/tumor agent, or a biologic, such as an antibody, cytokine, or receptor extracellular domain-Fc fusion.

### V. ARTICLES OF MANUFACTURE OR KITS

Provided herein are articles of manufacture and kits comprising the provided compositions In some embodiments, the kit comprises any of the provided compositions and instructions for administering the composition to a subject.

Kits can optionally include one or more components such as instructions for use, devices and additional reagents (e.g., sterilized water or saline solutions for dilution of the compositions and/or reconstitution of lyophilized protein), and components, such as tubes, containers and syringes for practice of the methods. In some embodiments, the kits can further contain reagents for collection of samples, preparation and processing of samples, and/or reagents for quantitating the amount of one or more surface markers in a sample, such as, but not limited to, detection reagents, such as antibodies, buffers, substrates for enzymatic staining, chromagens or other materials, such as slides, containers, microtiter plates, and optionally, instructions for performing the methods. Those of skill in the art will recognize many other possible containers and plates and reagents that can be used in accord with the provided methods.

In some embodiments, the kits can be provided as articles of manufacture that include packing materials for the packaging of the cells, antibodies or reagents, or compositions thereof, or one or more other components. For example, the kits can contain containers, bottles, tubes, vial and any packaging material suitable for separating or organizing the components of the kit. The one or more containers may be formed from a variety of materials such as glass or plastic. In some embodiments, the one or more containers hold a composition comprising cells or an antibody or other reagents for use in the methods. The article of manufacture or kit herein may comprise the cells, antibodies or reagents in separate containers or in the same container.

In some embodiments, the one or more containers holding the composition may be a single-use vial or a multi-use vial, which, in some cases, may allow for repeat use of the composition. In some embodiments, the article of manufacture or kit may further comprise a second container comprising a suitable diluent. The article of manufacture or kit may further include other materials desirable from a commercial, therapeutic, and user standpoint, including other buffers, diluents, filters, needles, syringes, therapeutic agents and/or package inserts with instructions for use.

In some embodiments, the kit can, optionally, include instructions. Instructions typically include a tangible expression describing the cell composition, reagents and/or antibodies and, optionally, other components included in the kit, and methods for using such. In some embodiments, the instructions indicate methods for using the cell compositions and antibodies for administration to a subject for treating a disease or condition, such as in accord with any of the provided embodiments. In some embodiments, the instructions are provided as a label or a package insert, which is on or associated with the container. In some embodiments, the instructions may indicate directions for reconstitution and/or use of the composition.

### VI. EXAMPLES

The following examples are included for illustrative purposes only and are not intended to limit the scope of the invention.

### Example 1: Generation of Anti-CD3 Antibodies

Fully human anti-CD3 antibodies were generated by immunizing human IgG transgenic Kirin-Medarex (KM) mice as described in Lonberg et al. (1994) Nature 368 (6474): 856-9. Enrichment for antibodies with cross-reactivity to both human and cynomologus (cyno) CD3-epsilon was achieved by footpad immunization of KM mice with a Keyhole limpet hemocyanin-fused N-terminal 14-amino acid peptide of CD3-epsilon that shares 13/14 amino acids between human and cyno CD3-epsilon (i.e., ZDGNEEMGGITQTP (SEQ ID NO: 127)), where Z represents pyroglutamic acid (PCA), a chemically modified version of the amino acid glutamine (“Q”)). The specific antibody response against CD3-epsilon in these mice was monitored over time by ELISA-based screening of serum, and mice with sufficient titers of anti-CD3-epsilon antibodies were selected for subsequent fusions. Lymph nodes and spleens from these mice were harvested, and hybridomas were generated via an electroporation unit to provide a pulse of electricity to increase permeability of the cell membrane, allowing for fusion of antibody secreting plasma cells with immortalized myeloma cells.

### Example 2: Identification of Anti-CD3-Epsilon-Specific Antibodies

Hybridoma supernatants from the hybridomas generated in Example 1 were screened by ELISA to identify antibodies specific for the immunizing peptide (i.e., ZDGNEEMGGITQTP (SEQ ID NO: 127)). A total of 87 hybridoma fusions were performed, which yielded more than 1000 antibody clones with specificity for CD3-epsilon. ELISA-positive clones were subsequently screened by flow-cytometry for cell-binding to endogenously expressed human and cyno CD3 on immortalized Jurkat cells, primary human pan-T cells (isolated from peripheral blood mononuclear cells from healthy donors), and cyno pan-T cells (isolated from peripheral blood mononuclear cells). Binding of antibodies to cell surface CD3-epsilon was measured with a fluorescently-conjugated secondary anti-human IgG antibody (data not shown). Relative cell binding was measured as mean-fluorescent intensity and compared to a positive control anti-human/cyno crossreactive anti-CD3 antibody (data not shown). Secondary Fluorescence-activated cell sorting (FACS) analysis yielded approximately 475 clones that bound to membrane-expressed human and cyno CD3-epsilon. FACS-positive clones were subsequently purified from hybridoma supernatants for functional characterization.

### Example 3: Functional Characterization of Anti-CD3 Antibodies

The ELISA and FACS-positive anti-CD3 clones identified in Example 2 were analyzed to determine their ability to induce proliferation of primary human and cyno pan-T cells. Antibodies were immobilized on wells of 96-well non-tissue culture treated flat-bottom plates at concentrations between 0.1 and 10 μg/milliliter in phosphate buffered saline by overnight incubation at 4° Celsius. Unbound antibody was subsequently washed away, and 1×10⁵ purified primary human or cyno T cells were added directly to antibody-coated wells in 200 μl of RMPI-1640 media containing 10% fetal bovine serum. T-cell proliferation was measured after 72 hours by incubation of cells with 0.5 mCi/well of 3H-thymidine for the final 6 hours of culture. Cells in each well were then lysed, and DNA was captured onto a glass-fiber filter. T-cell proliferation was determined by measuring the amount of radioactive thymidine incorporated into the DNA of the cells (data not shown). This functional analysis identified a total of 32 clones that were able to induce proliferation of primary human and cyno T cells with similar potency to a positive control anti-CD3 cyno/human crossreactive (SP34), or at least 1.5-fold increase in proliferation compared to an isotype control antibody (data not shown).

### Example 4: Binding Affinity of Anti-CD3 Antibodies to Human and Cyno CD3 Determined by Surface Plasmon Resonance (SPR)

The functionally active anti-CD3 antibodies identified in Example 3 were sequenced by next-generation sequencing and then produced recombinantly in HEK cells by transient expression in HEK cells. The binding affinities and kinetic constants of the recombinantly produced antibodies for human CD3 and cyno CD3-epsilon were then determined by surface plasmon resonance (SPR) at 37 °C using an antibody-capture format, multi-cycle kinetics on a Biacore T200 instrument.

A Biacore CM4 Series S sensor chip (Cytiva) was selected for kinetic measurements, and the system was primed with HBS-T+ (10 mM HEPES, 150 mM NaCl, 0.05% Tween-20, pH 7.4 buffer (Corning)) for the immobilization. The sensor temperature was adjusted to 37 °C prior to chip insertion, and the sample compartment was maintained at 15 °C. Upon priming, the chip was activated using a fresh 1:1 mixture of 0.4 M EDC and 0.1 M NHS (Cytiva) for seven minutes at 20 μl/ml flow rate across all four flow cells. Goat F(ab′)2 anti-human Kappa or goat anti-human IgG Fc polyclonal antibodies (Southern Biotech) were diluted to 60 μg/mL in 10 mM sodium acetate, pH 4.5 (Cytiva) and amine-coupled to all flow cells for 7 minutes at 20 μL/min. Finally, to block the activated chip surface, 0.1 M ethylenediamine in 150 mM sodium borate, pH 8.5 buffer was injected over all flow cells for 7 minutes at 20 μl/minute. Surfaces were regenerated with two 30-second injections of 0.425% phosphoric acid (Sigma-Aldrich) at 30 μL/min.

Anti-CD3 antibodies were measured for their binding affinity and kinetics to human and cyno CD3εδ-muFcG1 in a multi-cycle capture kinetics experiment. Running buffer was switched to HBS-T+ (10 mM HEPES, 150 mM NaCl, 0.05% Tween-20, pH 7.4 buffer (Corning) with 0.1% BSA (Sigma-Aldrich) and the system was thoroughly primed in this buffer. Anti-CD3 antibodies were diluted to 1-10 μg/mL into running buffer and served as ligand. Human and cyno CD3εδ-muFcG1 proteins were serially diluted to 20 nM, 100 nM, and 500 nM concentrations with running buffer. Three startup cycles, which are abbreviated analysis cycles of shortened injections but with complete regeneration, were introduced prior to the analysis experiment. In each analysis cycle, individual anti-CD3 antibodies were separately captured as ligand to flow cells 2, 3 or 4 at 10 μL/min for 30 to 90 seconds to achieve an Rₘₐₓ of approximately 50 RUs. Flow cell 1 remained unaddressed and served as the reference surface. A one-minute stabilization period was introduced after the last ligand capture onto flow cell 4. A single analyte sample was injected per cycle over all four flow cells for 2 min at 30 μL/min with 5-minute dissociation. Analyte samples comprised of the human and cyno CD3εδ-muFcG1 serial dilutions and were injected in order from lowest to highest concentration with buffer blanks flanking each concentration series. Amine-coupled goat F(ab′)2 anti-human Kappa or goat anti-human IgG Fc polyclonal antibody surfaces were regenerated at the end of each cycle with two 30-second injections of 0.425% phosphoric acid (Sigma-Aldrich) at 30 μL/min. Cycles were repeated until every antibody was tested for binding to every analyte sample at all concentrations including buffer blanks.

Finally, the data was analyzed using Biacore T200 evaluation software. The rate of complex formation (kₐ) and dissociation (k_{d}) as well as overall dissociation constant (K_{D}) for selected anti-CD3 antibodies are shown in Table E1. The V_{H} and V_{L} sequences of parental anti-CD3 antibody 1-CD3 are shown in Table E2.

TABLE E1
Kinetics and affinity of binding of
the parental anti-CD3 antibody 1-CD3

| Antibody | Antigen | kₐ (M⁻¹s⁻¹) | k_{d} (s⁻¹) | K_{D} (nM) |
|---|---|---|---|---|
| 1-CD3 | human CD3-epsilon | 3.6E+05 | 2.9E−03 | 8.3 |
| 1-CD3 | cyno CD3-epsilon | 4.5E+05 | 3.2E−03 | 7.1 |

TABLE E2
VH/VL Sequences of the parental anti-
CD3 antibody 1-CD3

| | | SEQ |
|---|---|---|
| | | ID |
| Description | Sequence | NO: |
| 1-CD3 | QVQLVESGGGVVQPGRSLRLSCAASGFTF | 24 |
| Variable | SRYGIHWVRQAPGKGLEWVAAIWYDGSKK | |
| heavy (VH) | YYADSVKGRFTISRDNSKNTLDLQMNSLR | |
| | AEDTAVYYCARSNYDILTRYYQYGINVWG | |
| | QGTTVTVSS | |
| 1-CD3 | AIQLTQSPSSLSASVGDRVTITCRASQGI | 39 |
| Variable | SSALAWYQQKPGKAPKLLIYDASSLENGV | |
| light (VL) | PSRFSGSGSGTDFTLTISSLQPEDFATYY | |
| | CQQFKSYPRTFGQGTKVEIK | |

### Example 5: Engineering the Affinity of the CD3 Binding Arm of Anti-CD3 Antibodies to Modulate Binding Activity

Previous work in mice has shown that anti-CD3-bispecific antibodies with high affinity (<1 nM) for CD3-epsilon accumulate primarily in secondary lymphoid organs, where most T cells reside (Mandikian et. al (2018) Mol Cancer Ther. 17 (4): 776-785).

To generate affinity engineered CD3 binders, residues involved in CD3 antigen binding were identified by comparing structural models of the 1-CD3 parental antibody with structural models of clones with sequences similar to the 1-CD3 sequence but having lower affinity binding to CD3 (identified in Example 2, data not shown). The residues identified in this analysis were then substituted to produce seventeen (17) heavy chain variants (identified as 1-CD3 M1-M3, M5-M11, and M16-M22) and five (5) light chain variants (identified as 1-CD3 M4 and M12-M15) having modulated affinity for CD3. Framework mutations in these modulated affinity variants were subsequently converted back to germline sequence to produce 1-CD3 heavy chain variants C1, C5, C6, C8, C9, C11, C16, C17, C21, and C22 and light chain variant C12. The sequences of the affinity variant binders and framework fixed chains are shown in Table E3 (V_{H} sequences) and Table E4 (V_{L} sequences).

TABLE E3
Sequences of 1-CD3-VH variants having modulated affinity

| | | SEQ ID |
|---|---|---|
| Description | Amino acid Sequence | NO: |
| 1-CD3-M1 | QVQLVESGGGVVQPGRSLRLSCAASGFTFSRYGIHWVRQA | 4 |
| | PGKGLEWVAAIWYDGSKKYYADSVKGRFTISRDNSKNTLY | |
| | LQMNSLRAEDTAVYYCARSNYDILTRYYQYGINVWGQGT | |
| | TVTVSS | |
| 1-CD3- M2 | QVQLVESGGGVVQPGRSLRLSCAASGFTFSRYGIHWVRQA | 53 |
| | PGKGLEWVAAIWYDSSKKYYADSVKGRFTISRDNSKNTLD | |
| | LQMNSLRAEDTAVYYCARSNYDILTRYYQYGINVWGQGT | |
| | TVTVSS | |
| 1-CD3-M3 | QVQLVESGGGVVQPGRSLRLSCAASGFTFSRYGIHWVRQA | 54 |
| | PGKGLEWVAAIWYDASKKYYADSVKGRFTISRDNSKNTLD | |
| | LQMNSLRAEDTAVYYCARSNYDILTRYYQYGINVWGQGT | |
| | TVTVSS | |
| 1-CD3-M5 | QVQLVESGGGVVQPGRSLRLSCAASGFTFSRYGIHWVRQA | 55 |
| | PGKGLEWVAAIWYDGSHKYYADSVKGRFTISRDNSKNTLD | |
| | LQMNSLRAEDTAVYYCARSNYDILTRYYQYGINVWGQGT | |
| | TVTVSS | |
| 1-CD3-M6 | QVQLVESGGGVVQPGRSLRLSCAASGFTFSRYGIHWVRQA | 56 |
| | PGKGLEWVAAIWYDGSNKYYADSVKGRFTISRDNSKNTLD | |
| | LQMNSLRAEDTAVYYCARSNYDILTRYYQYGINVWGQGT | |
| | TVTVSS | |
| 1-CD3-M7 | QVQLVESGGGVVQPGRSLRLSCAASGFTFSRYGIHWVRQA | 57 |
| | PGKGLEWVAAIWYDGSKKYYADSVKGRFTISRDNSKNTLD | |
| | LQMNSLRAEDTAVYYCARSNYDILTRHYQYGINVWGQGT | |
| | TVTVSS | |
| 1-CD3-M8 | QVQLVESGGGVVQPGRSLRLSCAASGFTFSRYGIHWVRQA | 58 |
| | PGKGLEWVAAIWYDGSKKYYADSVKGRFTISRDNSKNTLD | |
| | LQMNSLRAEDTAVYYCARSNYDILTGHYQYGINVWGQGT | |
| | TVTVSS | |
| 1-CD3-M9 | QVQLVESGGGVVQPGRSLRLSCAASGFTFSRYGIHWVRQA | 59 |
| | PGKGLEWVAAIWYDGSKKYYADSVKGRFTISRDNSKNTLD | |
| | LQMNSLRAEDTAVYYCARSNYDILTGYYQYGINVWGQGT | |
| | TVTVSS | |
| 1-CD3-M10 | QVQLVESGGGVVQPGRSLRLSCAASGFTFSRYGIHWVRQA | 60 |
| | PGKGLEWVAAIWYDGSKKYYADSVKGRFTISRDNSKNTLD | |
| | LQMNSLRAEDTAVYYCARSNYDILTRYYQHGINVWGQGT | |
| | TVTVSS | |
| 1-CD3-M11 | QVQLVESGGGVVQPGRSLRLSCAASGFTFSRYGIHWVRQA | 61 |
| | PGKGLEWVAAIWYDGSKKYYADSVKGRFTISRDNSKNTLD | |
| | LQMNSLRAEDTAVYYCARSNYDILTRYYSHGINVWGQGTT | |
| | VTVSS | |
| 1-CD3-M16 | QVQLVESGGGVVQPGRSLRLSCAASGFTFSNYGIHWVRQA | 62 |
| | PGKGLEWVAAIWYDGSKKYYADSVKGRFTISRDNSKNTLD | |
| | LQMNSLRAEDTAVYYCARSNYDILTRYYQYGINVWGQGT | |
| | TVTVSS | |
| 1-CD3-M17 | QVQLVESGGGVVQPGRSLRLSCAASGFTFSNHGIHWVRQA | 63 |
| | PGKGLEWVAAIWYDGSKKYYADSVKGRFTISRDNSKNTLD | |
| | LQMNSLRAEDTAVYYCARSNYDILTRYYQYGINVWGQGT | |
| | TVTVSS | |
| 1-CD3-M18 | QVQLVESGGGVVQPGRSLRLSCAASGFTFSRHGIHWVRQA | 64 |
| | PGKGLEWVAAIWYDGSKKYYADSVKGRFTISRDNSKNTLD | |
| | LQMNSLRAEDTAVYYCARSNYDILTRYYQYGINVWGQGT | |
| | TVTVSS | |
| 1-CD3-M19 | QVQLVESGGGVVQPGRSLRLSCAASGFTFSRYGIHWVRQA | 65 |
| | PGKGLEWVAAIWYDGSKKYYADSVKGRFTISRDNSKNTLD | |
| | LQMNSLRAEDTAVYYCARSNFDILTRYYQYGINVWGQGTT | |
| | VTVSS | |
| 1-CD3-M20 | QVQLVESGGGVVQPGRSLRLSCAASGFTFSRYGIHWVRQA | 66 |
| | PGKGLEWVAAIWYDGSKKYYADSVKGRFTISRDNSKNTLD | |
| | LQMNSLRAEDTAVYYCARSNFDVLTRYYQYGINVWGQGT | |
| | TVTVSS | |
| 1-CD3-M21 | QVQLVESGGGVVQPGRSLRLSCAASGFTFSRYGIHWVRQA | 67 |
| | PGKGLEWVAAIWYDGSKKYYADSVKGRFTISRDNSKNTLD | |
| | LQMNSLRAEDTAVYYCARSNYDVLTRYYQYGINVWGQGT | |
| | TVTVSS | |
| 1-CD3-M22 | QVQLVESGGGVVQPGRSLRLSCAASGFTFSRYGIHWVRQA | 68 |
| | PGKGLEWVAAIWYDGSKKYYADSVKGRFTISRDNSKNTLD | |
| | LQMNSLRAEDTAVYYCARSNYDILTRYYQYGLDVWGQGT | |
| | TVTVSS | |
| 1-CD3-C1 | QVQLVESGGGVVQPGRSLRLSCAASGFTFSRYGIHWVRQA | 69 |
| | PGKGLEWVAAIWYDASKKYYADSVKGRFTISRDNSKNTLY | |
| | LQMNSLRAEDTAVYYCARSNYDILTRYYQYGINVWGQGT | |
| | TVTVSS | |
| 1-CD3-C5 | QVQLVESGGGVVQPGRSLRLSCAASGFTFSRYGIHWVRQA | 70 |
| | PGKGLEWVAAIWYDASHKYYADSVKGRFTISRDNSKNTLY | |
| | LQMNSLRAEDTAVYYCARSNYDILTRYYQYGINVWGQGT | |
| | TVTVSS | |
| 1-CD3-C6 | QVQLVESGGGVVQPGRSLRLSCAASGFTFSRYGIHWVRQA | 71 |
| | PGKGLEWVAAIWYDASNKYYADSVKGRFTISRDNSKNTLY | |
| | LQMNSLRAEDTAVYYCARSNYDILTRYYQYGINVWGQGT | |
| | TVTVSS | |
| 1-CD3-C8 | QVQLVESGGGVVQPGRSLRLSCAASGFTFSRYGIHWVRQA | 72 |
| | PGKGLEWVAAIWYDASKKYYADSVKGRFTISRDNSKNTLY | |
| | LQMNSLRAEDTAVYYCARSNYDILTGHYQYGINVWGQGT | |
| | TVTVSS | |
| 1-CD3-C9 | QVQLVESGGGVVQPGRSLRLSCAASGFTFSRYGIHWVRQA | 73 |
| | PGKGLEWVAAIWYDASKKYYADSVKGRFTISRDNSKNTLY | |
| | LQMNSLRAEDTAVYYCARSNYDILTGYYQYGINVWGQGT | |
| | TVTVSS | |
| 1-CD3-C11 | QVQLVESGGGVVQPGRSLRLSCAASGFTFSRYGIHWVRQA | 27 |
| | PGKGLEWVAAIWYDASKKYYADSVKGRFTISRDNSKNTLY | |
| | LQMNSLRAEDTAVYYCARSNYDILTRYYSHGINVWGQGTT | |
| | VTVSS | |
| 1-CD3-C16 | QVQLVESGGGVVQPGRSLRLSCAASGFTFSNYGIHWVRQA | 25 |
| | PGKGLEWVAAIWYDASKKYYADSVKGRFTISRDNSKNTLY | |
| | LQMNSLRAEDTAVYYCARSNYDILTRYYQYGINVWGQGT | |
| | TVTVSS | |
| 1-CD3-C17 | QVQLVESGGGVVQPGRSLRLSCAASGFTFSNHGIHWVRQA | 74 |
| | PGKGLEWVAAIWYDASKKYYADSVKGRFTISRDNSKNTLY | |
| | LQMNSLRAEDTAVYYCARSNYDILTRYYQYGINVWGQGT | |
| | TVTVSS | |
| 1-CD3-C21 | QVQLVESGGGVVQPGRSLRLSCAASGFTFSRYGIHWVRQA | 75 |
| | PGKGLEWVAAIWYDASKKYYADSVKGRFTISRDNSKNTLY | |
| | LQMNSLRAEDTAVYYCARSNYDVLTRYYQYGINVWGQGT | |
| | TVTVSS | |
| 1-CD3-C22 | QVQLVESGGGVVQPGRSLRLSCAASGFTFSRYGIHWVRQA | 26 |
| | PGKGLEWVAAIWYDASKKYYADSVKGRFTISRDNSKNTLY | |
| | LQMNSLRAEDTAVYYCARSNYDILTRYYQYGLDVWGQGT | |
| | TVTVSS | |

TABLE E4
Sequences of 1-CD3-VL variants having modulated affinity

| | | SEQ ID |
|---|---|---|
| Description | Amino acid Sequence | NO: |
| 1-CD3-M4 | AIQLTQSPSSLSASVGDRVTITCRASQGISSALAWYQQKPG | 76 |
| | KAPKLLIYDASSLESGVPSRFSGSGSGTDFTLTISSLQPEDF | |
| | ATYYCQQFKSYPRTFGQGTKVEIK | |
| 1-CD3-M12 | AIQLTQSPSSLSASVGDRVTITCRASQGISSALAWYQQKPG | 77 |
| | KAPKLLIYDASSLENGVPSRFSGSGSGTDFTLTISSLQPEDF | |
| | ATYYCQQFNSYPRTFGQGTKVEIK | |
| 1-CD3-M13 | AIQLTQSPSSLSASVGDRVTITCRASQGISSALAWYQQKPG | 78 |
| | KAPKLLIYDASSLESGVPSRFSGSGSGTDFTLTISSLQPEDF | |
| | ATYYCQQFNSYPRTFGQGTKVEIK | |
| 1-CD3-M14 | AIQLTQSPSSLSASVGDRVTITCRASQGISSALAWYQQKPG | 79 |
| | KAPKLLIYDASGLENGVPSRFSGSGSGTDFTLTISSLQPEDF | |
| | ATYYCQQFKSYPRTFGQGTKVEIK | |
| 1-CD3-M15 | AIQLTQSPSSLSASVGDRVTITCRASQGISSALAWYQQKPG | 80 |
| | KAPKLLIYDASGLESGVPSRFSGSGSGTDFTLTISSLQPEDF | |
| | ATYYCQQFNSYPRTFGQGTKVEIK | |
| 1-CD3-C12 | AIQLTQSPSSLSASVGDRVTITCRASQGISSALAWYQQKPG | 81 |
| | KAPKLLIYDASSLESGVPSRFSGSGSGTDFTLTISSLQPEDF | |
| | ATYYCQQFNSYPRTFGQGTKVEIK | |

Next, a library of anti-CD3 antibodies having a range of anti-CD3 affinities was created by pairing the 18 different heavy chains (wild type 1-CD3 and 17 heavy chain affinity variants 1-CD3-M1, 1-CD3-M2, 1-CD3-M3, 1-CD3-M5, 1-CD3-M6, 1-CD3-M7, 1-CD3-M8, 1-CD3-M9, 1-CD3-M10, 1-CD3-M11, 1-CD3-M16, 1-CD3-M17, 1-CD3-M18, 1-CD3-M19, 1-CD3-M20, 1-CD3-M21, and 1-CD3-M22 as shown in Table E3) with 6 different light chain (wild type 1-CD3 and 5 affinity variants 1-CD3-M4, 1-CD3-M12, 1-CD3-M13, 1-CD3-M14 and 1-CD3-M15 as shown in Table E4) to create 107 variants of the 1-CD3 parental antibody. These engineered 1-CD3 antibody variants were expressed recombinantly, and the affinity of each antibody to human and/or cyno CD3εδ-muFcG1 was assessed by SPR as described in the working examples disclosed here, e.g., Example 4 and Example 6. The binding affinity for the 107 1-CD3 variants ranged from 5 nM to 150 nM.

In order to select the CD3 binding arm with the most favorable affinity profile, 1-CD3 variants having a range of affinities from 10 to 70 nM were selected from the library described above plus incorporation of the framework fixed chains, and the affinities are shown in Table E5.

TABLE E5
CD3 affinities of 1-CD3 wild type and affinity variant antibodies

| Antibody | CD3 affinity K_{D} (nM) |
|---|---|
| 1-CD3 (parental) | 7 |
| 2-CD3 | 11 |
| 3-CD3 | 5 |
| 4-CD3 | 21 |

A summary of the CD3 binding domains (1-CD3, 2-CD3, 3-CD3 and 4-CD3) is provided below.

### 1-CD3 Binder

The heavy chain variable region (VH) amino acid sequence of 1-CD3 is described in SEQ ID NO: 24.

(SEQ ID NO: 24; CDR domains are in bold)
QVQLVESGGGVVQPGRSLRLSCAASGFTFSRYGIHWVRQAPGK
GLEWVAAIWYDGSKKYYADSVKGRFTISRDNSKNTLDLQMNSL
RAEDTAVYYCARSNYDILTRYYQYGINVWGQGTTVTVSS

The VH CDR amino acid sequences of 1-CD3 are as follows: RYGIH (VH CDR1; SEQ ID NO: 9); AIWYDGSKKYYADSVKG (VH CDR2; SEQ ID NO: 10); SNYDILTRYYQYGINV (VH CDR3; SEQ ID NO: 11).

The light chain variable region (VL) amino acid sequence of 1-CD3 is described in SEQ ID NO: 39.

(SEQ ID NO: 39: CDR domains are in bold)
AIQLTQSPSSLSASVGDRVTITCRASQGISSALAWYQQKPGKA
PKLLIYDASSLENGVPSRFSGSGSGTDFTLTISSLQPEDFATY
YCQQFKSYPRTFGQGTKVEIK

The VL CDR amino acid sequences of 1-CD3 are as follows: RASQGISSALA (VL CDR1; SEQ ID NO: 19); DASSLEN (VL CDR2; SEQ ID NO: 20); QQFKSYPRT (VL CDR3; SEQ ID NO: 21).

### 2-CD3 Binder

The heavy chain variable region (VH) amino acid sequence of 2-CD3 is described in SEQ ID NO: 25 (note: VH amino acid changes when compared to the VH of 1-CD3 binder (i.e., SEQ ID NO: 24) are R31N, G55A, D80Y)

(SEQ ID NO: 25; CDR domains are in bold)
QVQLVESGGGVVQPGRSLRLSCAASGFTFSNYGIHWVRQAPGKG
LEWVAAIWYDASKKYYADSVKGRFTISRDNSKNTLYLQMNSLRA
EDTAVYYCARSNYDILTRYYQYGINVWGQGTTVTVSS

The VH CDR amino acid sequences of 2-CD3 are as follows: NYGIH (VH CDR1; SEQ ID NO: 12); AIWYDASKKYYADSVKG (VH CDR2; SEQ ID NO: 13); SNYDILTRYYQYGINV (VH CDR3; SEQ ID NO: 11).

The light chain variable region (VL) amino acid sequence of 2-CD3 is described in SEQ ID NO: 40 (note: VL amino acid change when compared to the VL of 1-CD3 binder (i.e., SEQ ID NO: 39) is N56S).

(SEQ ID NO: 40: CDR domains are in bold)
AIQLTQSPSSLSASVGDRVTITCRASQGISSALAWYQQKPGKAP
KLLIYDASSLESGVPSRFSGSGSGTDFTLTISSLQPEDFATYYC
QQFKSYPRTFGQGTKVEIK

The VL CDR amino acid sequences of 2-CD3 are as follows: RASQGISSALA (VL CDR1; SEQ ID NO: 19); DASSLES (VL CDR2; SEQ ID NO: 22); QQFKSYPRT (VL CDR3; SEQ ID NO: 21).

### 3-CD3 Binder

The heavy chain variable region (VH) amino acid sequence of 3-CD3 is described in SEQ ID NO: 26 (note: VH amino acid changes when compared to the VH of 1-CD3 binder (i.e., SEQ ID NO: 24) are G55A, D80Y, I112L, N113D).

(SEQ ID NO: 26; CDR domains are in bold)
QVQLVESGGGVVQPGRSLRLSCAASGFTFSRYGIHWVRQAPGKGLEWVAA
IWYDASKKYYADSVKGRFTISRDNSKNTLYLQMNSLRAEDTAVYYCARSN
YDILTRYYQYGLDVWGQGTTVTVSS

The VH CDR amino acid sequences of 3-CD3 are as follows: RYGIH (VH CDR1; SEQ ID NO: 9); AIWYDASKKYYADSVKG (VH CDR2; SEQ ID NO: 13); SNYDILTRYYQYGLDV (VH CDR3; SEQ ID NO: 14).

The light chain variable region (VL) amino acid sequence of 3-CD3 is described in SEQ ID NO: 40 (note: VL amino acid change when compared to the VL of 1-CD3 binder (i.e., SEQ ID NO: 39) is N56S).

(SEQ ID NO: 40: CDR domains are in bold)
AIQLTQSPSSLSASVGDRVTITCRASQGISSALAWYQQKPGKAPKLLIYD
ASSLESGVPSRFSGSGSGTDFTLTISSLQPEDFATYYCQQFKSYPRTFGQ
GTKVEIK

The VL CDR amino acid sequences of 3-CD3 are as follows: RASQGISSALA (VL CDR1; SEQ ID NO: 19); DASSLES (VL CDR2; SEQ ID NO: 22); QQFKSYPRT (VL CDR3; SEQ ID NO: 21).

### 4-CD3 Binder

The heavy chain variable region (VH) amino acid sequence of 4-CD3 is described in SEQ ID NO: 27 (note: VH amino acid changes when compared to the VH of 1-CD3 binder (i.e., SEQ ID NO: 24) are G55A, D80Y, Q109S, Y110H)

(SEQ ID NO: 27; CDR domains are in bold)
QVQLVESGGGVVQPGRSLRLSCAASGFTFSRYGIHWVRQAPGKGLEWVAA
IWYDASKKYYADSVKGRFTISRDNSKNTLYLQMNSLRAEDTAVYYCARSN
YDILTRYYSHGINVWGQGTTVTVSS

The VH CDR amino acid sequences of 4-CD3 are as follows: RYGIH (VH CDR1; SEQ ID NO: 9); AIWYDASKKYYADSVKG (VH CDR2; SEQ ID NO: 13); SNYDILTRYYSHGINV (VH CDR3; SEQ ID NO: 11).

The light chain variable region (VL) amino acid sequence of 4-CD3 is described in SEQ ID NO: 41 (note: VL amino acid changes when compared to the VL of 1-CD3 binder (i.e., SEQ ID NO: 39) are N56S and K92N).

(SEQ ID NO: 41: CDR domains are in bold)
AIQLTQSPSSLSASVGDRVTITCRASQGISSALAWYQQKPGKAPKLLIYD
ASSLESGVPSRFSGSGSGTDFTLTISSLQPEDFATYYCQQFNSYPRTFGQ
GTKVEIK

The VL CDR amino acid sequences of 4-CD3 are as follows: RASQGISSALA (VL CDR1; SEQ ID NO: 19); DASSLES (VL CDR2; SEQ ID NO: 22); QQFNSYPRT (VL CDR3; SEQ ID NO: 23).

### Example 6. Extracellular Matrix (ECM) ELISA Analysis of TCEs

This Example performed extracellular matrix ELISA analysis of multiple binding molecules which contained a CD3-targeted antibody binder containing an identified CD3 V_{H} and CD3 V_{L} binding domain. Several 96-well Corning Thin-Layer Matrigel Matrix pre-coated ECM plates (Catalog No. 354607) were incubated for one hour at room temperature with 300 μl of blocking buffer (FCS GIBCO; Catalog No. 26010, diluted to 10% in PBS). After incubation, the blocking buffer was removed, and the samples were mixed with fresh blocking buffer in 1:1 ratio in a total volume of 100 μl to yield final concentrations of 1 μM, 0.5 μM, 0.25 μM and 0.05 μM. Six wells had no sample addition for background measurement. After one hour of sample incubation, samples were removed and plates were washed with PBS-T wash buffer 3 times. A 10 ng/ml amount of goat anti-human IgG-HRP conjugated detection antibody (Jackson ImmunoResearch; Catalog No. 109-035-008) was added at 100 μl to all wells. After another one-hour incubation at room temperature, the wells were washed three times with PBS-T wash buffer. After washing, 100 μl of TMB substrate (Surmodics; Catalog No. TMBW-1000-01) was added to each well and allowed to react for 15 minutes, followed by addition of 100 μl of BioFX stop solution (Surmodics; Product code STPR).

Absorbance was then read on a microplate reader at 450 nm referenced at 620 nm. The absorbance values of the samples were divided by the average absorbance value of an internal high control antibody to calculate the % High control values. A lower ECM value was generally desirable because it indicated less non-specific binding to extracellular matrix proteins. A level of 12 as % High control value was chosen as a cutoff since this assay value corresponded to the worst 10% of a set of clinically approved antibodies that were tested using the same protocol. Results for two different sets of exemplary bispecific binding molecules that had one of the CD3 binders 1-CD3, 2-CD3, 3-CD3, and 4-CD3 are summarized in Table E7 and Table E8. The results demonstrated elevated ECM ELISA binding, suggesting electrostatically dominated non-specific interaction liability (i.e., risk or tendency for proteins to interact with each other due to non-specific electrical charges). Biophysical engineering of 3-CD3 was conducted in order reduce this potential liability while maintaining the desired binding and functional properties of the assembled bispecific construct.

TABLE E7
ECM ELISA analysis of first binding molecule
with CD3-targeting antibody binders

| CD3 Binding | CD3 VH binders | CD3 VL binders | Median ECM % |
|---|---|---|---|
| domain | (SEQ ID NO:) | (SEQ ID NO:) | high control |
| 1-CD3 | 24 | 39 | 101.61 |
| 2-CD3 | 25 | 40 | 92.18 |
| 3-CD3 | 26 | 40 | 101.65 |
| 4-CD3 | 27 | 41 | 87.98 |

TABLE E8
ECM ELISA analysis of second binding molecule
with CD3-targeting antibody binders

| CD3 Binding | CD3 VH binders | CD3 VL binders | Median ECM % |
|---|---|---|---|
| domain | (SEQ ID NO:) | (SEQ ID NO:) | high control |
| 1-CD3 | 24 | 39 | 96.9 |
| 2-CD3 | 25 | 40 | 37.13 |
| 3-CD3 | 26 | 40 | 96.74 |
| 4-CD3 | 27 | 41 | 49.35 |

The parental CD3 binder (i.e., 3-CD3; SEQ ID Nos: 26 and 40 for the V_{H} and V_{L}, respectively) demonstrated higher than desired non-specific charged based interactions, as determined by the ECM ELISA assay. Structural analysis for the Fab indicated three charged amino acid positions R16, K58 and K76 that were solvent exposed and expected to be contributing to overall positive surface charge of the molecule. The following mutagenesis strategy was employed to evaluate the biophysical characteristics of single, double and triple amino acid substitutions: R16E or R16G, K58T or K58D, and K76S or K76E. The resultant engineered molecules were evaluated for the ECM ELISA binding in addition to SPR analysis (K_{D}) using CD3ε as an antigen and cellular binding (e.g., EC₅₀).

Biacore C1 Series S sensor (Cytiva) was docked into a Biacore 8K+ instrument and the system was primed with HBS-T+ (10 mM HEPES, 150 mM NaCl, 0.05% Tween-20, pH 7.4 buffer (Corning). The C1 chip was conditioned with 0.1 M glycine-NaOH, 0.3% Triton X-100, pH 12 according to manufacturer guidelines. Running buffer was switched to 25 mM MES, pH5.5, (Alfa Aesar) 0.01% Tween-20 (Thermo Scientific) for the immobilization. The sensor temperature was adjusted to 37 °C, and the sample compartment was maintained at 15 °C. Upon priming, the chip was activated using a fresh 1:1 mixture of 0.4 M EDC and 0.1 M sulfo-NHS (Thermo Scientific) for seven minutes at 10 μl/ml flow rate across all eight channels. Rabbit anti-mouse IgG polyclonal antibody (Cytiva) was diluted to 50 g/mL in 10 mM sodium acetate, pH 5.0 (Cytiva) and amine-coupled to all flow cells within each of the eight channels for 15 minutes at 5 μL/min. Finally, to block the activated chip surface, 0.1 M ethylenediamine in 250 mM sodium borate, pH 8.5 buffer was injected over all flow cells within each of the eight channels for 7 minutes at 10 μl/minute. Surfaces were regenerated with two 30-second injections of 10 mM glycine, pH 1.7 (Cytiva) at 30 μL/min.

Engineered CD3-targeting antibodies were measured for their binding affinity to human CD3εδ-muFcG1 in a multi-cycle capture kinetics experiment. Running buffer was switched to HBS-T+ (10 mM HEPES, 150 mM NaCl, 0.05% Tween-20, pH 7.4 buffer (Corning) with 0.1% BSA (Sigma-Aldrich) and the system was thoroughly primed in this buffer. Human CD3εδ-muFcG1 was diluted to 15 μg/mL in running buffer and served as ligand. Five-point, five-fold serial dilutions of each monovalent bispecific CD3-targeting antibody at 100 nM top concentration were prepared in running buffer. Three startup cycles, which are abbreviated analysis cycles of shortened injections but with complete regeneration, were introduced prior to the analysis experiment. In each analysis cycle, human CD3εδ-muFcG1 was captured as ligand to only flow cell 2 of each channel at 5 L/min for 2 minutes. Flow cell 1 of each channel remained unaddressed and served as the reference surface. A two-minute stabilization period was introduced after every ligand capture. A single analyte sample was injected per cycle over each channel for 2 minutes at 30 μL/min with 10 min dissociation. Analyte samples comprised of the monovalent, bispecific CD3-targeting antibody serial dilutions and were injected in order from lowest to highest concentration with buffer blanks flanking the concentration series. Amine-coupled rabbit anti-mouse IgG polyclonal antibody surfaces were regenerated at the end of each cycle with one injection of 10 mM glycine, pH 1.7 at 30 μL/min for one minute.

Data are shown in Table E9. The combination of point mutations R16G and K58T significantly reduced the observed ECM ELISA liability while maintaining similar binding affinity (K_{D}).

These mutants, in conjunction with disulfide introduction in the scFv (data not shown) were used to generate 5-CD3.

TABLE E9
Biophysical analysis of CD3 binders

| | ECM % High | | | K_{D} | | | |
|---|---|---|---|---|---|---|---|
| | 1 μM | 0.2 μM | 0.04 μM | (nM) | R16 | K58 | K76 |
| Parental | 65.8 | 24.7 | 8.5 | 4.3 | R | K | K |
| 3-CD3 | | | | | | | |
| 9-CD3 | 36.1 | 12.7 | 5.6 | 5.0 | G | K | K |
| 10-CD3 | 16.3 | 8.2 | 5.3 | 5.5 | E | K | K |
| 6-CD3 | 7.1 | 5.3 | 4.8 | 12 | G | T | K |
| 11-CD3 | 5.6 | 4.8 | 5.0 | 20 | E | D | K |
| 12-CD3 | 7.2 | 6.1 | 5.6 | >50 | G | T | S |
| 13-CD3 | 5.3 | 4.8 | 3.8 | >50 | E | D | E |
| 14-CD3 | 6.5 | 4.7 | 4.1 | 9.7 | G | K | S |
| 15-CD3 | 9.6 | 6.3 | 5.6 | >50 | E | K | E |
| low control | 3.8 | | | | | | |
| high control | 100.0 | | | | | | |

A summary of the 5-CD3 through 16-CD3 binding domains is provided below.

### 5-CD3 Binder

The heavy chain variable region (VH) amino acid sequence of 5-CD3 is described in SEQ ID NO: 28 (note: VH amino acid changes when compared to the VH of 1-CD3 binder (i.e., SEQ ID NO: 24) are R16G, K58T, G44C, G55A, D80Y, I112L, N113D).

(SEQ ID NO: 28; CDR domains are in bold)
QVQLVESGGGVVQPGGSLRLSCAASGFTFSRYGIHWVRQAPGKCLEWVAA
IWYDASKTYYADSVKGRFTISRDNSKNTLYLQMNSLRAEDTAVYYCARSN
YDILTRYYQYGLDVWGQGTTVTVSS

The VH CDR amino acid sequences of 5-CD3 are as follows: RYGIH (VH CDR1; SEQ ID NO: 9); AIWYDASKTYYADSVKG (VH CDR2; SEQ ID NO: 15); SNYDILTRYYQYGLDV (VH CDR3; SEQ ID NO: 14).

The light chain variable region (VL) amino acid sequence of 5-CD3 is described in SEQ ID NO: 42 (note: VL amino acid changes when compared to the VL of 1-CD3 binder (i.e., SEQ ID NO: 39) are N56S, Q100C).

(SEQ ID NO: 42: CDR domains are in bold)
AIQLTQSPSSLSASVGDRVTITCRASQGISSALAWYQQKPGKAPKLLIYD
ASSLESGVPSRFSGSGSGTDFTLTISSLQPEDFATYYCQQFKSYPRTFGC
GTKVEIK

The VL CDR amino acid sequences of 5-CD3 are as follows: RASQGISSALA (VL CDR1; SEQ ID NO: 19); DASSLES (VL CDR2; SEQ ID NO: 22); QQFKSYPRT (VL CDR3; SEQ ID NO: 21).

### 6-CD3 Binder

The heavy chain variable region (VH) amino acid sequence of 6-CD3 is described in SEQ ID NO: 29 (note: VH amino acid changes when compared to the VH of 1-CD3 binder (i.e., SEQ ID NO: 24) are R16G, K58T, G55A, D80Y, I112L, N113D)

(SEQ ID NO: 29; CDR domains are in bold)
QVQLVESGGGVVQPGGSLRLSCAASGFTFSRYGIHWVRQAPGKGLEWVAA
IWYDASKTYYADSVKGRFTISRDNSKNTLYLQMNSLRAEDTAVYYCARSN
YDILTRYYQYGLDVWGQGTTVTVSS

The VH CDR amino acid sequences of 6-CD3 are as follows: RYGIH (VH CDR1; SEQ ID NO: 9); AIWYDASKTYYADSVKG (VH CDR2; SEQ ID NO: 15); SNYDILTRYYQYGLDV (VH CDR3; SEQ ID NO: 14).

The light chain variable region (VL) amino acid sequence of 6-CD3 is described in SEQ ID NO: 40 (note: VL amino acid change when compared to the VL of 1-CD3 binder (i.e., SEQ ID NO: 39) is N56S).

(SEQ ID NO: 40: CDR domains are in bold)
AIQLTQSPSSLSASVGDRVTITCRASQGISSALAWYQQKPGKAPKLLIYD
ASSLESGVPSRESGSGSGTDFTLTISSLQPEDFATYYCQQFKSYPRTFGQ
GTKVEIK

The VL CDR amino acid sequences of 6-CD3 are as follows: RASQGISSALA (VL CDR1; SEQ ID NO: 19); DASSLES (VL CDR2; SEQ ID NO: 22); QQFKSYPRT (VL CDR3; SEQ ID NO: 21).

### 7-CD3 Binder

The heavy chain variable region (VH) amino acid sequence of 7-CD3 is described in SEQ ID NO: 30 (note: VH amino acid changes when compared to the VH of 1-CD3 binder (i.e., SEQ ID NO: 24) are R16G, K58T, G55A, D80Y, Q109S, Y110H)

(SEQ ID NO: 30; CDR domains are in bold)
QVQLVESGGGVVQPGGSLRLSCAASGFTFSRYGIHWVRQAPGKGLEWVAA
IWYDASKTYYADSVKGRFTISRDNSKNTLYLQMNSLRAEDTAVYYCARSN
YDILTRYYSHGINVWGQGTTVTVSS

The VH CDR amino acid sequences of 7-CD3 are as follows: RYGIH (VH CDR1; SEQ ID NO: 9); AIWYDASKTYYADSVKG (VH CDR2; SEQ ID NO: 15); SNYDILTRYYSHGINV (VH CDR3; SEQ ID NO: 16).

The light chain variable region (VL) amino acid sequence of 7-CD3 is described in SEQ ID NO: 41 (note: VL amino acid change when compared to the VL of 1-CD3 binder (i.e., SEQ ID NO: 39) is N56S and K92N).

(SEQ ID NO: 41: CDR domains are in bold)
AIQLTQSPSSLSASVGDRVTITCRASQGISSALAWYQQKPGKAPKLLIYD
ASSLESGVPSRFSGSGSGTDFTLTISSLQPEDFATYYCQQFNSYPRTFGQ
GTKVEIK

The VL CDR amino acid sequences of 7-CD3 are as follows: RASQGISSALA (VL CDR1; SEQ ID NO: 19); DASSLES (VL CDR2; SEQ ID NO: 22); QQFNSYPRT (VL CDR3; SEQ ID NO: 23).

### 8-CD3 Binder

The heavy chain variable region (VH) amino acid sequence of 8-CD3 is described in SEQ ID NO: 31 (note: VH amino acid changes when compared to the VH of 1-CD3 binder (i.e., SEQ ID NO: 24) are R16G, K58T, G44C, G55A, D80Y, Q109S, Y110H)

(SEQ ID NO: 31; CDR domains are in bold)
QVQLVESGGGVVQPGGSLRLSCAASGFTESRYGIHWVRQAPGKCLEWVAA
IWYDASKTYYADSVKGRFTISRDNSKNTLYLQMNSLRAEDTAVYYCARSN
YDILTRYYSHGINVWGQGTTVTVSS

The VH CDR amino acid sequences of 8-CD3 are as follows: RYGIH (VH CDR1; SEQ ID NO: 9); AIWYDASKTYYADSVKG (VH CDR2; SEQ ID NO: 15); SNYDILTRYYSHGINV (VH CDR3; SEQ ID NO: 16).

The light chain variable region (VL) amino acid sequence of 8-CD3 is described in SEQ ID NO: 43 (note: VL amino acid change when compared to the VL of 1-CD3 binder (i.e., SEQ ID NO: 39) is N56S, K92N and Q100C).

(SEQ ID NO: 43: CDR domains are in bold)
AIQLTQSPSSLSASVGDRVTITCRASQGISSALAWYQQKPGKAPKLLIYD
ASSLESGVPSRFSGSGSGTDFTLTISSLQPEDFATYYCQQFNSYPRTFGC
GTKVEIK

The VL CDR amino acid sequences of 8-CD3 are as follows: RASQGISSALA (VL CDR1; SEQ ID NO: 19); DASSLES (VL CDR2; SEQ ID NO: 22); QQFNSYPRT (VL CDR3; SEQ ID NO: 23).

### 9-CD3 Binder

The heavy chain variable region (VH) amino acid sequence of 9-CD3 is described in SEQ ID NO: 32 (note: VH amino acid changes when compared to the VH of 1-CD3 binder (i.e., SEQ ID NO: 24) are R16G, G55A, D80Y, I112L, N113D)

(SEQ ID NO: 32; CDR domains are in bold)
QVQLVESGGGVVQPGGSLRLSCAASGFTFSRYGIHWVRQAPGKGLEWVAA
IWYDASKKYYADSVKGRFTISRDNSKNTLYLQMNSLRAEDTAVYYCARSN
YDILTRYYQYGLDVWGQGTTVTVSS

The VH CDR amino acid sequences of 9-CD3 are as follows: RYGIH (VH CDR1; SEQ ID NO: 9); AIWYDASKKYYADSVKG (VH CDR2; SEQ ID NO: 13); SNYDILTRYYQYGLDV (VH CDR3; SEQ ID NO: 14).

The light chain variable region (VL) amino acid sequence of 9-CD3 is described in SEQ ID NO: 40 (note: VL amino acid change when compared to the VL of 1-CD3 binder (i.e., SEQ ID NO: 39) is N56S).

(SEQ ID NO: 40: CDR domains are in bold)
AIQLTQSPSSLSASVGDRVTITCRASQGISSALAWYQQKPGKAPKLLIYD
ASSLESGVPSRFSGSGSGTDFTLTISSLQPEDFATYYCQQFKSYPRTFGQ
GTKVEIK

The VL CDR amino acid sequences of 9-CD3 are as follows: RASQGISSALA (VL CDR1; SEQ ID NO: 19); DASSLES (VL CDR2; SEQ ID NO: 22); QQFKSYPRT (VL CDR3; SEQ ID NO: 21).

### 10-CD3 Binder

The heavy chain variable region (VH) amino acid sequence of 10-CD3 is described in SEQ ID NO: 126 (note: VH amino acid changes when compared to the VH of 1-CD3 binder (i.e., SEQ ID NO: 24) are R16E, G55A, D80Y, I112L, N113D)

(SEQ ID NO: 126; CDR domains are in bold)
QVQLVESGGGVVQPGESLRLSCAASGFTFSRYGIHWVRQAPGKGLEWVAA
IWYDASKKYYADSVKGRFTISRDNSKNTLYLQMNSLRAEDTAVYYCARSN
YDILTRYYQYGLDVWGQGTTVTVSS

The VH CDR amino acid sequences of 10-CD3 are as follows: RYGIH (VH CDR1; SEQ ID NO: 9); AIWYDASKKYYADSVKG (VH CDR2; SEQ ID NO: 13); SNYDILTRYYQYGLDV (VH CDR3; SEQ ID NO: 14).

The light chain variable region (VL) amino acid sequence of 10-CD3 is described in SEQ ID NO: 40 (note: VL amino acid change when compared to the VL of 1-CD3 binder (i.e., SEQ ID NO: 39) is N56S).

(SEQ ID NO: 40: CDR domains are in bold)
AIQLTQSPSSLSASVGDRVTITCRASQGISSALAWYQQKPGKAPKLLIYD
ASSLESGVPSRFSGSGSGTDFTLTISSLQPEDFATYYCQQFKSYPRTFGQ
GTKVEIK

The VL CDR amino acid sequences of 10-CD3 are as follows: RASQGISSALA (VL CDR1; SEQ ID NO: 19); DASSLES (VL CDR2; SEQ ID NO: 22); QQFKSYPRT (VL CDR3; SEQ ID NO: 21).

### 11-CD3 Binder

The heavy chain variable region (VH) amino acid sequence of 11-CD3 is described in SEQ ID NO: 33 (note: VH amino acid changes when compared to the VH of 1-CD3 binder (i.e., SEQ ID NO: 24) are R16E, K58D, G55A, D80Y, I112L, N113D)

(SEQ ID NO: 33; CDR domains are in bold)
QVQLVESGGGVVQPGESLRLSCAASGFTFSRYGIHWVRQAPGKGLEWVAA
IWYDASKDYYADSVKGRFTISRDNSKNTLYLQMNSLRAEDTAVYYCARSN
YDILTRYYQYGLDVWGQGTTVTVSS

The VH CDR amino acid sequences of 11-CD3 are as follows: RYGIH (VH CDR1; SEQ ID NO: 9); AIWYDASKDYYADSVKG (VH CDR2; SEQ ID NO: 17); SNYDILTRYYQYGLDV (VH CDR3; SEQ ID NO: 14).

The light chain variable region (VL) amino acid sequence of 11-CD3 is described in SEQ ID NO: 40 (note: VL amino acid change when compared to the VL of 1-CD3 binder (i.e., SEQ ID NO: 39) is N56S).

(SEQ ID NO: 40: CDR domains are in bold)
AIQLTQSPSSLSASVGDRVTITCRASQGISSALAWYQQKPGKAPKLLIYD
ASSLESGVPSRFSGSGSGTDFTLTISSLQPEDFATYYCQQFKSYPRTFGQ
GTKVEIK

The VL CDR amino acid sequences of 11-CD3 are as follows: RASQGISSALA (VL CDR1; SEQ ID NO: 19); DASSLES (VL CDR2; SEQ ID NO: 22); QQFKSYPRT (VL CDR3; SEQ ID NO: 21).

### 12-CD3 Binder

The heavy chain variable region (VH) amino acid sequence of 12-CD3 is described in SEQ ID NO: 34 (note: VH amino acid changes when compared to the VH of 1-CD3 binder (i.e., SEQ ID NO: 24) are R16G, K58T, K76S, G55A, D80Y, I112L, N113D)

(SEQ ID NO: 34; CDR domains are in bold)
QVQLVESGGGVVQPGGSLRLSCAASGFTFSRYGIHWVRQAPGKGLEWVAA
IWYDASKTYYADSVKGRFTISRDNSSNTLYLQMNSLRAEDTAVYYCARSN
YDILTRYYQYGLDVWGQGTTVTVSS

The VH CDR amino acid sequences of 12-CD3 are as follows: RYGIH (VH CDR1; SEQ ID NO: 9); AIWYDASKTYYADSVKG (VH CDR2; SEQ ID NO: 15); SNYDILTRYYQYGLDV (VH CDR3; SEQ ID NO: 14).

The light chain variable region (VL) amino acid sequence of 12-CD3 is described in SEQ ID NO: 40 (note: VL amino acid change when compared to the VL of 1-CD3 binder (i.e., SEQ ID NO: 39) is N56S).

(SEQ ID NO: 40: CDR domains are in bold)
AIQLTQSPSSLSASVGDRVTITCRASQGISSALAWYQQKPGKAPKLLIYD
ASSLESGVPSRFSGSGSGTDFTLTISSLQPEDFATYYCQQFKSYPRTFGQ
GTKVEIK

The VL CDR amino acid sequences of 12-CD3 are as follows: RASQGISSALA (VL CDR1; SEQ ID NO: 19); DASSLES (VL CDR2; SEQ ID NO: 22); QQFKSYPRT (VL CDR3; SEQ ID NO: 21).

### 13-CD3 Binder

The heavy chain variable region (VH) amino acid sequence of 13-CD3 is described in SEQ ID NO: 35 (note: VH amino acid changes when compared to the VH of 1-CD3 binder (i.e., SEQ ID NO: 24) are R16E, K58D, K76S, G55A, D80Y, I112L, N113D)

(SEQ ID NO: 35; CDR domains are in bold)
QVQLVESGGGVVQPGESLRLSCAASGFTFSRYGIHWVRQAPGKGLEWVAA
IWYDASKDYYADSVKGRFTISRDNSENTLYLQMNSLRAEDTAVYYCARSN
YDILTRYYQYGLDVWGQGTTVTVSS

The VH CDR amino acid sequences of 13-CD3 are as follows: RYGIH (VH CDR1; SEQ ID NO: 9); AIWYDASKDYYADSVKG (VH CDR2; SEQ ID NO: 17); SNYDILTRYYQYGLDV (VH CDR3; SEQ ID NO: 14).

The light chain variable region (VL) amino acid sequence of 13-CD3 is described in SEQ ID NO: 40 (note: VL amino acid change when compared to the VL of 1-CD3 binder (i.e., SEQ ID NO: 39) is N56S).

(SEQ ID NO: 40: CDR domains are in bold)
AIQLTQSPSSLSASVGDRVTITCRASQGISSALAWYQQKPGKAPKLLIYDA
SSLESGVPSRFSGSGSGTDFTLTISSLQPEDFATYYCQQFKSYPRTFGQGT
KVEIK

The VL CDR amino acid sequences of 13-CD3 are as follows: RASQGISSALA (VL CDR1; SEQ ID NO: 19); DASSLES (VL CDR2; SEQ ID NO: 22); QQFKSYPRT (VL CDR3; SEQ ID NO: 21).

### 14-CD3 Binder

The heavy chain variable region (VH) amino acid sequence of 14-CD3 is described in SEQ ID NO: 36 (note: VH amino acid changes when compared to the VH of 1-CD3 binder (i.e., SEQ ID NO: 24) are R16G, K76S, G55A, D80Y, I112L, N113D)

(SEQ ID NO: 36; CDR domains are in bold)
QVQLVESGGGVVQPGGSLRLSCAASGFTFSRYGIHWVRQAPGKGLEWVAA
IWYDASKKYYADSVKGRFTISRDNSSNTLYLQMNSLRAEDTAVYYCARSN
YDILTRYYQYGLDVWGQGTTVTVSS

The VH CDR amino acid sequences of 14-CD3 are as follows: RYGIH (VH CDR1; SEQ ID NO: 9); AIWYDASKKYYADSVKG (VH CDR2; SEQ ID NO: 13); SNYDILTRYYQYGLDV (VH CDR3; SEQ ID NO: 14).

The light chain variable region (VL) amino acid sequence of 14-CD3 is described in SEQ ID NO: 40 (note: VL amino acid change when compared to the VL of 1-CD3 binder (i.e., SEQ ID NO: 39) is N56S).

(SEQ ID NO: 40: CDR domains are in bold)
AIQLTQSPSSLSASVGDRVTITCRASQGISSALAWYQQKPGKAPKLLIYD
ASSLESGVPSRESGSGSGTDFTLTISSLQPEDFATYYCQQFKSYPRTFGQ
GTKVEIK

The VL CDR amino acid sequences of 14-CD3 are as follows: RASQGISSALA (VL CDR1; SEQ ID NO: 19); DASSLES (VL CDR2; SEQ ID NO: 22); QQFKSYPRT (VL CDR3; SEQ ID NO: 21).

### 15-CD3 Binder

The heavy chain variable region (VH) amino acid sequence of 15-CD3 is described in SEQ ID NO: 37 (note: VH amino acid changes when compared to the VH of 1-CD3 binder (i.e., SEQ ID NO: 24) are R16E, K76E, G55A, D80Y, I112L, N113D)

(SEQ ID NO: 37; CDR domains are in bold)
QVQLVESGGGVVQPGESLRLSCAASGFTFSRYGIHWVRQAPGKGLEWVAA
IWYDASKKYYADSVKGRFTISRDNSENTLYLQMNSLRAEDTAVYYCARSN
YDILTRYYQYGLDVWGQGTTVTVSS

The VH CDR amino acid sequences of 15-CD3 are as follows: RYGIH (VH CDR1; SEQ ID NO: 9); AIWYDASKKYYADSVKG (VH CDR2; SEQ ID NO: 13); SNYDILTRYYQYGLDV (VH CDR3; SEQ ID NO: 14).

The light chain variable region (VL) amino acid sequence of 15-CD3 is described in SEQ ID NO: 40 (note: VL amino acid change when compared to the VL of 1-CD3 binder (i.e., SEQ ID NO: 39) is N56S).

(SEQ ID NO: 40: CDR domains are in bold)
AIQLTQSPSSLSASVGDRVTITCRASQGISSALAWYQQKPGKAPKLLIYD
ASSLESGVPSRFSGSGSGTDFTLTISSLQPEDFATYYCQQFKSYPRTFGQ
GTKVEIK

The VL CDR amino acid sequences of 15-CD3 are as follows: RASQGISSALA (VL CDR1; SEQ ID NO: 19); DASSLES (VL CDR2; SEQ ID NO: 22); QQFKSYPRT (VL CDR3; SEQ ID NO: 21).

### 16-CD3 Binder

The heavy chain variable region (VH) amino acid sequence of 16-CD3 is described in SEQ ID NO: 38 (note: VH amino acid changes when compared to the VH of 1-CD3 binder (i.e., SEQ ID NO: 24) are R16G, K58T, G55A, D80Y, R106G, Y107H)

(SEQ ID NO: 38; CDR domains are in bold)
QVQLVESGGGVVQPGGSLRLSCAASGFTFSRYGIHWVRQAPGKGLEWVAA
IWYDASKTYYADSVKGRFTISRDNSKNTLYLQMNSLRAEDTAVYYCARSN
YDILTGHYQYGINVWGQGTTVTVSS

The VH CDR amino acid sequences of 16-CD3 are as follows: RYGIH (VH CDR1; SEQ ID NO: 9); AIWYDASKTYYADSVKG (VH CDR2; SEQ ID NO: 15); SNYDILTGHYQYGINV (VH CDR3; SEQ ID NO: 18).

The light chain variable region (VL) amino acid sequence of 16-CD3 is described in SEQ ID NO: 41 (note: VL amino acid change when compared to the VL of 1-CD3 binder (i.e., SEQ ID NO: 39) is N56S and K92N).

(SEQ ID NO: 41: CDR domains are in bold)
AIQLTQSPSSLSASVGDRVTITCRASQGISSALAWYQQKPGKAPKLLIYD
ASSLESGVPSRESGSGSGTDFTLTISSLQPEDFATYYCQQFNSYPRTFGQ
GTKVEIK

The VL CDR amino acid sequences of 16-CD3 are as follows: RASQGISSALA (VL CDR1; SEQ ID NO: 19); DASSLES (VL CDR2; SEQ ID NO: 22); QQFNSYPRT (VL CDR3; SEQ ID NO: 23).

### Example 7: Epitopes and Paratopes for the Parental 1-CD3 Anti-CD3 Epsilon Antibody

The crystal structure of an antigen-binding fragment of 1-CD3 in complex with a human CD3 epsilon peptide “ZDGNEEMGGITQTP” (SEQ ID NO: 127) where Z represents pyroglutamic acid (PCA), a chemically modified version of Q, was determined at 2.8 Å. A “Protein interfaces, surfaces and assemblies” (PISA) analysis of the structure was used to determine the residues of 1-CD3 interacting with the CD3 epsilon peptide (European Bioinformatics Institute, http://www.ebi.ac.uk/pdbe/prot_int/pistart.html, Krissinel and Henrick (2007) J. Mol. Biol. 372, 774-797.) Based on this analysis, residues Z1, D2, G3, N4, E5, E6, M7 are interacting with the heavy chain of 1-CD3, and residues Z1, D2, E5, E6 are interacting with the light chain of 1-CD3.

Paratope residues that are within 4.5 Å of the CD3 antigen in the 1-CD3 antibody/antigen complex were identified. The key paratope residues for human CD3 epsilon in the 1-CD3 antibody were determined to be residues G33, H35, 151, W52, Y53, S99, Y101, I103, R106, Q109, and Y110 in the 1-CD3 heavy chain variable region (SEQ ID NO: 24) and residues S30, F91, K92, Y94, and R96 in the 1-CD3 light chain variable region (SEQ ID NO: 39).

The binding epitope for an exemplary bispecific antibody with the antigen-binding fragment of 1-CD3 in complex with a human CD3 epsilon peptide was further characterized by hydrogen-deuterium exchange, fast photochemical oxidation of proteins (FPOP) and glycine ethyl ester (GEE) labeling mass spectrometry.

Based on these analyses, E5, E6 and M7 in the N-terminal region of mature human CD3 epsilon domain (SEQ ID NO:82 in Table E11) were shown to interact with 1-CD3.

TABLE E11
Sequence of mature human CD3 epsilon

| | | |
|---|---|---|
| Mature | QDGNEEMGGITQTPYKVSISGTTVILTCPQYPGSEILWQH | SEQ ID NO: |
| human | NDKNIGGDEDDKNIGSDEDHLSLKEFSELEQSGYYVCYP | 82 |
| CD3 | RGSKPEDANFYLYLRARVCENCMEMDVMSVATIVIVDIC | |
| epsilon | ITGGLLLLVYYWSKNRKAKAKPVTRGAGAGGRQRGQNK | |
| | ERPPPVPNPDYEPIRKGQRDLYSGLNQRRI | |

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

TABLE E12
WITH AMINO ACID SEQUENCES AND NUCLEIC ACID SEQUENCES

| SEQ | | |
|---|---|---|
| ID | | |
| NO: | Sequence | Description |
| 1 | X₂YGIH | Consensus CDR1 |
| | | VH; X₂ = R or N |
| 2 | AIWYDX₄SKX₅YYADSVKG | Consensus CDR2 |
| | | VH; X₄ = G or A; |
| | | X₅ = K or T or D |
| 3 | SNYDILTX₈X₉YX₁₀X₁₁GX₁₂X₁₃V | Consensus CDR3 |
| | | VH; X₈ = R or G; |
| | | X₉ = Y or H; X₁₀ = Q |
| | | or S; X₁₁ = Y or H; |
| | | X₁₂ = I or L; X₁₃ = N |
| | | or D |
| 4 | QVQLVESGGGVVQPGRSLRLSCAASGFTFSRYGIHWVRQAPGK | 1-CD3-M1 |
| | GLEWVAAIWYDGSKKYYADSVKGRFTISRDNSKNTLYLQMNS | |
| | LRAEDTAVYYCARSNYDILTRYYQYGINVWGQGTTVTVSS | |
| 5 | DASSLEX₁ | Consensus CDR2 |
| | | VL |
| | | X₁ = N or S |
| 6 | QQFX₂SYPRT | Consensus CDR3 |
| | | VL |
| | | X₂ = K or N |
| 7 | QVQLVESGGGVVQPGX₁SLRLSCAASGFTFSX₂YGIHWVRQAP | Consensus VH |
| | GKX₃LEWVAAIWYDX₄SKX₅YYADSVKGRFTISRDNSX₆NTLX₇L | X₁ = R or G or E |
| | QMNSLRAEDTAVYYCARSNYDILTX₈X₉YX₁₀X₁₁GX₁₂X₁₃VWGQ | X₂ = R or N |
| | GTTVTVSS | X₃ = G or C |
| | | X₄ = G or A |
| | | X₅ = K or T or D |
| | | X₆ = K or E or S |
| | | X₇ = Y or D |
| | | X₈ = R or G |
| | | X₉ = Y or H |
| | | X₁₀ = Q or S X11 = Y or H |
| | | X₁₂ = I or L |
| | | X₁₃ = N or D |
| 8 | AIQLTQSPSSLSASVGDRVTITCRASQGISSALAWYQQKPGKAP | Consensus VL |
| | KLLIYDASSLEX₁GVPSRFSGSGSGTDFTLTISSLQPEDFATYYCQ | X₁ = N or S |
| | QFX₂SYPRTFGX₃GTKVEIK | X₂ = K or N |
| | | X₃ = Q or C |
| 9 | RYGIH | 1, 3, 4, 5, 6, 7, 8, 9, |
| | | 10, 11, 12, 13, 14, 15, |
| | | 16-CD3-VH-CDR1 |
| 10 | AIWYDGSKKYYADSVKG | 1-CD3-VH-CDR2 |
| 11 | SNYDILTRYYQYGINV | 1, 2, 4-CD3-VH- |
| | | CDR3 |
| 12 | NYGIH | 2-CD3-VH-CDR1 |
| 13 | AIWYDASKKYYADSVKG | 2, 3, 4, 9, 10, 14, 15- |
| | | CD3-VH-CDR2 |
| 14 | SNYDILTRYYQYGLDV | 3, 5, 6, 9, 10, 11, 12, |
| | | 13, 14, 15-CD3-VH- |
| | | CDR3 |
| 15 | AIWYDASKTYYADSVKG | 5, 6, 7, 8, 12, 16-CD3- |
| | | VH-CDR2 |
| 16 | SNYDILTRYYSHGINV | 7, 8-CD3-VH-CDR3 |
| 17 | AIWYDASKDYYADSVKG | 11, 13-CD3-VH- |
| | | CDR2 |
| 18 | SNYDILTGHYQYGINV | 16-CD3-VH-CD3 |
| 19 | RASQGISSALA | 1, 2, 3, 4, 5, 6, 7, 8, |
| | | 9, 10, 11, 12, 13, |
| | | 14, 15, 16- |
| | | CD3-VL-CDR1 |
| 20 | DASSLEN | 1-CD3-VL-CDR2 |
| 21 | QQFKSYPRT | 1, 2, 3, 5, 6-CD3-VL- |
| | | CDR3 |
| 22 | DASSLES | 2, 3, 4, 5, 6, 7, 8, 9, |
| | | 10, 11, 12, 13, 14, 15, |
| | | 16-CD3-VL-CDR2 |
| 23 | QQFNSYPRT | 4, 7, 8, 9, 10, 11, 12, |
| | | 13, 14, 15, 16-CD3- |
| | | VL-CDR3 |
| 24 | QVQLVESGGGVVQPGRSLRLSCAASGFTFSRYGIHWVRQAPGK | 1-CD3-VH |
| | GLEWVAAIWYDGSKKYYADSVKGRFTISRDNSKNTLDLQMNS | |
| | LRAEDTAVYYCARSNYDILTRYYQYGINVWGQGTTVTVSS | |
| 25 | QVQLVESGGGVVQPGRSLRLSCAASGFTFSNYGIHWVRQAPG | 2-CD3-VH |
| | KGLEWVAAIWYDASKKYYADSVKGRFTISRDNSKNTLYLQMN | |
| | SLRAEDTAVYYCARSNYDILTRYYQYGINVWGQGTTVTVSS | |
| 26 | QVQLVESGGGVVQPGRSLRLSCAASGFTFSRYGIHWVRQAPGK | 3, 10-CD3-VH |
| | GLEWVAAIWYDASKKYYADSVKGRFTISRDNSKNTLYLQMNS | |
| | LRAEDTAVYYCARSNYDILTRYYQYGLDVWGQGTTVTVSS | |
| 27 | QVQLVESGGGVVQPGRSLRLSCAASGFTFSRYGIHWVRQAPGK | 4-CD3-VH |
| | GLEWVAAIWYDASKKYYADSVKGRFTISRDNSKNTLYLQMNS | |
| | LRAEDTAVYYCARSNYDILTRYYSHGINVWGQGTTVTVSS | |
| 28 | QVQLVESGGGVVQPGGSLRLSCAASGFTFSRYGIHWVRQAPGK | 5-CD3-VH |
| | CLEWVAAIWYDASKTYYADSVKGRFTISRDNSKNTLYLQMNS | |
| | LRAEDTAVYYCARSNYDILTRYYQYGLDVWGQGTTVTVSS | |
| 29 | QVQLVESGGGVVQPGGSLRLSCAASGFTFSRYGIHWVRQAPGK | 6-CD3-VH |
| | GLEWVAAIWYDASKTYYADSVKGRFTISRDNSKNTLYLQMNS | |
| | LRAEDTAVYYCARSNYDILTRYYQYGLDVWGQGTTVTVSS | |
| 30 | QVQLVESGGGVVQPGGSLRLSCAASGFTFSRYGIHWVRQAPGK | 7-CD3-VH |
| | GLEWVAAIWYDASKTYYADSVKGRFTISRDNSKNTLYLQMNS | |
| | LRAEDTAVYYCARSNYDILTRYYSHGINVWGQGTTVTVSS | |
| 31 | QVQLVESGGGVVQPGGSLRLSCAASGFTFSRYGIHWVRQAPGK | 8-CD3-VH |
| | CLEWVAAIWYDASKTYYADSVKGRFTISRDNSKNTLYLQMNS | |
| | LRAEDTAVYYCARSNYDILTRYYSHGINVWGQGTTVTVSS | |
| 32 | QVQLVESGGGVVQPGGSLRLSCAASGFTFSRYGIHWVRQAPGK | 9-CD3-VH |
| | GLEWVAAIWYDASKKYYADSVKGRFTISRDNSKNTLYLQMNS | |
| | LRAEDTAVYYCARSNYDILTRYYQYGLDVWGQGTTVTVSS | |
| 33 | QVQLVESGGGVVQPGESLRLSCAASGFTFSRYGIHWVRQAPGK | 11-CD3-VH |
| | GLEWVAAIWYDASKDYYADSVKGRFTISRDNSKNTLYLQMNS | |
| | LRAEDTAVYYCARSNYDILTRYYQYGLDVWGQGTTVTVSS | |
| 34 | QVQLVESGGGVVQPGGSLRLSCAASGFTFSRYGIHWVRQAPGK | 12-CD3-VH |
| | GLEWVAAIWYDASKTYYADSVKGRFTISRDNSSNTLYLQMNSL | |
| | RAEDTAVYYCARSNYDILTRYYQYGLDVWGQGTTVTVSS | |
| 35 | QVQLVESGGGVVQPGESLRLSCAASGFTFSRYGIHWVRQAPGK | 13-CD3-VH |
| | GLEWVAAIWYDASKDYYADSVKGRFTISRDNSENTLYLQMNS | |
| | LRAEDTAVYYCARSNYDILTRYYQYGLDVWGQGTTVTVSS | |
| 36 | QVQLVESGGGVVQPGGSLRLSCAASGFTFSRYGIHWVRQAPGK | 14-CD3-VH |
| | GLEWVAAIWYDASKKYYADSVKGRFTISRDNSSNTLYLQMNS | |
| | LRAEDTAVYYCARSNYDILTRYYQYGLDVWGQGTTVTVSS | |
| 37 | QVQLVESGGGVVQPGESLRLSCAASGFTFSRYGIHWVRQAPGK | 15-CD3-VH |
| | GLEWVAAIWYDASKKYYADSVKGRFTISRDNSENTLYLQMNS | |
| | LRAEDTAVYYCARSNYDILTRYYQYGLDVWGQGTTVTVSS | |
| 38 | QVQLVESGGGVVQPGGSLRLSCAASGFTFSRYGIHWVRQAPGK | 16-CD3-VH |
| | GLEWVAAIWYDASKTYYADSVKGRFTISRDNSKNTLYLQMNS | |
| | LRAEDTAVYYCARSNYDILTGHYQYGINVWGQGTTVTVSS | |
| 39 | AIQLTQSPSSLSASVGDRVTITCRASQGISSALAWYQQKPGKAP | 1-CD3-VL |
| | KLLIYDASSLENGVPSRFSGSGSGTDFTLTISSLQPEDFATYYCQ | |
| | QFKSYPRTFGQGTKVEIK | |
| 40 | AIQLTQSPSSLSASVGDRVTITCRASQGISSALAWYQQKPGKAP | 2, 3, 6, 9, 10, 11, |
| | KLLIYDASSLESGVPSRFSGSGSGTDFTLTISSLQPEDFATYYCQ | 12, 13, 14, 15-CD3-VL |
| | QFKSYPRTFGQGTKVEIK | |
| 41 | AIQLTQSPSSLSASVGDRVTITCRASQGISSALAWYQQKPGKAP | 4, 7, 16-CD3-VL |
| | KLLIYDASSLESGVPSRFSGSGSGTDFTLTISSLQPEDFATYYCQ | |
| | QFNSYPRTFGQGTKVEIK | |
| 42 | AIQLTQSPSSLSASVGDRVTITCRASQGISSALAWYQQKPGKAP | 5, -CD3-VL |
| | KLLIYDASSLESGVPSRFSGSGSGTDFTLTISSLQPEDFATYYCQ | |
| | QFKSYPRTFGCGTKVEIK | |
| 43 | AIQLTQSPSSLSASVGDRVTITCRASQGISSALAWYQQKPGKAP | 8-CD3-VL |
| | KLLIYDASSLESGVPSRFSGSGSGTDFTLTISSLQPEDFATYYCQ | |
| | QFNSYPRTFGCGTKVEIK | |
| 44 | DKTHTCPPCPAPEAEGAPSVFLFPPKPKDTLMISRTPEVTCVVV | Fc - IgG1.3f-Knob |
| | DVSHEDPEVKFNWYVDGVEVHNAKTKPREEQYNSTYRVVSV | L234A, L235E, |
| | LTVLHQDWLNGKEYKCKVSNKALPAPIEKTISKAKGQPREPQV | G237A; |
| | YTLPPCREEMTKNQVSLWCLVKGFYPSDIAVEWESNGQPENN | D356E, L358M; |
| | YKTTPPVLDSDGSFFLYSKLTVDKSRWQQGNVFSCSVMHEAL | S354C, T366W |
| | HNHYTQKSLSLSPG | |
| 45 | DKTHTCPPCPAPEAEGAPSVFLFPPKPKDTLMISRTPEVTCVVV | Fc - IgG1.3f-Hole |
| | DVSHEDPEVKFNWYVDGVEVHNAKTKPREEQYNSTYRVVSV | L234A, L235E, |
| | LTVLHQDWLNGKEYKCKVSNKALPAPIEKTISKAKGQPREPQV | G237A; |
| | CTLPPSREEMTKNQVSLSCAVKGFYPSDIAVEWESNGQPENNY | D356E, L358M; |
| | KTTPPVLDSDGSFFLVSKLTVDKSRWQQGNVFSCSVMHEALH | Y349C, T366S, |
| | NHYTQKSLSLSPG | L368A, Y407V |
| 46 | DKTHTCPPCPAPEAAGGPSVFLFPPKPKDTLMISRTPEVTCVVV | Fc - IgGI KEMA |
| | SVSHEDPEVKFNWYVDGVEVHNAKTKPREEQYNSTYRVVSVL | AAS-Knob |
| | TVLHQDWLNGKEYKCKVSNKALPAPIEKTISKAKGQPREPQV | L234A, L235A, |
| | YTLPPCREEMTKNQVSLWCLVKGFYPSDIA VEWESNGQPENN | D265S; |
| | YKTTPPVLDSDGSFFLYSKLTVDKSRWQQGNVFSCSVMHEAL | D356E, L358M; |
| | HNHYTQKSLSLSPG | S354C, T366W |
| 47 | DKTHTCPPCPAPEAAGGPSVFLFPPKPKDTLMISRTPEVTCVVV | Fc - IgGI KEMA |
| | SVSHEDPEVKFNWYVDGVEVHNAKTKPREEQYNSTYRVVSVL | AAS-HoleCC |
| | TVLHQDWLNGKEYKCKVSNKALPAPIEKTISKAKGQPREPQVC | L234A, L235A, |
| | TLPPSREEMTKNQVSLSCAVKGFYPSDIAVEWESNGQPENNYK | D265S; |
| | TTPPVLDSDGSFFLVSKLTVDKSRWQQGNVFSCSVMHEALHN | D356E, L358M; |
| | HYTQKSLSLSPG | Y349C, T366S, |
| | | L368A, Y407V |
| 48 | ERKSCVECPPCPAPPVAGKSVFLFPPKPKDTLMISRTPEVTCVV | Fc- IgG2.3 P238K- |
| | VDVSHEDPEVQFNWYVDGVEVHNAKTKPREEQFNSTFRVVSV | Knob |
| | LTVVHQDWLNGKEYKCKVSNKGLPAPIEKTISKTKGQPREPQV | P238K; |
| | YTLPPCREEMTKNQVSLWCLVKGFYPSDIAVEWESNGQPENN | D356E, L358M; |
| | YKTTPPMLDSDGSFFLYSKLTVDKSRWQQGNVFSCSVMHEAL | S354C, T366W |
| | HNHYTQKSLSLSPG | |
| 49 | ERKSCVECPPCPAPPVAGKSVFLFPPKPKDTLMISRTPEVTCVV | Fc - IgG2.3 P238K- |
| | VDVSHEDPEVQFNWYVDGVEVHNAKTKPREEQFNSTFRVVSV | Hole |
| | LTVVHQDWLNGKEYKCKVSNKGLPAPIEKTISKTKGQPREPQV | P238K; |
| | CTLPPSREEMTKNQVSLSCAVKGFYPSDIAVEWESNGQPENNY | D356E, L358M; |
| | KTTPPMLDSDGSFFLVSKLTVDKSRWQQGNVFSCSVMHEALH | Y349C, T366S, |
| | NHYTQKSLSLSPG | L368A, Y407V |
| 50 | ASTKGPSVFPLAPSSKSTSGGTAALGCLVKDYFPEPVTVSWNS | IgG1 CH1 |
| | GALTSGVHTFPAVLQSSGLYSLSSVVTVPSSSLGTQTYICNVNH | |
| | KPSNTKVDKKVEPKSC | |
| 51 | ASTKGPSVFPLAPCSRSTSESTAALGCLVKDYFPEPVTVSWNSG | IgG2 CH1 |
| | ALTSGVHTFPAVLQSSGLYSLSSVVTVPSSNFGTQTYTCNVDH | |
| | KPSNTKVDKTV | |
| 52 | RTVAAPSVFIFPPSDEQLKSGTASVVCLLNNFYPREAKVQWKV | Constant Kappa |
| | DNALQSGNSQESVTEQDSKDSTYSLSSTLTLSKADYEKHKVYA | |
| | CEVTHQGLSSPVTKSFNRGEC | |
| 53 | QVQLVESGGGVVQPGRSLRLSCAASGFTFSRYGIHWVRQAPG | 1-CD3- M2 |
| | KGLEWVAAIWYDSSKKYYADSVKGRFTISRDNSKNTLDLQMN | |
| | SLRAEDTAVYYCARSNYDILTRYYQYGINVWGQGTTVTVSS | |
| 54 | QVQLVESGGGVVQPGRSLRLSCAASGFTFSRYGIHWVRQAPG | 1-CD3- M3 |
| | KGLEWVAAIWYDASKKYYADSVKGRFTISRDNSKNTLDLQM | |
| | NSLRAEDTAVYYCARSNYDILTRYYQYGINVWGQGTTVTVSS | |
| 55 | QVQLVESGGGVVQPGRSLRLSCAASGFTFSRYGIHWVRQAPG | 1-CD3- M5 |
| | KGLEWVAAIWYDGSHKYYADSVKGRFTISRDNSKNTLDLQM | |
| | NSLRAEDTAVYYCARSNYDILTRYYQYGINVWGQGTTVTVSS | |
| 56 | QVQLVESGGGVVQPGRSLRLSCAASGFTFSRYGIHWVRQAPG | 1-CD3- M6 |
| | KGLEWVAAIWYDGSNKYYADSVKGRFTISRDNSKNTLDLQM | |
| | NSLRAEDTAVYYCARSNYDILTRYYQYGINVWGQGTTVTVSS | |
| 57 | QVQLVESGGGVVQPGRSLRLSCAASGFTFSRYGIHWVRQAPG | 1-CD3-M7 |
| | KGLEWVAAIWYDGSKKYYADSVKGRFTISRDNSKNTLDLQM | |
| | NSLRAEDTAVYYCARSNYDILTRHYQYGINVWGQGTTVTVSS | |
| 58 | QVQLVESGGGVVQPGRSLRLSCAASGFTFSRYGIHWVRQAPG | 1-CD3-M8 |
| | KGLEWVAAIWYDGSKKYYADSVKGRFTISRDNSKNTLDLQM | |
| | NSLRAEDTAVYYCARSNYDILTGHYQYGINVWGQGTTVTVSS | |
| 59 | QVQLVESGGGVVQPGRSLRLSCAASGFTFSRYGIHWVRQAPG | 1-CD3-M9 |
| | KGLEWVAAIWYDGSKKYYADSVKGRFTISRDNSKNTLDLQM | |
| | NSLRAEDTAVYYCARSNYDILTGYYQYGINVWGQGTTVTVSS | |
| 60 | QVQLVESGGGVVQPGRSLRLSCAASGFTFSRYGIHWVRQAPG | 1-CD3-M10 |
| | KGLEWVAAIWYDGSKKYYADSVKGRFTISRDNSKNTLDLQM | |
| | NSLRAEDTAVYYCARSNYDILTRYYQHGINVWGQGTTVTVSS | |
| 61 | QVQLVESGGGVVQPGRSLRLSCAASGFTFSRYGIHWVRQAPG | 1-CD3-M11 |
| | KGLEWVAAIWYDGSKKYYADSVKGRFTISRDNSKNTLDLQM | |
| | NSLRAEDTAVYYCARSNYDILTRYYSHGINVWGQGTTVTVSS | |
| 62 | QVQLVESGGGVVQPGRSLRLSCAASGFTFSNYGIHWVRQAPG | 1-CD3-M16 |
| | KGLEWVAAIWYDGSKKYYADSVKGRFTISRDNSKNTLDLQM | |
| | NSLRAEDTAVYYCARSNYDILTRYYQYGINVWGQGTTVTVSS | |
| 63 | QVQLVESGGGVVQPGRSLRLSCAASGFTFSNHGIHWVRQAPG | 1-CD3-M17 |
| | KGLEWVAAIWYDGSKKYYADSVKGRFTISRDNSKNTLDLQM | |
| | NSLRAEDTAVYYCARSNYDILTRYYQYGINVWGQGTTVTVSS | |
| 64 | QVQLVESGGGVVQPGRSLRLSCAASGFTFSRHGIHWVRQAPG | 1-CD3-M18 |
| | KGLEWVAAIWYDGSKKYYADSVKGRFTISRDNSKNTLDLQM | |
| | NSLRAEDTAVYYCARSNYDILTRYYQYGINVWGQGTTVTVSS | |
| 65 | QVQLVESGGGVVQPGRSLRLSCAASGFTFSRYGIHWVRQAPG | 1-CD3-M19 |
| | KGLEWVAAIWYDGSKKYYADSVKGRFTISRDNSKNTLDLQM | |
| | NSLRAEDTAVYYCARSNFDILTRYYQYGINVWGQGTTVTVSS | |
| 66 | QVQLVESGGGVVQPGRSLRLSCAASGFTFSRYGIHWVRQAPG | 1-CD3-M20 |
| | KGLEWVAAIWYDGSKKYYADSVKGRFTISRDNSKNTLDLQM | |
| | NSLRAEDTAVYYCARSNFDVLTRYYQYGINVWGQGTTVTVSS | |
| 67 | QVQLVESGGGVVQPGRSLRLSCAASGFTFSRYGIHWVRQAPG | 1-CD3-M21 |
| | KGLEWVAAIWYDGSKKYYADSVKGRFTISRDNSKNTLDLQM | |
| | NSLRAEDTAVYYCARSNYDVLTRYYQYGINVWGQGTTVTVSS | |
| 68 | QVQLVESGGGVVQPGRSLRLSCAASGFTFSRYGIHWVRQAPG | 1-CD3-M22 |
| | KGLEWVAAIWYDGSKKYYADSVKGRFTISRDNSKNTLDLQM | |
| | NSLRAEDTAVYYCARSNYDILTRYYQYGLDVWGQGTTVTVSS | |
| 69 | QVQLVESGGGVVQPGRSLRLSCAASGFTFSRYGIHWVRQAPG | 1-CD3-C1 |
| | KGLEWVAAIWYDASKKYYADSVKGRFTISRDNSKNTLYLQM | |
| | NSLRAEDTAVYYCARSNYDILTRYYQYGINVWGQGTTVTVSS | |
| 70 | QVQLVESGGGVVQPGRSLRLSCAASGFTFSRYGIHWVRQAPG | 1-CD3-C5 |
| | KGLEWVAAIWYDASHKYYADSVKGRFTISRDNSKNTLYLQM | |
| | NSLRAEDTAVYYCARSNYDILTRYYQYGINVWGQGTTVTVSS | |
| 71 | QVQLVESGGGVVQPGRSLRLSCAASGFTFSRYGIHWVRQAPG | 1-CD3-C6 |
| | KGLEWVAAIWYDASNKYYADSVKGRFTISRDNSKNTLYLQM | |
| | NSLRAEDTAVYYCARSNYDILTRYYQYGINVWGQGTTVTVSS | |
| 72 | QVQLVESGGGVVQPGRSLRLSCAASGFTFSRYGIHWVRQAPG | 1-CD3-C8 |
| | KGLEWVAAIWYDASKKYYADSVKGRFTISRDNSKNTLYLQM | |
| | NSLRAEDTAVYYCARSNYDILTGHYQYGINVWGQGTTVTVSS | |
| 73 | QVQLVESGGGVVQPGRSLRLSCAASGFTFSRYGIHWVRQAPG | 1-CD3-C9 |
| | KGLEWVAAIWYDASKKYYADSVKGRFTISRDNSKNTLYLQM | |
| | NSLRAEDTAVYYCARSNYDILTGYYQYGINVWGQGTTVTVSS | |
| 74 | QVQLVESGGGVVQPGRSLRLSCAASGFTFSNHGIHWVRQAPG | 1-CD3-C17 |
| | KGLEWVAAIWYDASKKYYADSVKGRFTISRDNSKNTLYLQM | |
| | NSLRAEDTAVYYCARSNYDILTRYYQYGINVWGQGTTVTVSS | |
| 75 | QVQLVESGGGVVQPGRSLRLSCAASGFTFSRYGIHWVRQAPG | 1-CD3-C21 |
| | KGLEWVAAIWYDASKKYYADSVKGRFTISRDNSKNTLYLQM | |
| | NSLRAEDTAVYYCARSNYDVLTRYYQYGINVWGQGTTVTVSS | |
| 76 | AIQLTQSPSSLSASVGDRVTITCRASQGISSALAWYQQKPGKAP | 1-CD3-M4 |
| | KLLIYDASSLESGVPSRFSGSGSGTDFTLTISSLQPEDFATYYCQ | |
| | QFKSYPRTFGQGTKVEIK | |
| 77 | AIQLTQSPSSLSASVGDRVTITCRASQGISSALAWYQQKPGKAP | 1-CD3-M12 |
| | KLLIYDASSLENGVPSRFSGSGSGTDFTLTISSLQPEDFATYYCQ | |
| | QFNSYPRTFGQGTKVEIK | |
| 78 | AIQLTQSPSSLSASVGDRVTITCRASQGISSALAWYQQKPGKAP | 1-CD3-M13 |
| | KLLIYDASSLESGVPSRFSGSGSGTDFTLTISSLQPEDFATYYCQ | |
| | QFNSYPRTFGQGTKVEIK | |
| 79 | AIQLTQSPSSLSASVGDRVTITCRASQGISSALAWYQQKPGKAP | 1-CD3-M14 |
| | KLLIYDASGLENGVPSRFSGSGSGTDFTLTISSLQPEDFATYYCQ | |
| | QFKSYPRTFGQGTKVEIK | |
| 80 | AIQLTQSPSSLSASVGDRVTITCRASQGISSALAWYQQKPGKAP | 1-CD3-M15 |
| | KLLIYDASGLESGVPSRFSGSGSGTDFTLTISSLQPEDFATYYCQ | |
| | QFNSYPRTFGQGTKVEIK | |
| 81 | AIQLTQSPSSLSASVGDRVTITCRASQGISSALAWYQQKPGKAP | 1-CD3-C12 |
| | KLLIYDASSLESGVPSRFSGSGSGTDFTLTISSLQPEDFATYYCQ | |
| | QFNSYPRTFGQGTKVEIK | |
| 82 | QDGNEEMGGITQTPYKVSISGTTVILTCPQYPGSEILWQHNDKN | Mature human CD3 |
| | IGGDEDDKNIGSDEDHLSLKEFSELEQSGYYVCYPRGSKPEDA | epsilon |
| | NFYLYLRARVCENCMEMDVMSVATIVIVDICITGGLLLLVYY | |
| | WSKNRKAKAKPVTRGAGAGGRQRGQNKERPPPVPNPDYEPIR | |
| | KGQRDLYSGLNQRRI | |
| 83 | QVQLVESGGGVVQPGX₁SLRLSCAASGFTFSX₂YGIHWVRQAP | Consensus VH |
| | GKX₃LEWVAAIWYDASKX₄YYADSVKGRFTISRDNSX₅NTLYLQ | X₁ = R or G or E |
| | MNSLRAEDTAVYYCARSNYDILTX₆X₇YX₈X₉GX₁₀X11VWGQGTT | X₂ = R or N |
| | VTVSS | X₃ = G or C |
| | | X₄ = K or T or D |
| | | X₅ = K or E or S |
| | | X₆ = R or G |
| | | X₇ = Y or H |
| | | X₈ = Q or S |
| | | X₉ = Y or H |
| | | X₁₀ = I or L |
| | | X₁₁ = N or D |
| 84 | AIQLTQSPSSLSASVGDRVTITCRASQGISSALAWYQQKPGKAP | Consensus VL |
| | KLLIYDASSLESGVPSRFSGSGSGTDFTLTISSLQPEDFATYYCQ | X₁ = K or N |
| | QFX₁SYPRTFGX₂GTKVEIK | X₂ = Q or C |
| 85 | AIWYDASKX₄YYADSVKG | CDR-H2 Consensus |
| | | X₄ = K or T or D |
| 86 | EPKSCDKTHTCPPCPAPELLGGPSVFLFPPKPKDTLMISRTPEVT | wildtype IgG1 Fc |
| | CVVVDVSHEDPEVKFNWYVDGVEVHNAKTKPREEQYNSTYR | |
| | VVSVLTVLHQDWLNGKEYKCKVSNKALPAPIEKTISKAKGQPR | |
| | EPQVYTLPPSREEMTKNQVSLTCLVKGFYPSDIAVEWESNGQPE | |
| | NNYKTTPPVLDSDGSFFLYSKLTVDKSRWQQGNVFSCSVMHE | |
| | ALHNHYTQKSLSLSPG | |
| 87 | EPKSCDKTHTCPPCPAPELLGGPSVFLFPPKPKDTLMISRTPEVT | wildtype IgG1 Fc |
| | CVVVDVSHEDPEVKFNWYVDGVEVHNAKTKPREEQYNSTYR | |
| | VVSVLTVLHQDWLNGKEYKCKVSNKALPAPIEKTISKAKGQPR | |
| | EPQVYTLPPSRDELTKNQVSLTCLVKGFYPSDIAVEWESNGQPE | |
| | NNYKTTPPVLDSDGSFFLYSKLTVDKSRWQQGNVFSCSVMHE | |
| | ALHNHYTQKSLSLSPG | |
| 88 | EPKSCDKTHTCPPCPAPELLGGPSVFLFPPKPKDTLMISRTPEVT | heterodimeric Fc |
| | CVVVDVSHEDPEVKFNWYVDGVEVHNAKTKPREEQYNSTYR | polypeptide 1 |
| | VVSVLTVLHQDWLNGKEYKCKVSNKALPAPIEKTISKAKGQPR | (T350V, L351Y, |
| | EPQVYVYPPSREEMTKNQVSLTCLVKGFYPSDIAVEWESNGQP | F405A and Y407V) |
| | ENNYKTTPPVLDSDGSFALVSKLTVDKSRWQQGNVFSCSVMHE | without effector |
| | ALHNHYTQKSLSLSPG | mutations |
| 89 | EPKSCDKTHTCPPCPAPELLGGPSVFLFPPKPKDTLMISRTPEVT | heterodimeric Fc |
| | CVVVDVSHEDPEVKFNWYVDGVEVHNAKTKPREEQYNSTYR | polypeptide 2 |
| | VVSVLTVLHQDWLNGKEYKCKVSNKALPAPIEKTISKAKGQPR | without effector |
| | EPQVYVLPPSREEMTKNQVSLLCLVKGFYPSDIAVEWESNGQP | mutations |
| | ENNYLTWPPVLDSDGSFFLYSKLTVDKSRWQQGNVFSCSVMH | |
| | EALHNHYTQKSLSLSPG | |
| 90 | EPKSCDKTHTCPPCPAPEAAGGPSVFLFPPKPKDTLMISRTPEVT | heterodimeric Fc |
| | CVVVSVSHEDPEVKFNWYVDGVEVHNAKTKPREEQYNSTYR | polypeptide 1 |
| | VVSVLTVLHQDWLNGKEYKCKVSNKALPAPIEKTISKAKGQPR | (T350V, L351Y, |
| | EPQVYVYPPSREEMTKNQVSLTCLVKGFYPSDIAVEWESNGQP | F405A and Y407V)) |
| | ENNYKTTPPVLDSDGSFALVSKLTVDKSRWQQGNVFSCSVMHE | with LALA effector |
| | ALHNHYTQKSLSLSPG | mutations and |
| | | D265S |
| 91 | EPKSCDKTHTCPPCPAPEAAGGPSVFLFPPKPKDTLMISRTPEVT | heterodimeric Fc |
| | CVVVSVSHEDPEVKFNWYVDGVEVHNAKTKPREEQYNSTYR | polypeptide 2 |
| | VVSVLTVLHQDWLNGKEYKCKVSNKALPAPIEKTISKAKGQPR | (T350V, T366L, |
| | EPQVYVLPPSREEMTKNQVSLLCLVKGFYPSDIAVEWESNGQP | K392L and T394W) |
| | ENNYLTWPPVLDSDGSFFLYSKLTVDKSRWQQGNVFSCSVMH | with LALA effector |
| | EALHNHYTQKSLSLSPG | mutations and |
| | | D265S |
| 92 | EPKSCDKTHTCPPCPAPEAAGGPSVFLFPPKPKDTLMISRTPEVT | heterodimeric Fc |
| | CVVVDVSHEDPEVKFNWYVDGVEVHNAKTKPREEQYNSTYR | polypeptide 1 |
| | VVSVLTVLHQDWLNGKEYKCKVSNKALGAPIEKTISKAKGQP | (T350V, L351Y, |
| | REPQVYVYPPSREEMTKNQVSLTCLVKGFYPSDIAVEWESNGQ | F405A and Y407V)) |
| | PENNYKTTPPVLDSDGSFALVSKLTVDKSRWQQGNVFSCSVMH | with LALA effector |
| | EALHNHYTQKSLSLSPG | mutations and |
| | | P329G |
| 93 | EPKSCDKTHTCPPCPAPEAAGGPSVFLFPPKPKDTLMISRTPEVT | heterodimeric Fc |
| | CVVVDVSHEDPEVKFNWYVDGVEVHNAKTKPREEQYNSTYR | polypeptide 2 |
| | VVSVLTVLHQDWLNGKEYKCKVSNKALGAPIEKTISKAKGQP | (T350V, T366L, |
| | REPQVYVLPPSREEMTKNQVSLLCLVKGFYPSDIAVEWESNGQ | K392L and T394W) |
| | PENNYLTWPPVLDSDGSFFLYSKLTVDKSRWQQGNVFSCSVMH | with LALA effector |
| | EALHNHYTQKSLSLSPG | mutations and |
| | | P329G |
| 94 | GGGGS | G4S linker |
| 95 | GGGGSGGGGS | (G4S)2 linker |
| 96 | GGGGSGGGGSGGGGS | (G4S)3 linker |
| 97 | GGGS | 3GS linker |
| 98 | GSTSGSGKPGSGEGSTKG | Linker |
| 99 | SRGGGGSGGGGSGGGGSLEMA | Linker |
| 100 | GSRGGGGSGGGGSGGGGSLEMA | linker |
| 101 | ASTKGPSVFPLAPSSKSTSGGTAWLGCEVTDYFPEPVTVSWNS | CH1 region |
| | GALTSGVHTFPAVLESSGLYSLSSVVTVPSSSLGTQTYICNVNHK | |
| | PSNTKVDKKV | |
| 102 | ASTKGPSVFPLAPSSKSTSGGTAALGCLVKDYFPEPVTVSWNSG | CH1 region |
| | ALTSGVHTFPAVLKSSGLYSLSSVVTVPSSSLGTQTYICNVNHKP | |
| | SNTKVDKKV | |
| 103 | QPKANPTVTLFPPSSEELQANKATLVCLISDFYPGAVTVAWKAD | Lambda constant |
| | GSPVKAGVETTKPSKQSNNKYAASSYLSLTPEQWKSHRSYSCQ | light |
| | VTHEGSTVEKTVAPTECS | |
| 104 | TVAAPSVFIFPPSDEQLKSGTASVVCLLNNFYPREAKVQWKVD | Kappa constant light |
| | NALQSGNSQESVTEQDSKDSTYSLSSTLTLSKADYEKHKVYAC | |
| | EVTHQGLSSPVTKSFNRGEC | |
| 105 | QVQLVESGGGVVQPGX₁SLRLSCAASGFTFSX₂X₃GIHWVRQAP | Consensus CD3 VH |
| | GKX₄LEWVAAIWYDX₅SX₆X₇YYADSVKGRFTISRDNSX₈NTLX₉ | (all sequences) |
| | LQMNSLRAEDTAVYYCARSNX₁₀DX₁₁LTX₁₂X₁₃YX₁₄X₁₅GX₁₆X₁₇V | amino acid sequence |
| | WGQGTTVTVSS | X₁ = R or G or E |
| | | X₂ = R or N |
| | | X₃ = Y or H |
| | | X₄ = G or C |
| | | X₅ = G or A |
| | | X₆ = K or H or N |
| | | X₇ = K or T or D |
| | | X₈ = K or E or S |
| | | X₉ = Y or D |
| | | X₁₀ = Y or F |
| | | X₁₁ = I or V |
| | | X₁₂ = R or G |
| | | X₁₃ = Y or H |
| | | X₁₄ = Q or S |
| | | X₁₅ = Y or H |
| | | X₁₆ = I or L |
| | | X₁₇ = N or D |
| 106 | AIQLTQSPSSLSASVGDRVTITCRASQGISSALAWYQQKPGKAP | Consensus CD3 VL |
| | KLLIYDASX₁LEX₂GVPSRFSGSGSGTDFTLTISSLQPEDFATYYC | (all sequences) |
| | QQFX₃SYPRTFGX₄GTKVEIK | amino acid sequence |
| | | X₁ = S or G |
| | | X₂ = N or S |
| | | X₃ = K or N |
| | | X₄ = Q or C |
| 107 | X₁X₂GIH | CD3 VH CDR1 |
| | | consensus (all |
| | | sequences) amino |
| | | acid sequence |
| | | X₁ = R or N |
| | | X₂ = Y or H |
| 108 | AIWYDX₁SX₂X₃YYADSVKG | CD3 VH CDR2 |
| | | consensus amino |
| | | acid sequence |
| | | X₁ = G or A |
| | | X₂ = K or H or N |
| | | X₃ = K or T or D |
| 109 | SNX₁DX₂LTX₃X₄YX₅X₆GX₇X₈V | CD3 VH CDR3 |
| | | consensus (all |
| | | sequences) amino |
| | | acid sequence |
| | | X₁ = Y or F |
| | | X₂ = I or V |
| | | X₃ = R or G |
| | | X₄ = Y or H |
| | | X₅ = Q or S |
| | | X₆ = Y or H |
| | | X₇ = I or L |
| | | X₈ = N or D |
| 110 | DASX₁LEX₂ | CD3 VL CDR2 |
| | | consensus (all |
| | | sequences) amino |
| | | acid sequence |
| | | X₁ = S or G |
| | | X₂ = N or S |
| 111 | NHGIH | 1-CD3-M17 VH |
| | | CDR1 |
| 112 | RHGIH | 1-CD3-M18 VH |
| | | CDR1 |
| 113 | AIWYDSSKKYYADSVKG | 1-CD3-M2 VH |
| | | CDR2 |
| 114 | AIWYDGSHKYYADSVKG | 1-CD3-M5 VH |
| | | CDR2 |
| 115 | AIWYDGSNKYYADSVKG | 1-CD3-M6 VH |
| | | CDR2 |
| 116 | AIWYDASHKYYADSVKG | 1-CD3-C5 VH |
| | | CDR2 |
| 117 | AIWYDASNKYYADSVKG | 1-CD3-C6 VH |
| | | CDR2 |
| 118 | SNYDILTRHYQYGINV | 1-CD3-M7 VH |
| | | CDR3 |
| 119 | SNYDILTGYYQYGINV | 1-CD3-M9 VH |
| | | CDR3 |
| 120 | SNYDILTRYYQHGINV | 1-CD3-M10 VH |
| | | CDR3 |
| 121 | SNFDILTRYYQYGINV | 1-CD3-M19 VH |
| | | CDR3 |
| 122 | SNFDVLTRYYQYGINV | 1-CD3-M20 VH |
| | | CDR3 |
| 123 | SNYDVLTRYYQYGINV | 1-CD3-M21 VH |
| | | CDR3 |
| 124 | DASGLEN | 1-CD3-M14 VL |
| | | CDR2 |
| 125 | DASGLES | 1-CD3-M15 VL |
| | | CDR2 |
| 126 | QVQLVESGGGVVQPGESLRLSCAASGFTFSRYGIHWVRQAPGK | 10-CD3-VH |
| | GLEWVAAIWYDASKKYYADSVKGRFTISRDNSKNTLYLQMNS | |
| | LRAEDTAVYYCARSNYDILTRYYQYGLDVWGQGTTVTVSS | |
| 127 | ZDGNEEMGGITQTP | human CD3 epsilon |
| | | peptide |
| 128 | QVQLVESGGGVVQPGRSLRLSCAASGFTFSRYGIHWVRQAPGK | 1-CD3 scFv |
| | GLEWVAAIWYDGSKKYYADSVKGRFTISRDNSKNTLDLQMNS | |
| | LRAEDTAVYYCARSNYDILTRYYQYGINVWGQGTTVTVSSGG | |
| | GGSGGGGSGGGGSAIQLTQSPSSLSASVGDRVTITCRASQGISSA | |
| | LAWYQQKPGKAPKLLIYDASSLENGVPSRFSGSGSGTDFTLTIS | |
| | SLQPEDFATYYCQQFKSYPRTFGQGTKVEIK | |
| 129 | QVQLVESGGGVVQPGRSLRLSCAASGFTFSRYGIHWVRQAPGK | 3-CD3 scFv |
| | GLEWVAAIWYDASKKYYADSVKGRFTISRDNSKNTLYLQMNS | |
| | LRAEDTAVYYCARSNYDILTRYYQYGLDVWGQGTTVTVSSGG | |
| | GGSGGGGSGGGGSAIQLTQSPSSLSASVGDRVTITCRASQGISSA | |
| | LAWYQQKPGKAPKLLIYDASSLESGVPSRFSGSGSGTDFTLTISS | |
| | LQPEDFATYYCQQFKSYPRTFGQGTKVEIK | |
| 130 | QVQLVESGGGVVQPGGSLRLSCAASGFTFSRYGIHWVRQAPGK | 5-CD3 scFv |
| | CLEWVAAIWYDASKTYYADSVKGRFTISRDNSKNTLYLQMNS | |
| | LRAEDTAVYYCARSNYDILTRYYQYGLDVWGQGTTVTVSSGG | |
| | GGSGGGGSGGGGSAIQLTQSPSSLSASVGDRVTITCRASQGISSA | |
| | LAWYQQKPGKAPKLLIYDASSLESGVPSRFSGSGSGTDFTLTISS | |
| | LQPEDFATYYCQQFKSYPRTFGCGTKVEIK | |
| 131 | QVQLVESGGGVVQPGGSLRLSCAASGFTFSRYGIHWVRQAPGK | 8-CD3 scFv |
| | CLEWVAAIWYDASKTYYADSVKGRFTISRDNSKNTLYLQMNS | |
| | LRAEDTAVYYCARSNYDILTRYYSHGINVWGQGTTVTVSSGGG | |
| | GSGGGGSGGGGSAIQLTQSPSSLSASVGDRVTITCRASQGISSAL | |
| | AWYQQKPGKAPKLLIYDASSLESGVPSRFSGSGSGTDFTLTISSL | |
| | QPEDFATYYCQQFNSYPRTFGCGTKVEIK | |
| 132 | ASTKGPSVFPLAPSSKSTSGGTAALGCLVKDYFPEPVTVSWNSG | wild-type IgG1 |
| | ALTSGVHTFPAVLQSSGLYSLSSVVTVPSSSLGTQTYICNVNHKP | constant heavy chain |
| | SNTKVDKKVEPKSCDKTHTCPPCPAPELLGGPSVFLFPPKPKDT | |
| | LMISRTPEVTCVVVDVSHEDPEVKFNWYVDGVEVHNAKTKPR | |
| | EEQYNSTYRVVSVLTVLHQDWLNGKEYKCKVSNKALPAPIEKT | |
| | ISKAKGQPREPQVYTLPPSREEMTKNQVSLTCLVKGFYPSDIAV | |
| | EWESNGQPENNYKTTPPVLDSDGSFFLYSKLTVDKSRWQQGN | |
| | VFSCSVMHEALHNHYTQKSLSLSPG | |
| 133 | QVQLVESGGGVVQPGRSLRLSCAASGFTFSRYGIHWVRQAPGK | 1-CD3 Heavy Chain |
| | GLEWVAAIWYDGSKKYYADSVKGRFTISRDNSKNTLDLQMNS | (HC constant region |
| | LRAEDTAVYYCARSNYDILTRYYQYGINVWGQGTTVTVSSAST | underlined) |
| | KGPSVFPLAPSSKSTSGGTAALGCLVKDYFPEPVTVSWNSGALT | |
| | SGVHTFPAVLQSSGLYSLSSVVTVPSSSLGTQTYICNVNHKPSNT | |
| | KVDKKVEPKSCDKTHTCPPCPAPELLGGPSVFLFPPKPKDTLMI | |
| | SRTPEVTCVVVDVSHEDPEVKFNWYVDGVEVHNAKTKPREEQ | |
| | YNSTYRVVSVLTVLHQDWLNGKEYKCKVSNKALPAPIEKTISK | |
| | AKGQPREPQVYTLPPSREEMTKNQVSLTCLVKGFYPSDIAVEW | |
| | ESNGQPENNYKTTPPVLDSDGSFFLYSKLTVDKSRWQQGNVFS | |
| | CSVMHEALHNHYTQKSLSLSPG | |
| 134 | AIQLTQSPSSLSASVGDRVTITCRASQGISSALAWYQQKPGKAP | 1-CD3 Light Chain |
| | KLLIYDASSLENGVPSRFSGSGSGTDFTLTISSLQPEDFATYYCQ | (LC constant region |
| | QFKSYPRTFGQGTKVEIKRTVAAPSVFIFPPSDEQLKSGTASVVC | underlined) |
| | LLNNFYPREAKVQWKVDNALQSGNSQESVTEQDSKDSTYSLS | |
| | STLTLSKADYEKHKVYACEVTHQGLSSPVTKSFNRGEC | |
| 135 | QVQLVESGGGVVQPGRSLRLSCAASGFTFSNYGIHWVRQAPG | 2-CD3 Heavy Chain |
| | KGLEWVAAIWYDASKKYYADSVKGRFTISRDNSKNTLYLQMN | (HC constant region |
| | SLRAEDTAVYYCARSNYDILTRYYQYGINVWGQGTTVTVSSAS | underlined) |
| | TKGPSVFPLAPSSKSTSGGTAALGCLVKDYFPEPVTVSWNSGAL | |
| | TSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLM | |
| | ISRTPEVTCVVVDVSHEDPEVKFNWYVDGVEVHNAKTKPREE | |
| | QYNSTYRVVSVLTVLHQDWLNGKEYKCKVSNKALPAPIEKTIS | |
| | KAKGQPREPQVYTLPPSREEMTKNQVSLTCLVKGFYPSDIAVE | |
| | WESNGQPENNYKTTPPVLDSDGSFFLYSKLTVDKSRWQQGNV | |
| | FSCSVMHEALHNHYTQKSLSLSPG | |
| 136 | AIQLTQSPSSLSASVGDRVTITCRASQGISSALAWYQQKPGKAP | 2-CD3 Light Chain; |
| | KLLIYDASSLESGVPSRFSGSGSGTDFTLTISSLQPEDFATYYCQ | 3-CD3 Light Chain; |
| | QFKSYPRTFGQGTKVEIKRTVAAPSVFIFPPSDEQLKSGTASVVC | 6-CD3 Light Chain; |
| | LLNNFYPREAKVQWKVDNALQSGNSQESVTEQDSKDSTYSLS | 9-CD3 Light Chain; |
| | STLTLSKADYEKHKVYACEVTHQGLSSPVTKSFNRGEC | 10-CD3 Light |
| | | Chain; 11-CD3 |
| | | Light Chain; 12- |
| | | CD3 Light Chain; |
| | | 13-CD3 Light |
| | | Chain; 14-CD3 |
| | | Light Chain; 15- |
| | | CD3 Light Chain; |
| | | (LC constant region |
| | | underlined) |
| 137 | QVQLVESGGGVVQPGRSLRLSCAASGFTFSRYGIHWVRQAPGK | 3-CD3 Heavy Chain |
| | GLEWVAAIWYDASKKYYADSVKGRFTISRDNSKNTLYLQMNS | (HC constant region |
| | LRAEDTAVYYCARSNYDILTRYYQYGLDVWGQGTTVTVSSAS | underlined) |
| | TKGPSVFPLAPSSKSTSGGTAALGCLVKDYFPEPVTVSWNSGAL | |
| | TSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLM | |
| | ISRTPEVTCVVVDVSHEDPEVKFNWYVDGVEVHNAKTKPREE | |
| | QYNSTYRVVSVLTVLHQDWLNGKEYKCKVSNKALPAPIEKTIS | |
| | KAKGQPREPQVYTLPPSREEMTKNQVSLTCLVKGFYPSDIAVE | |
| | WESNGQPENNYKTTPPVLDSDGSFFLYSKLTVDKSRWQQGNV | |
| | FSCSVMHEALHNHYTQKSLSLSPG | |
| 138 | QVQLVESGGGVVQPGRSLRLSCAASGFTFSRYGIHWVRQAPGK | 4-CD3 Heavy Chain |
| | GLEWVAAIWYDASKKYYADSVKGRFTISRDNSKNTLYLQMNS | (HC constant region |
| | LRAEDTAVYYCARSNYDILTRYYSHGINVWGQGTTVTVSSAST | underlined) |
| | KGPSVFPLAPSSKSTSGGTAALGCLVKDYFPEPVTVSWNSGALT | |
| | SGVHTFPAVLQSSGLYSLSSVVTVPSSSLGTQTYICNVNHKPSNT | |
| | KVDKKVEPKSCDKTHTCPPCPAPELLGGPSVFLFPPKPKDTLMI | |
| | SRTPEVTCVVVDVSHEDPEVKFNWYVDGVEVHNAKTKPREEQ | |
| | YNSTYRVVSVLTVLHQDWLNGKEYKCKVSNKALPAPIEKTISK | |
| | AKGQPREPQVYTLPPSREEMTKNQVSLTCLVKGFYPSDIAVEW | |
| | ESNGQPENNYKTTPPVLDSDGSFFLYSKLTVDKSRWQQGNVES | |
| | CSVMHEALHNHYTQKSLSLSPG | |
| 139 | AIQLTQSPSSLSASVGDRVTITCRASQGISSALAWYQQKPGKAP | 4-CD3 Light Chain; |
| | KLLIYDASSLESGVPSRFSGSGSGTDFTLTISSLQPEDFATYYCQ | 7-CD3 Light Chain; |
| | QFNSYPRTFGQGTKVEIKRTVAAPSVFIFPPSDEQLKSGTASVVC | 16-CD3 Light |
| | LLNNFYPREAKVQWKVDNALQSGNSQESVTEQDSKDSTYSLS | Chain; |
| | STLTLSKADYEKHKVYACEVTHQGLSSPVTKSFNRGEC | (LC constant region |
| | | underlined) |
| 140 | QVQLVESGGGVVQPGGSLRLSCAASGFTFSRYGIHWVRQAPGK | 5-CD3 Heavy Chain |
| | CLEWVAAIWYDASKTYYADSVKGRFTISRDNSKNTLYLQMNS | (HC constant region |
| | LRAEDTAVYYCARSNYDILTRYYQYGLDVWGQGTTVTVSSAS | underlined) |
| | TKGPSVFPLAPSSKSTSGGTAALGCLVKDYFPEPVTVSWNSGAL | |
| | TSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLM | |
| | ISRTPEVTCVVVDVSHEDPEVKFNWYVDGVEVHNAKTKPREE | |
| | QYNSTYRVVSVLTVLHQDWLNGKEYKCKVSNKALPAPIEKTIS | |
| | KAKGQPREPQVYTLPPSREEMTKNQVSLTCLVKGFYPSDIAVE | |
| | WESNGQPENNYKTTPPVLDSDGSFFLYSKLTVDKSRWQQGNV | |
| | FSCSVMHEALHNHYTQKSLSLSPG | |
| 141 | AIQLTQSPSSLSASVGDRVTITCRASQGISSALAWYQQKPGKAP | 5-CD3 Light Chain |
| | KLLIYDASSLESGVPSRFSGSGSGTDFTLTISSLQPEDFATYYCQ | (LC constant region |
| | QFKSYPRTFGCGTKVEIKRTVAAPSVFIFPPSDEQLKSGTASVVC | underlined) |
| | LLNNFYPREAKVQWKVDNALQSGNSQESVTEQDSKDSTYSLS | |
| | STLTLSKADYEKHKVYACEVTHQGLSSPVTKSFNRGEC | |
| 142 | QVQLVESGGGVVQPGGSLRLSCAASGFTFSRYGIHWVRQAPGK | 6-CD3 Heavy Chain |
| | GLEWVAAIWYDASKTYYADSVKGRFTISRDNSKNTLYLQMNS | (HC constant region |
| | LRAEDTAVYYCARSNYDILTRYYQYGLDVWGQGTTVTVSSAS | underlined) |
| | TKGPSVFPLAPSSKSTSGGTAALGCLVKDYFPEPVTVSWNSGAL | |
| | TSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLM | |
| | ISRTPEVTCVVVDVSHEDPEVKFNWYVDGVEVHNAKTKPREE | |
| | QYNSTYRVVSVLTVLHQDWLNGKEYKCKVSNKALPAPIEKTIS | |
| | KAKGQPREPQVYTLPPSREEMTKNQVSLTCLVKGFYPSDIAVE | |
| | WESNGQPENNYKTTPPVLDSDGSFFLYSKLTVDKSRWQQGNV | |
| | FSCSVMHEALHNHYTQKSLSLSPG | |
| 143 | QVQLVESGGGVVQPGGSLRLSCAASGFTFSRYGIHWVRQAPGK | 7-CD3 Heavy Chain |
| | GLEWVAAIWYDASKTYYADSVKGRFTISRDNSKNTLYLQMNS | (HC constant region |
| | LRAEDTAVYYCARSNYDILTRYYSHGINVWGQGTTVTVSSAST | underlined) |
| | KGPSVFPLAPSSKSTSGGTAALGCLVKDYFPEPVTVSWNSGALT | |
| | SGVHTFPAVLQSSGLYSLSSVVTVPSSSLGTQTYICNVNHKPSNT | |
| | KVDKKVEPKSCDKTHTCPPCPAPELLGGPSVFLFPPKPKDTLMI | |
| | SRTPEVTCVVVDVSHEDPEVKFNWYVDGVEVHNAKTKPREEQ | |
| | YNSTYRVVSVLTVLHQDWLNGKEYKCKVSNKALPAPIEKTISK | |
| | AKGQPREPQVYTLPPSREEMTKNQVSLTCLVKGFYPSDIAVEW | |
| | ESNGQPENNYKTTPPVLDSDGSFFLYSKLTVDKSRWQQGNVFS | |
| | CSVMHEALHNHYTQKSLSLSPG | |
| 144 | QVQLVESGGGVVQPGGSLRLSCAASGFTFSRYGIHWVRQAPGK | 8-CD3 Heavy Chain |
| | CLEWVAAIWYDASKTYYADSVKGRFTISRDNSKNTLYLQMNS | (HC constant region |
| | LRAEDTAVYYCARSNYDILTRYYSHGINVWGQGTTVTVSSAST | underlined) |
| | KGPSVFPLAPSSKSTSGGTAALGCLVKDYFPEPVTVSWNSGALT | |
| | SGVHTFPAVLQSSGLYSLSSVVTVPSSSLGTQTYICNVNHKPSNT | |
| | KVDKKVEPKSCDKTHTCPPCPAPELLGGPSVFLFPPKPKDTLMI | |
| | SRTPEVTCVVVDVSHEDPEVKFNWYVDGVEVHNAKTKPREEQ | |
| | YNSTYRVVSVLTVLHQDWLNGKEYKCKVSNKALPAPIEKTISK | |
| | AKGQPREPQVYTLPPSREEMTKNQVSLTCLVKGFYPSDIAVEW | |
| | ESNGQPENNYKTTPPVLDSDGSFFLYSKLTVDKSRWQQGNVES | |
| | CSVMHEALHNHYTQKSLSLSPG | |
| 145 | AIQLTQSPSSLSASVGDRVTITCRASQGISSALAWYQQKPGKAP | 8-CD3 Light Chain |
| | KLLIYDASSLESGVPSRFSGSGSGTDFTLTISSLQPEDFATYYCQ | (LC constant region |
| | QFNSYPRTFGCGTKVEIKRTVAAPSVFIFPPSDEQLKSGTASVVC | underlined) |
| | LLNNFYPREAKVQWKVDNALQSGNSQESVTEQDSKDSTYSLS | |
| | STLTLSKADYEKHKVYACEVTHQGLSSPVTKSFNRGEC | |
| 146 | QVQLVESGGGVVQPGGSLRLSCAASGFTFSRYGIHWVRQAPGK | 9-CD3 Heavy Chain |
| | GLEWVAAIWYDASKKYYADSVKGRFTISRDNSKNTLYLQMNS | (HC constant region |
| | LRAEDTAVYYCARSNYDILTRYYQYGLDVWGQGTTVTVSSAS | underlined) |
| | TKGPSVFPLAPSSKSTSGGTAALGCLVKDYFPEPVTVSWNSGAL | |
| | TSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLM | |
| | ISRTPEVTCVVVDVSHEDPEVKFNWYVDGVEVHNAKTKPREE | |
| | QYNSTYRVVSVLTVLHQDWLNGKEYKCKVSNKALPAPIEKTIS | |
| | KAKGQPREPQVYTLPPSREEMTKNQVSLTCLVKGFYPSDIAVE | |
| | WESNGQPENNYKTTPPVLDSDGSFFLYSKLTVDKSRWQQGNV | |
| | FSCSVMHEALHNHYTQKSLSLSPG | |
| 147 | QVQLVESGGGVVQPGESLRLSCAASGFTFSRYGIHWVRQAPGK | 10-CD3 Heavy |
| | GLEWVAAIWYDASKKYYADSVKGRFTISRDNSKNTLYLQMNS | Chain |
| | LRAEDTAVYYCARSNYDILTRYYQYGLDVWGQGTTVTVSSAS | (HC constant region |
| | TKGPSVFPLAPSSKSTSGGTAALGCLVKDYFPEPVTVSWNSGAL | underlined) |
| | TSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLM | |
| | ISRTPEVTCVVVDVSHEDPEVKFNWYVDGVEVHNAKTKPREE | |
| | QYNSTYRVVSVLTVLHQDWLNGKEYKCKVSNKALPAPIEKTIS | |
| | KAKGQPREPQVYTLPPSREEMTKNQVSLTCLVKGFYPSDIAVE | |
| | WESNGQPENNYKTTPPVLDSDGSFFLYSKLTVDKSRWQQGNV | |
| | FSCSVMHEALHNHYTQKSLSLSPG | |
| 148 | QVQLVESGGGVVQPGESLRLSCAASGFTFSRYGIHWVRQAPGK | 11-CD3 Heavy |
| | GLEWVAAIWYDASKDYYADSVKGRFTISRDNSKNTLYLQMNS | Chain |
| | LRAEDTAVYYCARSNYDILTRYYQYGLDVWGQGTTVTVSSAS | (HC constant region |
| | TKGPSVFPLAPSSKSTSGGTAALGCLVKDYFPEPVTVSWNSGAL | underlined) |
| | TSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLM | |
| | ISRTPEVTCVVVDVSHEDPEVKFNWYVDGVEVHNAKTKPREE | |
| | QYNSTYRVVSVLTVLHQDWLNGKEYKCKVSNKALPAPIEKTIS | |
| | KAKGQPREPQVYTLPPSREEMTKNQVSLTCLVKGFYPSDIAVE | |
| | WESNGQPENNYKTTPPVLDSDGSFFLYSKLTVDKSRWQQGNV | |
| | FSCSVMHEALHNHYTQKSLSLSPG | |
| 149 | QVQLVESGGGVVQPGGSLRLSCAASGFTFSRYGIHWVRQAPGK | 12-CD3 Heavy |
| | GLEWVAAIWYDASKTYYADSVKGRFTISRDNSSNTLYLQMNSL | Chain |
| | RAEDTAVYYCARSNYDILTRYYQYGLDVWGQGTTVTVSSAST | (HC constant region |
| | KGPSVFPLAPSSKSTSGGTAALGCLVKDYFPEPVTVSWNSGALT | underlined) |
| | SGVHTFPAVLQSSGLYSLSSVVTVPSSSLGTQTYICNVNHKPSNT | |
| | KVDKKVEPKSCDKTHTCPPCPAPELLGGPSVFLFPPKPKDTLMI | |
| | SRTPEVTCVVVDVSHEDPEVKFNWYVDGVEVHNAKTKPREEQ | |
| | YNSTYRVVSVLTVLHQDWLNGKEYKCKVSNKALPAPIEKTISK | |
| | AKGQPREPQVYTLPPSREEMTKNQVSLTCLVKGFYPSDIAVEW | |
| | ESNGQPENNYKTTPPVLDSDGSFFLYSKLTVDKSRWQQGNVFS | |
| | CSVMHEALHNHYTQKSLSLSPG | |
| 150 | QVQLVESGGGVVQPGESLRLSCAASGFTFSRYGIHWVRQAPGK | 13-CD3 Heavy |
| | GLEWVAAIWYDASKDYYADSVKGRFTISRDNSENTLYLQMNS | Chain |
| | LRAEDTAVYYCARSNYDILTRYYQYGLDVWGQGTTVTVSSAS | (HC constant region |
| | TKGPSVFPLAPSSKSTSGGTAALGCLVKDYFPEPVTVSWNSGAL | underlined) |
| | TSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLM | |
| | ISRTPEVTCVVVDVSHEDPEVKFNWYVDGVEVHNAKTKPREE | |
| | QYNSTYRVVSVLTVLHQDWLNGKEYKCKVSNKALPAPIEKTIS | |
| | KAKGQPREPQVYTLPPSREEMTKNQVSLTCLVKGFYPSDIAVE | |
| | WESNGQPENNYKTTPPVLDSDGSFFLYSKLTVDKSRWQQGNV | |
| | FSCSVMHEALHNHYTQKSLSLSPG | |
| 151 | QVQLVESGGGVVQPGGSLRLSCAASGFTFSRYGIHWVRQAPGK | 14-CD3 Heavy |
| | GLEWVAAIWYDASKKYYADSVKGRFTISRDNSSNTLYLQMNS | Chain |
| | LRAEDTAVYYCARSNYDILTRYYQYGLDVWGQGTTVTVSSAS | (HC constant region |
| | TKGPSVFPLAPSSKSTSGGTAALGCLVKDYFPEPVTVSWNSGAL | underlined) |
| | TSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLM | |
| | ISRTPEVTCVVVDVSHEDPEVKFNWYVDGVEVHNAKTKPREE | |
| | QYNSTYRVVSVLTVLHQDWLNGKEYKCKVSNKALPAPIEKTIS | |
| | KAKGQPREPQVYTLPPSREEMTKNQVSLTCLVKGFYPSDIAVE | |
| | WESNGQPENNYKTTPPVLDSDGSFFLYSKLTVDKSRWQQGNV | |
| | FSCSVMHEALHNHYTQKSLSLSPG | |
| 152 | QVQLVESGGGVVQPGESLRLSCAASGFTFSRYGIHWVRQAPGK | 15-CD3 Heavy |
| | GLEWVAAIWYDASKKYYADSVKGRFTISRDNSENTLYLQMNS | Chain |
| | LRAEDTAVYYCARSNYDILTRYYQYGLDVWGQGTTVTVSSAS | (HC constant region |
| | TKGPSVFPLAPSSKSTSGGTAALGCLVKDYFPEPVTVSWNSGAL | underlined) |
| | TSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLM | |
| | ISRTPEVTCVVVDVSHEDPEVKFNWYVDGVEVHNAKTKPREE | |
| | QYNSTYRVVSVLTVLHQDWLNGKEYKCKVSNKALPAPIEKTIS | |
| | KAKGQPREPQVYTLPPSREEMTKNQVSLTCLVKGFYPSDIAVE | |
| | WESNGQPENNYKTTPPVLDSDGSFFLYSKLTVDKSRWQQGNV | |
| | FSCSVMHEALHNHYTQKSLSLSPG | |
| 153 | QVQLVESGGGVVQPGGSLRLSCAASGFTFSRYGIHWVRQAPGK | 16-CD3 Heavy |
| | GLEWVAAIWYDASKTYYADSVKGRFTISRDNSKNTLYLQMNS | Chain |
| | LRAEDTAVYYCARSNYDILTGHYQYGINVWGQGTTVTVSSAST | (HC constant region |
| | KGPSVFPLAPSSKSTSGGTAALGCLVKDYFPEPVTVSWNSGALT | underlined) |
| | SGVHTFPAVLQSSGLYSLSSVVTVPSSSLGTQTYICNVNHKPSNT | |
| | KVDKKVEPKSCDKTHTCPPCPAPELLGGPSVFLFPPKPKDTLMI | |
| | SRTPEVTCVVVDVSHEDPEVKFNWYVDGVEVHNAKTKPREEQ | |
| | YNSTYRVVSVLTVLHQDWLNGKEYKCKVSNKALPAPIEKTISK | |
| | AKGQPREPQVYTLPPSREEMTKNQVSLTCLVKGFYPSDIAVEW | |
| | ESNGQPENNYKTTPPVLDSDGSFFLYSKLTVDKSRWQQGNVFS | |
| | CSVMHEALHNHYTQKSLSLSPG | |
| 154 | AIWYDX₁SX₂X₃YYADSVKG, X₁ = G or A or S, X₂ = K or H or | CDR-H2 consensus |
| | N, X₃ = K or T or D | |
| 155 | QQFX₂SYPRT, X₂ = R or N or K | CDR-L3 consensus |
| 156 | QVQLVESGGGVVQPGXISLRLSCAASGFTFSX2X3GIHWVRQA | CD3 VH consensus |
| | PGKX4LEWVAAIWYDX5SX6X7YYADSVKGRFTISRDNSX8NTL | |
| | X9LQMNSLRAEDTAVYYCARSNX10DX11LTX12X13YX14X15G | |
| | X16X17VWGQGTTVTVSS, wherein X1 is R, G, or E; | |
| | X2 is R or N; X3 is Y or H, X4 is G or C; X5 is | |
| | G or A or S; X6 is K, H, or N, X7 is K, T, or | |
| | D; X8 is K, E, or S; X9 is Y or D; X10 is Y or | |
| | F, X11 is I or V, X12 is R or G; X13 is Y or H; | |
| | X14 is Q or S; X15 is Y or H; X16 is I or L; | |
| | and X17 is N or D | |

What is claimed:

1. A binding molecule comprising a CD3 binding domain comprising a heavy chain variable (VH) region and a light chain variable (VL) region, wherein:
the VH region comprises a heavy chain complementarity determining region 1 (CDR-H1) set forth in SEQ ID NO: 107, a heavy chain complementarity determining region 2 (CDR-H2) set forth in SEQ ID NO: 154, and a heavy chain complementarity determining region 3 (CDR-H3) set forth in SEQ ID NO:109; and
the VL region comprises a light chain complementarity determining region 1 (CDR-L1) set forth in SEQ ID NO: 19, a light chain complementarity determining region 2 (CDR-L2) set forth in SEQ ID NO: 110, and a light chain complementarity determining region 3 (CDR-L3) set forth within SEQ ID NO: 155.

2. The binding molecule of claim 1, wherein:
the VH region comprises a heavy chain complementarity determining region 1 (CDR-H1) set forth in SEQ ID NO: 107, a heavy chain complementarity determining region 2 (CDR-H2) set forth in SEQ ID NO: 108, and a heavy chain complementarity determining region 3 (CDR-H3) set forth in SEQ ID NO:109; and
the VL region comprises a light chain complementarity determining region 1 (CDR-L1) set forth in SEQ ID NO: 19, a light chain complementarity determining region 2 (CDR-L2) set forth in SEQ ID NO: 110, and a light chain complementarity determining region 3 (CDR-L3) set forth within SEQ ID NO: 6.

3. The binding molecule of claim 1 or claim 2, wherein the VH region of the CD3 binding domain and the VL region of the CD3 binding domain comprises, respectively:
an amino acid sequence of the consensus sequence set forth in QVQLVESGGGVVQPGX₁SLRLSCAASGFTFSX₂X₃GIHWVRQAPGKX₄LEWVAAIWYD X₅SX₆X₇YYADSVKGRFTISRDNSX₈NTLX₉LQMNSLRAEDTAVYYCARSNX₁₀DX₁₁LTX₁₂X₁₃YX14X₁₅GX₁₆X₁₇VWGQGTTVTVSS, wherein X₁ is R, G, or E; X₂ is R or N; X₃ is Y or H, X₄ is G or C; X₅ is G or A or S; X₆ is K, H, or N, X₇ is K, T, or D; X₈ is K, E, or S; X₉ is Y or D; X₁₀ is Y or F, X₁₁ is I or V, X₁₂ is R or G; X₁₃ is Y or H; X₁₄ is Q or S; X₁₅ is Y or H; X₁₆ is I or L; and X₁₇ is N or D; (SEQ ID NO: 156); and
an amino acid sequence of the consensus sequence set forth in AIQLTQSPSSLSASVGDRVTITCRASQGISSALAWYQQKPGKAPKLLIYDASX₁LEX₂G VPSRFSGSGSGTDFTLTISSLQPEDFATYYCQQFX₃SYPRTFGX4GTKVEIK, wherein X₁ is S or G, X₂ is N or S; X₃ is K or N; and X₄ is Q or C; (SEQ ID NO: 106).

4. The binding molecule of any one of claims 1-3, wherein the VH region of the CD3 binding domain and the VL region of the CD3 binding domain comprises, respectively:
an amino acid sequence of the consensus sequence set forth in QVQLVESGGGVVQPGX₁SLRLSCAASGFTFSX2X₃GIHWVRQAPGKX₄LEWVAAIWY DX₅SX₆X₇YYADSVKGRFTISRDNSX₈NTLX₉LQMNSLRAEDTAVYYCARSNX₁₀DX₁₁LT X₁₂X₁₃YX₁₄X₁₅GX₁₆X₁₇VWGQGTTVTVSS, wherein X₁ is R, G, or E; X₂ is R or N; X₃ is Y or H, X₄ is G or C; X₅ is G or A or S; X₆ is K, H, or N, X₇ is K, T, or D; X₈ is K, E, or S; X₉ is Y or D; X₁₀ is Y or F, X₁₁ is I or V, X₁₂ is R or G; X₁₃ is Y or H; X₁₄ is Q or S; X₁₅ is Y or H; X₁₆ is I or L; and X₁₇ is N or D; (SEQ ID NO: 105); and
an amino acid sequence of the consensus sequence set forth in AIQLTQSPSSLSASVGDRVTITCRASQGISSALAWYQQKPGKAPKLLIYDASX1LEX2G VPSRFSGSGSGTDFTLTISSLQPEDFATYYCQQFX3SYPRTFGX4GTKVEIK, wherein X₁ is S or G, X₂ is N or S; X₃ is K or N; and X₄ is Q or C; (SEQ ID NO: 106).

5. The binding molecule of any one of claims 1-4, wherein:
the CDR-H1 comprises the sequence set forth in any one of SEQ ID NOs: 9, 12, 111, or 112, the CDR-H2 comprises the sequence set forth in any one of SEQ ID NOs: 10, 13, 15, 17, or 113-117, and the CDR-H3 comprises the sequence set forth in any one of SEQ ID NOs: 11, 14, 16, 18, 118-123; and
the CDR-L1 comprises the sequence set forth in SEQ ID NOs: 19, the CDR-L2 comprises the sequence set forth in any one of SEQ ID NOs: 20, 22, 124, or 125, and the CDR-L3 comprises the sequence set forth in any one of SEQ ID NOs: 21 or 23.

6. The binding molecule of any one of claims 1-5, wherein:
the VH region comprises the CDR-H1, the CDR-H2, and the CDR-H3 contained within SEQ ID NO: 24, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 25, and the VL region comprises the CDR-L1, the CDR-L2, and the CDR-L3 contained within SEQ ID NO: 40;
the VH region comprises the CDR-H1, the CDR-H2, and the CDR-H3 contained within SEQ ID NO: 26, and the VL region comprises the CDR-L1, the CDR-L2, and the CDR-L3 contained within SEQ ID NO: 40;
the VH region comprises the CDR-H1, the CDR-H2, and the CDR-H3 contained within SEQ ID NO: 27, and the VL region comprises the CDR-L1, the CDR-L2, and the CDR-L3 contained within SEQ ID NO: 41;
the VH region comprises the CDR-H1, the CDR-H2, and the CDR-H3 contained within SEQ ID NO: 28, and the VL region comprises the CDR-L1, the CDR-L2, and the CDR-L3 contained within SEQ ID NO: 42;
the VH region comprises the CDR-H1, the CDR-H2, and the CDR-H3 contained within SEQ ID NO: 29, and the VL region comprises the CDR-L1, the CDR-L2, and the CDR-L3 contained within SEQ ID NO: 40;
the VH region comprises the CDR-H1, the CDR-H2, and the CDR-H3 contained within SEQ ID NO: 30, and the VL region comprises the CDR-L1, the CDR-L2, and the CDR-L3 contained within SEQ ID NO: 41;
the VH region comprises the CDR-H1, the CDR-H2, and the CDR-H3 contained within SEQ ID NO: 31, and the VL region comprises the CDR-L1, the CDR-L2, and the CDR-L3 contained within SEQ ID NO: 43;
the VH region comprises the CDR-H1, the CDR-H2, and the CDR-H3 contained within SEQ ID NO: 32, and the VL region comprises the CDR-L1, the CDR-L2, and the CDR-L3 contained within SEQ ID NO: 40;
the VH region comprises the CDR-H1, the CDR-H2, and the CDR-H3 contained within SEQ ID NO: 33, and the VL region comprises the CDR-L1, the CDR-L2, and the CDR-L3 contained within SEQ ID NO: 40;
the VH region comprises the CDR-H1, the CDR-H2, and the CDR-H3 contained within SEQ ID NO: 34, and the VL region comprises the CDR-L1, the CDR-L2, and the CDR-L3 contained within SEQ ID NO: 40;
the VH region comprises the CDR-H1, the CDR-H2, and the CDR-H3 contained within SEQ ID NO: 35, and the VL region comprises the CDR-L1, the CDR-L2, and the CDR-L3 contained within SEQ ID NO: 40;
the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NO: 40;
the VH region comprises the CDR-H1, the CDR-H2, and the CDR-H3 contained within SEQ ID NO: 37, and the VL region comprises the CDR-L1, the CDR-L2, and the CDR-L3 contained within SEQ ID NO: 40;
the VH region comprises the CDR-H1, the CDR-H2, and the CDR-H3 contained within SEQ ID NO: 38, and the VL region comprises the CDR-L1, the CDR-L2, and the CDR-L3 contained within SEQ ID NO: 41;
the VH region comprises the CDR-H1, CDR-H2 and the CDR-H3 contained within SEQ ID NO: 126, and the VL region comprises the CDR-L1, the CDR-L2, and the CDR-L3 contained within SEQ ID NO: 40;
the VH region comprises the CDR-H1, CDR-H2 and the CDR-H3 contained within SEQ ID NO: 4, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 53, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 54, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 55, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 56, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 57, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 58, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 59, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 60, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 61, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 62, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 63, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 64, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 65, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 66, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 67, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 68, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 69, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 70, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 71, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 72, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 73, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 74, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 75, and the VL region comprises the CDR-L1, the CDR-L2, and the CDR-L3 contained within SEQ ID NOs: 39, 40, or 41;
the VH region comprises the CDR-H1, CDR-H2 and the CDR-H3 contained within SEQ ID NO: 24, and the VL region comprises the CDR-L1, the CDR-L2, and the CDR-L3 contained within SEQ ID NOs: 40;
the VH region comprises the CDR-H1, CDR-H2 and the CDR-H3 contained within SEQ ID NO: 24, and the VL region comprises the CDR-L1, the CDR-L2, and the CDR-L3 contained within SEQ ID NOs: 77;
the VH region comprises the CDR-H1, CDR-H2 and the CDR-H3 contained within SEQ ID NO: 24, and the VL region comprises the CDR-L1, the CDR-L2, and the CDR-L3 contained within SEQ ID NOs: 78;
the VH region comprises the CDR-H1, CDR-H2 and the CDR-H3 contained within SEQ ID NO: 24, and the VL region comprises the CDR-L1, the CDR-L2, and the CDR-L3 contained within SEQ ID NOs: 79;
the VH region comprises the CDR-H1, CDR-H2 and the CDR-H3 contained within SEQ ID NO: 24, and the VL region comprises the CDR-L1, the CDR-L2, and the CDR-L3 contained within SEQ ID NOs: 80;
the VH region comprises the CDR-H1, the CDR-H2, and the CDR-H3 contained within SEQ ID NO: 27, and the VL region comprises the CDR-L1, the CDR-L2, and the CDR-L3 contained within SEQ ID NOs: 39;
the VH region comprises the CDR-H1, the CDR-H2, and the CDR-H3 contained within SEQ ID NO: 25, and the VL region comprises the CDR-L1, the CDR-L2, and the CDR-L3 contained within SEQ ID NOs: 39; or
the VH region comprises the CDR-H1, the CDR-H2, and the CDR-H3 contained within SEQ ID NO: 26, and the VL region comprises the CDR-L1, the CDR-L2, and the CDR-L3 contained within SEQ ID NOs: 39.

7. The binding molecule of any one of claims 1-6, wherein the VH region of the CD3 binding domain and the VL region of the CD3 binding domain comprises:
the CDR-H1, the CDR-H2, and the CDR-H3 comprising the sequence set forth in SEQ ID NOs: 9, 10, and 11, respectively, and the CDR-L1, the CDR-L2, and the CDR-L3 comprising the sequence set forth in SEQ ID NOs: 19, 20, and 21, respectively;
the CDR-H1, the CDR-H2, and the CDR-H3 comprising the sequence set forth in SEQ ID NOs: 12, 13, and 11, respectively, and t the CDR-L1, the CDR-L2, and the CDR-L3 comprising the sequence set forth in SEQ ID NOs: 19, 22, and 21, respectively;
the CDR-H1, the CDR-H2, and the CDR-H3 comprising the sequence set forth in SEQ ID NOs: 9, 13, and 14, respectively, and the CDR-L1, the CDR-L2, and the CDR-L3 comprising the sequence set forth in SEQ ID NOs: 19, 22, and 21, respectively;
the CDR-H1, the CDR-H2, and the CDR-H3 comprising the sequence set forth in SEQ ID NOs: 9, 13, and 11, respectively, and the CDR-L1, the CDR-L2, and the CDR-L3 comprising the sequence set forth in SEQ ID NOs: 19, 22, and 23, respectively;
the CDR-H1, the CDR-H2, and the CDR-H3 comprising the sequence set forth in SEQ ID NOs: 9, 15, and 14, respectively, and the CDR-L1, the CDR-L2, and the CDR-L3 comprising the sequence set forth in SEQ ID NOs: 19, 22, and 21, respectively;
the CDR-H1, the CDR-H2, and the CDR-H3 comprising the sequence set forth in SEQ ID NOs: 9, 15, and 16, respectively, and the CDR-L1, the CDR-L2, and the CDR-L3 comprising the sequence set forth in SEQ ID NOs: 19, 22, and 23, respectively;
the CDR-H1, the CDR-H2, and the CDR-H3 comprising the sequence set forth in SEQ ID NOs: 9, 17, and 14, respectively, and the CDR-L1, the CDR-L2, and the CDR-L3 comprising the sequence set forth in SEQ ID NOs: 19, 22, and 21, respectively;
the CDR-H1, the CDR-H2, and the CDR-H3 comprising the sequence set forth in SEQ ID NOs: 9, 15, and 18, respectively, and the CDR-L1, the CDR-L2, and the CDR-L3 comprising the sequence set forth in SEQ ID NOs: 19, 22, and 23, respectively;
the CDR-H1, CDR-H2 and the CDR-H3 comprising the sequence set forth in SEQ ID NOs: 9, 10, and 11, respectively, and the VL region comprises the CDR-L1, the CDR-L2, and the CDR-L3 comprising the sequence set forth in SEQ ID NOs: 19, 20, and 21, respectively;
the VH region comprises the CDR-H1, CDR-H2 and the CDR-H3 comprising the sequence set forth in SEQ ID NOs: 9, 113, and 11, respectively, and the VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 9, 13, and 11, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 9, 114, and 11, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 9, 115, and 11, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 9, 10, and 118, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 9, 10, and 18, respectively, and VL region the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 9, 10, and 119, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 9, 10, and 120, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 9, 10, and 16, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 12, 10, and 11, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 111, 10, and 11, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 112, 10, and 11, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 9, 10, and 121, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 9, 10, and 122, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 9, 10, and 123, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 9, 10, and 14, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 9, 116, and 11, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 9, 117, and 11, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 9, 13, and 11, respectively, and VL region comprises the CDR-L1, the CDR-L2, and the CDR-L3 comprising the sequence set forth in SEQ ID NOs: 19, 20, and 21, respectively;
the CDR-H1, the CDR-H2, and the CDR-H3 comprising the sequence set forth in SEQ ID NOs: 9, 13, and 18, respectively, and the VL region comprises the CDR-L1, the CDR-L2, and the CDR-L3 comprising the sequence set forth in SEQ ID NOS: 19, 20, and 21, respectively;
the CDR-H1, CDR-H2 and the CDR-H3 comprising the sequence set forth in SEQ ID NOs: 9, 13, and 119, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 111, 13, and 11, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 9, 13, and 123, respectively, and the VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 9, 10, and 11, respectively, and VL region comprises the CDR-L1, the CDR-L2, and the CDR-L3 comprising the sequence set forth in SEQ ID NOs: 19, 22, and 21, respectively;
the CDR-H1, CDR-H2 and the CDR-H3 comprising the sequence set forth in SEQ ID NOs: 9, 10, and 11, respectively, and VL region comprises the CDR-L1, the CDR-L2, and the CDR-L3 comprising the sequence set forth in SEQ ID NOs: 19, 20, and 23, respectively;
the CDR-H1, CDR-H2 and the CDR-H3 comprising the sequence set forth in SEQ ID NOs: 9, 10, and 11, respectively, and VL region comprises the CDR-L1, the CDR-L2, and the CDR-L3 comprising the sequence set forth in SEQ ID NOs: 19, 22, and 23, respectively;
the CDR-H1, CDR-H2 and the CDR-H3 comprising the sequence set forth in SEQ ID NOs: 9, 10, and 11, respectively, and VL region comprises the CDR-L1, the CDR-L2, and the CDR-L3 comprising the sequence set forth in SEQ ID NOs: 19, 124, and 21, respectively;
the CDR-H1, CDR-H2 and the CDR-H3 comprising the sequence set forth in SEQ ID NOs: 9, 10, and 11, respectively, and VL region comprises the CDR-L1, the CDR-L2, and the CDR-L3 comprising the sequence set forth in SEQ ID NOs: 19, 125, and 23, respectively;
the CDR-H1, CDR-H2 and the CDR-H3 comprising the sequence set forth in SEQ ID NOs: 9, 13, and 16, respectively, and VL region comprises the CDR-L1, the CDR-L2, and the CDR-L3 comprising the sequence set forth in SEQ ID NOs: 19, 20, and 21, respectively;
the CDR-H1, CDR-H2 and the CDR-H3 comprising the sequence set forth in SEQ ID NOs: 12, 13, and 11, respectively, and VL region comprises the CDR-L1, the CDR-L2, and the CDR-L3 comprising the sequence set forth in SEQ ID NOs: 19, 20, and 21, respectively; or
the CDR-H1, CDR-H2 and the CDR-H3 comprising the sequence set forth in SEQ ID NOs: 9, 13, and 14, respectively, and VL region comprises the CDR-L1, the CDR-L2, and the CDR-L3 comprising the sequence set forth in SEQ ID NOs: 19, 20, and 21, respectively.

8. The binding molecule of any one of claims 1-7, wherein in the CD3 binding domain:
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 25, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 26, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 27, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 28, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 42;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 29, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 30, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 31, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 32, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 33, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 34, and the VL region comprises an amino acid sequence that has at least at or about 85%, 86%, 87%, 88%, 89%, 90%, 91%, 92%, 93%, 94%, 95%, 96%, 97%, 98% or 99% sequence identity to ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 35, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 38, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 126, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 4, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 53, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 54, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 55, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 56, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 57, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 58, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 59, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 60, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 61, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 62, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 63, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 64, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 65, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 66, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 67, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 68, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 69, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 70, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 71, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 72, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 73, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 74, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 75, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 39, 40, or 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 77;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 76;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 80;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 27, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39; or
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 26, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39.

9. The binding molecule of any one of claims 1-8, wherein in the CD3 binding domain:
the VH region and the VL region comprise the sequences set forth in SEQ ID NO: 24 and 39, respectively;
the VH region and the VL region comprise the sequences set forth in SEQ ID NO: 24 and 40, respectively;
the VH region and the VL region comprise the sequences set forth in SEQ ID NO: 26 and 40, respectively;
the VH region and the VL region comprise the sequences set forth in SEQ ID NO: 27 and 41, respectively;
the VH region and the VL region comprise the sequences set forth in SEQ ID NO: 28 and 42, respectively;
the VH region and the VL region comprise the sequences set forth in SEQ ID NO: 29 and 40, respectively;
the VH region and the VL region comprise the sequences set forth in SEQ ID NO: 30 and 41, respectively;
the VH region and the VL region comprise the sequences set forth in SEQ ID NO: 31 and 43, respectively;
the VH region and the VL region comprise the sequences set forth in SEQ ID NO: 32 and 40, respectively;
the VH region and the VL region comprise the sequences set forth in SEQ ID NO: 33 and 40, respectively;
the VH region and the VL region comprise the sequences set forth in SEQ ID NO: 34 and 40, respectively;
the VH region and the VL region comprise the sequences set forth in SEQ ID NO: 35 and 40, respectively;
the VH region and the VL region comprise the sequences set forth in SEQ ID NO: 36 and 40, respectively;
the VH region and the VL region comprise the sequences set forth in SEQ ID NO: 37 and 40, respectively;
the VH region and the VL region comprise the sequences set forth in SEQ ID NO: 38 and 41, respectively
the VH region and the VL region comprise the sequences set forth in SEQ ID NO: 126 and 40, respectively;
the VH region and the VL region comprise the sequences set forth in SEQ ID NO: 4 and 39, respectively;
the VH region and the VL region comprise the sequences set forth in SEQ ID NO: 83 and 39, respectively;
the VH region and the VL region comprise the sequences set forth in SEQ ID NO: 54 and 39, respectively;
the VH region and the VL region comprise the sequences set forth in SEQ ID NO: 55 and 39, respectively;
the VH region and the VL region comprise the sequences set forth in SEQ ID NO: 56 and 39, respectively;
the VH region and the VL region comprise the sequences set forth in SEQ ID NO: 57 and 39, respectively;
the VH region and the VL region comprise the sequences set forth in SEQ ID NO: 58 and 39, respectively;
the VH region and the VL region comprise the sequences set forth in SEQ ID NO: 59 and 39, respectively;
the VH region and the VL region comprise the sequences set forth in SEQ ID NO: 60 and 39, respectively;
the VH region and the VL region comprise the sequences set forth in SEQ ID NO: 61 and 39, respectively;
the VH region and the VL region comprise the sequences set forth in SEQ ID NO: 62 and 39, respectively;
the VH region and the VL region comprise the sequences set forth in SEQ ID NO: 63 and 39, respectively;
the VH region and the VL region comprise the sequences set forth in SEQ ID NO: 64, and 39, respectively;
the VH region and the VL region comprise the sequences set forth in SEQ ID NO: 65 and 39, respectively;
the VH region and the VL region comprise the sequences set forth in SEQ ID NO: 66 and 39, respectively;
the VH region and the VL region comprise the sequences set forth in SEQ ID NO: 67 and 39, respectively;
the VH region and the VL region comprise the sequences set forth in SEQ ID NO: 68 and 39, respectively;
the VH region and the VL region comprise the sequences set forth in SEQ ID NO: 69 and 39, respectively;
the VH region and the VL region comprise the sequences set forth in SEQ ID NO: 70 and 39, respectively;
the VH region and the VL region comprise the sequences set forth in SEQ ID NO: 71 and 39, respectively;
the VH region and the VL region comprise the sequences set forth in SEQ ID NO: 72 and 39, respectively;
the VH region and the VL region comprise the sequences set forth in SEQ ID NO: 73 and 39, respectively;
the VH region and the VL region comprise the sequences set forth in SEQ ID NO: 74 and 39, respectively;
the VH region and the VL region comprise the sequences set forth in SEQ ID NO: 75 and 39, respectively;
the VH region and the VL region comprise the sequences set forth in SEQ ID NO: 24 and 40, respectively;
the VH region and the VL region comprise the sequences set forth in SEQ ID NO: 24 and 77, respectively;
the VH region and the VL region comprise the sequences set forth in SEQ ID NO: 24 and 41, respectively;
the VH region and the VL region comprise the sequences set forth in SEQ ID NO: 24 and 76, respectively;
the VH region and the VL region comprise the sequences set forth in SEQ ID NO: 24 and 80, respectively;
the VH region and the VL region comprise the sequences set forth in SEQ ID NO: 27 and 39, respectively;
the VH region and the VL region comprise the sequences set forth in SEQ ID NO: 24 and 39, respectively; or
the VH region and the VL region comprise the sequences set forth in SEQ ID NO: 26 and 39, respectively.

10. The binding molecule of claim 1, wherein:
the VH region comprises a sequence of the consensus sequence set forth in QVQLVESGGGVVQPGX₁SLRLSCAASGFTFSX₂YGIHWVRQAPGKX₃LEWVAAIWYD X₄SKX₅YYADSVKGRFTISRDNSX₆NTLX₇LQMNSLRAEDTAVYYCARSNYDILTX₈X₉Y X₁₀X₁₁GX₁₂X₁₃VWGQGTTVTVSS, wherein X₁ is R, G, or E; X₂ is R or N; X₃ is G or C; X₄ is G or A; X₅ is K, T, or D; X₆ is K, E, or S; X₇ is Y or D; X₈ is R or G; X₉ is Y or H; X₁₀ is Q or S; X₁₁ is Y or H; X₁₂ is I or L; and X₁₃ is N or D; (SEQ ID NO:7); and
the VL region comprises a sequence of the consensus sequence set forth in AIQLTQSPSSLSASVGDRVTITCRASQGISSALAWYQQKPGKAPKLLIYDASSLEX₁GV PSRFSGSGSGTDFTLTISSLQPEDFATYYCQQFX₂SYPRTFGX₃GTKVEIK, wherein X₁ is N or S; X₂ is K or N; X₃ is Q or C; (SEQ ID NO:8).

11. The binding molecule of any one of claims 1-10, wherein:
the VH region comprises a sequence of the consensus sequence set forth in QVQLVESGGGVVQPGX₁SLRLSCAASGFTFSX₂YGIHWVRQAPGKX₃LEWVAAIWYD ASKX₄YYADSVKGRFTISRDNSX₅NTLYLQMNSLRAEDTAVYYCARSNYDILTX₆X₇YX₈X₉GX₁₀X₁₁VWGQGTTVTVSS, wherein X₁=R or G or E; X₂=R or N; X₃=G or C; X₄=K or T or D; X₅=K or E or S; X₆=R or G; X₇=Y or H; X₈=Q or S; X₉=Y or H; X₁₀=I or L; and X₁₁=N or D (SEQ ID NO: 83); and
the VL region comprises a sequence of the consensus sequence set forth in AIQLTQSPSSLSASVGDRVTITCRASQGISSALAWYQQKPGKAPKLLIYDASSLESGVP SRFSGSGSGTDFTLTISSLQPEDFATYYCQQFX₁SYPRTFGX₂GTKVEIK (SEQ ID NO: 84), wherein X₁=K or N; X₂=Q or C.

12. The binding molecule of any one of claims 1-11, wherein the binding molecule is a bispecific antibody comprising the CD3 binding domain and a second antigen binding domain.

13. A binding molecule comprising a CD3 binding domain and a second antigen binding domain, wherein the binding molecule is bispecific for binding CD3 and the second antigen, wherein:
(a) the CD3 binding domain comprises a heavy chain variable (VH) region and a light chain variable (VL) region, wherein:
the VH region comprises a heavy chain complementarity determining region 1 (CDR-H1) set forth in SEQ ID NO: 1, a heavy chain complementarity determining region 2 (CDR-H2) set forth in SEQ ID NO: 2, and a heavy chain complementarity determining region 3 (CDR-H3) set forth in SEQ ID NO:3, and
the VL region comprises a light chain complementarity determining region 1 (CDR-L1) set forth in SEQ ID NO: 19, a light chain complementarity determining region 2 (CDR-L2) set forth in SEQ ID NO: 5, and a light chain complementarity determining region 3 (CDR-L3) set forth within SEQ ID NO: 6; and
(b) the second antigen binding domain comprises a means for binding a second antigen, optionally wherein the second antigen is on a tumor.

14. The binding molecule of claim 13, wherein:
the VH region comprises a CDR-H1 set forth in SEQ ID NO: 1, a CDR-H2 set forth in SEQ ID NO: 85, and a CDR-H3 set forth in SEQ ID NO:3; and
the VL region comprises a CDR-L1 set forth in SEQ ID NO: 19, a CDR-L2 set forth in SEQ ID NO: 22, and a CDR-L3 set forth within SEQ ID NO: 6.

15. The binding molecule of claim 13 or claim 14, wherein:
the VH region comprises a sequence of the consensus sequence set forth in QVQLVESGGGVVQPGX₁SLRLSCAASGFTFSX₂YGIHWVRQAPGKX₃LEWVAAIWYD X₄SKX₅YYADSVKGRFTISRDNSX₆NTLX₇LQMNSLRAEDTAVYYCARSNYDILTX₈X₉Y X₁₀X₁₁GX₁₂X₁₃VWGQGTTVTVSS, wherein X₁ is R, G, or E; X₂ is R or N; X₃ is G or C; X₄ is G or A; X₅ is K, T, or D; X₆ is K, E, or S; X₇ is Y or D; X₈ is R or G; X₉ is Y or H; X₁₀ is Q or S; X₁₁ is Y or H; X₁₂ is I or L; and X₁₃ is N or D; (SEQ ID NO:7); and
the VL region comprising a sequence of the consensus sequence set forth in AIQLTQSPSSLSASVGDRVTITCRASQGISSALAWYQQKPGKAPKLLIYDASSLEX₁GV PSRFSGSGSGTDFTLTISSLQPEDFATYYCQQFX₂SYPRTFGX₃GTKVEIK, wherein X₁ is N or S; X₂ is K or N; X₃ is Q or C; (SEQ ID NO:8).

16. The binding molecule of claim 15, wherein:
the VH region comprises a sequence of the consensus sequence set forth in QVQLVESGGGVVQPGX₁SLRLSCAASGFTFSX₂YGIHWVRQAPGKX₃LEWVAAIWYD ASKX₄YYADSVKGRFTISRDNSX₅NTLYLQMNSLRAEDTAVYYCARSNYDILTX₆X₇YX₈X₉GX₁₀X₁₁VWGQGTTVTVSS, wherein X₁=R or G or E; X₂=R or N; X₃=G or C; X₄=K or Tor D; X₅=K or E or S; X₆=R or G; X₇=Y or H; X₈=Q or S; X₉=Y or H; X₁₀=I or L; and X₁₁=N or D (SEQ ID NO: 83); and
the VL region comprises a sequence of the consensus sequence set forth in AIQLTQSPSSLSASVGDRVTITCRASQGISSALAWYQQKPGKAPKLLIYDASSLESGVP SRFSGSGSGTDFTLTISSLQPEDFATYYCQQFX₁SYPRTFGX₂GTKVEIK (SEQ ID NO: 84), wherein X₁=K or N; X₂=Q or C.

17. The binding molecule of any of claims 13-16, wherein the CD3 binding domain binds to CD3 epsilon with a dissociation constant (KD) of 5 nM or more.

18. The binding molecule of any of claims 1-17, wherein the CD3 binding domain binds to CD3 epsilon with a dissociation constant (KD) of 10 nM to 70 nM.

19. The binding molecule of any of claims 1-18, wherein the VH region comprises one or more amino acid residues selected from the group consisting of: an asparagine (N) at position 31, an alanine (A) at position 55, a tyrosine (Y) at position 80, a glycine (G) at position 106, a histidine (H) at position 107, a serine(S) at position 109, a histidine (H) at position 110, a leucine (L) at position 112, and an aspartic acid (D) at position 113 or any combination thereof, wherein the numbering is relative to SEQ ID NO: 24.

20. The binding molecule of any of claims 1-19, wherein the VH region comprises:
(a) amino acid residues asparagine (N) at position 31, alanine (A) at position 55, and tyrosine (Y) at position 80, wherein the numbering is relative to SEQ ID NO: 24;
(b) alanine (A) at position 55, tyrosine (Y) at position 80, leucine (L) at position 112, and aspartic acid (D) at position 113, wherein the numbering is relative to SEQ ID NO: 24; or
(c) alanine (A) at position 55, tyrosine (Y) at position 80, serine(S) at position 109, and histidine (H) at position 110, wherein the numbering is relative to SEQ ID NO: 24.

21. The binding molecule of any of claims 1-20, wherein the VL region comprises one or more amino acid residues selected from the group consisting of: a serine(S) at position 56, an asparagine (N) at position 92, and a cysteine (C) at position 100, an asparagine (N) at position 108, or any combination thereof, wherein the numbering is relative to SEQ ID NO: 39.

22. The binding molecule of any of claims 1-21, wherein the VL region comprises:
(a) serine(S) at position 56, wherein the numbering is relative to SEQ ID NO:39; or
(b) serine(S) at position 56 and asparagine (N) at position 92, wherein the numbering is relative to SEQ ID NO: 39.

23. The binding molecule of any of claims 1-22, wherein the CD3 binding domain exhibits low polyreactivity to extracellular matrix proteins.

24. The binding molecule of claim 23, wherein the CD3 binding domain exhibits an extracellular matrix (ECM) score of less than 14, less than 13, less than 12, less than 11, or less than 10.

25. The binding molecule of any of claims 1-24, wherein the VH region comprises glycine (G) at position 16, a glutamic acid (E) at position 16, a threonine (T) at position 58, an aspartic acid (D) at position 58, a glutamic acid (E) at position 76, a serine(S) at position 76 in relation to SEQ ID NO: 24.

26. The binding molecule of any of claims 1-25, wherein the VH region comprises glycine (G) at position 16 and/or threonine (T) at position 58, optionally glycine (G) at position 16 and threonine (T) at position 58 in relation to SEQ ID NO: 24.

27. The binding molecule of any of claims 1-26, wherein the CD3 binding domain is an antigen binding antibody fragment.

28. The binding molecule of claim 27, wherein the CD3 binding domain is a scFv, is a Fab or is an Fv.

29. The binding molecule of claim 27, wherein the second binding domain is an scFv.

30. The binding molecule of any of claims 1-29, wherein the CD3 binding domain is an scFv that is a disulfide-stabilized scFv.

31. The binding molecule of any of claims 1-30, wherein the CD3 binding domain is an scFv that comprises a VH-linker-VL arrangement.

32. The binding molecule of any of claims 1-31, wherein the CD3 binding domain is an scFv that comprises a VL-linker-VH arrangement.

33. The binding molecule of any of claims 1-32, wherein the CD3 binding domain is an scFv that comprises a peptide linker comprising (Gly4Ser)3 (SEQ ID NO: 96).

34. The binding molecule of any of claims 1-24 and 27-33, wherein the VH region of the CD3 binding domain comprises cysteine (C) at position 44 and the VL region of the CD3 binding domain comprises a cysteine (C) at position 100 in relation to SEQ ID NO: 39.

35. The binding molecule of any of claims 1-18, wherein the VH region comprises the CDR-H1, the CDR-H2, and the CDR-H3 contained within any one of SEQ ID NOs: 24-38, and the VL region comprises the CDR-L1, the CDR-L2, and the CDR-L3 contained within any one of SEQ ID NOs: 39-43.

36. The binding molecule of any of claims 1-18, wherein:
the CDR-H1 comprises the sequence set forth in any one of SEQ ID NOs: 9 or 12, the CDR-H2 comprises the sequence set forth in any one of SEQ ID NOs: 10, 13, 15, or 17, and the CDR-H3 comprises the sequence set forth in any one of SEQ ID NOs: 11, 14, 16, or 18; and
the CDR-L1 comprises the sequence set forth in SEQ ID NOs: 19, the CDR-L2 comprises the sequence set forth in any one of SEQ ID NOs: 20 or 22, and the CDR-L3 comprises the sequence set forth in any one of SEQ ID NOs: 21 or 23.

37. The binding molecule of any of claims 1-18, wherein:
the VH region comprises the CDR-H1, the CDR-H2, and the CDR-H3 contained within SEQ ID NO: 24, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 25, and the VL region comprises the CDR-L1, the CDR-L2, and the CDR-L3 contained within SEQ ID NO: 40;
the VH region comprises the CDR-H1, the CDR-H2, and the CDR-H3 contained within SEQ ID NO: 26, and the VL region comprises the CDR-L1, the CDR-L2, and the CDR-L3 contained within SEQ ID NO: 40;
the VH region comprises the CDR-H1, the CDR-H2, and the CDR-H3 contained within SEQ ID NO: 27, and the VL region comprises the CDR-L1, the CDR-L2, and the CDR-L3 contained within SEQ ID NO: 41;
the VH region comprises the CDR-H1, the CDR-H2, and the CDR-H3 contained within SEQ ID NO: 28, and the VL region comprises the CDR-L1, the CDR-L2, and the CDR-L3 contained within SEQ ID NO: 42;
the VH region comprises the CDR-H1, the CDR-H2, and the CDR-H3 contained within SEQ ID NO: 29, and the VL region comprises the CDR-L1, the CDR-L2, and the CDR-L3 contained within SEQ ID NO: 40;
the VH region comprises the CDR-H1, the CDR-H2, and the CDR-H3 contained within SEQ ID NO: 30, and the VL region comprises the CDR-L1, the CDR-L2, and the CDR-L3 contained within SEQ ID NO: 41;
the VH region comprises the CDR-H1, the CDR-H2, and the CDR-H3 contained within SEQ ID NO: 31, and the VL region comprises the CDR-L1, the CDR-L2, and the CDR-L3 contained within SEQ ID NO: 43;
the VH region comprises the CDR-H1, the CDR-H2, and the CDR-H3 contained within SEQ ID NO: 32, and the VL region comprises the CDR-L1, the CDR-L2, and the CDR-L3 contained within SEQ ID NO: 40;
the VH region comprises the CDR-H1, the CDR-H2, and the CDR-H3 contained within SEQ ID NO: 33, and the VL region comprises the CDR-L1, the CDR-L2, and the CDR-L3 contained within SEQ ID NO: 40;
the VH region comprises the CDR-H1, the CDR-H2, and the CDR-H3 contained within SEQ ID NO: 34, and the VL region comprises the CDR-L1, the CDR-L2, and the CDR-L3 contained within SEQ ID NO: 40;
the VH region comprises the CDR-H1, the CDR-H2, and the CDR-H3 contained within SEQ ID NO: 35, and the VL region comprises the CDR-L1, the CDR-L2, and the CDR-L3 contained within SEQ ID NO: 40;
the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NO: 40;
the VH region comprises the CDR-H1, the CDR-H2, and the CDR-H3 contained within SEQ ID NO: 37, and the VL region comprises the CDR-L1, the CDR-L2, and the CDR-L3 contained within SEQ ID NO: 40;
the VH region comprises the CDR-H1, the CDR-H2, and the CDR-H3 contained within SEQ ID NO: 38, and the VL region comprises the CDR-L1, the CDR-L2, and the CDR-L3 contained within SEQ ID NO: 41; or
the VH region comprises the CDR-H1, the CDR-H2, and the CDR-H3 contained within SEQ ID NO: 126, and the VL region comprises the CDR-L1, the CDR-L2, and the CDR-L3 contained within SEQ ID NO: 40.

38. The binding molecule of any of claims 1-18, wherein:
the VH region comprises the CDR-H1, the CDR-H2, and the CDR-H3 comprising the sequence set forth in SEQ ID NOs: 9, 10, and 11, respectively, and the VL region comprises the CDR-L1, the CDR-L2, and the CDR-L3 comprising the sequence set forth in SEQ ID NOs: 19, 20, and 21, respectively;
the VH region comprises the CDR-H1, the CDR-H2, and the CDR-H3 comprising the sequence set forth in SEQ ID NOs: 12, 13, and 11, respectively, and the VL region comprises the CDR-L1, the CDR-L2, and the CDR-L3 comprising the sequence set forth in SEQ ID NOs: 19, 22, and 21, respectively;
the VH region comprises the CDR-H1, the CDR-H2, and the CDR-H3 comprising the sequence set forth in SEQ ID NOs: 9, 13, and 14, respectively, and the VL region comprises the CDR-L1, the CDR-L2, and the CDR-L3 comprising the sequence set forth in SEQ ID NOs: 19, 22, and 21, respectively;
the VH region comprises the CDR-H1, the CDR-H2, and the CDR-H3 comprising the sequence set forth in SEQ ID NOs: 9, 13, and 11, respectively, and the VL region comprises the CDR-L1, the CDR-L2, and the CDR-L3 comprising the sequence set forth in SEQ ID NOs: 19, 22, and 23, respectively;
the VH region comprises the CDR-H1, the CDR-H2, and the CDR-H3 comprising the sequence set forth in SEQ ID NOs: 9, 15, and 14, respectively, and the VL region comprises the CDR-L1, the CDR-L2, and the CDR-L3 comprising the sequence set forth in SEQ ID NOs: 19, 22, and 21, respectively;
the VH region comprises the CDR-H1, the CDR-H2, and the CDR-H3 comprising the sequence set forth in SEQ ID NOs: 9, 15, and 16, respectively, and the VL region comprises the CDR-L1, the CDR-L2, and the CDR-L3 comprising the sequence set forth in SEQ ID NOs: 19, 22, and 23, respectively;
the VH region comprises the CDR-H1, the CDR-H2, and the CDR-H3 comprising the sequence set forth in SEQ ID NOs: 9, 17, and 14, respectively, and the VL region comprises the CDR-L1, the CDR-L2, and the CDR-L3 comprising the sequence set forth in SEQ ID NOs: 19, 22, and 21, respectively;
the VH region comprises the CDR-H1, the CDR-H2, and the CDR-H3 comprising the sequence set forth in SEQ ID NOs: 9, 15, and 18, respectively, and the VL region comprises the CDR-L1, the CDR-L2, and the CDR-L3 comprising the sequence set forth in SEQ ID NOs: 19, 22, and 23, respectively.

39. The binding molecule of any of claims 1-18, wherein:
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 24, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 25, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 26, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 27, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 28, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 42;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 29, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 30, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 31, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 32, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 33, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 34, and the VL region comprises an amino acid sequence that has at least at or about 85%, 86%, 87%, 88%, 89%, 90%, 91%, 92%, 93%, 94%, 95%, 96%, 97%, 98% or 99% sequence identity to ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 35, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 38, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41; or
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 126, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40.

40. The binding molecule of any of claims 1-18, wherein:
the VH region and the VL region comprise the sequences set forth in SEQ ID NO: 24 and 39, respectively;
the VH region and the VL region comprise the sequences set forth in SEQ ID NO: 25 and 40, respectively;
the VH region and the VL region comprise the sequences set forth in SEQ ID NO: 26 and 40, respectively;
the VH region and the VL region comprise the sequences set forth in SEQ ID NO: 27 and 41, respectively;
the VH region and the VL region comprise the sequences set forth in SEQ ID NO: 28 and 42, respectively;
the VH region and the VL region comprise the sequences set forth in SEQ ID NO: 29 and 40, respectively;
the VH region and the VL region comprise the sequences set forth in SEQ ID NO: 30 and 41, respectively;
the VH region and the VL region comprise the sequences set forth in SEQ ID NO: 31 and 43, respectively;
the VH region and the VL region comprise the sequences set forth in SEQ ID NO: 32 and 40, respectively;
the VH region and the VL region comprise the sequences set forth in SEQ ID NO: 33 and 40, respectively;
the VH region and the VL region comprise the sequences set forth in SEQ ID NO: 34 and 40, respectively;
the VH region and the VL region comprise the sequences set forth in SEQ ID NO: 35 and 40, respectively;
the VH region and the VL region comprise the sequences set forth in SEQ ID NO: 36 and 40, respectively;
the VH region and the VL region comprise the sequences set forth in SEQ ID NO: 37 and 40, respectively;
the VH region and the VL region comprise the sequences set forth in SEQ ID NO: 38 and 41, respectively; or
the VH region and the VL region comprise the sequences set forth in SEQ ID NO: 126 and 40, respectively.

41. The binding molecule of any one of claims 1-40, wherein the second binding domain is an scFv comprising:
(a) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 130;
(b) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 131;
(c) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 128; or
(d) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 129.

42. The binding molecule of any one of claims 1-41, wherein the second binding domain is an scFv comprising:
(a) the amino acid sequence of SEQ ID NO: 130;
(b) the amino acid sequence of SEQ ID NO: 131;
(c) the amino acid sequence of SEQ ID NO: 128; or
(d) the amino acid sequence of SEQ ID NO: 129.

43. The binding molecule of claim 1, wherein the CD3 binding domain comprises the VH region that comprises the CDR-H1, a CDR-H2, and a CDR-H3 comprising the sequence set forth in SEQ ID NOs: 9, 15, and 14, respectively, and the VL region comprises a CDR-L1, a CDR-L2, and a CDR-L3 comprising the sequence set forth in SEQ ID NOs: 19, 22, and 21, respectively.

44. The binding molecule of claim 43, wherein the CD3 binding domain comprises the VH region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 28, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 42.

45. The binding molecule of claim 43 or claim 44, wherein the CD3 binding domain comprises the VH region and the VL region that comprises the sequence or are the sequence set forth in SEQ ID NO: 28 and 42, respectively.

46. The binding molecule of claim 43, wherein the CD3 binding domain comprises the VH region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 29, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 40.

47. The binding molecule of claim 43 or claim 46, wherein the CD3 binding domain comprises the VH region and the VL region that comprises the sequence or are the sequence set forth in SEQ ID NO: 29 and 40, respectively.

48. The binding molecule of claim 1, wherein the CD3 binding domain comprises the VH region that comprises the CDR-H1, a CDR-H2, and a CDR-H3 comprising the sequence set forth in SEQ ID NOs: 9, 15, and 16, respectively, and the VL region comprises a CDR-L1, a CDR-L2, and a CDR-L3 comprising the sequence set forth in SEQ ID NOs: 19, 22, and 23, respectively.

49. The binding molecule of claim 48, wherein the CD3 binding domain comprises the VH region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 30, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 41.

50. The binding molecule of claim 48 or claim 49, wherein the CD3 binding domain comprises the VH region and the VL region that comprises the sequence or are the sequence set forth in SEQ ID NO: 30 and 41, respectively.

51. The binding molecule of claim 48, wherein the CD3 binding domain comprises the VH region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 31, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 43.

52. The binding molecule of claim 48 or claim 51, wherein the CD3 binding domain comprises the VH region and the VL region that comprises the sequence or are the sequence set forth in SEQ ID NO: 31 and 43, respectively.

53. The binding molecule of any of claims 1-52, wherein the binding molecule is a full length antibody.

54. The binding molecule of claim 53, wherein the full length antibody comprises a constant region (Fc) from an IgA1, IgA2, IgD, IgE, IgG1, IgG2, IgG3, IgG4, or IgM.

55. The binding molecule of claim 54, wherein the constant region is a human IgG1 heavy chain constant region.

56. The binding molecule of claim 54 or 55, wherein the constant region comprises the amino acid sequence set forth in any of SEQ ID NOs: 44-47.

57. The binding molecule of claim 54 or 55, wherein the constant region comprises the amino acid sequence set forth in any of SEQ ID NO: 48-51.

58. A polynucleotide comprising a nucleic acid encoding the binding molecule of any of claims 1-57.

59. A vector comprising the polynucleotide of claim 58.

60. The vector of claim 59, wherein the vector is a viral vector.

61. The vector of claim 60, wherein the viral vector is a retroviral vector or a lentiviral vector.

62. A cell comprising the polynucleotide of claim 58, or the vector of any of claims 59-61.

63. The cell of claim 62, wherein the cell is a mammalian cell.

64. A method of producing an antibody comprising culturing the cell of claim 62 or 63 under a condition that produces the antibody.

65. The method of claim 64, further comprising recovering the antibody produced by the cell.

66. An antibody or antigen-binding fragment thereof produced by the method of claim 64 or claim 65.

67. A composition comprising the binding molecule of any of claims 1-57.

68. The composition of claim 67, further comprising a pharmaceutically acceptable excipient.

69. A method for treating a subject with a disease or condition, the method comprising administering a therapeutically effective amount of the binding molecule of any of claims 1-57.

70. The method of claim 69, wherein the disease or condition is an infectious disease or disorder, an autoimmune disease, an inflammatory disease, or a tumor or a cancer.

71. The method of claim 69 or 70, wherein the disease or condition is a tumor or a cancer.

72. The kit comprising the binding molecule of any of claims 1-57 and, optionally, instructions for use.
